(19) United States
(12) Patent Application Publication
(10) Pub. No.: US 2022/0062258 A1
(43) Pub. Date: Mar. 3, 2022

(54) Heterocyclic derivatives

(71) Applicant: Merck Patent GmbH, Darmstadt (DE)

(72) Inventors: Timo Heinrich, Freigericht (DE); Sarah Schlesiger, Darmstadt (DE)

(73) Assignee: Merck Patent GmbH, Darmstadt (DE)

(21) Appl. No.: 17/424,240

(22) PCT Filed: Jan. 20, 2020
(86) PCT No.: PCT/EP2020/051210

§ 371 Date: Jul. 20, 2021

(30) Foreign Application Priority Data
Jan. 22, 2019 (EP) 19152928.8

(51) Int. Cl. A61K 31/454; C07D 401/14; C07D 417/14; C07D 471/10; A61K 31/496

(52) U.S. Cl. CPC A61K 31/454; C07D 401/14; A61K 31/496; C07D 471/10; C07D 417/14

(57) ABSTRACT

Compounds of the formula I

Q¹-Q²-Q³ I

corresponding to E3 ligase binding compounds can degrade target proteins, and can be employed, inter alia, for the treatment of diseases such as cancer, multiple sclerosis, cardiovascular diseases, central nervous system injury and different forms of inflammation.

### CROSS-REFERENCE TO RELATED APPLICATIONS

This application is a National Stage entry under § 371 of International Application No. PCT/EP2020/051210, filed on Jan. 20, 2020, and which claims the benefit of European App. No. 19152928.8, filed on Jan. 22, 2019. The content of each of these applications is hereby incorporated by reference in its entirety.

### BACKGROUND OF THE INVENTION

### Field of the Invention

The invention had the object of finding novel compounds having valuable properties, in particular those which can be used for the preparation of medicaments.

The present invention relates to E3 ligase binding compounds which degrade target proteins, preferably MetAP-2.

The compounds of this invention are heterocyclic derivatives and are useful in treating diseases such as of tumours, tumour metastases, proliferative diseases of the mesangial cells, haemangioma, proliferative retinopathy, rheumatoid arthritis, atherosclerotic neovascularisation, psoriasis, ocular neovascularisation, osteoporosis, diabetes and obesity, lymphoid leukaemia, lymphoma, malaria and prostate hypertrophy. The present invention also provides methods for preparing these compounds and pharmaceutical compositions comprising these compounds.

### Description of Related Art

Small molecule degraders are increasingly utilized as tools to examine the functional roles of proteins and emerged as a novel therapeutic modality. Operating at the post-translational level, these molecules provide the potential for differentiated biological responses in comparison to classical inhibitors and expand the repertoire of methods for protein knock down beyond genetic approaches (e.g.: knock-out, siRNA).

Degrader molecules provide an example of a chemical genetic technique capable of more generally targeting the proteome. These chimeric molecules are designed to induce the degradation of their target proteins via the ubiquitin proteasome system (UPS), thereby eliminating pre-existing proteins. The UPS is the major intracellular pathway for protein degradation in which a series of enzymes known as E1s (ubiquitin activating enzymes), E2s (ubiquitin conjugating enzymes) and E3s (ubiquitin ligases) carry out covalent linkage of the 9 kDa, 76 amino acid protein ubiquitin to a target protein. Subsequent enzymatic reactions result in the formation of a polyubiquitin chain, which targets the protein for degradation by the 26S proteasome.

Bifunctional degraders comprise an E3 ligase-binding motif that is linked to a target protein binding moiety. Consequently, these molecules hijack the cell's own degradation machinery by recruiting an E3 ligase in vicinity of the target protein. The spatial proximity enables ubiquitination of the protein and subsequent recognition and depletion by the UPS through the formation of a stable ternary complex.

Specificity for a particular target protein is associated with the E3 ligase (Li W, et al. PLoS One. 2008; 3:e1487) that facilitates the final step of ubiquitin attachment to the target protein. While the first generation degraders were successfully developed using peptides as an E3 ubiquitin ligase-recognizing motif, they were either not cell-permeable or made cell-permeable by adding a cell-permeating motif such as the TAT peptide (Sakamoto K M, et al Proc Natl Acad Sci USA. 2001; 98:8554-8559; Zhang D, et al. Bioorg Med Chem Lett. 2004; 14:645-648; Schneekloth J S Jr. et al. J Am Chem Soc. 2004; 126:3748-3754.). The poor cell permeability of the first generation of bifunctional degraders was significantly improved by the discovery of small molecules that bind to E3 ligases such as the Von Hippel Lindau (VHL) ligand binding to VHL ligase (Buckley et al, J. Am. Chem. Soc., 2012, 134 (10), pp 4465-4468) or thalidomide derivatives binding to the CRBN or Cereblon E3 Liaase (Winter et al. Science 19 Jun. 2015: Vol. 348. Issue 6241. pp. 1376-1381). Having all small molecule degraders in hand enabled scientists to optimize those tool compounds into relevant therapeutic compounds.

So far, androgen receptor (AR) and estrogen receptor (ER) targeting degraders have been developed into clinical candidates, demonstrating the potential applications of these molecules in the treatment of prostate and breast cancers (Rodriguez-Gonzalez A, et al. Oncogene. 2008; 27:7201-7211; Cyrus K, et al. Chembiochem. 2010; 11:1531-1534).

Next to peptidic degraders targeting methionine aminopeptidase-2 (MetAP-2) (Sakamoto K M, et al. Proc Natl Acad Sci USA. 2001; 98:8554-8559; WO2002020740) a range of other bifunctional degraders have been developed targeting proteins ranging from kinases, signaling proteins as well as cytosolic proteins and membrane receptors (examples in Ottis et al ACS Chem. Biol., 2017, 12 (4), pp 892-898).

The synthesis of degrader compounds that mediate the degradation of MetAP2 by recruiting VHL E3 ligase is reported in (WO2002020740). This approach is focused on MetAP-2 ligands that bind the target protein in a covalent fashion and a peptidic sequence targetic VHL. A covalent mode of action drastically impairs the putative catalytic function of degrader molecules.

In this invention, the target protein MetAP-2 is bound by reversible binding ligands described for example in WO2013/149704. The E3 ligase is recognized by known motives as described for example in WO2018/033556 or CN107540608.

Other heterocyclic amide derivatives as inhibitors of the activity of target proteins are disclosed in WO 2018/033556 A1.

Cyclic amides are described as MetAP-2 inhibitors in WO 2012/048775 A1, WO 2013/149704 A1 and WO 2016/020031 A1.

### SUMMARY OF THE INVENTION

It has been found that the compounds according to the invention and salts thereof have very valuable pharmacological properties while being well tolerated.

Surprisingly, we found that compounds of the invention degrade MetAP-2 in a dose dependent manner in disease relevant cell lines.

Compounds of the invention inhibit the MetAP-2 enzyme with nanomolar concentration and inhibit HUVEC cell proliferation with micromolar IC50s as given in the attached table.

This inhibition of MetAP-2 in the enzymatic as well as the cellular assays is comparable to the MetAP-2 inhibition demonstrated with compounds from the Merck applications WO 2012/048775, WO 2013/149704, WO 2016/020031. Accordingly, compounds described in embodiments herein are useful for the treatment of diseases as described in WO 2012/048775, WO 2013/149704, WO 2016/020031.

The present invention specifically relates to compounds of the formula I which degrade the target protein MetAP-2, to compositions which comprise these compounds, and to processes for the use thereof for the treatment of diseases and complaints.

The host or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

The susceptibility of a particular cell to treatment with the compounds according to the invention can be determined by in vitro tests. Typically, a culture of the cell is combined with a compound according to the invention at various concentrations for a period of time which is sufficient to allow active agents such as anti IgM to induce a cellular response such as expression of a surface marker, usually between about one hour and one week. In vitro testing can be carried out using cultivated cells from blood or from a biopsy sample. The amount of surface marker expressed is assessed by flow cytometry using specific antibodies recognising the marker.

The dose varies depending on the specific compound used, the specific disease, the patient status, etc. A therapeutic dose is typically sufficient considerably to reduce the undesired cell population in the target tissue while the viability of the patient is maintained. The treatment is generally continued until a considerable reduction has occurred, for example an at least about 50% reduction in the cell burden, and may be continued until essentially no more undesired cells are detected in the body.

### BRIEF DESCRIPTION OF THE DRAWINGS

FIG. 1 shows how the two compounds “A1” and “A2” degrade MetAP-2 over time.

FIG. 2 shows a quantitative comparison of how the two compounds “A1” and “A2” degrade MetAP-2 over time. It shows a comparison of MetAP2 protein levels in HTC116 cells after compound treatments at different concentrations and time points. As control, DMSO was used and its values set as “1”. MetAp2 values <1 means degradation.

FIG. 3 shows a quantitative comparison of how the two compounds “A1” and “A2” degrade MetAP-2 over time. It shows a comparison of MetAP2 protein levels in HTC116 cells after compound treatments at different concentrations and time points. As control, DMSO was used and its values set as “1”. MetAp2 values <1 means degradation.

### DETAILED DESCRIPTION OF THE INVENTION

The invention relates to compounds of the formula I

Q¹-Q²-Q³ I

in which
Q¹ denotes

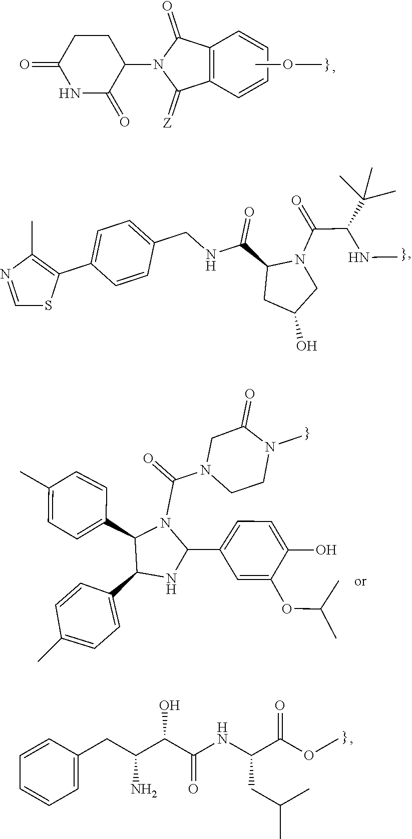

Z denotes O or CH₂,
Q denotes O, NH, NCH₃ or CH₂,
Q² denotes unbranched alkylene having 4-25 C atoms, in which 1-8 nonadjacent CH₂ groups may be replaced by 0, CONH and/or NHCO and in which one CH₂ group may be replaced by

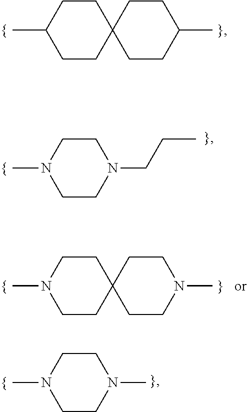

Q³ denotes

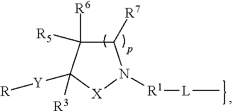

L denotes NR⁴CO, CONR⁴, NH, O, CO, S, SO₂, SO(═NH), NHCONH, SO₂NH or NHSO₂,
R denotes NR²R⁴, Alk, C(═CH₂)[C(R⁴)₂]ₙAr², Het², O[C(R⁴)₂]ₙAr² or OA,
X denotes CO or CH₂,
Y denotes CO or CH₂,
R¹ denotes (CH₂)ₙ, [C(R⁴)₂]ₙAr¹—, (CH₂)ₙHet-, (CH₂)ₙCyc-, [C(R⁴)₂]ₙCONHAr¹—, [C(R⁴)₂]ₙNA-, O[C(R⁴)₂]ₙAr¹— or [C(R⁴)₂]ₙCOO(CH₂)ₙAr¹—,
   wherein substituent L directly is connected to Ar¹, Het or Cyc,
R² denotes H, [C(R⁴)₂]ₙAr², (CH₂)ₙCOHet¹, (CH₂)ₙCOAr², (CH₂)ₘNA₂, (CH₂)ₙCyc or (CH₂)ₙHet¹,
R³ denotes OH or OCOA,
R⁴ denotes H or alkyl having 1, 2, 3 or 4 C-atoms,
R² and R⁴ together also denote alkylene having 2, 3, 4 or 5 C-atoms, where a CH₂ group may also be replaced by N(CH₂)ₘOH or SO₂,
R⁵, R⁶ each, independently of one another H, F or A,
R⁵ and R⁶ together also denote alkylene having 2, 3, 4 or 5 C-atoms, where a CH₂ group may also be replaced by NCOA or O,
R⁷ denotes H, Hal or A,
Ar¹ denotes phenyl which is unsubstituted or mono-, di- tri-, tetra- or pentasubstituted by Hal, OH, OA, CONH₂, CONHA, CONA₂, NHSO₂A, CONHCyc, NHSO₂Cyc, CONHAr², Het¹, COHet¹ and/or NASO₂A,
Ar² denotes phenyl which is unsubstituted or mono-, di-, tri-, tetra- or pentasubstituted by Hal, A, CONH₂, and/or OAr³,
Ar³ denotes phenyl which is unsubstituted or monosubstituted by NH₂,
Het denotes a mono- or bicyclic saturated, unsaturated or aromatic heterocycle having 1 to 4 N, and/or O and/or S atoms which is unsubstituted or mono-, di- or trisubstituted by Hal, A, OA, CN, NH₂, NHA, NA₂, NO₂, CN, COOH, COOA, (CH₂)ₙCONH₂, (CH₂)ₙCONHA, (CH₂)ₙCONA₂, NHCOA, COA, CHO, Het¹, SO₂A, SO₂NH₂, SO₂NHA, SO₂NA₂, CONHNH₂, CONHAr³, ═O and/or Ar³,
Het¹ denotes pyridazinyl, pyrazolyl, pyridyl, piperazinyl, morpholinyl, pyrimidinyl, furyl, thienyl, imidazolyl, pyrrolyl, oxazolyl, oxadiazolyl, isoxazolyl, thiazolyl, triazolyl, tetrazolyl, thiadiazole, piperidin-1-yl, pyrrolidin-1-yl, tetrahydropyranyl, 1,2-oxazinan-2-yl, 1,2,5-oxadiazinan-2-yl, 1,3-oxazinan-3-yl or hexahydropyrimidinyl, each of which is unsubstituted or mono-, di- or trisubstituted by A and/or OA,
Het² denotes isoindolyl,
A denotes unbranched or branched alkyl having 1-10 C atoms, in which 1-7H atoms may be replaced by F, Cl, Br, OH, CHO, COA, COOA, CN, CONA₂, CONHA and/or CONH₂,
   and/or in which one or two non-adjacent CH and/or CH₂ groups may be replaced by C,
   or Cyc,
Alk denotes alkenyl having 2, 3, 4, 5 or 6 C atoms
Cyc denotes cyclic alkyl having 3-7 C atoms which is unsubstituted or mono-, di- or trisubstituted by NHCOA, NHSO₂, OH, OA, A, NH₂, NHA, NA₂, COOA, COOH and/or CONHA,
Hal denotes F, Cl, Br or I,
m denotes 1, 2, 3 or 4,
n denotes 0, 1, 2, 3 or 4,
p denotes 1, 2 or 3,
   and pharmaceutically acceptable salts, tautomers and stereoisomers thereof, including mixtures thereof in all ratios.

The invention also relates to the optically active forms (stereoisomers), the enantiomers, the racemates, the diastereomers and the hydrates and solvates of these compounds.

Moreover, the invention relates to pharmaceutically acceptable derivatives of compounds of formula I.

The term solvates of the compounds is taken to mean adductions of inert solvent molecules onto the compounds which form owing to their mutual attractive force. Solvates are, for example, mono- or dihydrates or alkoxides.

It is understood, that the invention also relates to the solvates of the salts. The term pharmaceutically acceptable derivatives is taken to mean, for example, the salts of the compounds according to the invention and also so-called prodrug compounds.

As used herein and unless otherwise indicated, the term “prodrug” means a derivative of a compound of formula I that can hydrolyze, oxidize, or otherwise react under biological conditions (in vitro or in vivo) to provide an active compound, particularly a compound of formula I. Examples of prodrugs include, but are not limited to, derivatives and metabolites of a compound of formula I that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods.

The expression “effective amount” denotes the amount of a medicament or of a pharmaceutical active ingredient which causes in a tissue, system, animal or human a biological or medical response which is sought or desired, for example, by a researcher or physician.

In addition, the expression “therapeutically effective amount” denotes an amount which, compared with a corresponding subject who has not received this amount, has the following consequence:

improved treatment, healing, prevention or elimination of a disease, syndrome, condition, complaint, disorder or side-effects or also the reduction in the advance of a disease, complaint or disorder.

The expression “therapeutically effective amount” also encompasses the amounts which are effective for increasing normal physiological function.

The invention also relates to the use of mixtures of the compounds of the formula I, for example mixtures of two diastereomers, for example in the ratio 1:1, 1:2, 1:3, 1:4, 1:5, 1:10, 1:100 or 1:1000.

These are particularly preferably mixtures of stereoisomeric compounds. “Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution.

The invention relates to the compounds of the formula I and salts thereof and to a process for the preparation of compounds of the formula I, wherein L denotes CONR⁴, and pharmaceutically acceptable salts, solvates, tautomers and stereoisomers thereof,

characterised in that a compound of formula II

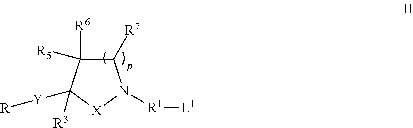

in which X, R, R¹, R³, R⁵, R⁶, R⁷ and p have the meanings indicated in formula I,
and L¹ denotes Cl, Br, I or a free or reactively functionally modified OH group,
is reacted with a compound of the formula III

Q¹-Q²-NH₂ III

in which Q¹ and Q² have the meanings indicated in formula I,
and/or
a base or acid of the formula I is converted into one of its salts.

Above and below, the radicals R¹, R², R³ have the meanings indicated for the formula I, unless explicitely stated otherwise.

A denotes alkyl, this is unbranched (linear) or branched, and has 1, 2, 3, 4, 5, 6, 7 or 8 C atoms. A preferably denotes methyl, ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl. A very particularly preferably denotes alkyl having 2, 3, 4, 5 or 6 C atoms, preferably ethyl, propyl, isopropyl, butyl, isobutyl, sec-butyl, tert-butyl, pentyl, hexyl, trifluoromethyl, pentafluoroethyl or 1,1,1-trifluoroethyl. Moreover, A denotes preferably CH₂OCH₃, CH₂CH₂OH or CH₂CH₂OCH₃. Cyclic alkyl preferably denotes cyclopropyl, cyclobutyl, cyclopentyl, cyclohexyl or cycloheptyl.

R preferably denotes NR²R⁴, furthermore Alk, C(═CH₂)[C(R⁴)₂]ₙAr² or Het².

R particularly preferably denotes NR²R⁴, very particularly preferably NHCH₂Ar².

X preferably denotes CO, furthermore CH₂.

Y preferably denotes CO, furthermore CH₂.

R¹ preferably denotes (CH₂)ₙ, [C(R⁴)₂]ₙAr¹—, (CH₂)ₙHet- or (CH₂)ₙCyc-, furthermore [C(R⁴)₂]ₙCONHAr¹ or [C(R⁴)₂]ₙNA-.

Substituent L is directly connected to Ar¹, Het or Cyc and not to the (CH₂)ₙ or [C(R⁴)₂]n moiety.

R¹ particularly preferably denotes o-, m- or p-phenylen, indole-diyl or benzimidazole-diyl.

R² preferably denotes [C(R⁴)₂]ₙAr², (CH₂)ₙCyc or (CH₂)ₙHet¹.

R³ preferably denotes OH.

R⁴ preferably denotes H, methyl, ethyl or propyl, very particularly preferably H or methyl.

R⁵, R⁶ preferably denote H.

R⁷ preferably denotes H, F or CH₃.

Ar¹ preferably denotes phenyl, o-, m- or p-fluorophenyl, o-, m- or p-bromophenyl, o-, m- or p-chlorophenyl, o-, m- or p-hydroxyphenyl, o-, m- or p-methoxyphenyl, o-, m- or p-aminocarbonylphenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3,4-, 2,3,5-, 2,3,6-, 2,4,6- or 3,4,5-trichlorophenyl, p-iodophenyl, 4-fluoro-3-chlorophenyl, 2-fluoro-4-bromophenyl or 2,5-difluoro-4-bromophenyl.

Ar¹ preferably denotes phenyl.

Ar² preferably denotes phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-trifluoromethylphenyl, o-, m- or p-fluorophenyl, o-, m- or p-bromophenyl, o-, m- or p-chlorophenyl, o-, m- or p-aminocarbonylphenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3,4-, 2,3,5-, 2,3,6-, 2,4,6- or 3,4,5-trichlorophenyl, p-iodophenyl, 4-fluoro-3-chlorophenyl, 2-fluoro-4-bromophenyl, 2,5-difluoro-4-bromophenyl or 2,5-dimethyl-4-chlorophenyl.

Ar² preferably denotes phenyl which is unsubstituted or mono-, di-, tri- or tetra-substituted by Hal.

Ar² furthermore particularly preferably denotes phenyl which is mono- or disubstituted by Hal.

Irrespective of further substitutions, Het preferably denotes 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 2-, 3-, 4-, 5-, 6- or 7-indazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4- or -5-yl or 2,1,3-benzoxadiazol-5-yl.

The heterocyclic radicals may also be partially or fully hydrogenated. Unsubstituted Het can thus also denote, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, further preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl or 2,3-dihydro-2-oxofuranyl.

Het furthermore preferably denotes pyrazinyl, pyrazolyl, benzimidazolyl, pyridyl, indolyl, dihydroindolyl, benzofuranyl, tetrahydropyranyl, dihydroquinolinyl, dihydroisoquinolinyl, tetrahydroquinolinyl, tetrahydroisoquinolinyl, indazolyl, imidazolyl, pyrrolyl, oxazolyl, oxadiazolyl, isoxazolyl, benzothiazolyl, piperidin-1-yl, pyrrolidin-1-yl, 3,4-dihydro-2H-pyrido[3,2-b]-1,4-oxazinyl, 3,4-dihydro-2H-benzo-1,4-oxazinyl, benzofuranyl, azetidinyl, 3-azabicylo[3.2.0]hexyl, pyrrolo[2,3-b]pyridinyl, tetrahydrofuranyl, tetrahydro-1,8-naphthyridinyl, 2,3-dihydrobenzoisothiazolyl, 1,2,3,4-tetrahydrobenzothiazinyl or hexahydrobenzo-1,3-dioxolyl, each of which is unsubstituted or mono-, di- or trisubstituted by Hal, A, OA, CN, NH₂, NHA, NA₂, NO₂, CN, COOH, COOA, (CH₂)ₙCONH₂, (CH₂)ₙCONHA, (CH₂)ₙCONA₂, NHCOA, COA, CHO, Het¹, SO₂A, SO₂NH₂, SO₂NHA, SO₂NA₂, CONHNH₂, CONHAr³, ═O and/or Ar³.

Het furthermore preferably denotes benzimidazolyl or indolyl, each of which is unsubstituted or monosubstituted by Hal.

Het¹ preferably denotes pyridazinyl, pyrazolyl, pyridyl, piperazinyl, morpholinyl, pyrimidinyl, furyl, thienyl, imidazolyl, pyrrolyl, oxazolyl, oxadiazolyl, isoxazolyl, thiazolyl, triazolyl, tetrazolyl, thiadiazole, piperidin-1-yl, pyrrolidin-1-yl, tetrahydropyranyl, 1,2-oxazinan-2-yl, 1,2,5-oxadiazinan-2-yl, 1,3-oxazinan-3-yl or hexahydropyrimidinyl, each of which is unsubstituted or monosubstituted by A and/or OA.

Het¹ furthermore preferably denotes pyridyl, pyrimidinyl, furyl, thienyl, imidazolyl, pyrrolyl, oxazolyl, isoxazolyl, thiazolyl, triazolyl or tetrazolyl.

Het¹ furthermore particularly preferably denotes pyridyl, furyl, thienyl, imidazolyl or pyrrolyl.

Q² preferably denotes CH₂OCH₂CH₂OCH₂CH₂O, (CH₂)₂O(CH₂)₂O(CH₂)₂O(CH₂)₂O(CH₂)₂O(CH₂)₂, OCH₂CH₂CH₂OCH₂CH₂CH₂, (CH₂)₅, (CH₂)₆, (CH₂)₇, (CH₂)₈, (CH₂)₃O(CH₂)₄, (CH₂)₂O(CH₂)₄O, wherein one CH₂ group may be replaced by a group such as

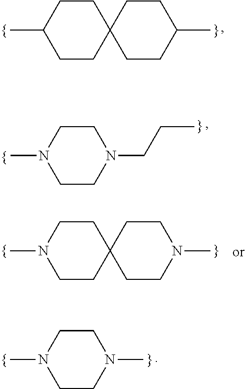

Throughout the invention, all radicals which occur more than once may be identical or different, i.e. are independent of one another.

The compounds of the formula I may have one or more chiral centres and can therefore occur in various stereoisomeric forms. The formula I encompasses all these forms.

Accordingly, the invention relates, in particular, to the compounds of the formula I in which at least one of the said radicals has one of the preferred meanings indicated above. Some preferred groups of compounds may be expressed by the following sub-formulae Ia to Ie, which conform to the formula I and in which the radicals not designated in greater detail have the meaning indicated for the formula I, but in which
in Ia Het denotes pyrazinyl, pyrazolyl, benzimidazolyl, pyridyl, thienyl, furanyl, indolyl, dihydroindolyl, benzofuranyl, tetrahydropyranyl, dihydroquinolinyl, dihydroisoquinolinyl, tetrahydroquinolinyl, tetrahydroisoquinolinyl, indazolyl, imidazolyl, pyrrolyl, oxazolyl, oxadiazolyl, isoxazolyl, benzothiazolyl, piperidin-1-yl, pyrrolidin-1-yl, 3,4-dihydro-2H-pyrido[3,2-b]-1,4-oxazinyl, 3,4-dihydro-2H-benzo-1,4-oxazinyl, benzofuranyl, azetidinyl, 1H-pyrrolo[2,3-b]pyridinyl, 2H-chromenyl, 3-azabicylo[3.2.0]hexyl, pyrrolo[2,3-b]pyridinyl, tetrahydrofuranyl, tetrahydro-1,8-naphthyridinyl 2,3-dihydro-benzoisothiazolyl, 1,2,3,4-tetrahydrobenzothiazinyl or hexahydrobenzo-1.3-dioxolyl, each of which is unsubstituted or mono-, di- or trisubstituted by Hal, A, OA, CN, NH₂, NHA, NA₂, NO₂, CN, COOH, COOA, (CH₂)ₙCONH₂, (CH₂)ₙCONHA, (CH₂)ₙCONA₂, NHCOA, COA, CHO, Het¹, SO₂A, SO₂NH₂, SO₂NHA, SO₂NA₂, CONHNH₂, CONHAr³, ═O and/or Ar³;
in Ib Het denotes benzimidazolyl or indolyl, each of which is unsubstituted or monosubstituted by Hal;
in Ic Q¹ denotes

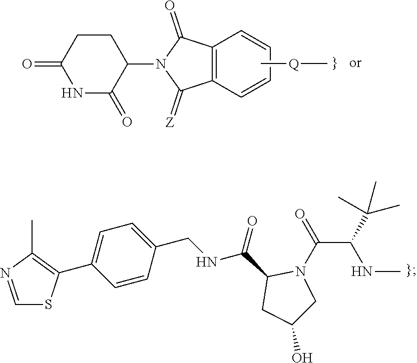

in Id R denotes NR²R⁴;
in Ie R¹ denotes (CH₂)ₙ, [C(R⁴)₂]ₙAr¹— or (CH₂)ₙHet-;
in If Ar² denotes phenyl which is unsubstituted or mono-, di-, tri- or tetra-substituted by Hal;
in Ig R² denotes [C(R⁴)₂]ₙAr², (CH₂)ₙCyc or (CH₂)ₙHet¹;
in Ih A denotes unbranched or branched alkyl having 1-6 C atoms, in which 1-5H atoms may be replaced by F, Cl and/or OH;
in Ii Q¹ denotes

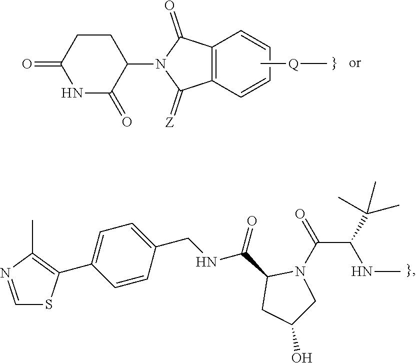

Z denotes O or CH₂,
Q denotes O, NH, NCH₃ or CH₂,
Q² denotes unbranched alkylene having 4-25 C atoms, in which 1-8 non-adjacent CH₂ groups may be replaced by O, CONH and/or NHCO and in which one CH₂ group may be replaced by

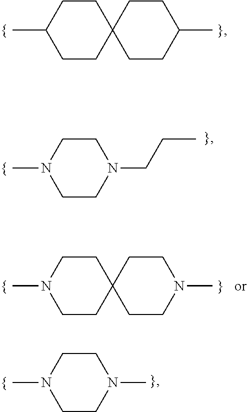

Q³ denotes

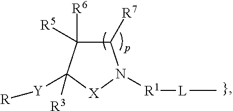

L denotes NR⁴CO, CONR⁴, NH, O, CO, S, SO₂, SO(═NH), NHCONH, SO₂NH or NHSO₂,
R denotes NR²R⁴,
X denotes CO or CH₂,
Y denotes CO or CH₂,
R¹ denotes (CH₂)ₙ, [C(R⁴)₂]ₙAr¹— or (CH₂)ₙHet-,
   wherein substituent L directly is connected to Ar¹, Het or Cyc,
R² denotes [C(R⁴)₂]ₙAr², (CH₂)ₙCyc or (CH₂)ₙHet¹,
R³ denotes OH,
R⁴ denotes H or alkyl having 1, 2, 3 or 4 C-atoms,
R⁵, R⁶ denote H,
R⁷ denotes H, Hal or A,
Ar¹ denotes phenyl,
Ar² denotes phenyl which is unsubstituted or mono-, di-, tri- or tetra-substituted by Hal,
Het denotes pyrazinyl, pyrazolyl, benzimidazolyl, pyridyl, thienyl, furanyl, indolyl, dihydroindolyl, benzofuranyl, tetrahydropyranyl, dihydroquinolinyl, dihydroisoquinolinyl, tetrahydroquinolinyl, tetrahydroisoquinolinyl, indazolyl, imidazolyl, pyrrolyl, oxazolyl, oxadiazolyl, isoxazolyl, benzothiazolyl, piperidin-1-yl, pyrrolidin-1-yl, 3,4-dihydro-2H-pyrido[3,2-b]-1,4-oxazinyl, 3,4-dihydro-2H-benzo-1,4-oxazinyl, benzofuranyl, azetidinyl, 1H-pyrrolo[2,3-b]-pyridinyl, 2H-chromenyl, 3-azabicylo[3.2.0]hexyl, pyrrolo[2,3-b]pyridinyl, tetrahydrofuranyl, tetrahydro-1,8-naphthyridinyl 2,3-dihydro-benzoisothiazolyl, 1,2,3,4-tetrahydrobenzothiazinyl or hexahydrobenzo-1,3-dioxolyl, each of which is unsubstituted or mono-, di- or trisubstituted by Hal, A, OA, CN, NH₂, NHA, NA₂, NO₂, CN, COOH, COOA, (CH₂)ₙCONH₂, (CH₂)ₙCONHA, (CH₂)ₙCONA₂, NHCOA, COA, CHO, Het¹, SO₂A, SO₂NH₂, SO₂NHA, SO₂NA₂, CONHNH₂, CONHAr³, ═O and/or Ar³,
Het¹ denotes pyridyl, furyl, thienyl, imidazolyl or pyrrolyl,
A denotes unbranched or branched alkyl having 1-6 C atoms, in which 1-5H atoms may be replaced by F, Cl and/or OH,
Cyc denotes cyclic alkyl having 3-7 C atoms,
Hal denotes F, Cl, Br or I,
n denotes 0, 1, 2, 3 or 4,
p denotes 1, 2 or 3;
   and pharmaceutically acceptable salts, tautomers and stereoisomers thereof, including mixtures thereof in all ratios.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants known per se which are not mentioned here in greater detail.

Compounds of the formula I can preferably be obtained by reacting compounds of the formula II with a compound of the formula III.

The compounds of the formula II and of the formula III are generally known. If they are novel, however, they can be prepared by methods known per se.

In the compounds of the formula II, L preferably denotes Cl, Br, I or a free or a reactively modified OH group, such as, for example, an activated ester, an imidazolide or alkylsulfonyloxy having 1-6 C atoms (preferably methylsulfonyloxy or trifluoromethylsulfonyloxy) or arylsulfonyloxy having 6-10 C atoms (preferably phenyl- or p-tolylsulfonyloxy).

The reaction preferably succeeds in the presence of a dehydrating agent, such as, for example, a carbodiimide, such as N,N′-dicyclohexylcarbodiimide (“DCCI”), 1,1′-carbonyldiimidazole or N-3-dimethylaminopropyl-N′-ethylcarbodiimide (“DAPECI”), furthermore propanephosphonic anhydride T3P (cf. Angew. Chem. 92, 129 (1980)), diphenylphosphoryl azide or 2-ethoxy-N-ethoxycarbonyl-1,2-dihydroquinoline, optionally in the presence of N-hydroxybenzotriaole;

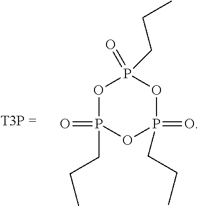 [SMILES: CCCP1(=O)OP(=O)(CCC)OP(=O)(CCC)O1]

Moreover, preferably preferred is HATU [O-(7-Azabenzotriazol-1-yl)-N,N,N′,N′-tetramethyluronium-hexafluorphosphat]

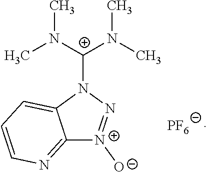 [SMILES: CN(C)[C+](N(C)C)n1n[n+]([O-])c2ncccc21 ; "PF6"]

The reaction is carried out in an inert solvent and is generally carried out in the presence of an acid-binding agent, preferably an organic base, such as DIPEA, 4-methylmorpholine, triethylamine, dimethylaniline, pyridine or quinoline.

The addition of an alkali or alkaline-earth metal hydroxide, carbonate or bicarbonate or another salt of a weak acid of the alkali or alkaline-earth metals, preferably of potassium, sodium, calcium or caesium, may also be favourable.

Depending on the conditions used, the reaction time is between a few minutes and 14 days, the reaction temperature is between about −15° and 150°, normally between 0° and 120°, particularly preferably between 20° and 40° C.

Suitable inert solvents are, for example, hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Particular preference is given to glycol ethers, such as ethylene glycol monomethyl ether, THF, dichloromethane and/or DMF.

Pharmaceutical Salts and Other Forms

The said compounds according to the invention can be used in their final non-salt form. On the other hand, the present invention also encompasses the use of these compounds in the form of their pharmaceutically acceptable salts, which can be derived from various organic and inorganic acids and bases by procedures known in the art. Pharmaceutically acceptable salt forms of the compounds of the formula I are for the most part prepared by conventional methods. If the compound of the formula I contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example potassium ethoxide and sodium propoxide; and various organic bases, such as piperidine, diethanolamine and N-methylglutamine. The aluminium salts of the compounds of the formula I are likewise included. In the case of certain compounds of the formula I, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of the compounds of the formula I include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, formate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, but this does not represent a restriction.

Furthermore, the base salts of the compounds according to the invention include aluminium, ammonium, calcium, copper, iron(III), iron(II), lithium, magnesium, manganese(III), manganese(II), potassium, sodium and zinc salts, but this is not intended to represent a restriction. Of the above-mentioned salts, preference is given to ammonium; the alkali metal salts sodium and potassium, and the alkaline earth metal salts calcium and magnesium. Salts of the compounds of the formula I which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris(hydroxymethyl)methylamine (tromethamine), but this is not intended to represent a restriction.

Compounds of the present invention which contain basic nitrogen-containing groups can be quaternised using agents such as (C₁-C₄)alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, dodecyl, lauryl, myristyl and stearyl chloride, bromide and iodide; and aryl(C₁-C₄)alkyl halides, for example benzyl chloride and phenethyl bromide. Both water- and oil-soluble compounds according to the invention can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.

Particular preference is given to hydrochloride, dihydrochloride, hydrobromide, maleate, mesylate, phosphate, sulfate and succinate.

The acid-addition salts of basic compounds of the formula I are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

As mentioned, the pharmaceutically acceptable base-addition salts of the compounds of the formula I are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.

The base-addition salts of acidic compounds according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

If a compound according to the invention contains more than one group which is capable of forming pharmaceutically acceptable salts of this type, the invention also encompasses multiple salts. Typical multiple salt forms include, for example, bitartrate, diacetate, difumarate, dimeglumine, diphosphate, disodium and trihydrochloride, but this is not intended to represent a restriction.

With regard to that stated above, it can be seen that the expression “pharmaceutically acceptable salt” in the present connection is taken to mean an active ingredient which comprises a compound of the formula I in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

Isotopes

There is furthermore intended that a compound of the formula I includes isotope-labelled forms thereof. An isotope-labelled form of a compound of the formula I is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound of the formula I by well-known methods include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively. A compound of the formula I, a prodrug, thereof or a pharmaceutically acceptable salt of either which contains one or more of the above-mentioned isotopes and/or other iso-topes of other atoms is intended to be part of the present invention. An isotope-labelled compound of the formula I can be used in a number of beneficial ways. For example, an isotope-labelled compound of the formula I into which, for example, a radioisotope, such as ³H or ¹⁴C, has been incorporated is suitable for medicament and/or substrate tissue distribution assays. These radioisotopes, i.e. tritium (³H) and carbon-14 (¹⁴C), are particularly preferred owing to simple preparation and excellent detectability. Incorporation of heavier isotopes, for example deuterium (²H), into a compound of the formula I has therapeutic advantages owing to the higher metabolic stability of this isotope-labelled compound. Higher metabolic stability translates directly into an increased in vivo half-life or lower dosages, which under most circumstances would represent a preferred embodiment of the present invention. An isotope-labelled compound of the formula I can usually be prepared by carrying out the procedures disclosed in the synthesis schemes and the related description, in the example part and in the preparation part in the present text, replacing a non-isotope-labelled reactant by a readily available isotope-labelled reactant.

Deuterium (²H) can also be incorporated into a compound of the formula I for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus cause a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a compound of the formula I that is susceptible to oxidation, the profile of this compound in vivo can be drastically modified and result in improved pharmacokinetic properties.

When discovering and developing therapeutic agents, the person skilled in the art attempts to optimise pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds of the formula I with improved stability through resistance to such oxidative meta-bolism. Significant improvements in the pharmacokinetic profiles of compounds of the formula I are thereby obtained, and can be expressed quantitatively in terms of increases in the in vivo half-life (t½), concen-tra-tion at maximum therapeutic effect (Cₘₐₓ), area under the dose response curve (AUC), and F; and in terms of reduced clearance, dose and materi-als costs.

The following is intended to illustrate the above: a compound of the formula I which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favourable and accurate determination of the extent of the extent to which the improve-ment in resistance to oxidative metabolism has improved. In this way, it is deter-mined that the half-life of the parent compound can be extended by up to 100% as the result of deuterium-hydrogen exchange of this type.

Deuterium-hydrogen exchange in a compound of the formula I can also be used to achieve a favourable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

The invention furthermore relates to medicaments comprising at least one compound of the formula I and/or pharmaceutically acceptable derivatives, solvates and stereoisomers thereof, including mixtures thereof in all ratios, and optionally excipients and/or adjuvants.

Pharmaceutical formulations can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tabletting machine, giving lumps of non-uniform shape, which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a prespecified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds of the formula I and pharmaceutically salts, tautomers and stereoisomers thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds of the formula I and the salts, tautomers and stereoisomers thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxy-ethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis.

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base.

Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose.

Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula I depends on a number of factors, including, for example, the age and weight of the animal, the precise condition that requires treatment, and its severity, the nature of the formulation and the method of administration, and is ultimately determined by the treating doctor or vet. However, an effective amount of a compound according to the invention is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as a single dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of other conditions mentioned above.

A combined treatment of this type can be achieved with the aid of simultaneous, consecutive or separate dispensing of the individual components of the treatment. Combination products of this type employ the compounds according to the invention.

The invention furthermore relates to medicaments comprising at least one compound of the formula I and/or pharmaceutically acceptable salts, tautomers and stereoisomers thereof, including mixtures thereof in all ratios, and at least one further medicament active ingredient.

The invention also relates to a set (kit) consisting of separate packs of
(a) an effective amount of a compound of the formula I and/or pharmaceutically acceptable salts, tautomers and stereoisomers thereof, including mixtures thereof in all ratios, and
(b) an effective amount of a further medicament active ingredient.

The set comprises suitable containers, such as boxes, individual bottles, bags or ampoules. The set may, for example, comprise separate ampoules, each containing an effective amount of a compound of the formula I and/or pharmaceutically acceptable salts, tautomers and stereoisomers thereof, including mixtures thereof in all ratios,

and an effective amount of a further medicament active ingredient in dissolved or lyophilised form.

“Treating” as used herein, means an alleviation, in whole or in part, of symptoms associated with a disorder or disease, or slowing, or halting of further progression or worsening of those symptoms, or prevention or prophylaxis of the disease or disorder in a subject at risk for developing the disease or disorder.

The term “effective amount” in connection with a compound of formula (I) can mean an amount capable of alleviating, in whole or in part, symptoms associated with a disorder or disease, or slowing or halting further progression or worsening of those symptoms, or preventing or providing prophylaxis for the disease or disorder in a subject having or at risk for developing a disease disclosed herein, such as inflammatory conditions, immunological conditions, cancer or metabolic conditions.

Use

The present invention specifically relates to compounds of the formula I and pharmaceutically acceptable salts, tautomers and stereoisomers thereof, including mixtures thereof in all ratios,

for the use for the treatment of diseases in which the degradation and/or modulation of MetAP-2 plays a role.

The present invention specifically relates to compounds of the formula I and pharmaceutically acceptable salts, tautomers and stereoisomers thereof, including mixtures thereof in all ratios, for the use for the degradation and/or modulation of MetAP-2.

The present invention specifically relates to compounds of the formula I and pharmaceutically acceptable salts, tautomers and stereoisomers thereof, including mixtures thereof in all ratios, for use for the treatment and control of diseases.

These diseases include the proliferation of tumour cells, pathological neovascularisation (or angiogenesis), which promotes the growth of solid tumours, neovascularisation in the eye (diabetic retinopathy, age-induced macular degeneration and the like) and inflammation (psoriasis, rheumatoid arthritis and the like), and proliferative diseases of the mesangial cells.

The invention relates to compounds for use of the formula I and pharmaceutically acceptable salts, solvates, tautomers and stereoisomers thereof, including mixtures thereof in all ratios, for the treatment and/or prevention of tumours, tumour metastases, proliferative diseases of the mesangial cells, haemangioma, proliferative retinopathy, rheumatoid arthritis, atherosclerotic neovascularisation, psoriasis, ocular neovascularisation, osteoporosis, diabetes and obesity, lymphoid leukaemia, lymphoma, malaria and prostate hypertrophy.

The invention relates to compounds for use where the tumour disease is selected from the group of the squamous epithelium, of the bladder, of the stomach, of the kidneys, of head and neck, of the oesophagus, of the cervix, of the thyroid, of the intestine, of the liver, of the brain, of the prostate, of the urogenital tract, of the lymphatic system, of the stomach, of the larynx, of the lung, of the skin, monocytic leukaemia, lung adenocarcinoma, small-cell lung carcinoma, pancreatic cancer, glioblastoma, breast carcinoma, acute myeloid leukaemia, chronic myeloid leukaemia, acute lymphatic leukaemia, chronic lymphatic leukaemia, Hodgkin's lymphoma, non-Hodgkin's lymphoma.

The present invention encompasses the use of the compounds of the formula I and/or physiologically acceptable salts and solvates thereof for the preparation of a medicament for the treatment or prevention of tumours, tumour diseases and/or tumour metastases.

The tumour disease is preferably selected from the group tumour of the squamous epithelium, the bladder, the stomach, the kidneys, of head and neck, the oesophagus, the cervix, the thyroid, the intestine, the liver, the brain, the prostate, the urogenital tract, the lymphatic system, the stomach, the larynx, the lung, the skin, monocytic leukaemia, lung adenocarcinoma, small-cell lung carcinoma, pancreatic cancer, glioblastoma, breast carcinoma, acute myeloid leukaemia, chronic myeloid leukaemia, acute lymphatic leukaemia, chronic lymphatic leukaemia, Hodgkin's lymphoma, non-Hodgkin's lymphoma.

Likewise encompassed is the use of the compounds according to formula I according to the invention and/or physiologically acceptable salts and solvates thereof for the preparation of a medicament for the treatment of osteoporosis, diabetes and obesity.

Likewise encompassed is the use of the compounds according to formula I according to the invention and/or physiologically acceptable salts and solvates thereof for the preparation of a medicament for the treatment or prevention of a disease in which angiogenesis is involved.

A disease of this type in which angiogenesis is involved is an eye disease, such as retina vascularisation, diabetic retinopathy, age-induced macular degeneration and the like.

The angiogenic disease is preferably selected from the group diabetic retinopathy, arthritis, cancer, psoriasis, Kaposi's sarcoma, haemangioma, myocardial angiogenesis, atherosclerotic plaque neovascularisation, angiogenic eye diseases, choroidal neovascularisation, retrolental fibroplasia, macular degeneration, corneal transplant rejection, rubeosis iridis, neuroscular glaucoma, Oster Webber syndrome.

The proliferative disease of the mesangial cells is preferably selected from the group glomerulonephritis, diabetic nephropathy, malignant nephrosclerosis, thrombotic microangiopathy syndrome, transplant rejection, glomerulopathy.

The use of compounds of the formula I and/or physiologically acceptable salts and solvates thereof for the preparation of a medicament for the treatment or prevention of inflammatory diseases likewise falls within the scope of the present invention. Examples of such inflammatory diseases include rheumatoid arthritis, psoriasis, contact dermatitis, delayed hyper-sensitivity reaction and the like.

The inflammatory disease is preferably selected from the group inflammatory bowel disease, arthritis, atherosclersosis, asthma, allergies, inflammatory kidney diseases, multiple sclerosis, chronic obstructive pulmonary disease, inflammatory skin diseases, pardontal diseases, psoriasis, T-cell-promoted immune disease.

The inflammatory bowel disease is preferably selected from the group ulcerative colitis, Crohn's disease, non-specific colitis.

The T-cell-promoted immune disease is preferably selected from the group allergic encephalomyelitis, allergic neuritis, transplant rejection, graft-versus-host reaction, myocarditis, thyroiditis, nephritis, systemic lupus erythematosus, insulin-dependent diabetes mellitus.

The arthritis disease is preferably selected from the group rheumatoid arthritis, osteoarthritis, Caplan's syndrome, Felty's syndrome, Sjogren's syndrome, spondylitis ankylosans, Still's disease, chondrocalcinosis, metabolic arthritis, rheumatic fever, Reiter's disease, Wissler's syndrome.

The inflammatory kidney disease is preferably selected from the group glomerulonephritis, glomerular injury, nephrotic syndrome, interstitial nephritis, lupus nephritis, Goodpasture's syndrome, Wegener's granulomatosis, renal vasculitis, IgA nephropathy, idiopatic glomerular disease.

The inflammatory skin disease is preferably selected from the group psoriasis, atopic dermatitis, contact sensitivity, acne.

Likewise encompassed is the use of the compounds of the formula I and/or physiologically acceptable salts and solvates thereof for the preparation of a medicament for the treatment or prevention of a disease or condition in a mammal, in which to this method a therapeutically effective amount of a compound according to the invention is administered to a sick mammal in need of such treatment. The therapeutic amount varies according to the specific disease and can be determined by the person skilled in the art without undue effort.

The present invention also encompasses the use compounds of the formula I and/or physiologically acceptable salts and solvates thereof for the preparation of a medicament for the treatment or prevention of retinal vascularisation.

Likewise encompassed is the use of the compounds of the formula I and/or physiologically acceptable salts thereof for the preparation of a medicament for the treatment and/or combating of a tumour-induced disease in a mammal, in which to this method a therapeutically effective amount of a compound according to the invention is administered to a sick mammal in need of such treatment. The therapeutic amount varies according to the specific disease and can be determined by the person skilled in the art without undue effort.

The disclosed compounds of the formula I can be administered in combination with other known therapeutic agents, including anticancer agents. As used here, the term “anticancer agent” relates to any agent which is administered to a patient with cancer for the purposes of treating the cancer.

The anti-cancer treatment defined above may be applied as a monotherapy or may involve, in addition to the herein disclosed compounds of formula I, conventional surgery or radiotherapy or medicinal therapy. Such medicinal therapy, e.g. a chemotherapy or a targeted therapy, may include one or more, but preferably one, of the following anti-tumor agents:

Alkylating Agents

such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-302⁴, VAL-083⁴; ⁴ no INN.

Platinum Compounds

such as carboplatin, cisplatin, eptaplatin, miriplatine hydrate, oxaliplatin, lobaplatin, nedaplatin, picoplatin, satraplatin;

lobaplatin, nedaplatin, picoplatin, satraplatin;

DNA altering agents

such as amrubicin, bisantrene, decitabine, mitoxantrone, procarbazine, trabectedin, clofarabine;

amsacrine, brostallicin, pixantrone, laromustine^{1,3}; ¹ Prop. INN (Proposed International Nonproprietary Name)³ USAN (United States Adopted Name)

Topoisomerase Inhibitors

such as etoposide, irinotecan, razoxane, sobuzoxane, teniposide, topotecan;

amonafide, belotecan, elliptinium acetate, voreloxin;

Microtubule Modifiers

such as cabazitaxel, docetaxel, eribulin, ixabepilone, paclitaxel, vinblastine, vincristine, vinorelbine, vindesine, vinflunine;

fosbretabulin, tesetaxel;

Antimetabolites

such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur;

doxifluridine, elacytarabine, raltitrexed, sapacitabine, tegafur^{2,3}, trimetrexate; ² Rec. INN (Recommended International Nonproprietary Names)

Anticancer Antibiotics

such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin;

aclarubicin, peplomycin, pirarubicin;

Hormones/Antagonists

such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol;

acolbifene, danazol, deslorelin, epitiostanol, orteronel, enzalutamide^{1,3};

Aromatase Inhibitors

such as aminoglutethimide, anastrozole, exemestane, fadrozole, letrozole, testolactone;

formestane;

Small Molecule Kinase Inhibitors

such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib;

afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib⁴, cabozantinib S-malate^{1,3}, ibrutinib^{1,3}, icotinib⁴, buparlisib², cipatinib⁴, cobimetinib^{1,3}, idelalisib^{1,3}, fedratinib¹, XL-647⁴; Photosensitizers

such as methoxsalen³;

porfimer sodium, talaporfin, temoporfin;

Antibodies

such as avelumab, alemtuzumab, besilesomab, brentuximab vedotin, cetuximab, denosumab, ipilimumab, ofatumumab, panitumumab, rituximab, tositumomab, trastuzumab, bevacizumab, pertuzumab^{2,3};

catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomab¹, tabalumab^{1,3}, EMD-525797⁴, nivolumab^{1,3};

Cytokines

such as aldesleukin, interferon alfa², interferon alfa2a³, interferon alfa2b^{2,3}; celmoleukin, tasonermin, teceleukin, oprelvekin^{1,3}, recombinant interferon beta-1a⁴;

Drug Conjugates

such as denileukin diftitox, ibritumomab tiuxetan, iobenguane 1123, prednimustine, trastuzumab emtansine, estramustine, gemtuzumab, ozogamicin, aflibercept;

cintredekin besudotox, edotreotide, inotuzumab ozogamicin, naptumomab estafenatox, oportuzumab monatox, technetium (99mTc) arcitumomab^{1,3}, vintafolide^{1,3};

Vaccines

such as sipuleucel³; vitespen³, emepepimut-S³, oncoVAX⁴, rindopepimut³, troVax⁴, MGN-1601⁴, MGN-1703⁴;

Miscellaneous

alitretinoin, bexarotene, bortezomib, everolimus, ibandronic acid, imiquimod, lenalidomide, lentinan, metirosine, mifamurtide, pamidronic acid, pegaspargase, pentostatin, sipuleucel³, sizofiran, tamibarotene, temsirolimus, thalidomide, tretinoin, vismodegib, zoledronic acid, vorinostat; celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴, picibanil⁴, reolysin⁴, retaspimycin hydrochloride^{1,3}, trebananib^{2,3}, virulizin⁴, carfilzomib^{1,3}, endostatin⁴, immucothel⁴, belinostat³, MGN-17034;

The following abbreviations refer respectively to the definitions below: aq (aqueous), h (hour), g (gram), L (liter), mg (milligram), MHz (Megahertz), min. (minute), mm (millimeter), mmol (millimole), mM (millimolar), m.p. (melting point), eq (equivalent), mL (milliliter), L (microliter), ACN (acetonitrile), AcOH (acetic acid), CDCl₃ (deuterated chloroform), CD₃OD (deuterated methanol), CH₃CN (acetonitrile), c-hex (cyclohexane), DCC (dicyclohexyl carbodiimide), DCM (dichloromethane), DIC (diisopropyl carbodiimide), DIEA (diisopropylethyl-amine), DMF (dimethylformamide), DMSO (dimethylsulfoxide), DMSO-d₆ (deuterated dimethylsulfoxide), EDC (1-(3-dimethyl-amino-propyl)-3-ethylcarbodiimide), ESI (Electro-spray ionization), EtOAc (ethyl acetate), Et₂O (diethyl ether), EtOH (ethanol), HATU (dimethylamino-([1,2,3]triazolo[4,5-b]pyridin-3-yloxy)-methylene]-dimethylammonium hexafluorophosphate), HPLC (High Performance Liquid Chromatography), i-PrOH (2-propanol), K₂CO₃ (potassium carbonate), LC (Liquid Chromatography), MecOH (methanol), MgSO₄ (magnesium sulfate), MS (mass spectrometry), MTBE (Methyl tert-butyl ether), NaHCO₃ (sodium bicarbonate), NaBH₄ (sodium borohydride), NMM (N-methyl morpholine), NMR (Nuclear Magnetic Resonance), PyBOP (benzotriazole-1-yl-oxy-trispyrrolidino-phosphonium hexafluorophosphate), RT (room temperature), Rt (retention time), SPE (solid phase extraction), TBTU (2-(1-H-benzotriazole-1-yl)-1,1,3,3-tetramethyluromium tetrafluoro borate), TEA (triethylamine), TFA (trifluoroacetic acid), THE (tetrahydrofuran), TLC (Thin Layer Chromatography), UV (Ultraviolet).

Above and below, all temperatures are indicated in ° C. In the following examples, “conventional work-up” means: water is added if necessary, the pH is adjusted, if necessary, to values between 2 and 10, depending on the constitution of the end product, the mixture is extracted with ethyl acetate or dichloromethane, the phases are separated, the organic phase is dried over sodium sulfate and evaporated, and the residue is purified by chromatography on silica gel and/or by crystallisation. Rf values on silica gel; eluent: ethyl acetate/methanol 9:1.

¹H NMR was recorded on Bruker DPX-300, DRX-400, AVII-400 or on a 500 MHz spectrometer, using residual signal of deuterated solvent as internal reference. Chemical shifts (δ) are reported in ppm relative to the residual solvent signal (δ=2.49 ppm for ¹H NMR in DMSO-d₆). ¹H NMR data are reported as follows: chemical shift (multiplicity, coupling constants, and number of hydrogens). Multiplicity is abbreviated as follows: s (singlet), d (doublet), t (triplet), q (quartet), m (multiplet), br (broad).

Biochemical Activity Testing of MetAP-2

MetAP-2 activity was determined by an enzyme-coupled assay using the tripeptide Met-Ala-Ser (MAS) as substrate and recombinant human MetAP-2 (His-Tev-MetAP-2, prepared at Merck). The released methionine is converted by L-amino acid oxidase (AAO) to oxidized Methionine and hydrogen peroxide is released. In a second step horse radish peroxidase catalyzes the oxidation of the leuko dye dianisidine to oxidized dianisidine using hydrogen peroxide as co-substrate. The produced dianisidine ox was detected photometrically as increase in absorbance at 450 nm. Met-AP2 activity was determined in a kinetic measurement mode. The release of one molecule methionine corresponds to the production of one molecule dianisidine ox. The MetAP2 enzymatic activity is directly corresponding to the increase in absorbance per time.

In detail, the assay was performed in 384 well microtiter plate (Greiner 78110 MTP, transparent) in a total reaction volume of 50 μl at 22° C. 0.35 μg of N-terminal His tag human rec MetAP2 (prepared in house, AA 2-478, final concentration (fc) 123 nM), 1 unit horse radish Peroxidase (Roche, Mannheim), 0.02 unit L-amino acid oxidase (Merck, Darmstadt), 0.6 mM dianisidine (Merck, Darmstadt, dissolved in 50 mM HCl, 10% DMSO) were incubated in the absence or presence of the test compound (10 dilution concentrations) in 100 mM Hepes, 50 mM NaCl, 50 μM MnCl₂ at pH 7.0 for 15 min at 22° C. The reaction was started by the addition of 500 μM (fc) MAS peptide (Merck, Darmstadt). After mixing the first absorbance measurement was performed on an Envision multimode reader (Perkin-Elmer, Waltham) at wavelength of 450 nm. The reaction was incubated at 22° C. for additional 45 min and the second absorbance measurement was performed. The increase of absorbance per time was determined. The control value used was the inhibitor-free reaction with 0.5% DMSO (fc). As pharmacological inhibitor control Fumagillin (Merck, Darmstadt) in a final concentration of 5 μM was used. The inhibitory values (IC₅₀) were determined using the program ASSAY ANALYZER® from GeneData (Basel, Switzerland).

HUVEC Proliferation Assay

Proliferation of HUVEC primary endothelial cells was used as cell-based mechanistical assay.²⁵ Applying the CyQUANT@ Direct Cell Proliferation Assay (Invitrogen C35011) which is based on a cell-permeant fluorescent DNA-binding dye, DNA content is used as a direct measure for cell number. Pooled HUVEC cells (Promocell C-12203) are cultivated in the medium supplied by Promocell (Cat No C-22020) for maximum 4 passages. For the assay 500 cells/well are seeded into black 384-well culture plates with clear bottom in 70 μl culture medium and incubated for 6 hours at 37° C., 5% CO₂. 10 μl prediluted test compounds are added and cells incubated for 3 days at 37° C., 5% CO₂ before measurement of DNA content. CyQUANT detection reagent is prepared according to the manufacturers protocol, 20 μl/well added and incubated at 37° C., 5% CO2 for at least 1 h before measurement of fluorescence at Envision multimode reader (Perkin-Elmer, Waltham) with excitation 480 nm and emission 535 nm (bottom read mode). The assay is performed as dose response with 10 compound dilutions. Inhibitory values (IC₅₀) were determined using the program ASSAY ANALYZER® from GeneData (Basel, Switzerland).

In addition, the degradation of MetAP-2 in HCT116 cells could be demonstrated. See scans below. This degradation suggests a prolonged/sustained effect as the target protein MetAP-2 is not only inhibited but even degraded/removed from the cell by the compounds and accordingly, the treatment of the indications should be even more efficacious. The initial MetAP-2 degradation results were obtained after 24 h incubation of the compounds in three different concentrations (0.1; 1; 10 μM), cell lysis, SDS-PAGE separation, blotting and antibody aided detection.

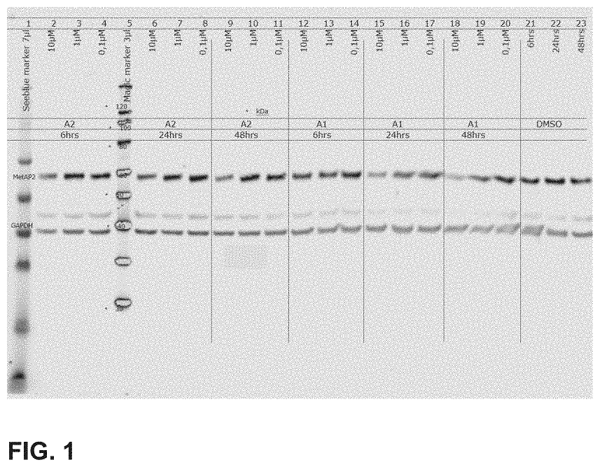

FIG. 1 shows how the two compounds “A1” and “A2” degrade MetAP-2 over the time.

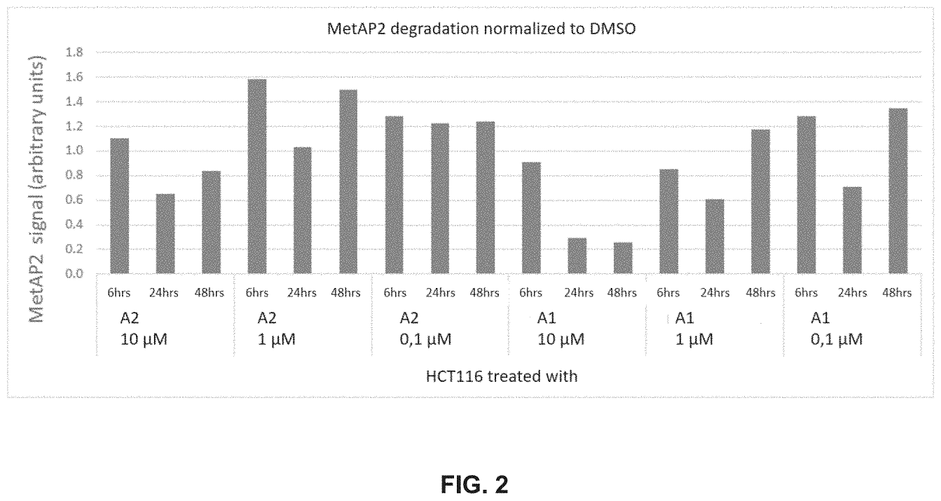

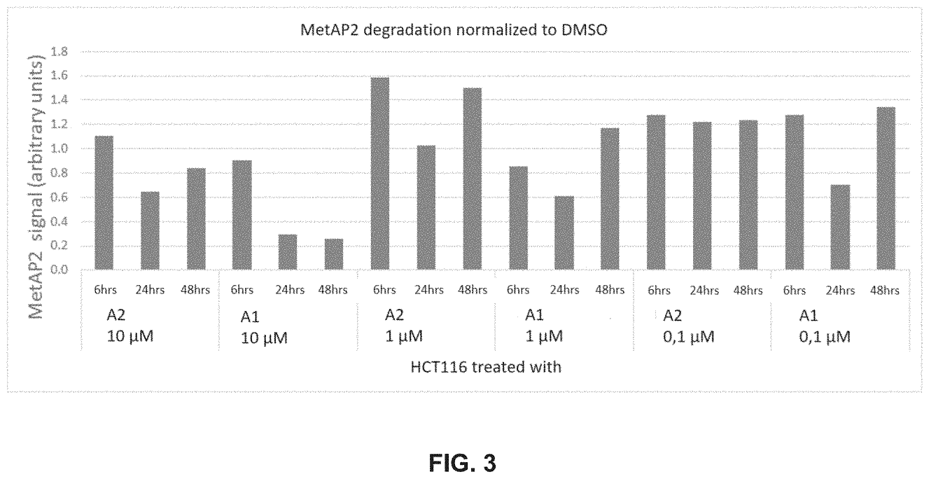

The following tables and FIGS. 2-3 contain a quantification of these results.

In summary:

“A2” showed activity at 10 μM (40% reduction of MetAP-2 protein after 24 h and 20% reduction after 48 h)

“A1” is more active: At 10 μM: 10% reduction after 6 h, 70% reduction after 24 h and again 70% after 48 h. At 1 μM: 15% reduction after 6 h, 40% reduction after 24 h. After 48 h, any reduction could be detected. At 0.1 μM: 30% reduction after 24 h.

Comparison of MetAP2 protein levels in HTC116 cells after compound treatments at different concentrations and time points. As control, DMSO was used and its values set as “1”. MetAp2 values <1 means degradation.

| | | | Signal MetAp2/ |
|---|---|---|---|
| | | | GAPDH normalized |
| | | | to untreated |
| | Sample | | (DMSO) |
| 10 μM | A2 | 6 hrs | 1.1 |
| | | 24 hrs | 0.6 |
| | | 48 hrs | 0.8 |
| 1 μM | A2 | 6 hrs | 1.6 |
| | | 24 hrs | 1.0 |
| | | 48 hrs | 1.5 |
| 0.1 μM | A21 | 6 hrs | 1.3 |
| | | 24 hrs | 1.2 |
| | | 48 hrs | 1.2 |
| 10 μM | A1 | 6 hrs | 0.9 |
| | | 24 hrs | 0.3 |
| | | 48 hrs | 0.3 |
| 1 μM | A1 | 6 hrs | 0.9 |
| | | 24 hrs | 0.6 |
| | | 48 hrs | 1.2 |
| 0.1 μM | A1 | 6 hrs | 1.3 |
| | | 24 hrs | 0.7 |
| | | 48 hrs | 1.3 |
| 10 μM | A2 | 6 hrs | 1.1 |
| | | 24 hrs | 0.6 |
| | | 48 hrs | 0.8 |
| 10 μM | A1 | 6 hrs | 0.9 |
| | | 24 hrs | 0.3 |
| | | 48 hrs | 0.3 |
| 1 μM | A2 | 6 hrs | 1.6 |
| | | 24 hrs | 1.0 |
| | | 48 hrs | 1.5 |
| 1 μM | A1 | 6 hrs | 0.9 |
| | | 24 hrs | 0.6 |
| | | 48 hrs | 1.2 |
| 0.1 μM | A2 | 6 hrs | 1.3 |
| | | 24 hrs | 1.2 |
| | | 48 hrs | 1.2 |
| 0.1 μM | A1 | 6 hrs | 1.3 |
| | | 24 hrs | 0.7 |
| | | 48 hrs | 1.3 |

The preferred examples shown below degrade MetAP-2 in HCT116 cells in a dose and time dependent manner.

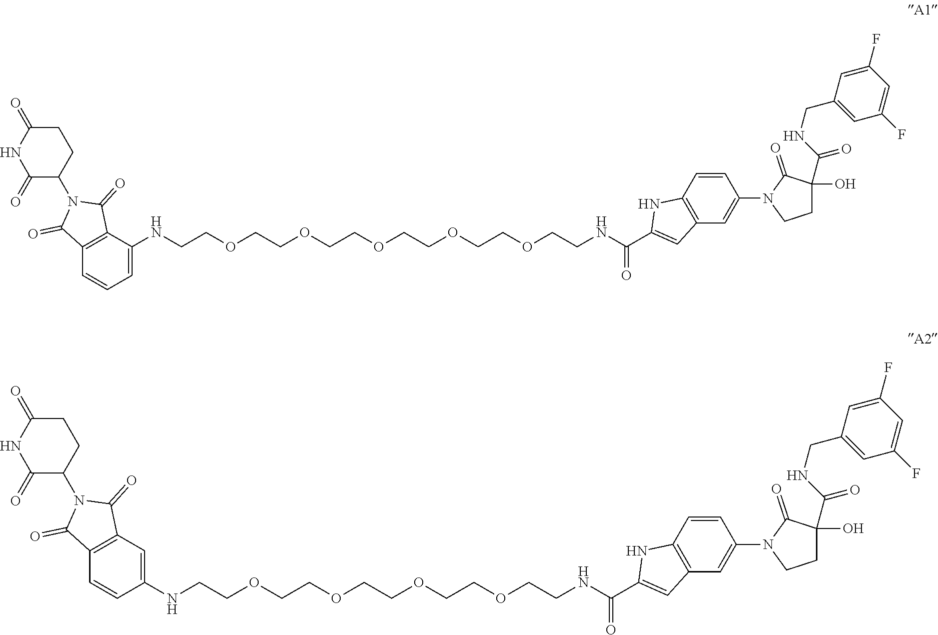 [SMILES: O=C1CCC(N2C(=O)c3cccc(NCCOCCOCCOCCOCCOCCNC(=O)c4cc5cc(N6CCC(O)(C(=O)NCc7cc(F)cc(F)c7)C6=O)ccc5[nH]4)c3C2=O)C(=O)N1 ; O=C1CCC(N2C(=O)c3ccc(NCCOCCOCCOCCOCCNC(=O)c4cc5cc(N6CCC(O)(C(=O)NCc7cc(F)cc(F)c7)C6=O)ccc5[nH]4)cc3C2=O)C(=O)N1]

1. Exemplary Synthesis of “A1”:

### 5-[3-[(3,5-difluorophenyl)methylcarbamoyl]-3-hydroxy-2-oxo-pyrrolidin-1-yl]-N-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethyl]-1H-indole-2-carboxamide

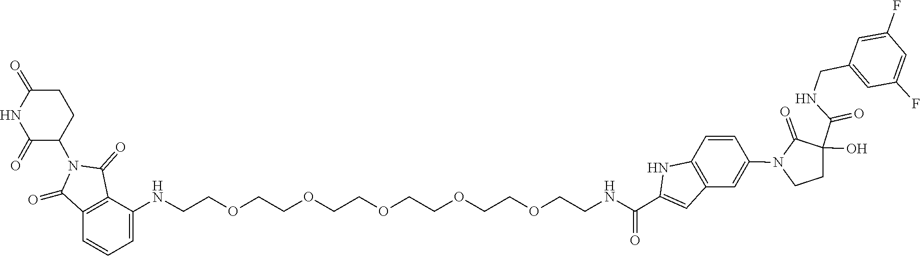 [SMILES: O=C1CCC(N2C(=O)c3cccc(NCCOCCOCCOCCOCCOCCNC(=O)c4cc5cc(N6CCC(O)(C(=O)NCc7cc(F)cc(F)c7)C6=O)ccc5[nH]4)c3C2=O)C(=O)N1]

The building blocks were prepared as known from the literature and linked in an amide forming reaction as shown below:

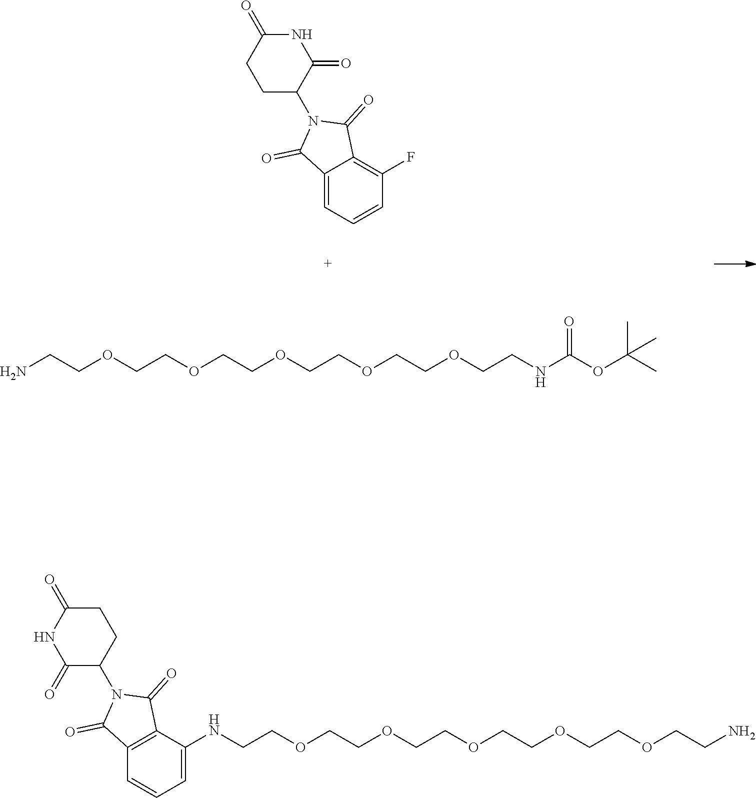 [SMILES: O=C1CCC(N2C(=O)c3cccc(F)c3C2=O)C(=O)N1 ; CC(C)(C)OC(=O)NCCOCCOCCOCCOCCOCCN ; NCCOCCOCCOCCOCCOCCNc1cccc2c1C(=O)N(C1CCC(=O)NC1=O)C2=O]

To a stirred solution of commercially available 2-(2,6-dioxopiperidin-3-yl)-4-fluoro-2,3-dihydro-1H-isoindole-1,3-dione (200 mg; 0.72 mmol) in N,N-Dimethylformamide (1.5 ml; 18 mmol) was added commercially available tertbutyl N-(17-amino-3,6,9,12,15-pentaoxaheptadecan-1-yl)carbamate (275 mg; 0.72 mmol) and Sodium carbonate anhydrous (153 mg; 1.45 mmol). The reaction mixture was heated to 90° C. for 14 h. After cooling to room temperature the reaction mixture was evaporated, dissolved in DMSO and purified by reverse phase chromatography. Fractions containing the product were evaporated. The compound tert-butyl N-(17-{[2-(2,6-dioxopiperidin-3-yl)-1,3-dioxo-2,3-dihydro-1H-isoindol-4-yl]amino}-3,6,9,12,15-pentaoxaheptadecan-1-yl)carbamate was dissolved in dichlormethane (4 ml) and treated with trifluoroacetic acid (1 ml) for 3 h. The reaction was evaporated to dryness, coevaporated twice with toluene and used directly in the next reaction without further purification.

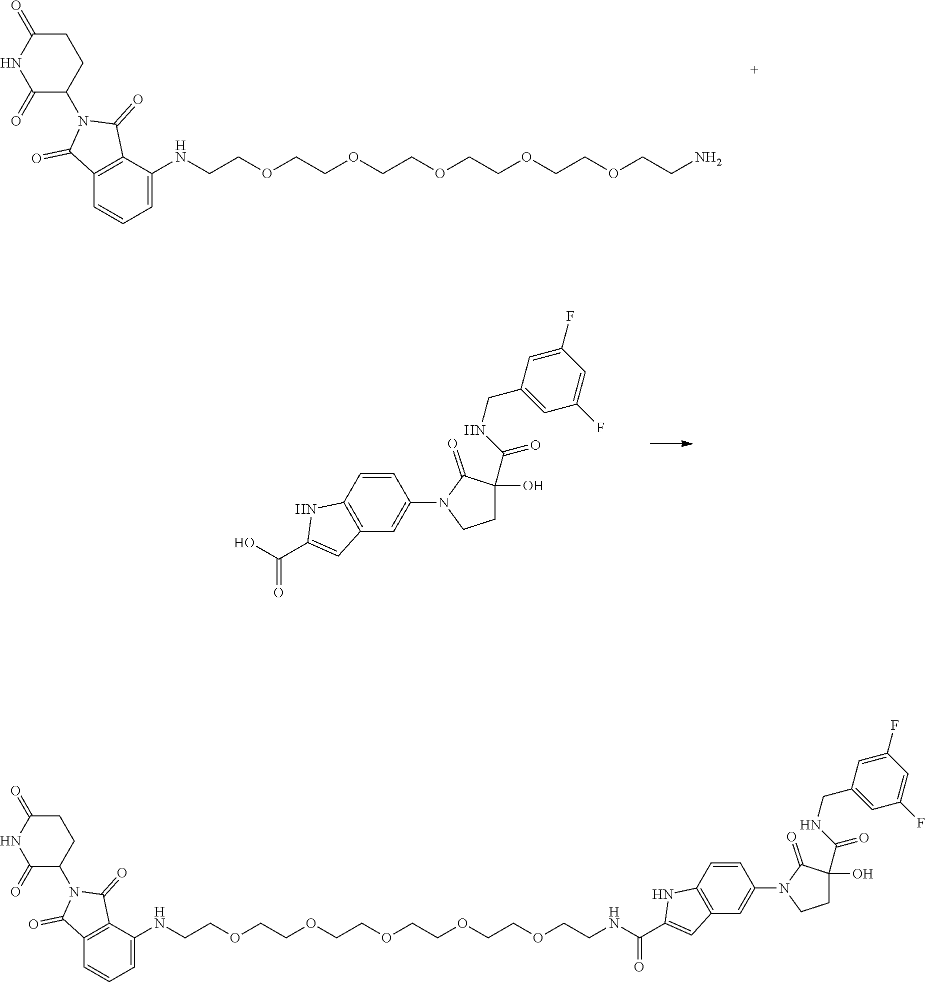 [SMILES: NCCOCCOCCOCCOCCOCCNc1cccc2c1C(=O)N(C1CCC(=O)NC1=O)C2=O ; O=C(O)c1cc2cc(N3CCC(O)(C(=O)NCc4cc(F)cc(F)c4)C3=O)ccc2[nH]1 ; O=C1CCC(N2C(=O)c3cccc(NCCOCCOCCOCCOCCOCCNC(=O)c4cc5cc(N6CCC(O)(C(=O)NCc7cc(F)cc(F)c7)C6=O)ccc5[nH]4)c3C2=O)C(=O)N1]

Procedure:

5-(3-{[(3,5-difluorophenyl)methyl]carbamoyl}-3-hydroxy-2-oxopyrrolidin-1-yl)-1H-indole-2-carboxylic acid (30 mg; 0.07 mmol), 5-(15-amino-4,7,10,13-tetraoxa-1-azapentadecan-1-yl)-2-(2,6-dioxopiperidin-3-yl)-2,3-dihydro-1H-isoindole-1,3-dione (40.5 mg; 0.07 mmol), HATU (31.9 mg; 0.08 mmol) and 4-methylmorpholine (31 μL; 0.28 mmol) were dissolved in N,N-dimethylformamide (5.4 mL) and stirred for 14 h at room temperature. After completion of the reaction indicated by LC-MS, the mixture was evaporated to dryness, re-dissolved in DMSO and purified by preparative HPLC. All fractions were checked by UPLC. yielding a pure product fraction 25.1 mg and an impure fraction. The impure fraction was dissolved in DCM/MeOH, absorbed on Isolute® and purified by column chromatography using DCM to DCM/20% MeOH as eluents. Pure fractions were combined.

The product was lyophilized from water/MeCN.

Analytical Data:

Yield: 42.2 mg (66%)

appearance: white solid

LC-MS: RT: 1.46 min; Area: 100%, m/z 452.8=[M+2H]²⁺; m/z 904.8=[M+H]⁺

NMR:

¹H NMR (700 MHz, DMSO-d₆) δ 11.62 (d, J=2.3 Hz, 1H), 11.05 (s, 1H), 8.69 (t, J=6.4 Hz, 1H), 8.55 (t, J=5.7 Hz, 1H), 7.78 (d, J=2.0 Hz, 1H), 7.61-7.51 (m, 2H), 7.43 (d, J=8.8 Hz, 1H), 7.17-7.10 (m, 2H), 7.06 (tt, J=9.4, 2.4 Hz, 1H), 7.00 (qd, J=6.4, 3.1 Hz, 4H), 6.88 (dd, J=8.5, 2.2 Hz, 1H), 6.70 (s, 1H), 5.03 (dd, J=12.8, 5.5 Hz, 1H), 4.41 (dd, J=15.8, 6.8 Hz, 1H), 4.27 (dd, J=15.8, 6.0 Hz, 1H), 3.89 (ddt, J=9.2, 5.8, 3.4 Hz, 2H), 3.59-3.45 (m, 18H), 3.44 (q, J=5.9 Hz, 2H), 3.36-3.33 (m, 2H), 2.87 (m, 1H), 2.65-2.51 (m, 4H), 2.17-2.10 (m, 1H), 1.99 (dtd, J=13.1, 5.4, 2.4 Hz, 1H).

The following compounds are prepared analogously:

### 5-[3-[(3,5-difluorophenyl)methylcarbamoyl]-3-hydroxy-2-oxo-pyrrolidin-1-yl]-N-[2-[2-[2-[2-[2-[[2-(2,6-dioxo-3-piperidyl)-1,3-dioxo-isoindolin-5-yl]amino]-ethoxy]ethoxy]ethoxy]ethoxy]ethyl]-1H-indole-2-carboxamide (“A2”)

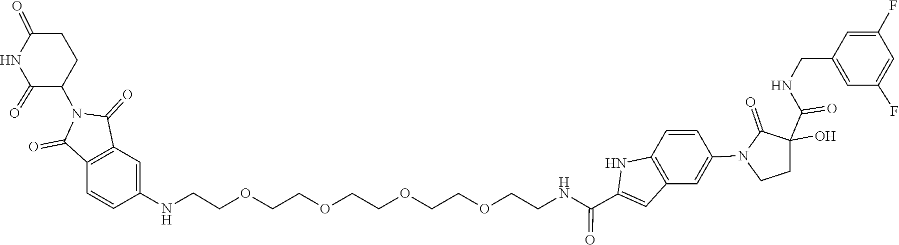 [SMILES: O=C1CCC(N2C(=O)c3ccc(NCCOCCOCCOCCOCCNC(=O)c4cc5cc(N6CCC(O)(C(=O)NCc7cc(F)cc(F)c7)C6=O)ccc5[nH]4)cc3C2=O)C(=O)N1]

LC-MS: RT: 1.46 min, Area: 100%, m/z 452.8=[M+2H]²⁺, m/z 904.8=[M+H]⁺.

¹H NMR (700 MHz, DMSO-d₆) δ 11.62 (d, J=2.3 Hz, 1H), 11.05 (s, 1H), 8.69 (t, J=6.4 Hz, 1H), 8.55 (t, J=5.7 Hz, 1H), 7.78 (d, J=2.0 Hz, 1H), 7.61-7.51 (m, 2H), 7.43 (d, J=8.8 Hz, 1H), 7.16-7.11 (m, 2H), 7.06 (tt, J=9.4, 2.4 Hz, 1H), 7.00 (qd, J=6.4, 3.1 Hz, 3H), 6.88 (dd, J=8.5, 2.2 Hz, 1H), 6.70 (s, 1H), 5.03 (dd, J=12.8, 5.5 Hz, 1H), 4.41 (dd, J=15.8, 6.8 Hz, 1H), 4.27 (dd, J=15.8, 6.0 Hz, 1H), 3.89 (ddt, J=9.2, 5.8, 3.4 Hz, 2H), 3.59-3.46 (m, 14H), 3.44 (q, J=5.9 Hz, 2H), 3.36-3.32 (m, 3H), 2.87 (m, 1H), 2.65-2.52 (m, 4H), 2.20-2.07 (m, 1H), 1.99 (dtd, J=13.1, 5.4, 2.4 Hz, 1H).

### (3RS)—N-[(3,5-difluorophenyl)methyl]-3-hydroxy-1-{2-[(5-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}pentyl)carbamoyl]-1H-indol-5-yl}-2-oxopyrrolidine-3-carboxamide (“A3”)

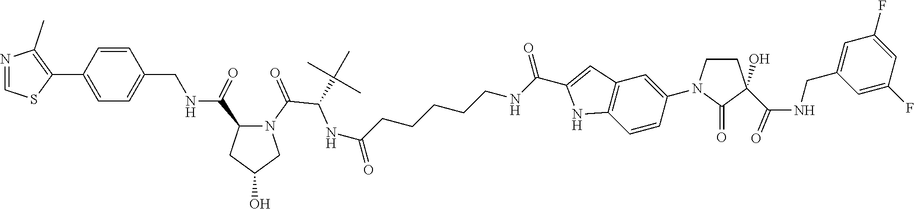 [SMILES: Cc1ncsc1-c1ccc(CNC(=O)[C@@H]2C[C@@H](O)CN2C(=O)[C@@H](NC(=O)CCCCCNC(=O)c2cc3cc(N4CC[C@@](O)(C(=O)NCc5cc(F)cc(F)c5)C4=O)ccc3[nH]2)C(C)(C)C)cc1]

LC-MS: RT: 1.51 min, Area: 96%, m/z 638.3=fragment.

¹H NMR (700 MHz, DMSO-d₆) δ 11.56 (d, J=2.1 Hz, 1H), 8.98 (s, 1H), 8.66 (t, J=6.4 Hz, 1H), 8.52 (t, J=6.0 Hz, 1H), 8.43 (t, J=5.8 Hz, 1H), 7.81 (d, J=9.4 Hz, 1H), 7.77 (d, J=2.0 Hz, 1H), 7.52 (dd, J=9.0, 2.1 Hz, 1H), 7.44-7.40 (m, 3H), 7.38 (d, J=8.2 Hz, 2H), 7.10 (d, J=2.0 Hz, 1H), 7.05 (tt, J=9.3, 2.6 Hz, 1H), 7.00 (h, J=4.7 Hz, 2H), 4.54 (d, J=9.4 Hz, 1H), 4.42 (dt, J=13.0, 7.0 Hz, 3H), 4.35 (dq, J=6.5, 3.5, 3.0 Hz, 1H), 4.27 (dd, J=15.8, 6.1 Hz, 1H), 4.22 (dd, J=15.9, 5.6 Hz, 1H), 3.91-3.87 (m, 2H), 3.69-3.62 (m, 2H), 3.26 (q, J=7.1 Hz, 2H), 2.62 (ddd, J=11.8, 6.6, 4.8 Hz, 1H), 2.27 (dt, J=14.6, 7.6 Hz, 1H), 2.14 (ddd, J=14.7, 9.9, 5.9 Hz, 2H), 2.03 (ddd, J=11.2, 7.9, 2.6 Hz, 1H), 1.90 (ddd, J=12.8, 8.5, 4.7 Hz, 1H), 1.59-1.47 (m, 4H), 1.35-1.26 (m, 2H), 0.93 (s, 9H).

### 5-[3-[(3,5-difluorophenyl)methylcarbamoyl]-3-hydroxy-2-oxo-pyrrolidin-1-yl]-N-[5-[[2-(2,6-dioxo-3-piperidyl)-1,3-dioxo-isoindolin-5-yl]amino]pentyl]-1H-indole-2-carboxamide (“A4”)

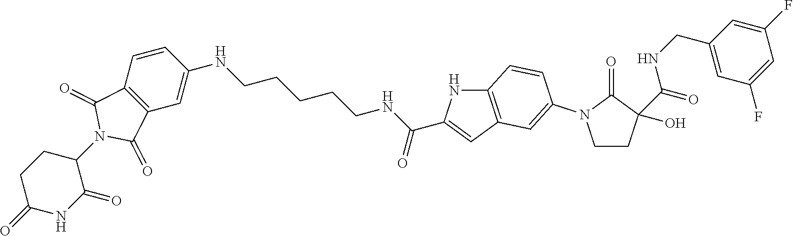 [SMILES: O=C1CCC(N2C(=O)c3ccc(NCCCCCNC(=O)c4cc5cc(N6CCC(O)(C(=O)NCc7cc(F)cc(F)c7)C6=O)ccc5[nH]4)cc3C2=O)C(=O)N1]

LC-MS: RT: 1.53 min, Area: 97%, m/z 770.4.

¹H NMR (500 MHz, DMSO-d₆) δ 11.56 (d, J=2.2 Hz, 1H), 11.02 (s, 1H), 8.66 (t, J=6.4 Hz, 1H), 8.46 (t, J=5.8 Hz, 1H), 7.77 (d, J=2.0 Hz, 1H), 7.55 (d, J=8.4 Hz, 1H), 7.52 (dd, J=8.9, 2.1 Hz, 1H), 7.43 (d, J=8.9 Hz, 1H), 7.12-7.02 (m, 2H), 6.95 (d, J=2.1 Hz, 1H), 6.85 (dd, J=8.4, 2.1 Hz, 1H), 5.02 (dd, J=12.7, 5.4 Hz, 1H), 4.42 (dd, J=15.8, 6.8 Hz, 1H), 4.27 (dd, J=15.8, 6.0 Hz, 1H), 3.93-3.86 (m, 2H), 3.31 (q, J=6.7 Hz, 2H), 3.17 (d, J=7.2 Hz, 2H), 2.87 (ddd, J=16.6, 13.7, 5.3 Hz, 1H), 2.66-2.57 (m, 2H), 2.54 (s, 4H), 2.14 (dt, J=12.8, 7.5 Hz, 1H), 1.99 (ddd, J=12.6, 5.6, 3.2 Hz, 1H), 1.62 (dp, J=15.0, 7.3 Hz, 4H), 1.45 (dt, J=13.8, 7.1 Hz, 2H).

### 5-[3-[(3,5-difluorophenyl)methylcarbamoyl]-3-hydroxy-2-oxo-pyrrolidin-1-yl]-N-[2-[2-[2-[[2-(2,6-dioxo-3-piperidyl)-1,3-dioxo-isoindolin-4-yl]amino]ethoxy]-ethoxy]ethyl]-1-H-indole-2-carboxamide (“A5”)

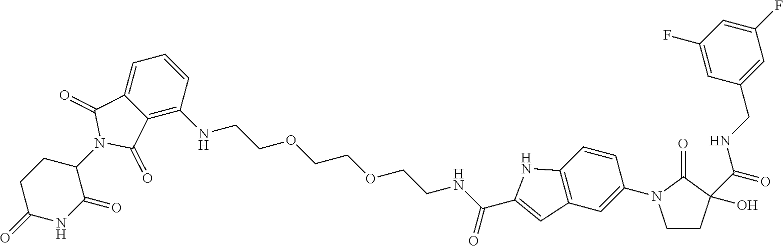 [SMILES: O=C1CCC(N2C(=O)c3cccc(NCCOCCOCCNC(=O)c4cc5cc(N6CCC(O)(C(=O)NCc7cc(F)cc(F)c7)C6=O)ccc5[nH]4)c3C2=O)C(=O)N1]

LC-MS: RT: 1.49 min, Area: 98%, m/z 816.4.

¹H NMR (500 MHz, DMSO-d₆) δ 11.58 (d, J=2.2 Hz, 1H), 11.06 (s, 1H), 8.66 (t, J=6.4 Hz, 1H), 8.50 (t, J=5.7 Hz, 1H), 7.76 (d, J=2.0 Hz, 1H), 7.57-7.51 (m, 2H), 7.42 (d, J=8.9 Hz, 1H), 7.11 (d, J=2.2 Hz, 1H), 7.10-7.04 (m, 2H), 7.01 (qd, J=6.9, 3.6 Hz, 4H), 6.67 (s, 1H), 6.58 (t, J=5.8 Hz, 1H), 5.04 (dd, J=12.8, 5.4 Hz, 1H), 4.42 (dd, J=15.8, 6.8 Hz, 1H), 4.27 (dd, J=15.8, 6.0 Hz, 1H), 3.89 (dd, J=8.3, 5.5 Hz, 2H), 3.67-3.49 (m, 8H), 3.43 (dq, J=11.1, 5.6 Hz, 4H), 2.87 (ddd, J=16.8, 13.7, 6.1 Hz, 1H), 2.66-2.53 (m, 4H), 2.14 (dt, J=12.9, 7.5 Hz, 1H), 2.08-1.94 (m, 1H), 1.24 (d, J=3.4 Hz, 1H).

### 5-[3-[(3,5-difluorophenyl)methylcarbamoyl]-3-hydroxy-2-oxo-pyrrolidin-1-yl]-N-[2-[2-[2-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-2-oxoethoxy]ethoxy]ethyl]-1H-indole-2-carboxamide (“A6”)

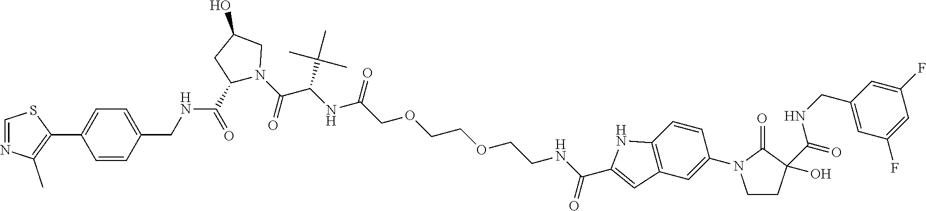 [SMILES: Cc1ncsc1-c1ccc(CNC(=O)[C@@H]2C[C@@H](O)CN2C(=O)[C@@H](NC(=O)COCCOCCNC(=O)c2cc3cc(N4CCC(O)(C(=O)NCc5cc(F)cc(F)c5)C4=O)ccc3[nH]2)C(C)(C)C)cc1]

LC-MS: RT: 1.48 min, Area: 91%, m/z 670.3=fragment.

¹H NMR (500 MHz, DMSO-d₆) δ 11.58 (d, J=2.2 Hz, 1H), 8.95 (s, 1H), 8.66 (t, J=6.5 Hz, 1H), 8.53 (dt, J=9.8, 5.9 Hz, 2H), 7.77 (t, J=1.4 Hz, 1H), 7.54 (dt, J=8.9, 1.8 Hz, 1H), 7.43 (dd, J=9.2, 5.3 Hz, 2H), 7.38 (s, 4H), 7.12 (d, J=2.5 Hz, 1H), 7.09-6.97 (m, 3H), 4.57 (d, J=9.6 Hz, 1H), 4.50-4.32 (m, 6H), 4.26 (dt, J=15.7, 6.4 Hz, 2H), 3.98 (d, J=1.3 Hz, 2H), 3.92-3.85 (m, 2H), 3.71-3.54 (m, 8H), 3.47 (q, J=5.6 Hz, 2H), 2.66-2.57 (m, 1H), 2.43 (s, 3H), 2.18-2.10 (m, 1H), 2.10-2.03 (m, 1H), 1.91 (ddd, J=13.0, 8.7, 4.6 Hz, 1H), 0.94 (s, 9H).

### 5-[3-[(3,5-difluorophenyl)methylcarbamoyl]-3-hydroxy-2-oxo-pyrrolidin-1-yl]-N-[2-[2-[2-[2-[2-[[(1S)-1-[(2S,4R)-4-hydroxy-2-[[4-(4-methylthiazol-5-yl)phenyl]-methylcarbamoyl]pyrrolidine-1-carbonyl]-2,2-dimethyl-propyl]amino]-2-oxoethoxy]ethoxy]ethoxy]ethoxy]ethyl]-1H-indole-2-carboxamide (“A7”)

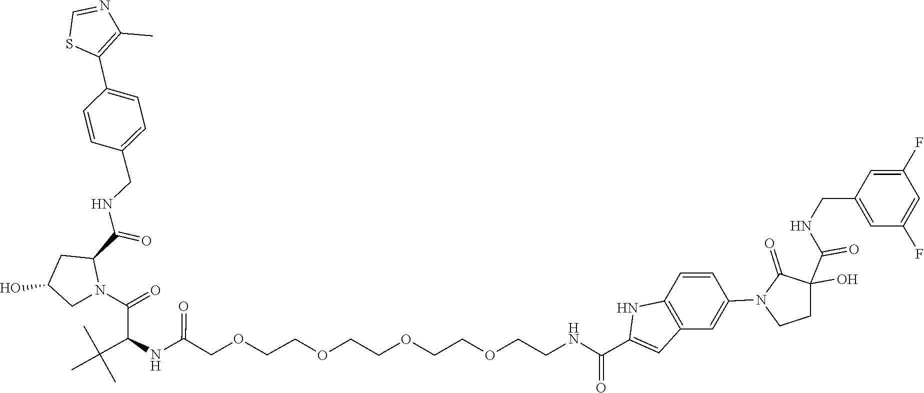 [SMILES: Cc1ncsc1-c1ccc(CNC(=O)[C@@H]2C[C@@H](O)CN2C(=O)[C@@H](NC(=O)COCCOCCOCCOCCNC(=O)c2cc3cc(N4CCC(O)(C(=O)NCc5cc(F)cc(F)c5)C4=O)ccc3[nH]2)C(C)(C)C)cc1]

LC-MS: RT: 1.49 min, Area: 98%, m/z 538.4=[M+2H]²⁺, m/z 1075.4=[M+H]⁺.

¹H NMR (700 MHz, DMSO-d₆) δ 11.62 (d, J=2.1 Hz, 1H), 8.98 (s, 1H), 8.69 (t, J=6.4 Hz, 1H), 8.57 (m, 2H), 7.77 (d, J=2.1 Hz, 1H), 7.54 (dd, J=9.0, 2.1 Hz, 1H), 7.48-7.33 (m, 6H), 7.13 (d, J=2.2 Hz, 1H), 7.06 (tt, J=9.3, 2.4 Hz, 1H), 7.00 (h, J=4.3 Hz, 2H), 6.70 (s, 1H), 5.14 (s, 1H), 4.56 (d, J=9.6 Hz, 1H), 4.46-4.33 (m, 4H), 4.26 (ddd, J=16.1, 11.0, 5.8 Hz, 2H), 3.95 (s, 2H), 3.90 (tq, J=9.1, 4.7, 4.0 Hz, 2H), 3.67 (dd, J=10.6, 4.0 Hz, 1H), 3.63-3.48 (m, 14H), 3.43 (q, J=5.9 Hz, 2H), 2.65-2.58 (m, 1H), 2.44 (s, 3H), 2.14 (dt, J=12.7, 7.5 Hz, 1H), 2.06 (ddd, J=9.1, 4.4, 2.2 Hz, 1H), 1.90 (ddd, J=13.1, 8.9, 4.6 Hz, 1H), 1.29-1.17 (m, 1H), 0.94 (s, 9H).

### 5-[3-[(3,5-difluorophenyl)methylcarbamoyl]-3-hydroxy-2-oxo-pyrrolidin-1-yl]-N-[2-[2-[2-[2-[2-[2-[[(1S)-1-[(2S,4R)-4-hydroxy-2-[[4-(4-methylthiazol-5-yl)phenyl]-methylcarbamoyl]pyrrolidine-1-carbonyl]-2,2-dimethyl-propyl]amino]-2-oxoethoxy]ethoxy]ethoxy]ethoxy]ethoxy]ethyl]-1H-indole-2-carboxamide (“A8”)

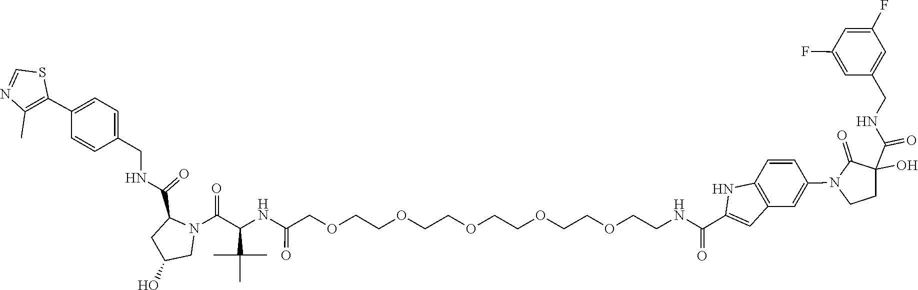 [SMILES: Cc1ncsc1-c1ccc(CNC(=O)[C@@H]2C[C@@H](O)CN2C(=O)[C@@H](NC(=O)COCCOCCOCCOCCOCCNC(=O)c2cc3cc(N4CCC(O)(C(=O)NCc5cc(F)cc(F)c5)C4=O)ccc3[nH]2)C(C)(C)C)cc1]

LC-MS: RT: 1.49 min, Area: 100%, m/z 560.4=[M+2H]²⁺, m/z 1119.4=[M+H]⁺.

¹H NMR (700 MHz, DMSO-d₆) δ 11.62 (d, J=2.1 Hz, 1H), 8.98 (s, 1H), 8.69 (t, J=6.5 Hz, 1H), 8.57 (m, 2H), 7.77 (d, J=2.0 Hz, 1H), 7.54 (dd, J=8.9, 2.1 Hz, 1H), 7.45-7.37 (m, 6H), 7.13 (d, J=2.1 Hz, 1H), 7.06 (tt, J=9.3, 2.4 Hz, 1H), 7.00 (h, J=4.3 Hz, 2H), 4.56 (d, J=9.6 Hz, 1H), 4.41 (m, 3H), 4.35 (tt, J=4.4, 2.1 Hz, 1H), 4.26 (m, 3H), 3.96 (m, 2H), 3.90 (m, 3H), 3.67 (dd, J=10.6, 4.0 Hz, 1H), 3.62-3.58 (m, 3H), 3.56-3.42 (m, 18H), 2.66-2.58 (m, 1H), 2.44 (s, 3H), 2.14 (m, 1H), 2.05 (ddt, J=12.0, 7.6, 1.9 Hz, 1H), 1.90 (ddd, J=13.0, 8.8, 4.5 Hz, 1H), 0.94 (s, 9H).

### 5-[3-[(3,5-difluorophenyl)methylcarbamoyl]-3-hydroxy-2-oxo-pyrrolidin-1-yl]-N-[2-[2-[2-[2-[2-[2-[[2-(2,6-dioxo-3-piperidyl)-1,3-dioxo-isoindolin-4-yl]-methylamino]ethoxy]ethoxy]ethoxy]ethoxy]ethoxy]ethyl]-1H-indole-2-carboxamide (“A9”)

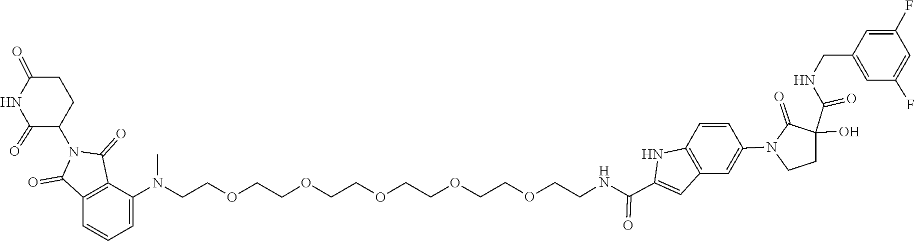 [SMILES: CN(CCOCCOCCOCCOCCOCCNC(=O)c1cc2cc(N3CCC(O)(C(=O)NCc4cc(F)cc(F)c4)C3=O)ccc2[nH]1)c1cccc2c1C(=O)N(C1CCC(=O)NC1=O)C2=O]

### 5-[3-[(3,5-difluorophenyl)methylcarbamoyl]-3-hydroxy-2-oxo-pyrrolidin-1-yl]-N-[2-[2-[2-[2-[2-[2-[2-(2,6-dioxo-3-piperidyl)-1,3-dioxo-isoindolin-4-yl]oxyethoxy]-ethoxy]ethoxy]ethoxy]ethoxy]ethyl]-1H-indole-2-carboxamide (“A10”)

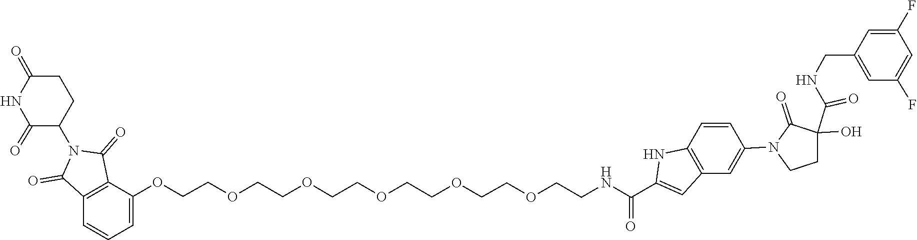 [SMILES: O=C1CCC(N2C(=O)c3cccc(OCCOCCOCCOCCOCCOCCNC(=O)c4cc5cc(N6CCC(O)(C(=O)NCc7cc(F)cc(F)c7)C6=O)ccc5[nH]4)c3C2=O)C(=O)N1]

### 5-[3-[(3,5-difluorophenyl)methylcarbamoyl]-3-hydroxy-2-oxo-pyrrolidin-1-yl]-N-[2-[2-[2-[2-[2-[2-[2-(2,6-dioxo-3-piperidyl)-1,3-dioxo-isoindolin-4-yl]ethoxy]-ethoxy]ethoxy]ethoxy]ethoxy]ethyl]-1H-indole-2-carboxamide (“A11”)

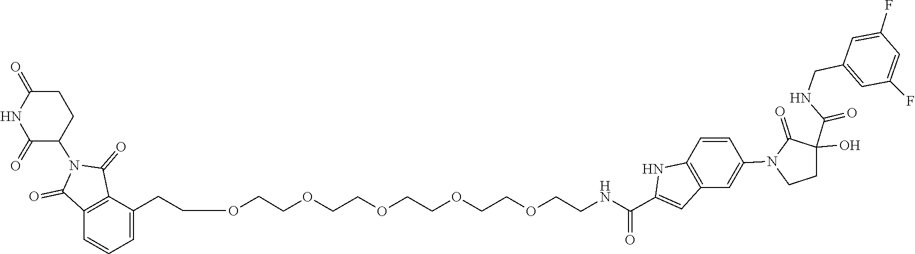 [SMILES: O=C1CCC(N2C(=O)c3cccc(CCOCCOCCOCCOCCOCCNC(=O)c4cc5cc(N6CCC(O)(C(=O)NCc7cc(F)cc(F)c7)C6=O)ccc5[nH]4)c3C2=O)C(=O)N1]

### 5-[3-[(3,5-difluorophenyl)methylcarbamoyl]-3-hydroxy-2-oxo-pyrrolidin-1-yl]-N-[2-[2-[2-[2-[2-[2-[2-(2,6-dioxo-3-piperidyl)-1,3-dioxo-isoindolin-4-yl]oxyethoxy]-ethoxy]ethoxy]ethoxy]ethoxy]ethyl]-N-methyl-1H-indole-2-carboxamide (“A12”)

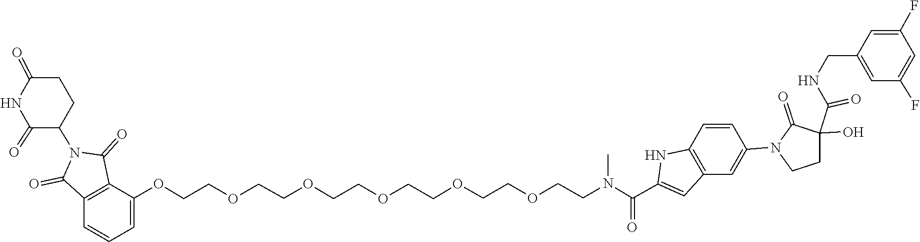 [SMILES: CN(CCOCCOCCOCCOCCOCCOc1cccc2c1C(=O)N(C1CCC(=O)NC1=O)C2=O)C(=O)c1cc2cc(N3CCC(O)(C(=O)NCc4cc(F)cc(F)c4)C3=O)ccc2[nH]1]

### 5-[3-[(3,5-difluorophenyl)methylcarbamoyl]-3-hydroxy-2-oxo-pyrrolidin-1-yl]-N-[5-[2-[2-[2-[2-[2-(2,6-dioxo-3-piperidyl)-1,3-dioxo-isoindolin-4-yl]oxyethoxy]-ethoxy]ethoxy]ethoxy]pentyl]-1H-indole-2-carboxamide (“A13”)

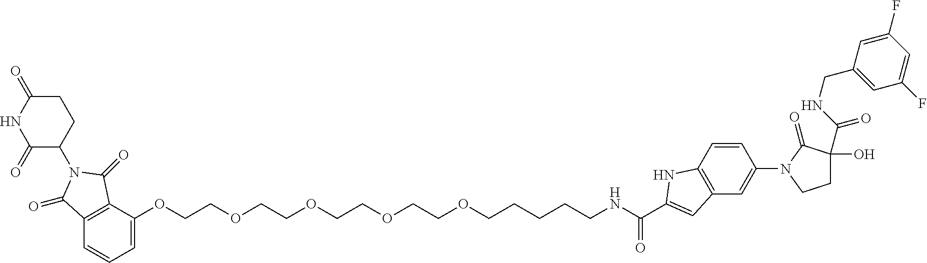 [SMILES: O=C1CCC(N2C(=O)c3cccc(OCCOCCOCCOCCOCCCCCNC(=O)c4cc5cc(N6CCC(O)(C(=O)NCc7cc(F)cc(F)c7)C6=O)ccc5[nH]4)c3C2=O)C(=O)N1]

### 5-[3-[(3,5-difluorophenyl)methylcarbamoyl]-3-hydroxy-2-oxo-pyrrolidin-1-yl]-N-[8-[2-[2-[2-[2-(2,6-dioxo-3-piperidyl)-1,3-dioxo-isoindolin-4-yl]oxyethoxy]-ethoxy]ethoxy]octyl]-1H-indole-2-carboxamide (“A14”)

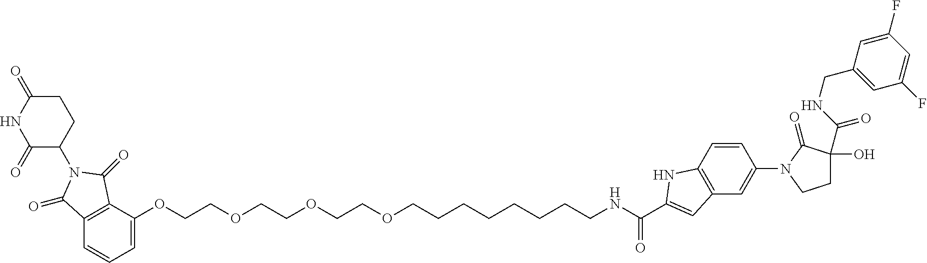 [SMILES: O=C1CCC(N2C(=O)c3cccc(OCCOCCOCCOCCCCCCCCNC(=O)c4cc5cc(N6CCC(O)(C(=O)NCc7cc(F)cc(F)c7)C6=O)ccc5[nH]4)c3C2=O)C(=O)N1]

### 5-[3-[(3,5-difluorophenyl)methylcarbamoyl]-3-hydroxy-2-oxo-pyrrolidin-1-yl]-N-[2-[2-[2-[2-[5-[[2-(2,6-dioxo-3-piperidyl)-1,3-dioxo-isoindolin-4-yl]amino]-pentoxy]ethoxy]ethoxy]ethoxy]ethyl]-1H-indole-2-carboxamide (“A15”)

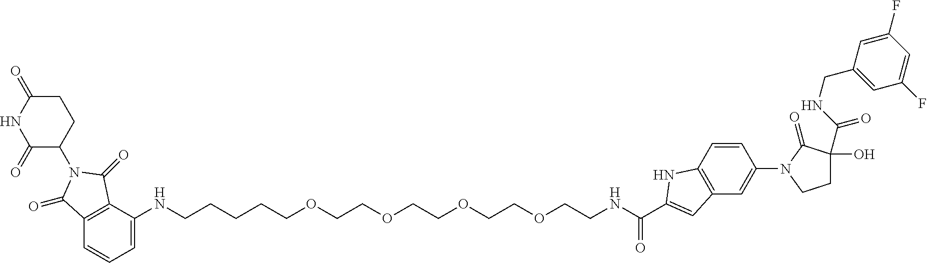 [SMILES: O=C1CCC(N2C(=O)c3cccc(NCCCCCOCCOCCOCCOCCNC(=O)c4cc5cc(N6CCC(O)(C(=O)NCc7cc(F)cc(F)c7)C6=O)ccc5[nH]4)c3C2=O)C(=O)N1]

### 5-[3-[(3,5-difluorophenyl)methylcarbamoyl]-3-hydroxy-2-oxo-pyrrolidin-1-yl]-N-[2-[2-[2-[8-[[2-(2,6-dioxo-3-piperidyl)-1,3-dioxo-isoindolin-4-yl]amino]octoxy]-ethoxy]ethoxy]ethyl]-1H-indole-2-carboxamide (“A16”)

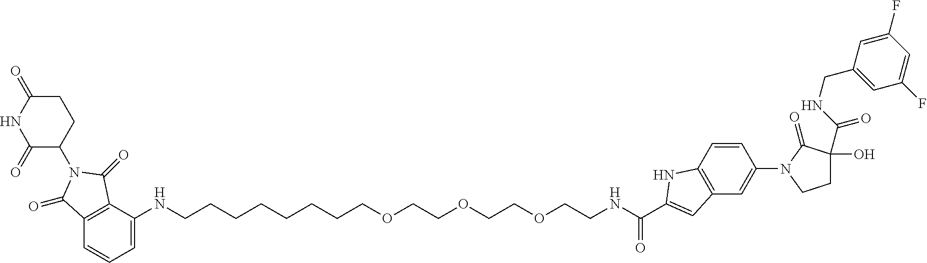 [SMILES: O=C1CCC(N2C(=O)c3cccc(NCCCCCCCCOCCOCCOCCNC(=O)c4cc5cc(N6CCC(O)(C(=O)NCc7cc(F)cc(F)c7)C6=O)ccc5[nH]4)c3C2=O)C(=O)N1]

### 5-[3-[(3,5-difluorophenyl)methylcarbamoyl]-3-hydroxy-2-oxo-pyrrolidin-1-yl]-N-[2-[2-[2-[2-[2-[2-[[2-(2,6-dioxo-3-piperidyl)-1,3-dioxo-isoindolin-4-yl]amino]-ethoxy]ethoxy]ethoxy]ethoxy]ethoxy]ethyl]-1H-benzimidazole-2-carboxamide (“A17”)

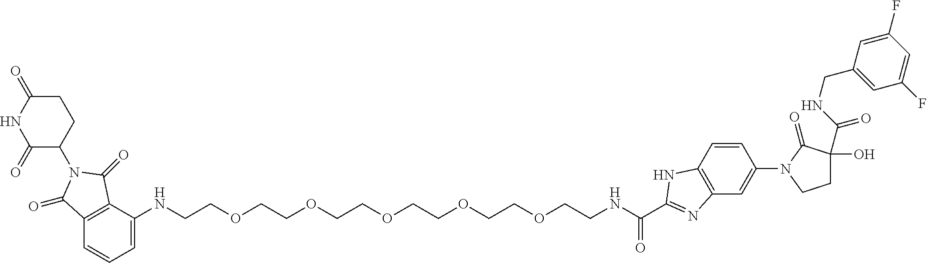 [SMILES: O=C1CCC(N2C(=O)c3cccc(NCCOCCOCCOCCOCCOCCNC(=O)c4nc5cc(N6CCC(O)(C(=O)NCc7cc(F)cc(F)c7)C6=O)ccc5[nH]4)c3C2=O)C(=O)N1]

### N-[(3,5-difluorophenyl)methyl]-1-[4-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethylcarbamoyl]phenyl]-3-hydroxy-2-oxo-pyrrolidine-3-carboxamide (“A18”)

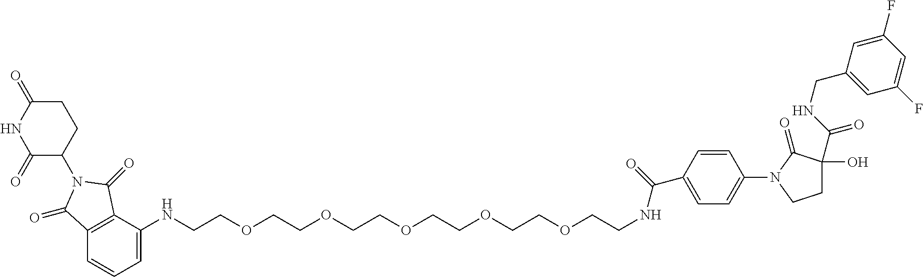 [SMILES: O=C1CCC(N2C(=O)c3cccc(NCCOCCOCCOCCOCCOCCNC(=O)c4ccc(N5CCC(O)(C(=O)NCc6cc(F)cc(F)c6)C5=O)cc4)c3C2=O)C(=O)N1]

### 5-[3-[(3-chloro-5-fluoro-phenyl)methylcarbamoyl]-3-hydroxy-2-oxo-pyrrolidin-1-yl]-N-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethyl]-1H-indole-2-carboxamide (“A19”)

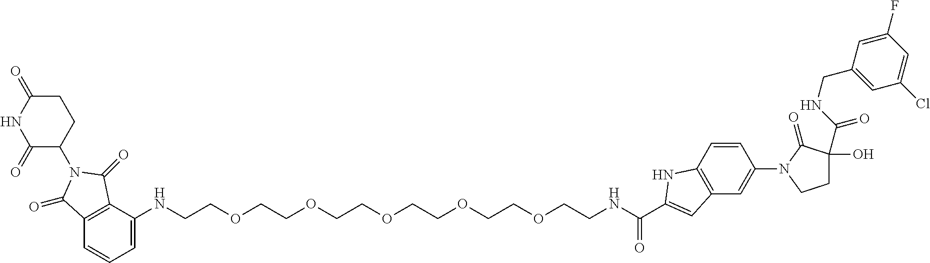 [SMILES: O=C1CCC(N2C(=O)c3cccc(NCCOCCOCCOCCOCCOCCNC(=O)c4cc5cc(N6CCC(O)(C(=O)NCc7cc(F)cc(Cl)c7)C6=O)ccc5[nH]4)c3C2=O)C(=O)N1]

### 5-[3-[(3-chloro-5-fluoro-phenyl)methylcarbamoyl]-3-hydroxy-5-methyl-2-oxopyrrolidin-1-yl]-N-[2-[2-[2-[2-[2-[2-[[2-(2,6-dioxo-3-piperidyl)-1,3-dioxoisoindolin-4-yl]amino]ethoxy]ethoxy]ethoxy]ethoxy]ethoxy]ethyl]-1H-indole-2-carboxamide (“A20”)

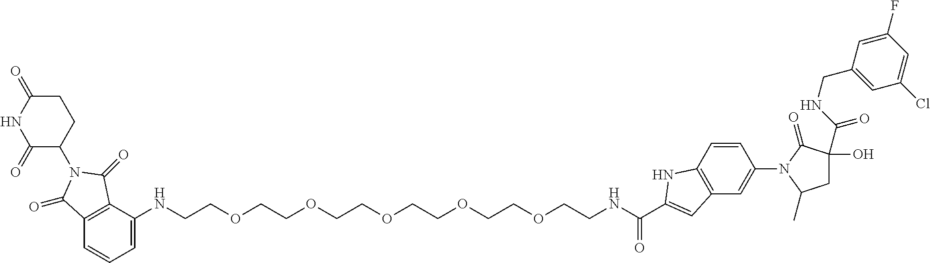 [SMILES: CC1CC(O)(C(=O)NCc2cc(F)cc(Cl)c2)C(=O)N1c1ccc2[nH]c(C(=O)NCCOCCOCCOCCOCCOCCNc3cccc4c3C(=O)N(C3CCC(=O)NC3=O)C4=O)cc2c1]

### 5-[3-[(3,5-difluorophenyl)methylcarbamoyl]-3,4-dihydroxy-2-oxo-pyrrolidin-1-yl]-N-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethyl]-1H-indole-2-carboxamide (“A21”)

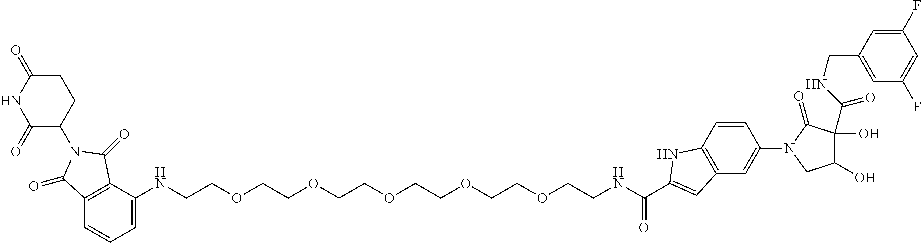 [SMILES: O=C1CCC(N2C(=O)c3cccc(NCCOCCOCCOCCOCCOCCNC(=O)c4cc5cc(N6CC(O)C(O)(C(=O)NCc7cc(F)cc(F)c7)C6=O)ccc5[nH]4)c3C2=O)C(=O)N1]

### 5-[3-[(3-chloro-5-fluoro-phenyl)methylcarbamoyl]-5-fluoro-3-hydroxy-2-oxopyrrolidin-1-yl]-N-[2-[2-[2-[2-[2-[2-[[2-(2,6-dioxo-3-piperidyl)-1,3-dioxoisoindolin-4-yl]amino]ethoxy]ethoxy]ethoxy]ethoxy]ethoxy]ethyl]-1H-indole-2-carboxamide (“A22”)

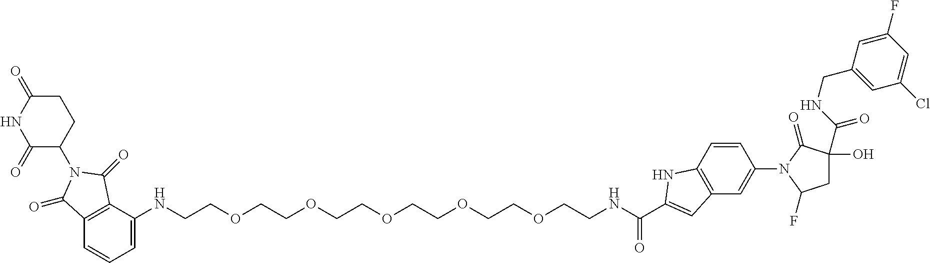 [SMILES: O=C1CCC(N2C(=O)c3cccc(NCCOCCOCCOCCOCCOCCNC(=O)c4cc5cc(N6C(=O)C(O)(C(=O)NCc7cc(F)cc(Cl)c7)CC6F)ccc5[nH]4)c3C2=O)C(=O)N1]

### 5-[3-[(3-chloro-5-fluoro-phenyl)methylcarbamoyl]-3-hydroxy-2-oxo-1-piperidyl]-N-[2-[2-[2-[2-[2-[2-[[2-(2,6-dioxo-3-piperidyl)-1,3-dioxo-isoindolin-4-yl]amino]-ethoxy]ethoxy]ethoxy]ethoxy]ethoxy]ethyl]-1H-indole-2-carboxamide (“A23”)

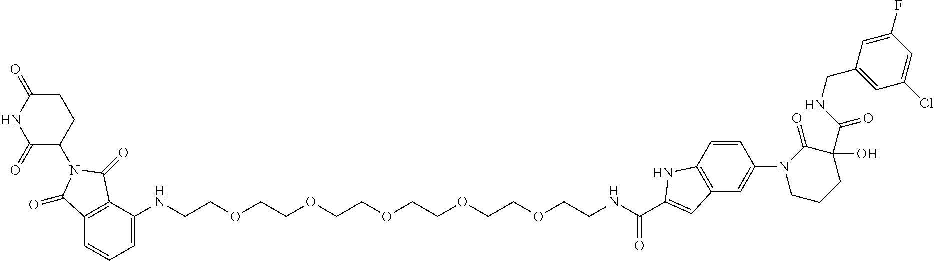 [SMILES: O=C1CCC(N2C(=O)c3cccc(NCCOCCOCCOCCOCCOCCNC(=O)c4cc5cc(N6CCCC(O)(C(=O)NCc7cc(F)cc(Cl)c7)C6=O)ccc5[nH]4)c3C2=O)C(=O)N1]

### 5-[3-[2-(3,5-difluorophenyl)ethylcarbamoyl]-3-hydroxy-2-oxo-pyrrolidin-1-yl]-N-[2-[2-[2-[2-[2-[2-[[2-(2,6-dioxo-3-piperidyl)-1,3-dioxo-isoindolin-4-yl]amino]-ethoxy]ethoxy]ethoxy]ethoxy]ethoxy]ethyl]-1H-indole-2-carboxamide (“A24”)

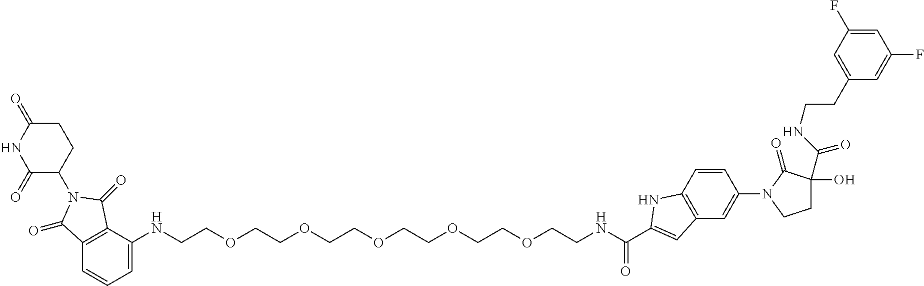 [SMILES: O=C1CCC(N2C(=O)c3cccc(NCCOCCOCCOCCOCCOCCNC(=O)c4cc5cc(N6CCC(O)(C(=O)NCCc7cc(F)cc(F)c7)C6=O)ccc5[nH]4)c3C2=O)C(=O)N1]

### N-[2-[2-[2-[2-[2-[2-[[2-(2,6-dioxo-3-piperidyl)-1,3-dioxo-isoindolin-4-yl]amino]-ethoxy]ethoxy]ethoxy]ethoxy]ethoxy]ethyl]-5-[3-hydroxy-2-oxo-3-[2-(2-thienyl)ethylcarbamoyl]pyrrolidin-1-yl]-1H-indole-2-carboxamide (“A25”)

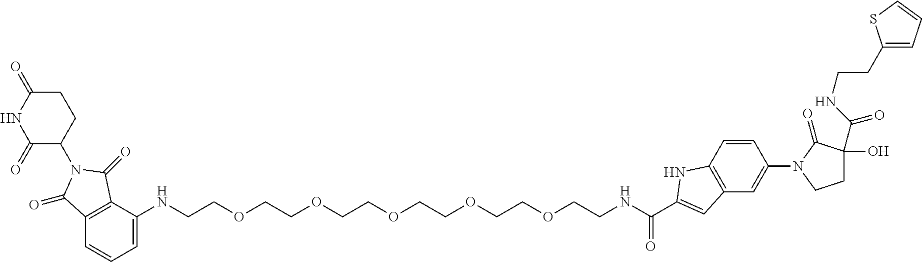 [SMILES: O=C1CCC(N2C(=O)c3cccc(NCCOCCOCCOCCOCCOCCNC(=O)c4cc5cc(N6CCC(O)(C(=O)NCCc7cccs7)C6=O)ccc5[nH]4)c3C2=O)C(=O)N1]

### N-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethyl]-5-[3-[2-(2-furyl)ethylcarbamoyl]-3-hydroxy-2-oxo-pyrrolidin-1-yl]-1H-indole-2-carboxamide (“A26”)

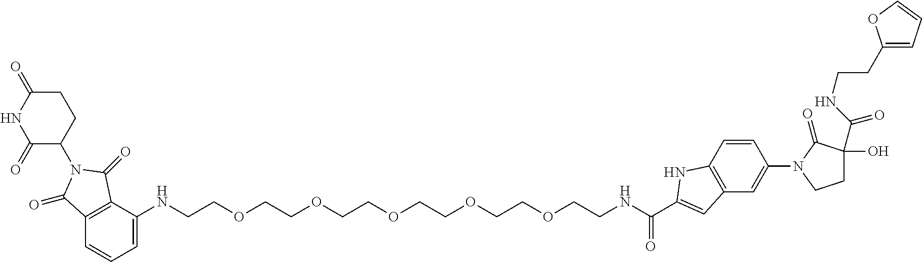 [SMILES: O=C1CCC(N2C(=O)c3cccc(NCCOCCOCCOCCOCCOCCNC(=O)c4cc5cc(N6CCC(O)(C(=O)NCCc7ccco7)C6=O)ccc5[nH]4)c3C2=O)C(=O)N1]

### 5-[3-(cyclohexylmethylcarbamoyl)-3-hydroxy-2-oxo-pyrrolidin-1-yl]-N-[2-[2-[2-[2-[2-[2-[[2-(2,6-dioxo-3-piperidyl)-1,3-dioxo-isoindolin-4-yl]amino]ethoxy]-ethoxy]ethoxy]ethoxy]ethoxy]ethyl]-1H-indole-2-carboxamide (“A27”)

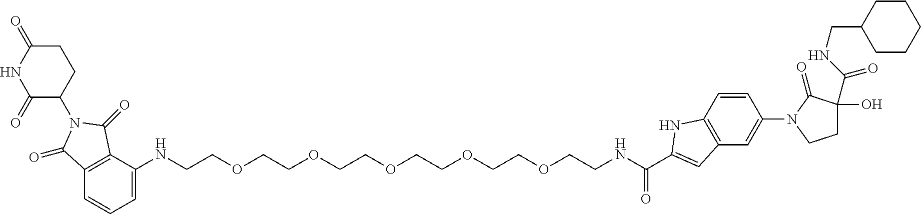 [SMILES: O=C1CCC(N2C(=O)c3cccc(NCCOCCOCCOCCOCCOCCNC(=O)c4cc5cc(N6CCC(O)(C(=O)NCC7CCCCC7)C6=O)ccc5[nH]4)c3C2=O)C(=O)N1]

### N-[(3,5-difluorophenyl)methyl]-1-[6-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]-ethylamino]-6-oxo-hexyl]-3-hydroxy-2-oxo-pyrrolidine-3-carboxamide (“A28”)

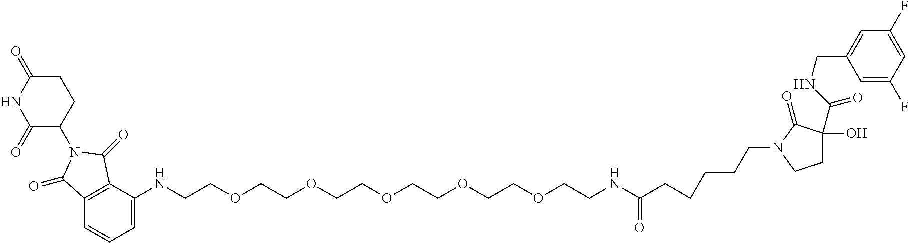 [SMILES: O=C(CCCCCN1CCC(O)(C(=O)NCc2cc(F)cc(F)c2)C1=O)NCCOCCOCCOCCOCCOCCNc1cccc2c1C(=O)N(C1CCC(=O)NC1=O)C2=O]

### N-[(3,5-difluorophenyl)methyl]-1-[2-[4-[2-[2-[2-[2-[2-[[2-(2,6-dioxo-3-piperidyl)-1,3-dioxo-isoindolin-4-yl]amino]ethoxy]ethoxy]ethoxy]ethoxy]ethyl]piperazine-1-carbonyl]-1H-indol-5-yl]-3-hydroxy-2-oxo-pyrrolidine-3-carboxamide (“A29”)

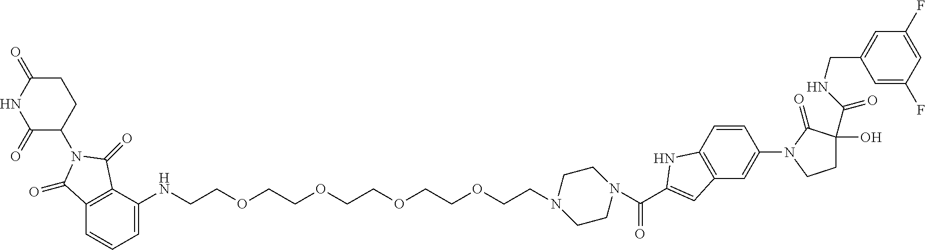 [SMILES: O=C1CCC(N2C(=O)c3cccc(NCCOCCOCCOCCOCCN4CCN(C(=O)c5cc6cc(N7CCC(O)(C(=O)NCc8cc(F)cc(F)c8)C7=O)ccc6[nH]5)CC4)c3C2=O)C(=O)N1]

### N-[(3,5-difluorophenyl)methyl]-1-[2-[9-[2-[2-[2-[2-[[2-(2,6-dioxo-3-piperidyl)-1,3-dioxo-isoindolin-4-yl]amino]ethoxy]ethoxy]ethoxy]ethyl]-3,9-diazaspiro[5.5]undecane-3-carbonyl]-1H-indol-5-yl]-3-hydroxy-2-oxopyrrolidine-3-carboxamide (“A30”)

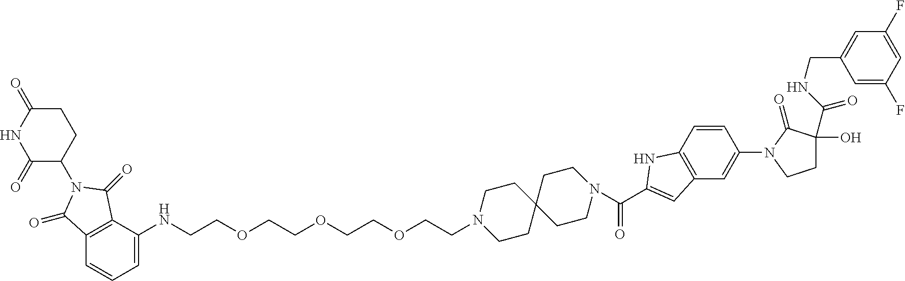 [SMILES: O=C1CCC(N2C(=O)c3cccc(NCCOCCOCCOCCN4CCC5(CC4)CCN(C(=O)c4cc6cc(N7CCC(O)(C(=O)NCc8cc(F)cc(F)c8)C7=O)ccc6[nH]4)CC5)c3C2=O)C(=O)N1]

### 5-[3-[(3,5-difluorophenyl)methylcarbamoyl]-3-hydroxy-2-oxo-pyrrolidin-1-yl]-N-[14-[[2-(2,6-dioxo-3-piperidyl)-1,3-dioxo-isoindolin-5-yl]amino]tetradecyl]-1H-indole-2-carboxamide (“A31”)

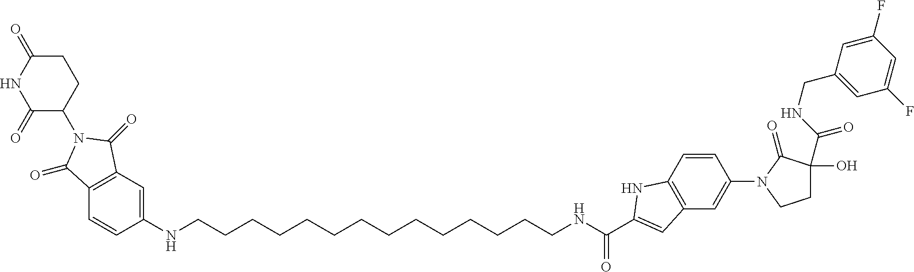 [SMILES: O=C1CCC(N2C(=O)c3ccc(NCCCCCCCCCCCCCCNC(=O)c4cc5cc(N6CCC(O)(C(=O)NCc7cc(F)cc(F)c7)C6=O)ccc5[nH]4)cc3C2=O)C(=O)N1]

### 5-[3-[(3,5-difluorophenyl)methylcarbamoyl]-3-hydroxy-2-oxo-pyrrolidin-1-yl]-N-[3-[3-[3-[[2-(2,6-dioxo-3-piperidyl)-1,3-dioxo-isoindolin-5-yl]amino]propoxy]-propoxy]propoxymethyl]-1H-indole-2-carboxamide (“A32”)

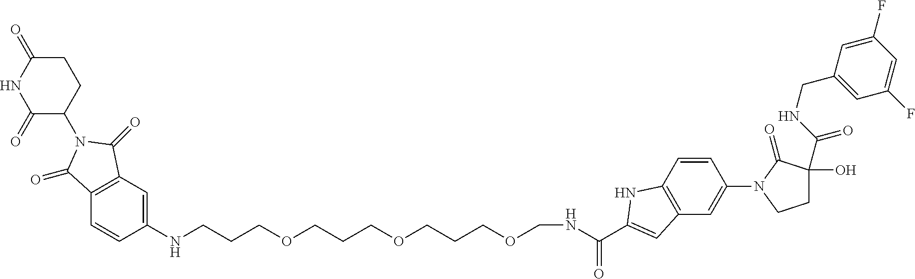 [SMILES: O=C1CCC(N2C(=O)c3ccc(NCCCOCCCOCCCOCNC(=O)c4cc5cc(N6CCC(O)(C(=O)NCc7cc(F)cc(F)c7)C6=O)ccc5[nH]4)cc3C2=O)C(=O)N1]

### 5-[3-[(3,5-difluorophenyl)methylcarbamoyl]-3-hydroxy-2-oxo-pyrrolidin-1-yl]-N-[4-[3-[4-[[2-(2,6-dioxo-3-piperidyl)-1,3-dioxo-isoindolin-5-yl]amino]butoxy]-propoxy]butyl]-1H-indole-2-carboxamide (“A33”)

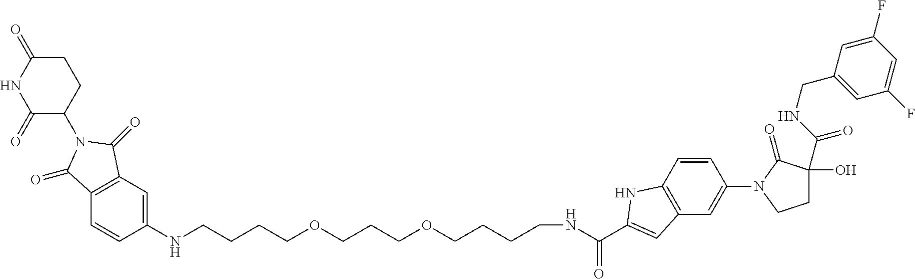 [SMILES: O=C1CCC(N2C(=O)c3ccc(NCCCCOCCCOCCCCNC(=O)c4cc5cc(N6CCC(O)(C(=O)NCc7cc(F)cc(F)c7)C6=O)ccc5[nH]4)cc3C2=O)C(=O)N1]

### 5-[3-[(3,5-difluorophenyl)methylcarbamoyl]-3-hydroxy-2-oxo-pyrrolidin-1-yl]-N-[2-[2-[2-[2-[[2-[2-(2,6-dioxo-3-piperidyl)-1,3-dioxo-isoindolin-5-yl]oxyacetyl]-amino]ethoxy]ethoxy]ethoxy]ethyl]-1H-indole-2-carboxamide (“A34”)

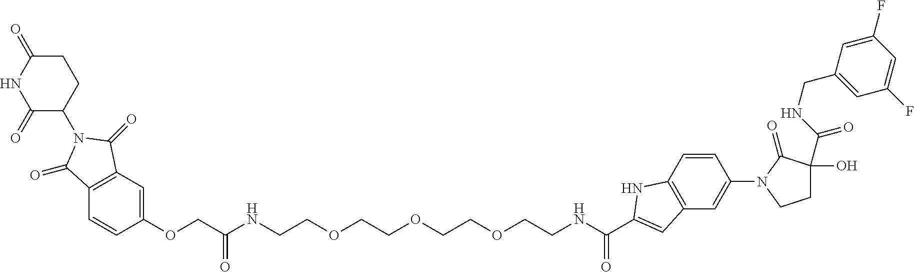 [SMILES: O=C(COc1ccc2c(c1)C(=O)N(C1CCC(=O)NC1=O)C2=O)NCCOCCOCCOCCNC(=O)c1cc2cc(N3CCC(O)(C(=O)NCc4cc(F)cc(F)c4)C3=O)ccc2[nH]1]

### N-[(3,5-difluorophenyl)methyl]-1-[2-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethylsulfamoyl]-1H-indol-5-yl]-3-hydroxy-2-oxo-pyrrolidine-3-carboxamide (“A35”)

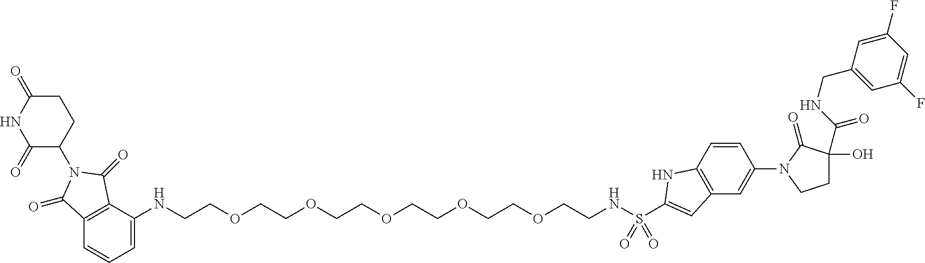 [SMILES: O=C1CCC(N2C(=O)c3cccc(NCCOCCOCCOCCOCCOCCNS(=O)(=O)c4cc5cc(N6CCC(O)(C(=O)NCc7cc(F)cc(F)c7)C6=O)ccc5[nH]4)c3C2=O)C(=O)N1]

### 2-[2-[2-[2-[2-[2-[[2-(2,6-dioxo-3-piperidyl)-1,3-dioxo-isoindolin-4-yl]amino]-ethoxy]ethoxy]ethoxy]ethoxy]ethoxy]ethyl 5-[3-[(3,5-difluorophenyl)methyl-carbamoyl]-3-hydroxy-2-oxo-pyrrolidin-1-yl]-1H-indole-2-sulfonate (“A36”)

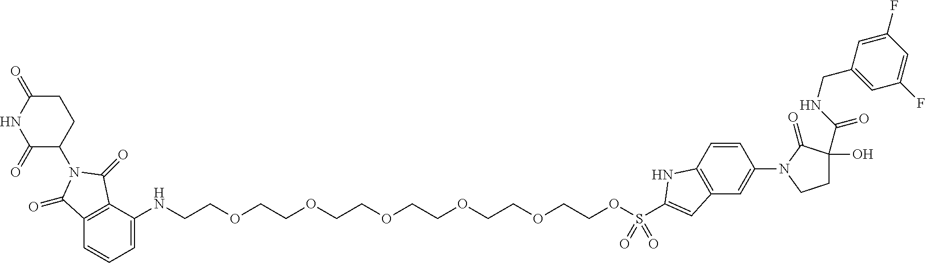 [SMILES: O=C1CCC(N2C(=O)c3cccc(NCCOCCOCCOCCOCCOCCOS(=O)(=O)c4cc5cc(N6CCC(O)(C(=O)NCc7cc(F)cc(F)c7)C6=O)ccc5[nH]4)c3C2=O)C(=O)N1]

### N-[(3,5-difluorophenyl)methyl]-1-[2-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethoxy-sulfonimidoyl]-1H-indol-5-yl]-3-hydroxy-2-oxo-pyrrolidine-3-carboxamide (“A37”)

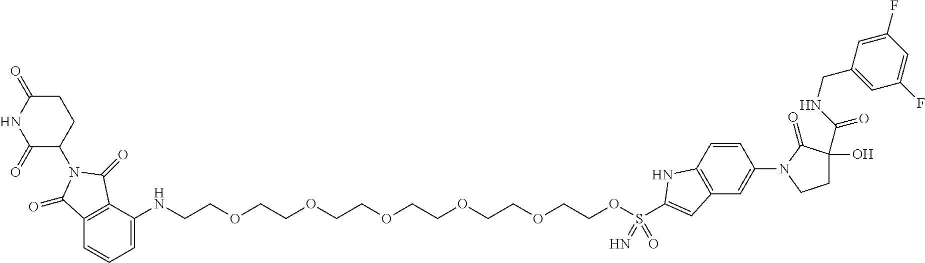 [SMILES: N=S(=O)(OCCOCCOCCOCCOCCOCCNc1cccc2c1C(=O)N(C1CCC(=O)NC1=O)C2=O)c1cc2cc(N3CCC(O)(C(=O)NCc4cc(F)cc(F)c4)C3=O)ccc2[nH]1]

### N-[(3,5-difluorophenyl)methyl]-1-[4-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethyl-carbamoylamino]phenyl]-3-hydroxy-2-oxo-pyrrolidine-3-carboxamide (“A38”)

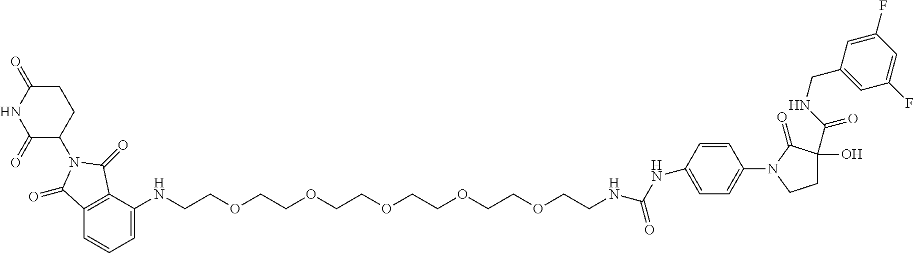 [SMILES: O=C1CCC(N2C(=O)c3cccc(NCCOCCOCCOCCOCCOCCNC(=O)Nc4ccc(N5CCC(O)(C(=O)NCc6cc(F)cc(F)c6)C5=O)cc4)c3C2=O)C(=O)N1]

### 2-[2-[2-[2-[2-[2-[[2-(2,6-dioxo-3-piperidyl)-1,3-dioxo-isoindolin-4-yl]amino]-ethoxy]ethoxy]ethoxy]ethoxy]ethoxy]ethyl N-[4-[3-[(3,5-difluorophenyl)methylcarbamoyl]-3-hydroxy-2-oxo-pyrrolidin-1-yl]phenyl]carbamate (“A39”)

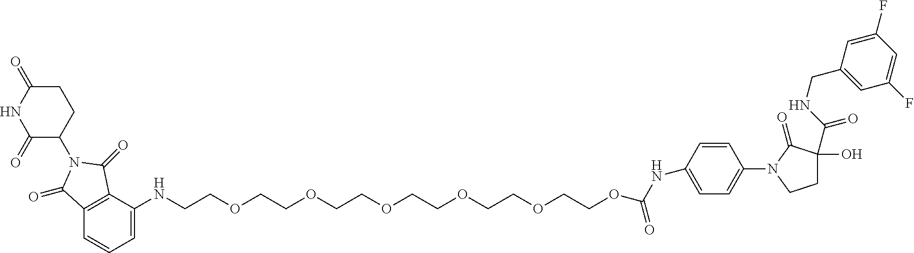 [SMILES: O=C1CCC(N2C(=O)c3cccc(NCCOCCOCCOCCOCCOCCOC(=O)Nc4ccc(N5CCC(O)(C(=O)NCc6cc(F)cc(F)c6)C5=O)cc4)c3C2=O)C(=O)N1]

### N-[(3,5-difluorophenyl)methyl]-1-[4-[2-[2-[2-[2-[2-[2-[2-[[2-(2,6-dioxo-3-piperidyl)-1,3-dioxo-isoindolin-4-yl]amino]ethoxy]ethoxy]ethoxy]ethoxy]-ethoxy]ethoxy]ethylsulfanyl]phenyl]-3-hydroxy-2-oxo-pyrrolidine-3-carboxamide (“A40”)

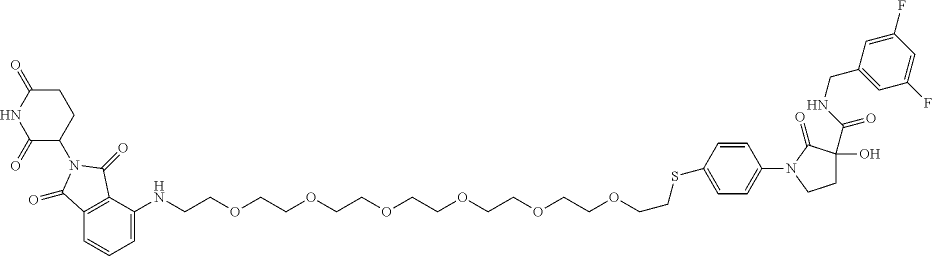 [SMILES: O=C1CCC(N2C(=O)c3cccc(NCCOCCOCCOCCOCCOCCOCCSc4ccc(N5CCC(O)(C(=O)NCc6cc(F)cc(F)c6)C5=O)cc4)c3C2=O)C(=O)N1]

### N-[(3,5-difluorophenyl)methyl]-1-[4-[2-[2-[2-[2-[2-[2-[2-[[2-(2,6-dioxo-3-piperidyl)-1,3-dioxo-isoindolin-4-yl]amino]ethoxy]ethoxy]ethoxy]ethoxy]-ethoxy]ethoxy]ethoxy]phenyl]-3-hydroxy-2-oxo-pyrrolidine-3-carboxamide (“A41”)

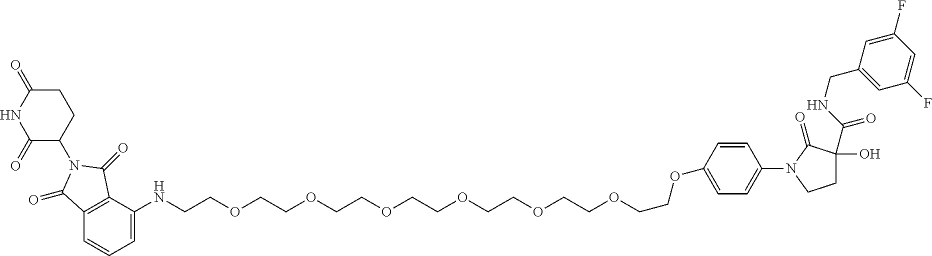 [SMILES: O=C1CCC(N2C(=O)c3cccc(NCCOCCOCCOCCOCCOCCOCCOc4ccc(N5CCC(O)(C(=O)NCc6cc(F)cc(F)c6)C5=O)cc4)c3C2=O)C(=O)N1]

### 5-[(3S)-3-{[(3,5-difluorophenyl)methyl]carbamoyl}-3-hydroxy-2-oxopyrrolidin-1-yl]-N-(2-{2-[2-(2-{[2-(2,6-dioxopiperidin-3-yl)-1,3-dioxo-2,3-dihydro-1H-isoindol-5-yl]amino}ethoxy)ethoxy]ethoxy}ethyl)-1H-indole-2-carboxamide (“A42”)

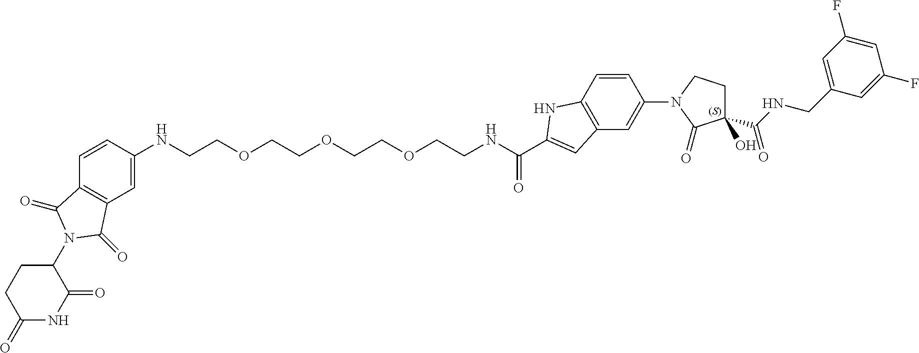

Method 1:

MS instrument type: SHIMADZU LCMS-2020; column: Kinetex EVO C18 30*2.1 mm, 5 um; mobile phase A: 0.0375% TFA in water (v/v), B: 0.01875% TFA in Acetonitrile (v/v); gradient: 0.0 min 5% B→0.80 min 95% B→1.20 min 95% B→1.21 min 5% B→1.55 min 5% B; flow rate: 1.5 (mL/min); oven temperature: 50° C.; UV detection: 220 nm & 254 nm.

Method 2:

HPLC instrument type: SHIMADZU LC-20AB; column: Kinetex C18 LC Column 4.6×50 mm, 5 um; mobile phase A: 0.0375% TFA in water (v/v), B: 0.01875% TFA in Acetonitrile (v/v); gradient: 0.0 min 10% B→2.40 min 80% B→3.70 min 80% B→3.71 min 10% B→4.00 min 10% B; oven temperature: 50° C.; UV detection: PDA (220 nm&215 nm&254 nm).

General Procedure for Preparation of Intermediate 3

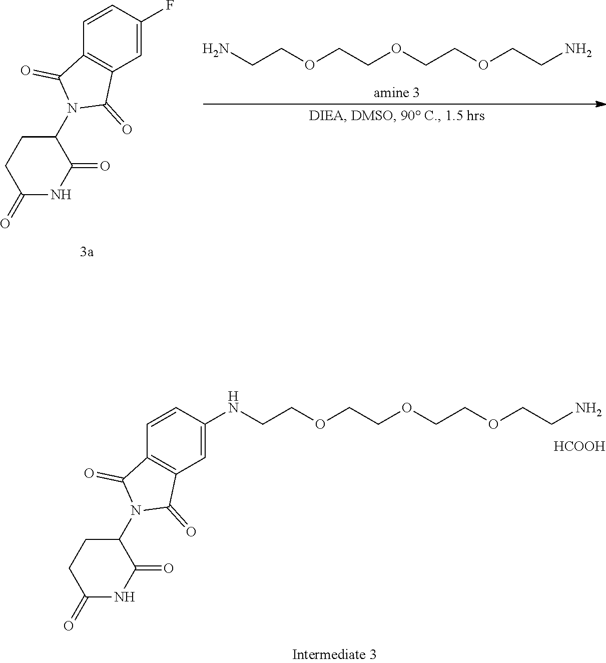 [SMILES: O=C1CCC(N2C(=O)c3ccc(F)cc3C2=O)C(=O)N1 ; NCCOCCOCCOCCN ; NCCOCCOCCOCCNc1ccc2c(c1)C(=O)N(C1CCC(=O)NC1=O)C2=O ; "HCOOH"]

A solution of compound 3a (70 mg, 253 umol), amine 3 (80 mg, 416 umol) and DIEA (222 mg, 1.72 mmol, 300 uL) in DMSO (3 mL) was stirred at 90° C. under N₂ for 1.5 hrs. LCMS (Rt=0.687 min, MS+1=449.2) showed MS of intermediate 3 was detected. The mixture was cooled to 25-30° C., pH value of the mixture was adjusted to 6-7 with AcOH. The mixture was purified by Prep-HPLC (column: Phenomenex Synergi C18 150*25*10 um; mobile phase: [water (0.225% FA)-ACN]; B %: 0%-30%, 10 min), the fraction was concentrated under reduced pressure to give intermediate 3 (50 mg, 96.4 umol, 12.7% yield, 95.3% purity, FA) as brown oil, which was confirmed by LCMS (Rt=0.698 min, MS+1=449.3).

LCMS: (Method 1), Rt=0.687 min, MS+1=449.2

General Procedure for Preparation of the Title Compound “A42”

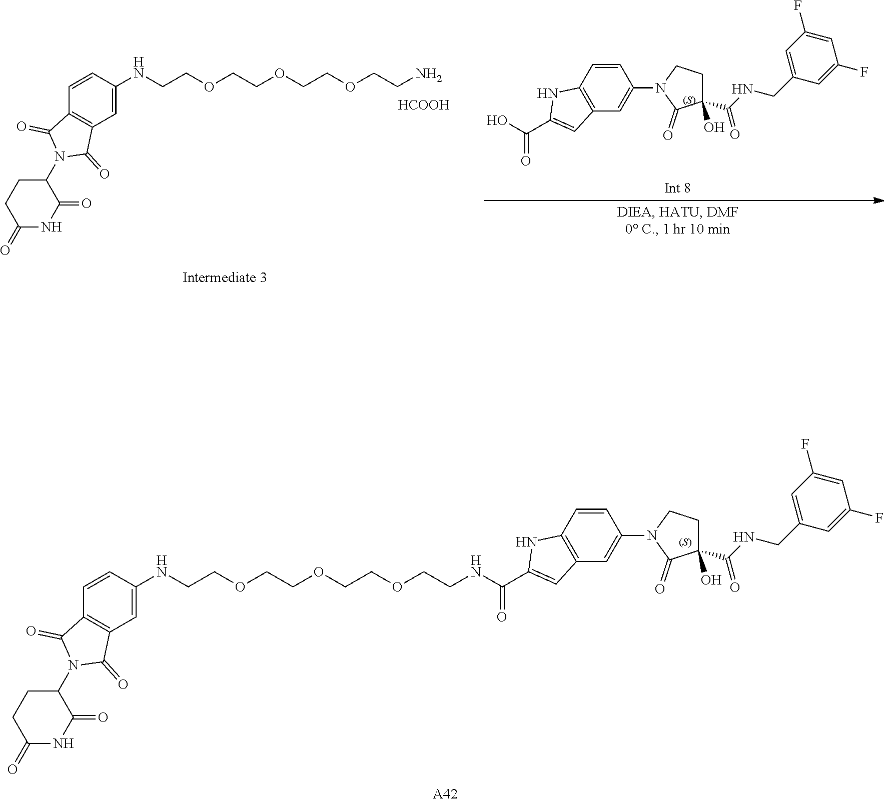

To a solution of Int 8 (35 mg, 81.5 umol) in DMF (2 mL) was successively added DIEA (30 mg, 232 umol) and HATU (35 mg, 92.0 umol) at 0° C. under N₂, the mixture was stirred at 0° C. for 10 min, then a solution of intermediate 3 (40 mg, 80.8 umol, 1.00 eq, FA) in DMF (2.00 mL) was added into the mixture at 0° C., it was stirred for 1 hr under N₂ at 0° C. The mixture was poured into icy water (20.0 mL), extracted with ethyl acetate (10.0 mL×2), the organic phase was separated and washed with brine (20.0 mL×2), dried with Na₂SO₄, filtered, the filtrate was concentrated under reduced pressure to give brown oil. It was purified by Prep-HPLC (column: Phenomenex Synergi C18 75*30*3 um; mobile phase: [water (0.225% FA)-ACN]; B %: 31%-51%, 8 min), solvent of the fraction was removed by lyophilization to A42 (23 mg, 24.52 umol, 30.32% yield) as yellow solid, which was confirmed by ¹H-NMR, LCMS and HPLC.

¹H NMR: 400 MHz, DMSO-d₆; 11.62 (s, 1H), 11.05 (s, 1H), 8.70 (t, J=6.4 Hz, 1H), 8.60-8.52 (m, 1H), 8.42 (s, 1H), 7.78 (s, 1H), 7.58-7.50 (m, 2H), 7.42 (d, J=8.9 Hz, 1H), 7.18-7.10 (m, 2H), 7.09-6.96 (m, 4H), 6.87 (d, J=8.4 Hz, 1H), 6.72 (s, 1H), 5.02 (d, J=8.8 Hz, 1H), 4.41 (d, J=7.6 Hz, 1H), 4.26 (d, J=8.8 Hz, 1H), 3.89 (t, J=7.2 Hz, 2H), 3.59-3.49 (m, 15H), 2.92-2.81 (m, 1H), 2.65-2.58 (m, 3H), 2.19-2.10 (m, 1H), 2.03-1.95 (m, 1H), 1.23 (s, 1H)

LCMS: Rt=0.720 min, MS+1=860.2;

HPLC: Rt=1.860 min

### 5-[(3S)-3-{[(3,5-difluorophenyl)methyl]carbamoyl}-3-hydroxy-2-oxopyrrolidin-1-yl]-N-{15-[2-(2,6-dioxopiperidin-3-yl)-1,3-dioxo-2,3-dihydro-1H-isoindol-5-yl]-3,6,9,12-tetraoxapentadec-14-yn-1-yl}-1H-indole-2-carboxamide (“A43”)

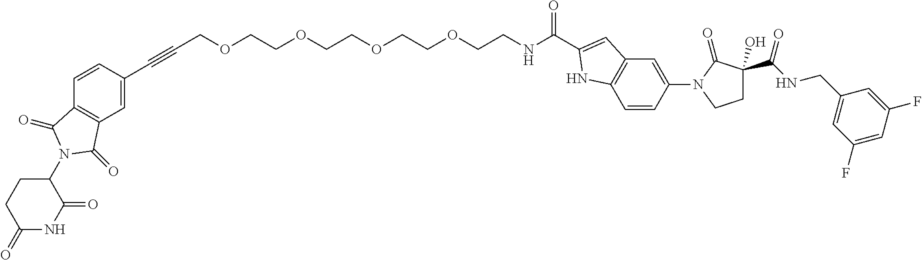 [SMILES: O=C1CCC(N2C(=O)c3ccc(C#CCOCCOCCOCCOCCNC(=O)c4cc5cc(N6CC[C@](O)(C(=O)NCc7cc(F)cc(F)c7)C6=O)ccc5[nH]4)cc3C2=O)C(=O)N1]

LC-MS or HPLC Method:

Method 1:

MS instrument type: SHIMADZU LCMS-2020, Column: Kinetex EVO C18 30*2.1 mm, 5 um, mobile phase A: 0.0375% TFA in water (v/v), B: 0.01875% TFA in Acetonitrile (v/v), gradient: 0.0 min 0% B→0.8 min 95% B→1.2 min 95% B→1.21 min 5% B→1.55 min 5% B, flow rate: 1.5 mL/min, oven temperature: 50° C.; UV detection: 220 nm & 254 nm.

Method 2:

MS instrument type: SHIMADZU LCMS-2020, Column: Kinetex EVO C18 30*2.1 mm, 5 um, mobile phase A: 0.0375% TFA in water (v/v), B: 0.01875% TFA in acetonitrile (v/v), gradient: 0.0 min 0% B→3.0 min 95% B→3.5 min 95% B→3.51 min 5% B→4.0 min 5% B, flow rate: 0.8 mL/min, oven temperature: 50° C.; UV detection: 220 nm & 254 nm.

General Procedure for Preparation of Compound 9c

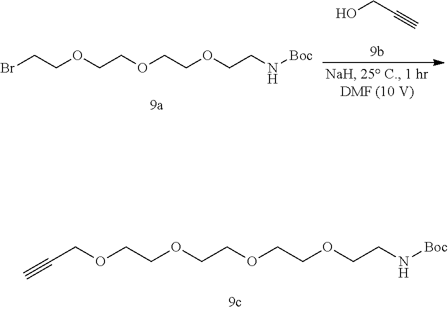

To a solution of Compound 9b (52 mg, 927 umol) in DMF (4 mL) was added NaH (70 mg, 1.75 mmol, 60% purity) at 20-25° C. under N₂ and stirred for 30 mins, then Compound 9a (300 mg, 842 umol, 1.00 eq) was added under N₂ at 20-25° C., after addition, the mixture solution was stirred at 20-25° C. for 1 hr. TLC (Petroleum ether: Ethyl acetate=1:1) showed the Compound 9a was consumed up (R_{f}=0.60), a new spot was detected (R_{f}=0.40). The reaction solution was poured into water (10 mL), then it was extracted with ethyl acetate (10 mL*2), the combined organic layer was washed with brine (20 mL), then it was dried over Na₂SO₄, filtrated and concentrated under reduce pressure to afford the crude product. The crude product was purified by silica gel column chromatography (SiO₂, Petroleum ether:ethyl acetate=20:1-10:1-3:1, the spot (R_{f}=0.40) was collected). Compound 9c (200 mg, 603 umol, 71.7% yield) was obtained as a colorless oil, which was confirmed by ¹H-NMR.

¹H NMR: 400 MHz, CDCl₃ δ 5.09 (s, 1H), 4.23 (d, J=2.4 Hz, 2H), 3.61-3.76 (m, 12H), 3.56 (t, J=5.2 Hz, 2H) 3.34 (d, J=5.2 Hz, 2H), 2.50-2.53 (m, 1H), 1.47 (s, 9H).

General Procedure for Preparation of Compound 9d

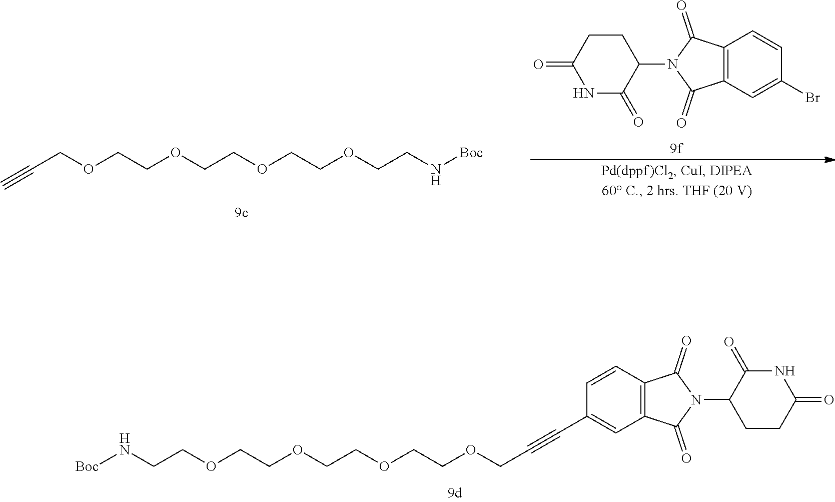 [SMILES: O=C1CCC(N2C(=O)c3ccc(Br)cc3C2=O)C(=O)N1 ; C#CCOCCOCCOCCOCCNC(=O)OC(C)(C)C ; CC(C)(C)OC(=O)NCCOCCOCCOCCOCC#Cc1ccc2c(c1)C(=O)N(C1CCC(=O)NC1=O)C2=O]

To a solution of Compound 9f (187 mg, 554 umol) in THE (5 mL) was added CuI (15 mg, 78.7 umol) and DIPEA (585 mg, 4.53 mmol) at 20-25° C. under N₂, then Pd(dppf)Cl₂.CH₂Cl₂ (38 mg, 46.5 umol) and Compound 9c (150 mg, 452 umol) was added to the solution at 20-25° C. under N₂, then it was stirred at 60-65° C. for 2 hrs. LCMS showed the MS of Compound 9d was detected (RT=0.903 min M/Z+1=588), TLC (Petroleum ether: Ethyl acetate=0:1) showed the Compound 9c (R_{f}=0.67) was consumed up, a new spot was detected (R_{f}=0.30). The reaction solution was cooled to 20-30° C., then it was quenched with acetic acid to pH=3-4 and poured into water (10 mL), then extracted with ethyl acetate (10 mL*2), the combined organic layer was washed with brine (5 mL) dried over Na₂SO₄, filtrated and concentrated under reduce pressure to afford the crude product. Then it was purified by silica gel column chromatography (SiO₂, Petroleum ether:ethyl acetate=10:1-5:1-0:1, the spot (R_{f}=0.30) was collected). Compound 9d (90 mg, 145 umol, 32.1% yield) was obtained as a colorless oil, which was confirmed by LCMS, RT=0.897 min, M/Z+1-100=488), ¹H-NMR.

¹H NMR: 400 MHz, CDCl₃ δ 7.94 (s, 1H), 7.81-7.88 (m, 1H), 7.70-7.79 (m, 1H), 7.26-7.48 (m, 1H), 4.91 (dd, J=12.4, 5.2 Hz, 1H), 4.94-5.00 (m, 1H), 4.40 (s, 1H), 4.21 (s, 1H), 4.13 (s, 1H), 3.61-3.75 (m, 12H), 3.55 (t, J=5.2 Hz, 2H), 3.26-3.36 (m, 2H), 2.71-2.95 (m, 2H), 2.13-2.23 (m, 1H), 1.37 (s, 9H).

LCMS: (Method 1), RT=0.903 min, M/Z+1=588

General Procedure for Preparation of Compound 9e

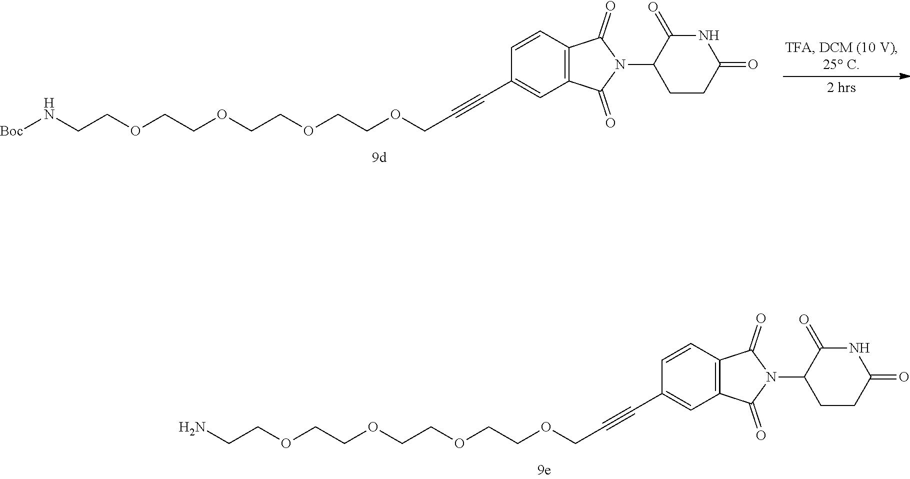 [SMILES: CC(C)(C)OC(=O)NCCOCCOCCOCCOCC#Cc1ccc2c(c1)C(=O)N(C1CCC(=O)NC1=O)C2=O ; NCCOCCOCCOCCOCC#Cc1ccc2c(c1)C(=O)N(C1CCC(=O)NC1=O)C2=O]

To a solution of Compound 9d (90 mg, 153 umol) in DCM (3 mL) was added drop wise TFA (174 mg, 1.53 mmol) at 20-25° C., after addition, the mixture was stirred at 20-25° C. for 2 hrs. TLC (Petroleum ether:Ethyl acetate=0:1) showed the Compound 9d was consumed up (R_{f}=0.60), a new spot was detected (R_{f}=0.00). The reaction solution was concentrated under reduce pressure at 30° C. to afford Compound 9e (90 mg, 110 umol, 72.3% yield, TFA) as a brown oil, which was confirmed by LCMS, RT=0.746 min, M/Z+1=488).

LCMS: (Method 1), RT=0.746 min, M/Z+1=488

General Procedure for Preparation of Title Compound “A43”

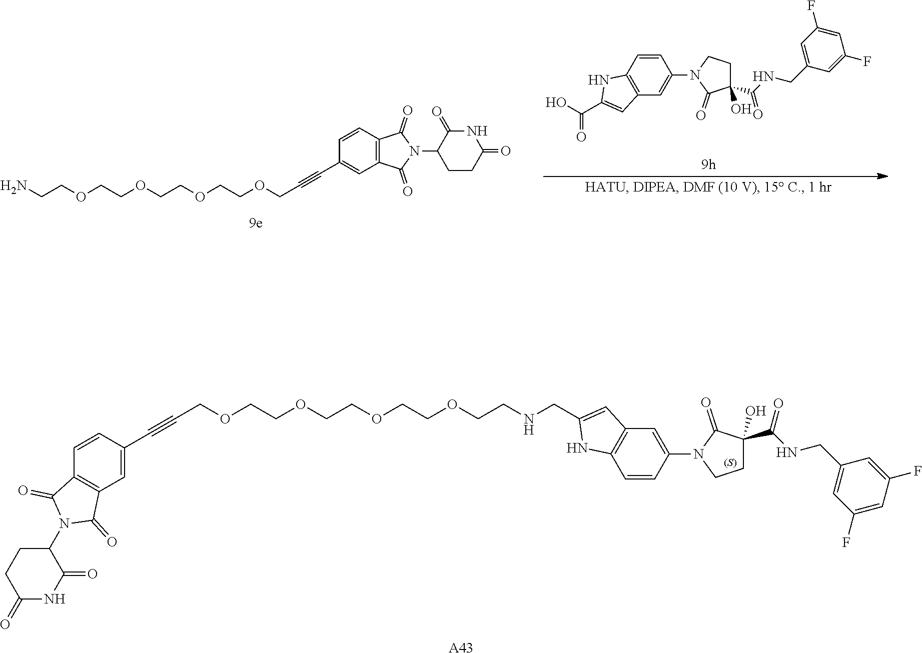

To a solution of Compound 9h (25 mg, 58.2 umol) in DMF (3 mL) was added DIPEA (38 mg, 294 umol) and HATU (34 mg, 89.4 umol) at 10-15° C. under N₂, then Compound 9e (42 mg, 69.8 umol, TFA) dissolved in DMF (1 mL) was added to the solution at 10-15° C., after addition, it was stirred at 10-15° C. for 1 hrs. The LCMS showed the MS of Compound 9 was detected (RT=0.917 min, M/Z+1=899). The reaction solution was quenched with AcOH (2 mL), then it was poured into water (10 mL) and extracted with ethyl acetate (10 mL*2), the combined organic layer was washed with brine (20 mL), then dried over Na₂SO₄, filtrated and concentrated under reduce pressure to afford the crude product. The crude was purified by pre-HPLC (column: Phenomenex Gemini-NX C18 75*30 mm*3 um; mobile phase: [water (0.1% TFA)-ACN]; B %: 35%-45%, 7 min), the solvent was concentrated under reduce pressure to remove the ACN, then it was extracted with ethyl acetate (10 mL*3) washed with brine (10 mL), then dried over Na₂SO₄, filtrated and concentrated under reduce pressure to afford the title compound (33.7 mg, 35.6 umol, 61.1% yield) as a white solid, which was confirmed by LCMS, RT=2.199 min, M/Z+1=899, ¹H-NMR, HPLC (RT=1.856 min).

¹H NMR: 400 MHz, DMSO-d₆ δ 11.62 (s, 1H), 11.14 (s, 1H), 8.48-8.77 (m, 2H), 7.88-8.03 (m, 3H), 7.78 (s, 1H), 7.40-7.59 (m, 2H), 7.00-7.25 (m, 4H), 6.71 (s, 1H), 5.15-5.28 (m, 1H), 4.37-4.54 (m, 2H), 4.24-4.35 (m, 1H), 3.90 (t, J=6.8 Hz, 3H), 3.63-3.71 (m, 2H), 3.48-3.60 (m, 14H), 3.40-3.48 (m, 2H), 2.82-2.99 (m, 1H), 2.59-2.66 (m, 2H), 1.96-2.23 (m, 2H).

LCMS: (Method 2), RT=2.199 min, M/Z+1=899

HPLC: (Method 2), RT=1.856 min

### 5-[(3S)-3-{[(3,5-difluorophenyl)methyl]carbamoyl}-3-hydroxy-2-oxopyrrolidin-1-yl]-N-(14-{[2-(2,6-dioxopiperidin-3-yl)-1,3-dioxo-2,3-dihydro-1H-isoindol-4-yl]amino}-3,6,9,12-tetraoxatetradecan-1-yl)-1H-indole-2-carboxamide (“A44”)

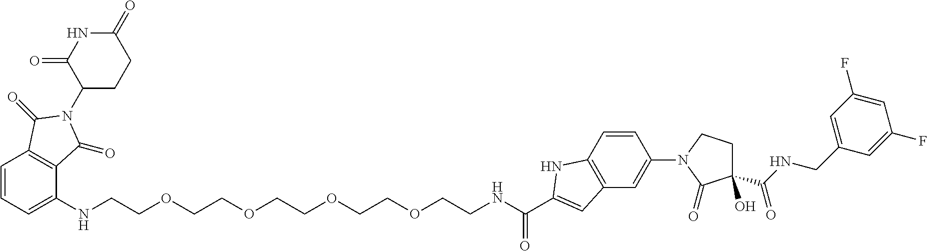 [SMILES: O=C1CCC(N2C(=O)c3cccc(NCCOCCOCCOCCOCCNC(=O)c4cc5cc(N6CC[C@](O)(C(=O)NCc7cc(F)cc(F)c7)C6=O)ccc5[nH]4)c3C2=O)C(=O)N1]

LC-MS or HPLC Method:

Method 1:

MS instrument type: SHIMADZU LCMS-2020; column: Kinetex EVO C18 30*2.1 mm, 5 um; mobile phase A: 0.0375% TFA in water (v/v), B: 0.01875% TFA in Acetonitrile (v/v); gradient: 0.0 min 5% B→0.80 min 95% B→1.20 min 95% B→1.21 min 5% B→1.55 min 5% B; flow rate: 1.5 mL/min; oven temperature: 50° C.; UV detection: 220 nm & 254 nm.

Method 2:

HPLC instrument type: SHIMADZU LC-20AB; column: Kinetex C18 LC Column 4.6×50 mm, 5 um; mobile phase A: 0.0375% TFA in water (v/v), B: 0.01875% TFA in Acetonitrile (v/v); gradient: 0.0 min 10% B→2.40 min 80% B→3.70 min 80% B→3.71 min 10% B→4.00 min 10% B; oven temperature: 50° C.; UV detection: PDA (220 nm&215 nm&254 nm).

General Procedure for Preparation of Compound 4a

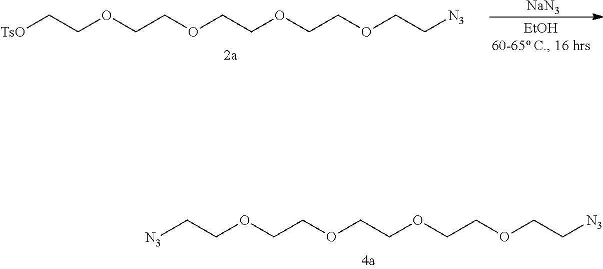 [SMILES: Cc1ccc(S(=O)(=O)OCCOCCOCCOCCOCCN=[N+]=[N-])cc1 ; [N-]=[N+]=NCCOCCOCCOCCOCCN=[N+]=[N-] ; "NaN3"]

To a solution of compound 2a (8.00 g, 19.2 mmol) in EtOH (60.0 mL) was added NaN₃ (2.49 g, 38.3 mmol). The mixture was stirred at 60-65° C. for 16 hrs under N₂. TLC (Petroleum ether/Ethyl acetate=1/2) showed compound 2a (R_{f}=0.75) was consumed up and a new spot (R_{f}=0.80) was formed. The reaction mixture was poured into saturated NaHCO₃ aqueous solution (150 mL), then extracted with MTBE (30.0 mL×5). The organic layer was used directly for next step.

General Procedure for Preparation of Amine 11

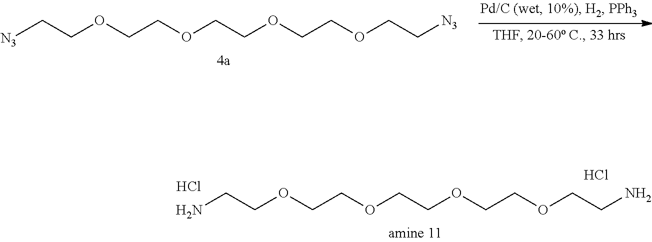 [SMILES: [N-]=[N+]=NCCOCCOCCOCCOCCN=[N+]=[N-] ; Cl ; Cl ; NCCOCCOCCOCCOCCN]

A mixture of compound 4a (2.76 g, MTBE solution, 9.57 mmol, 75.0 mL), Pd/C (200 mg, wet, 10% purity) in THF (75 mL) was degassed and purged with H₂ (15 psi) for 3 times at 20-25° C., then the mixture was stirred for 21 hrs while warming to 55-60° C. under H₂ (15 psi). 1H NMR showed compound 4a was remained. To the mixture was added PPh₃ (5.50 g, 21.0 mmol, 2.19 eq), the mixture was kept stirring at 35-40° C. for 12 hrs. ¹H-NMR showed compound 4a was consumed up and the compound amine 11 was formed. The reaction mixture was filtered over diatomite, the filtrate was concentrated under reduced pressure to give brown residue. Then HCl aqueous solution (1 M, 25 mL) was added into the residue, the mixture was washed with ethyl acetate (40 mL×2). The aqueous layer was separated and lyophilized to give compound amine 11 (1.30 g, 4.20 mmol, 2HCl) as yellow solid, which was confirmed by ¹H-NMR.

¹H NMR: 400 MHz, DMSO-d₆; δ 8.00 (s, 6H) 3.60-3.65 (m, 4H) 3.51-3.60 (m, 12H) 2.92-3.01 (m, 4H)

General Procedure for Preparation of Intermediate 11

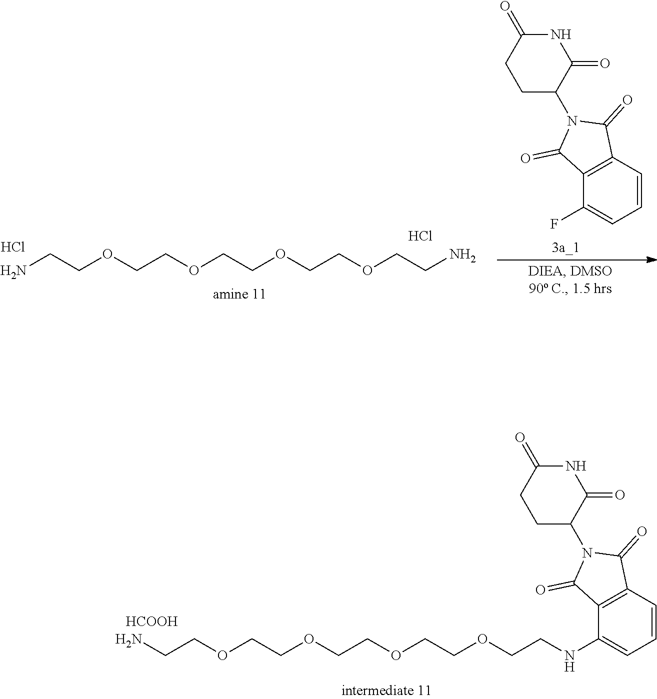 [SMILES: O=C1CCC(N2C(=O)c3cccc(F)c3C2=O)C(=O)N1 ; Cl ; Cl ; NCCOCCOCCOCCOCCN ; "HCOOH" ; NCCOCCOCCOCCOCCNc1cccc2c1C(=O)N(C1CCC(=O)NC1=O)C2=O]

A solution of compound amine 11 (100 mg, 323 umol, 2HCl), DIEA (222 mg, 1.72 mmol, 300 uL) and compound 3a_1 (80 mg, 289 umol) in DMSO (5.00 mL) was stirred at 90° C. under N₂ for 1.5 hrs. LCMS showed MS of compound intermediate 11 (Rt=0.748 min, MS+1=493.3) was detected. The mixture was cooled to 25-30° C. and pH value was adjusted to 5-6 with AcOH, then purified by prep-HPLC (column: Phenomenex Synergi C18 150*25*10 μm; mobile phase: [water (0.225% FA)-ACN]; B %: 7%-37%, 10 min, MS+1=493.3). The fraction was concentrated under reduced pressure to give compound intermediate 11 (45 mg) as brown oil, which was confirmed by next step.

LCMS: (Method 1), Rt=0.748 min, MS+1=493.3;

General Procedure for Preparation of the Title Compound “A44”

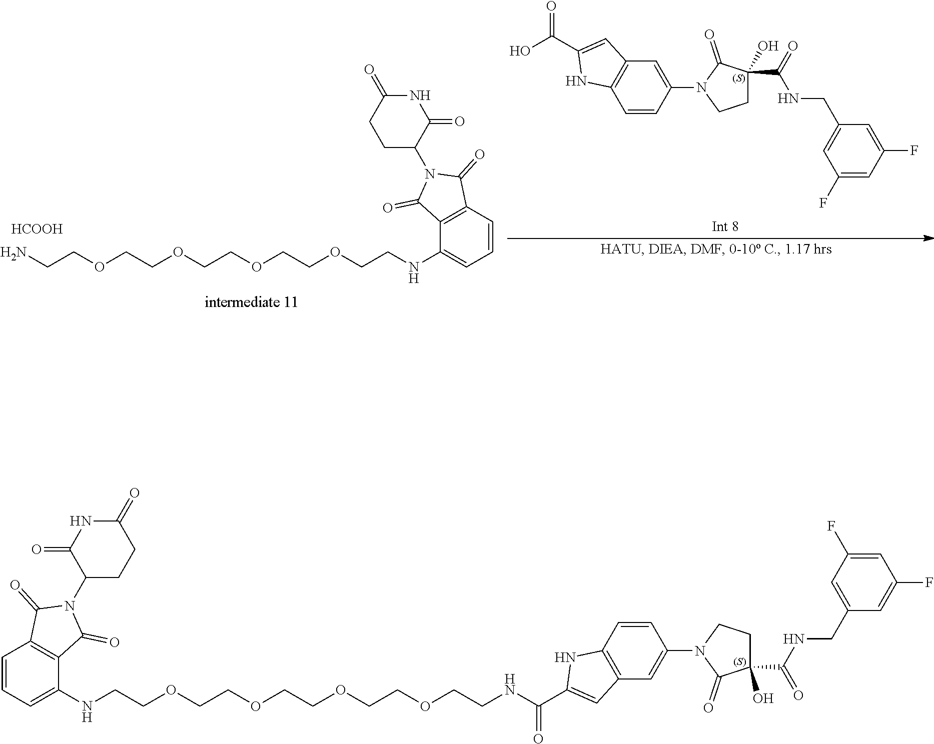

To a solution of compound Int 8 (36 mg, 83.8 umol) in DMF (1.00 mL) was successively added DIEA (35 mg, 271 umol) and HATU (40 mg, 105 umol) at 0-10° C. under N₂, the mixture was stirred at 0-10° C. for 10 mins, then a solution of compound intermediate 11 (45 mg, 83.6 umol, FA) in DMF (1.00 mL) was added into the mixture at 0-10° C., it was stirred for 1 hr under N₂ at 0-10° C. LCMS showed MS of A44 (Rt=0.878 min, MS+1=904.4) was detected. The mixture was poured into icy water (20 mL), extracted with ethyl acetate (10.0 mL×2), the organic phase was separated and washed with brine (20.0 mL×2), dried with Na₂SO₄, filtered, the filtrate was concentrated under reduced pressure to give brown oil. It was purified by prep-HPLC (column: Phenomenex Synergi C18 150*25*10 μm; mobile phase: [water (0.225% FA)-ACN]; B %: 29%-59%, 10 min), the fraction was concentrated under reduced pressure to give A44 (25.88 mg, 99.5% purity) as yellow solid, which was confirmed by ¹H-NMR, ¹⁹F-NMR, LCMS, (Rt=0.884 min, MS+1=904.4) and HPLC (Rt=1.974 min).

¹H NMR: 400 MHz, DMSO-d₆; δ 11.6 (s, 1H), 10.9-11.3 (m, 1H), 8.69 (t, J=6.0 Hz, 1H), 8.55 (t, J=5.2 Hz, 1H), 7.78 (s, 1H), 7.50-7.61 (m, 2H), 7.42 (d, J=8.8 Hz, 1H), 6.93-7.24 (m, 6H), 6.72 (s, 1H), 6.58 (t, J=4.8 Hz, 1H), 5.00-5.10 (m, 1H), 4.37-4.45 (m, 1H), 4.22-4.30 (m, 1H), 3.89 (t, J=6.4 Hz, 2H), 3.46-3.58 (m, 18H), 2.81-2.93 (m, 2H), 2.58 (d, J=15.2 Hz, 3H), 2.09-2.17 (m, 1H), 1.97-2.07 (m, 1H), 1.23 (s, 1H).

¹⁹F NMR: 400 MHz, DMSO-d₆; δ −110 0.29

LCMS: (Method 1), Rt=0.878 min, MS+1=904.4;

HPLC: (Method 2), Rt=1.974 min;

### 5-[(3S)-3-{[(3,5-difluorophenyl)methyl]carbamoyl}-3-hydroxy-2-oxopyrrolidin-1-yl]-N-{2-[2-(2,6-dioxopiperidin-3-yl)-1,3-dioxo-2,3-dihydro-1H-isoindol-4-yl]-5,8,11,14,17-pentaoxa-2-azanonadecan-19-yl}-N-methyl-1H-indole-2-carboxamide (“A45”)

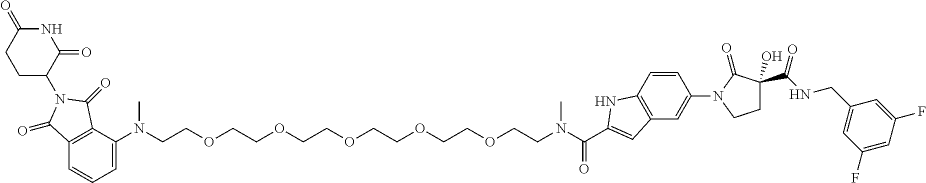 [SMILES: CN(CCOCCOCCOCCOCCOCCN(C)c1cccc2c1C(=O)N(C1CCC(=O)NC1=O)C2=O)C(=O)c1cc2cc(N3CC[C@](O)(C(=O)NCc4cc(F)cc(F)c4)C3=O)ccc2[nH]1]

LC-MS or HPLC Method:

Method 1:

MS instrument type: SHIMADZU LCMS-2020; column: Kinetex EVO C18 30*2.1 mm, 5 um; mobile phase A: 0.0375% TFA in water (v/v), B: 0.01875% TFA in Acetonitrile (v/v); gradient: 0.0 min 5% B→0.80 min 95% B→1.20 min 95% B→1.21 min 5% B→1.55 min 5% B; flow rate: 1.5 mL/min; oven temperature: 50° C.; UV detection: 220 nm & 254 nm.

Method 2:

HPLC instrument type: SHIMADZU LC-20AB; column: Kinetex C18 LC Column 4.6×50 mm, 5 um; mobile phase A: 0.0375% TFA in water (v/v), B: 0.01875% TFA in Acetonitrile (v/v); gradient: 0.0 min 10% B→4.20 min 80% B→5.30 min 80% B→5.31 min 10% B→6.00 min 10% B; oven temperature: 50° C.; UV detection: PDA (220 nm&215 nm&254 nm).

General Procedure for Preparation of Compound 12b

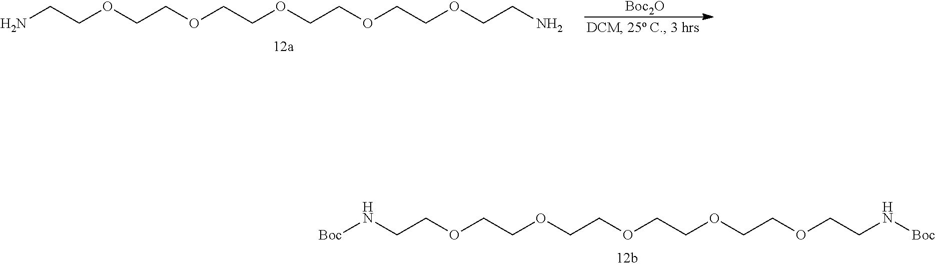 [SMILES: "Boc2O" ; NCCOCCOCCOCCOCCOCCN ; CC(C)(C)OC(=O)NCCOCCOCCOCCOCCOCCNC(=O)OC(C)(C)C]

To a solution of compound 12a (500 mg, 1.78 mmol) in DCM (10 mL) was added Boc₂O (807 mg, 3.70 mmol, 850 uL) at 0-10° C., the mixture was stirred for 3 hrs while warming to 25° C. under N₂. TLC (Petroleum ether/Ethyl acetate=0/1) showed compound 12a (R_{f}=0.00) was consumed up and a new spot (R_{f}=0.30) was formed. The mixture was poured into water (50 mL), it was extracted with ethyl acetate (20 mL×3), the organic phase was washed with brine (50 mL×4), then concentrated under reduced pressure to give brown oil, it was used directly for next step.

General Procedure for Preparation of Compound 12c

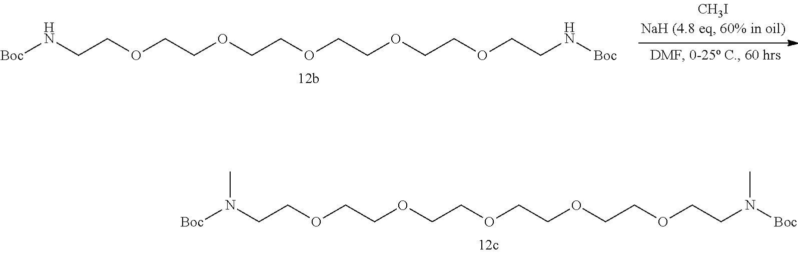 [SMILES: CC(C)(C)OC(=O)NCCOCCOCCOCCOCCOCCNC(=O)OC(C)(C)C ; CN(CCOCCOCCOCCOCCOCCN(C)C(=O)OC(C)(C)C)C(=O)OC(C)(C)C]

To a solution of compound 12b (1.00 g, 2.08 mmol) in DMF (10 mL) was added NaH (200 mg, 5.00 mmol, 60% purity) at 0-10° C. under N₂, it was stirred for 1 hr at 0-10° C. under N₂, then CH₃I (650 mg, 4.58 mmol, 285 uL, 2.20 eq) was added into the mixture, it was stirred for 10 hrs under N₂ while warming to 25° C. TLC (Petroleum ether/Ethyl acetate=0/1) showed compound 12b (R_{f}=0.30) was remain and a new spot (R_{f}=0.40) was formed. Then the mixture was cooled to 0-10° C., NaH (200 mg, 5.00 mmol, 60% purity, 2.40 eq) was added into the mixture under N₂, it was stirred for 1 hr under N₂ at 0-10° C., then CH₃I (1.50 g, 10.6 mmol, 660 uL, 5.08 eq) was added into the mixture at 0-10° C. under N₂, the mixture was stirred for 48 hrs under N₂ while warming to 25° C. TLC (Petroleum ether/Ethyl acetate=0/1) showed compound 12b (R_{f}=0.30) was consumed up and a new spot (R_{f}=0.40) was formed. LCMS (Rt=1.183 min, MS+23=531.4) showed MS of compound 12c was detected. The mixture was poured into water (30 mL), extracted with ethyl acetate (20 mL×3), the organic phase was washed with brine (50 mL×2), then separated and dried with Na₂SO₄, filtered, the filtrate was concentrated under reduced pressure to give brown oil. It was purified by column chromatography (SiO₂, Petroleum ether/Ethyl acetate, 1/0-0/1, R_{f}=0.40) to give compound 12c (400 mg, 786 umol) as a colourless oil, which was confirmed by ¹H-NMR.

¹H NMR: 400 MHz, CDCl₃; δ 3.48-3.68 (m, 20H), 3.37 (d, J=2.8 Hz, 4H), 2.89 (s, 6H), 1.43 (s, 18H)

LCMS: (Method 1), Rt=1.183 min, MS+23=531.4;

General Procedure for Preparation of Compound Amine 12

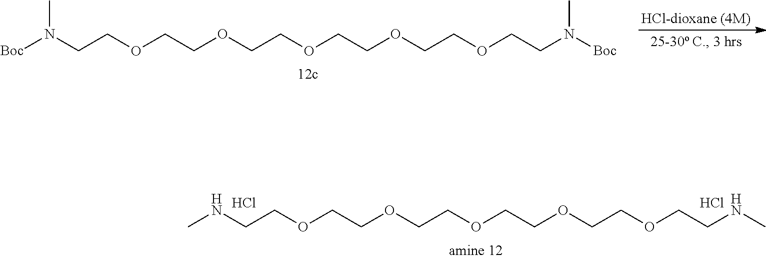

To HCl/dioxane (4 M, 20.0 mL) was added a solution of compound 12c (400 mg, 786 umol) in dioxane (10 mL) at 25-30° C. under N₂, the mixture was stirred at 25-30° C. under N₂ for 3 hrs. 300 μL reaction mixture was taken and concentrated under reduced pressure to give brown oil, it was sent for monitoring by ¹H-NMR. ¹H-NMR showed compound amine 12 was formed. The mixture was concentrated under reduced pressure to give compound amine 12 as brown oil, it was used directly for next step.

¹H NMR: 400 MHz, CDCl₃; δ 3.84-3.96 (m, 4H), 3.69 (s, 10H), 3.67 (s, 6H), 3.20 (s, 4H), 2.78 (t, J=5.6 Hz, 5H), 2.56 (s, 5H).

General Procedure for Preparation of Compound Intermediate 12

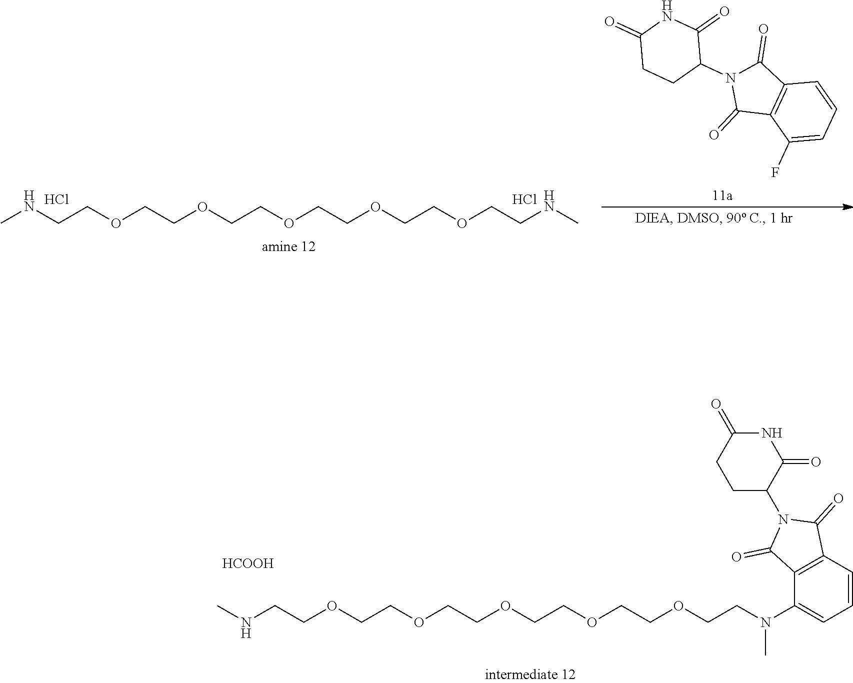

A solution of compound 11a (50 mg, 181 umol), compound amine 12 (200 mg, 524 umol, 2HCl) and DIEA (230 mg, 1.78 mmol, 310 uL) in DMSO (3 mL) was stirred at 90° C. for 1 hr under N₂. LCMS (Rt=0.770 min, MS+1=565.3) showed MS of compound intermediate 12 was detected. The mixture was cooled to 25-30° C., pH value of the mixture was adjusted to 5-6 with AcOH, then it was purified by Prep-HPLC (column: Phenomenex Synergi C18 150*25*10 μm; mobile phase: [water (0.225% FA)-ACN]; B %: 3%-33%, 10 min). The reaction was concentrated under reduced pressure to give compound intermediate 12 (50 mg, 81.9 umol, 45.2% yield, FA) as yellow oil, which was used directly for next step.

LCMS: (Method 1), Rt=0.770 min, MS+1=565.3;

General Procedure for Preparation of the Title Compound “A45”

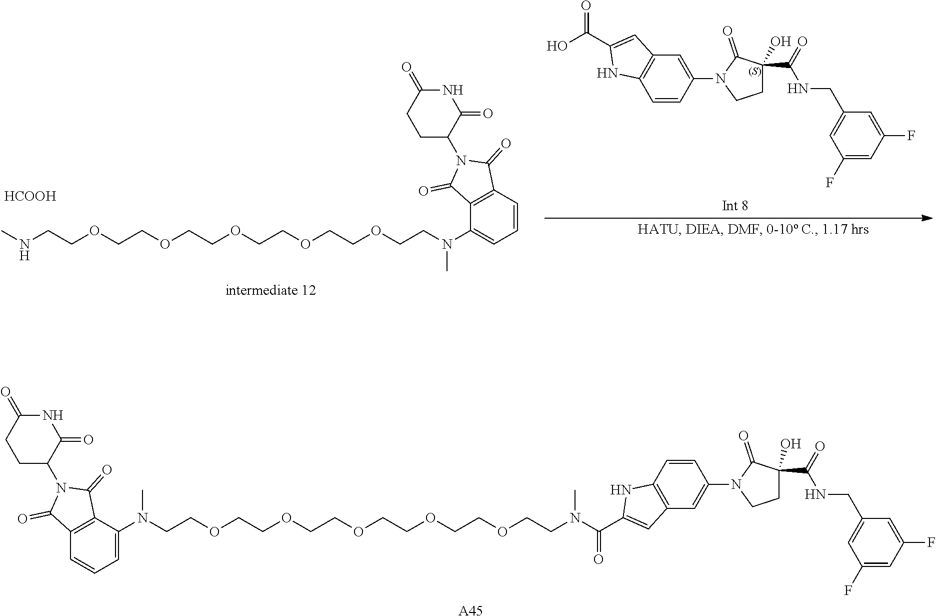

To a solution of compound Int 8 (30 mg, 70.0 umol) in DMF (1.00 mL) was successively added DIEA (37 mg, 287 umol, 50 uL) and HATU (40 mg, 105 umol) at 0-10° C. under N₂, the mixture was stirred at 0-10° C. for 10 mins, then a solution of compound intermediate 12 (45 mg, 73.7 umol, FA) in DMF (1.00 mL) was added into the mixture at 0-10° C., it was stirred for 1 hr under N₂ at 0-10° C. LCMS (Rt=0.897 min, MS+1=976.7) showed MS of A45 was detected. The mixture was poured into icy water (10 mL), extracted with ethyl acetate (20 mL×3), the organic phase was washed with brine (20 mL×3), dried with Na₂SO₄, filtered, the filtrate was concentrated under reduced pressure to give brown oil. It was purified by Prep-HPLC (column: Phenomenex Synergi C18 150*25*10 μm; mobile phase: [water (0.225% FA)-ACN]; B %: 30%-60%, 10 min), the fraction was concentrated under reduced pressure to give A45 (30.51 mg, 31.2 umol, 42.4% yield, 99.9% purity) as yellow solid, which was confirmed by ¹H-NMR, ¹⁹F-NMR, LCMS (Rt=0.901 min, MS+1=976.5) and HPLC (Rt=2.761 min).

¹H NMR: 400 MHz, DMSO-d₆; δ 11.6 (s, 1H), 11.1 (s, 1H), 8.70 (t, J=6.4 Hz, 1H), 7.78 (s, 1H), 7.52-7.61 (m, 2H), 7.42 (d, J=9.2 Hz, 1H), 7.19-7.30 (m, 2H), 6.95-7.11 (m, 3H), 6.89 (s, 1H), 6.72 (s, 1H), 5.08 (dd, J=13.2, 5.2 Hz, 1H), 4.41 (dd, J=15.6, 6.8 Hz, 1H), 4.21-4.29 (m, 1H), 3.89 (t, J=6.8 Hz, 3H), 3.64 (dd, J=7.6, 4.0 Hz, 10H), 3.53 (d, J=2.8 Hz, 3H), 3.47 (s, 2H), 3.41-3.43 (m, 4H), 3.31 (s, 4H), 3.03 (s, 3H), 2.81-2.95 (m, 3H), 2.58-2.65 (m, 4H), 2.10-2.18 (m, 1H), 1.96-2.06 (m, 1H).

¹⁹F NMR: 400 MHz, DMSO-d₆; δ −110.29

LCMS: (Method 1), Rt=0.897 min, MS+1=976.7;

HPLC: (Method 2), Rt=2.761 min.

### 5-[(3S)-3-{[(3,5-difluorophenyl)methyl]carbamoyl}-3-hydroxy-2-oxopyrrolidin-1-yl]-N-{18-[2-(2,6-dioxopiperidin-3-yl)-1,3-dioxo-2,3-dihydro-1H-isoindol-4-yl]-3,6,9,12,15-pentaoxaoctadec-17-yn-1-yl}-1H-indole-2-carboxamide (“A46”)

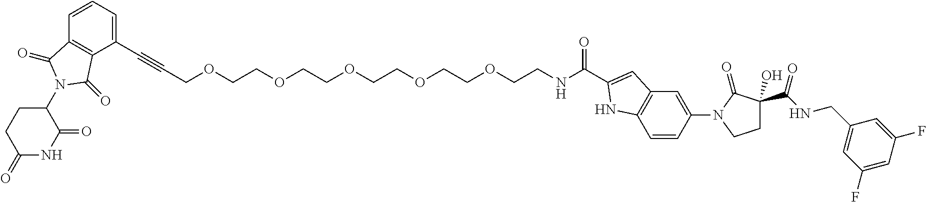 [SMILES: O=C1CCC(N2C(=O)c3cccc(C#CCOCCOCCOCCOCCOCCNC(=O)c4cc5cc(N6CC[C@](O)(C(=O)NCc7cc(F)cc(F)c7)C6=O)ccc5[nH]4)c3C2=O)C(=O)N1]

LC-MS or HPLC Method:

Method 1:

MS instrument type: SHIMADZU LCMS-2020; column: Kinetex EVO C18 30*2.1 mm, 5 um; mobile phase A: 0.0375% TFA in water (v/v), B: 0.01875% TFA in Acetonitrile (v/v); gradient: 0.0 min 5% B→0.80 min 95% B→1.20 min 95% B→1.21 min 5% B→1.55 min 5% B; flow rate: 1.5 (mL/min); oven temperature: 50° C.; UV detection: 220 nm & 254 nm.

Method 2:

HPLC instrument type: SHIMADZU LC-20AB; column: Kinetex C18 LC Column 4.6×50 mm, 5 um; mobile phase A: 0.0375% TFA in water (v/v), B: 0.01875% TFA in Acetonitrile (v/v); gradient: 0.0 min 10% B→4.20 min 80% B→5.30 min 80% B→5.31 min 10% B→6.00 min 10% B; oven temperature: 50° C.; UV detection: PDA (220 nm&215 nm&254 nm).

General Procedure for Preparation of Compound 14c

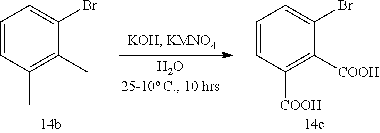

To a solution of compound 14b (10.0 g, 54.0 mmol, 7.30 mL) and KOH (2.39 g, 36.2 mmol, 85.0% purity) in H₂O (500 mL) was dropwise added a solution of KMnO₄ (40.0 g, 252 mmol) in H₂O (100 mL) at 25-30° C. over 0.5 hr, the mixture was stirred at 100° C. for 9.5 hrs. 0.5 mL mixture was taken and was added into 3 mL water, EtOH (1 mL) was added into the solution, pH value of the mixture was adjusted to 1 with 1 M HCl aqueous solution, extracted with ethyl acetate (1 mL), the organic phase was concentrated under reduced pressure to give white solid, it was sent for monitoring by HNMR and LCMS, ¹H-NMR showed compound 14b was consumed up and compound 14c was formed, LCMS showed no MS of compound 14c was detected. The mixture was cooled to 30° C., EtOH (200 mL) was dropwise added into the mixture at 30-40° C. under N₂, it was stirred for further 1 hr, then the mixture was extracted with ethyl acetate (400 mL×2), the organic phase was washed with brine (400 mL×3), dried with Na₂SO₄, filtered, the filtrate was concentrated under reduced pressure to give compound 14c as white solid, which was confirmed by ¹H-NMR.

¹H NMR: 400 MHz, DMSO-d₆; δ 8.00 (dd, J=7.6, 1.2 Hz, 1H), 7.76 (dd, J=8.0, 1.2 Hz, 1H), 7.32 (t, J=8.0 Hz, 1H)

General Procedure for Preparation of Compound 14d

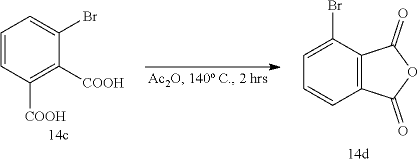 [SMILES: O=C(O)c1cccc(Br)c1C(=O)O ; O=C1OC(=O)c2c(Br)cccc21]

A solution of compound 14c (9.50 g, 38.8 mmol) in Ac₂O (80 mL) was stirred at 140° C. for 2 hrs under N₂. 0.2 mL mixture was taken and sent for monitoring by ¹H-NMR, ¹H-NMR showed compound 14c was consumed up and compound 14d was formed. The mixture was concentrated under reduced pressure to give compound 14d (8 g, crude) as brown solid, which was confirmed by ¹H-NMR.

¹H NMR: 400 MHz, DMSO-d₆; δ 8.18 (d, J=8.0 Hz, 1H), 8.06 (d, J=7.6 Hz, 1H), 7.78-7.90 (m, 1H)

General Procedure for Preparation of Compound 14a

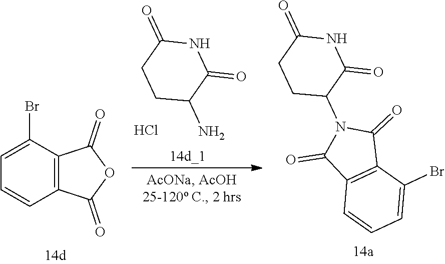

To a solution of compound 14d (650 mg, 2.86 mmol) and compound 14d_1 (630 mg, 3.83 mmol, HCl) in AcOH (15.0 mL) was added AcONa (380 mg, 4.63 mmol) at 25-30° C., the mixture was stirred at 120° C. for 2 hrs under N₂. 0.3 mL mixture was taken and concentrated under reduced pressure to give gray solid, it was sent for monitoring by NMR, ¹H-NMR showed the compound 14a was formed. The mixture was cooled to 25-30° C., then filtered, the filter cake was collected as gray solid. The solid was poured into 0.05 M HCl aqueous solution (50 mL) and stirred for 0.5 hrs, then filtered, the filter cake was washed with water (20 mL), collected and dried under reduced pressure (0.09 MPa) at 60° C. to give compound 14a (800 mg, 2.37 mmol, 82.9% yield) as gray solid, which was confirmed by ¹H-NMR.

¹H NMR: 400 MHz, DMSO-d₆; δ 11.15 (s, 1H), 8.06 (d, J=8.0 Hz, 1H), 7.93 (d, J=7.2 Hz, 1H), 7.72-7.82 (m, 1H), 5.17 (dd, J=12.8, 5.4 Hz, 1H), 2.80-2.97 (m, 1H), 2.52-2.70 (m, 2H), 1.96-2.15 (m, 1H).

General Procedure for Preparation of Compound 14a_1

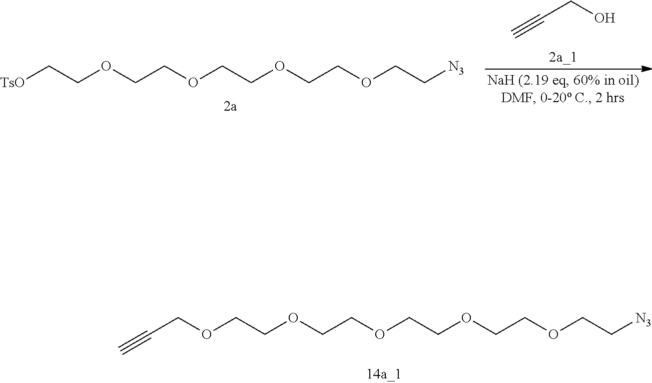

To a solution of compound 2a_1 (1.50 g, 26.7 mmol, 1.58 mL) in DMF (100 mL) was added NaH (2.10 g, 52.5 mmol, 60% purity) in batches at 0-10° C. under N₂, it was stirred for 0.5 hr at 0-10° C. under N₂, compound 2a (10.0 g, 23.9 mmol) was dropwise added into the mixture at 0-10° C. under N₂, the mixture was stirred for 1.5 hrs while warming to 15-20° C. under N₂. LCMS showed no MS of compound 14a_1 was detected. 0.5 mL mixture was taken and poured into icy-water (2 mL), extracted with ethyl acetate (2 mL), the organic phase was separated and concentrated under reduced pressure to give colorless oil, ¹H-NMR showed compound 2a was consumed up and compound 14a_1 was formed. The mixture was poured into water (200 mL), extracted with ethyl acetate (100 mL×2), the organic phase was washed with brine (100 mL×3), dried with Na₂SO₄, filtered, the filtrate was concentrated under reduced pressure to give compound 14a_1 (7.60 g, crude) as brown oil, it was used directly for next step.

¹H NMR: 400 MHz, CDCl₃; δ 4.16 (d, J=2.4 Hz, 2H), 3.59-3.67 (m, 18H), 3.35 (t, J=5.2 Hz, 2H), 2.41 (t, J=2.4 Hz, 1H)

General Procedure for Preparation of Compound 14a_2

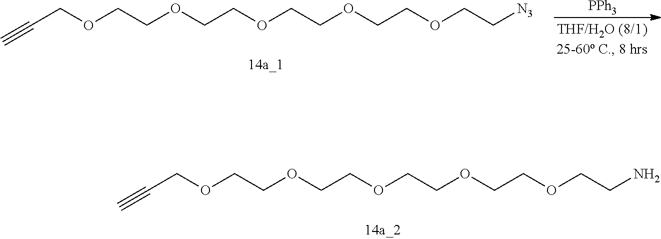 [SMILES: C#CCOCCOCCOCCOCCOCCN=[N+]=[N-] ; C#CCOCCOCCOCCOCCOCCN ; "PPh3"]

To a solution of compound 14a_1 (1.00 g, 3.32 mmol) in a mixed solvent of THE (16 mL) and H₂O (2 mL) was added PPh₃ (1.74 g, 6.64 mmol) at 25° C., the mixture was stirred at 60° C. for 8 hrs under N₂. LCMS (Rt=1.146 min, MS+1=263.2) showed MS of compound 14a_2 was detected. The mixture was poured into water (20 mL), pH value of the mixture was adjusted to 2-3 with 1 M HCl aqueous solution, it was extracted with ethyl acetate (20 mL×3), the aqueous solution was separated and pH value was adjusted to 10-11 with saturated Na₂CO₃ aqueous solution. It was concentrated under reduced pressure to give compound 14a_2 (1 g, crude) as brown oil, which was confirmed by ¹H-NMR.

¹H NMR: 400 MHz, CDCl₃; δ 4.89 (s, 2H), 4.13-4.26 (m, 2H), 3.61-3.69 (m, 16H), 3.49 (t, J=5.2 Hz, 2H), 2.77-2.92 (m, 2H), 2.42 (t, J=2.4 Hz, 1H)

LCMS: (Method 1), Rt=1.146 min, MS+1=263.2;

General Procedure for Preparation of Compound 14a_3

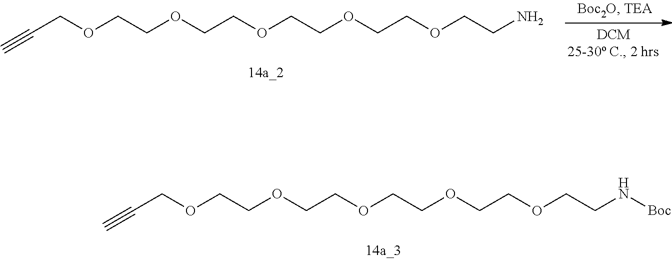 [SMILES: C#CCOCCOCCOCCOCCOCCN ; C#CCOCCOCCOCCOCCOCCNC(=O)OC(C)(C)C]

To a solution of 14a_2 (1 g, 3.63 mmol) in DCM (5.00 mL) was successively added TEA (1.10 g, 10.9 mmol, 1.52 mL) and a solution of Boc₂O (950 mg, 4.35 mmol, 1 mL) in DCM (5 mL) at 25-30° C., the mixture was stirred at 25-30° C. for 2 hrs under N₂. 0.5 mL mixture was taken and poured into water (2 mL), extracted with ethyl acetate (1 mL), the organic phase was separated, and washed with brine (2 mL×3), then separated and concentrated under reduced pressure to give brown oil, ¹H-NMR showed compound 14a_3 was formed. The mixture was poured into water (20 mL), extracted with ethyl acetate (40 mL), the organic phase was separated and washed with brine (30 mL×3), dried with Na₂SO₄, filtered, the filtrate was concentrated under reduced pressure to give brown oil. TLC (Petroleum ether/Ethyl acetate=0/1, R_{f}=0.70). The oil was purified by column chromatography (SiO₂, Petroleum ether/Ethyl acetate, 1/0-0/1, R_{f}=0.70) to give compound 14a_3 (750 mg, 2.00 mmol) as colourless oil, which was confirmed by ¹H-NMR.

¹H NMR: 400 MHz, CDCl₃; δ 5.09 (s, 1H), 4.18 (d, J=2.4 Hz, 2H), 3.59-3.68 (m, 16H), 3.51 (t, J=5.07 Hz, 2H), 3.21-3.35 (m, 2H), 2.41 (t, J=2.4 Hz, 1H), 1.42 (s, 9H).

General Procedure for Preparation of Compound 14a_4

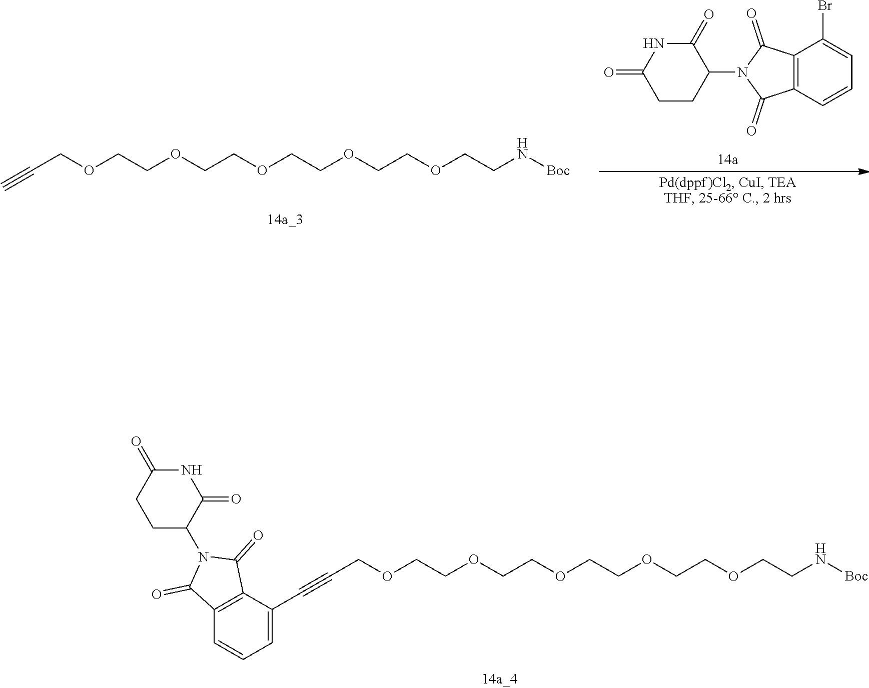 [SMILES: O=C1CCC(N2C(=O)c3cccc(Br)c3C2=O)C(=O)N1 ; C#CCOCCOCCOCCOCCOCCNC(=O)OC(C)(C)C ; "Pd(dppf)Cl2, CuI, TEATHF, 25-66° C., 2 hrs" ; CC(C)(C)OC(=O)NCCOCCOCCOCCOCCOCC#Cc1cccc2c1C(=O)N(C1CCC(=O)NC1=O)C2=O]

To a solution of compound 14a (400 mg, 1.19 mmol) and compound 14a_3 (500 mg, 1.33 mmol) in THF (10.0 mL) was successively added DIEA (1.48 g, 11.5 mmol, 2 mL), CuI (50 mg, 262 umol, 0.22 eq) and Pd(dppf)Cl₂ (100 mg, 136 umol) at 25-30° C., the mixture was stirred at 66° C. under N₂ for 2 hrs. LCMS (Rt=0.878 min, MS−100+1=532.3) showed MS of compound 14a_4 was detected. The mixture was cooled to 25-30° C., then poured into water (30 mL), extracted with ethyl acetate (30 mL×3), the organic phase was washed with brine (30 mL×3), then separated and dried with Na₂SO₄, filtered, the filtrate was concentrated under reduced pressure to give brown oil. The oil was purified by Prep-HPLC (reversed phase chromatography ACN (0%-50%)/H₂O (formic acid 0.1%)), the fraction was concentrated under reduced pressure to give compound 14a_4 (120 mg, 143 umol, 12.1% yield) as brown oil, which was confirmed by LCMS (Rt=0.878 min, MS−100+1=532.3).

LCMS: (Method 1), Rt=0.878 min, MS-100+1=532.3;

General Procedure for Preparation of Compound 14a_5

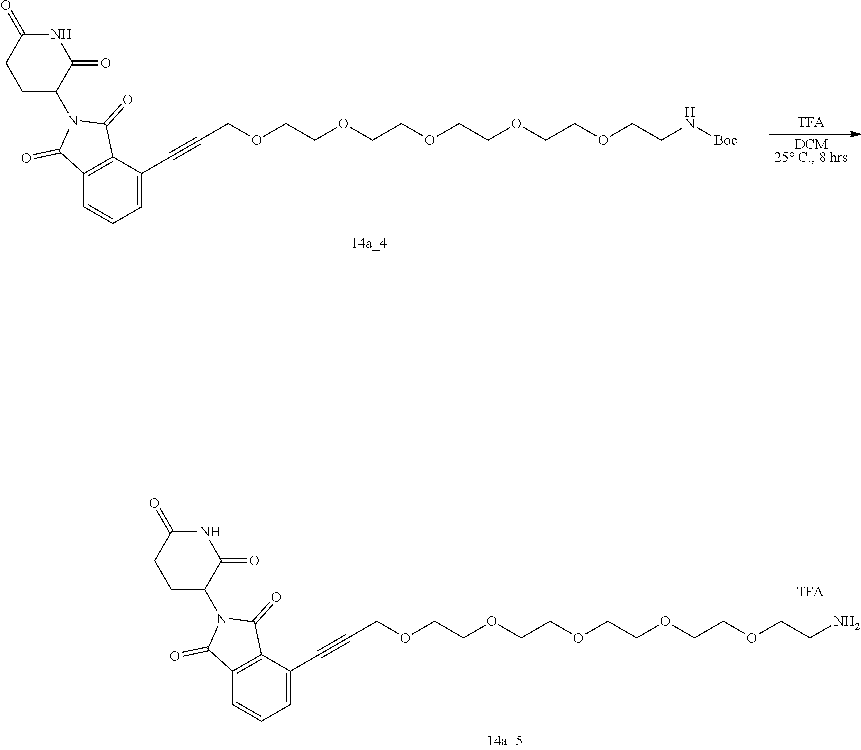 [SMILES: CC(C)(C)OC(=O)NCCOCCOCCOCCOCCOCC#Cc1cccc2c1C(=O)N(C1CCC(=O)NC1=O)C2=O ; "DCM25° C., 8 hrs" ; NCCOCCOCCOCCOCCOCC#Cc1cccc2c1C(=O)N(C1CCC(=O)NC1=O)C2=O ; "TFA" ; "TFA"]

To a solution of compound 14a_4 (120 mg, 143 umol) in DCM (10 mL) was added TFA (1.54 g, 13.5 mmol, 1 mL) at 25° C., the mixture was stirred at 25° C. under N₂ for 8 hrs. LCMS (Rt=0.737 min, MS1=532.3) showed MS of compound 14a_5 was detected. The mixture was concentrated under reduced pressure to give brown oil. The oil was purified by Prep-HPLC (column: Phenomenex luna C18 150*25 mm*10 um; mobile phase: [water (0.1% TFA)-ACN]; B %: 12%-42%, 9 min), the fraction was concentrated under reduced pressure to give compound 14a_5 (50 mg, 77.5 umol, 53.9% yield, TFA) as brown oil, which was confirmed by LCMS (Rt=0.761 min, MS+1=532.2).

LCMS: Rt=0.737 min, MS+1=532.3;

General Procedure for Preparation of the Title Compound “A46”

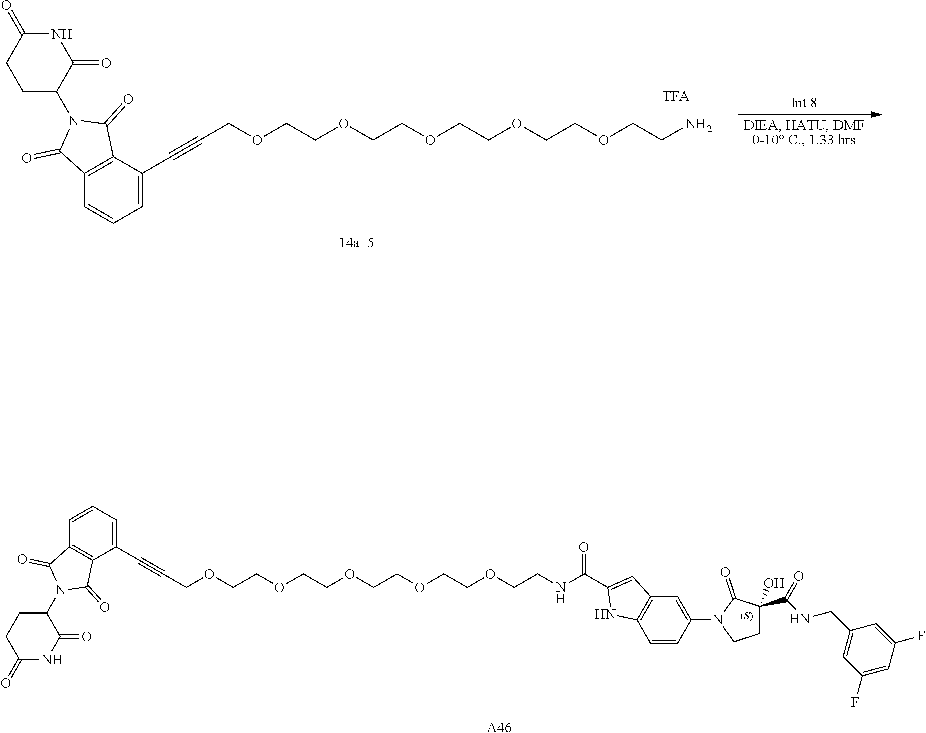

To a solution of compound Int 8 (30 mg, 69.9 umol) in DMF (2 mL) was successively added DIEA (58 mg, 449 umol, 80 uL) and HATU (46 mg, 121 umol) at 0-10° C. under N₂, it was stirred at 0-10° C. for 20 min, a solution of compound 14a_5 (50 mg, 85 umol, FA) in DMF (1.00 mL) was added into the mixture at 0-10° C. under N₂, it was stirred for further 1 hr at 0-10° C. under N₂. LCMS showed the MS value of the title compound (Rt=0.872 min, MS+1=943.4) was detected. The mixture was poured into icy water (20 mL), extracted with ethyl acetate (15 mL×2), the organic phase was washed with brine (30 mL×3), then separated and dried with Na₂SO₄, filtered, the filtrate was concentrated under reduced pressure to give brown oil. The oil was purified by Prep-HPLC (column: Phenomenex Synergi C18 150*25*10 μm; mobile phase: [water (0.225% FA)-ACN]; B %: 28%-58%, 10 min), the fraction was concentrated under reduced pressure to give A46 (13.19 mg, 14.0 μmol, 16.2% yield, 100% purity) as light yellow solid, which was confirmed by ¹H-NMR, ¹⁹F-NMR, LCMS (Rt=0.885 min, MS+1=943.4) and HPLC (Rt=2.671 min).

¹H NMR: 400 MHz, DMSO-d₆; δ 11.6 (s, 1H), 11.1 (s, 1H), 8.69 (t, J=6.4 Hz, 1H), 8.55 (t, J=5.2 Hz, 1H), 7.80-7.97 (m, 3H), 7.77 (s, 1H), 7.48-7.59 (m, 1H), 7.42 (d, J=9.2 Hz, 1H), 6.92-7.17 (m, 4H), 6.71 (s, 1H), 5.00-5.25 (m, 1H), 4.34-4.56 (m, 3H), 4.20-4.33 (m, 1H), 3.89 (t, J=6.8 Hz, 2H), 3.67-3.76 (m, 2H), 3.43-3.60 (m, 18H), 2.85-2.97 (m, 1H), 2.56-2.68 (m, 3H), 2.11-2.20 (m, 1H), 2.04 (s, 1H).

¹⁹F NMR: 400 MHz, DMSO-ds; δ −110.29

LCMS: (Method 1), Rt=0.872 min, MS+1=943.4

HPLC: (Method 2), Rt=2.671 min.

### 5-[(3S)-3-{[(3,5-difluorophenyl)methyl]carbamoyl}-3-hydroxy-2-oxopyrrolidin-1-yl]-N-{2-[2-(2,6-dioxopiperidin-3-yl)-1,3-dioxo-2,3-dihydro-1H-isoindol-5-yl]-5,8,11,14-tetraoxa-2-azahexadecan-16-yl}-N-methyl-1H-indole-2-carboxamide (“A47”)

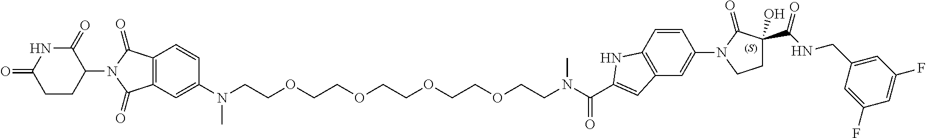

General Procedure for Preparation of Compound 4f;

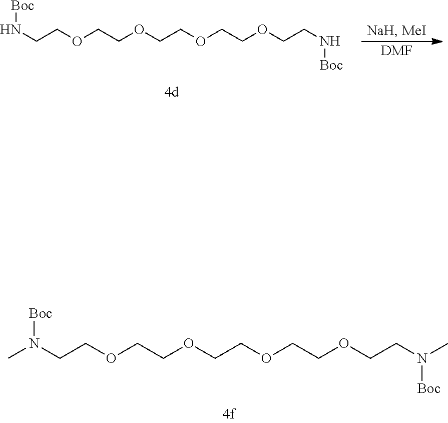

To a solution of compound 4d (500 mg, 1.15 mmol) in DMF (10 mL) was added NaH (230 mg, 5.75 mmol, 60% purity) at 0-10° C. under N₂, the mixture was stirred for 1 hr at 0-10° C. under N₂, then CH₃I (600 mg, 4.23 mmol, 263 uL) was drop-wise added into the mixture at 0-10° C., the mixture was kept stirring for 11 hours under N₂ while warming to 25° C. The mixture was poured into saturated NH₄Cl aqueous solution (30 mL), extracted with ethyl acetate (15 mL×2), the combined organic phase was washed with brine (30 mL×3), the organic phase was separated and dried with Na₂SO₄, then filtered, the filtrate was concentrated under reduced pressure to give brown oil. The oil was purified by column chromatography (SiO₂, Petroleum ether/Ethyl acetate=1/0-1/1, R_{f}=0.50). Compound 4f (500 mg, 1.08 mmol, 94% yield) was obtained as yellow oil.

¹H NMR: 400 MHz, CDCl₃; 3.67-3.54 (m, 16H), 3.39 (d, J=2.2 Hz, 4H), 2.91 (s, 6H), 1.46 (s, 18H)

General Procedure for Preparation of Amine 4

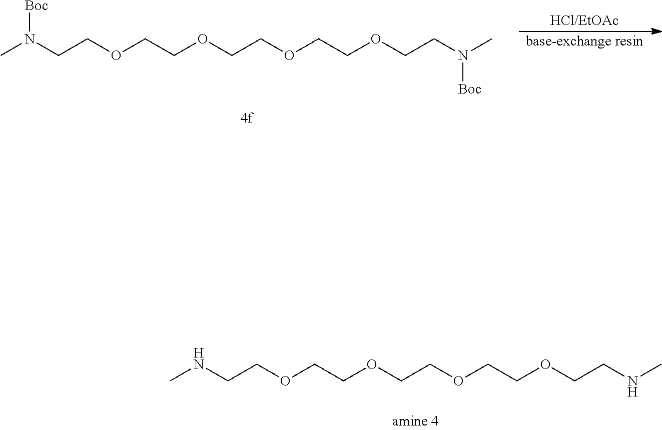

To a solution of compound 4f (500 mg, 1.08 mmol) in EtOAc (5 mL) was added HCl/EtOAc (4 M, 5 mL) at 0-10° C. under N₂, the mixture was stirred for 1 hr at 20° C. under N₂, TLC (Petrleum ether/Ethyl acetate=1/2) the compound 4f (R_{f}=0.4) was consumed up and a new spot (R_{f}=0) was formed. The mixture was concentrated, and diluted with MeOH (20 mL), the solution was alkalized by base-exchange resin, filtered and the filtrate was concentrated. Compound amine 4 (280 mg, crude) was obtained as yellow oil and confirmed with ¹H-NMR.

¹H NMR: 400 MHz, MeOH; δ 3.74-3.80 (m, 4H), 3.68 (d, 10H), 3.16-3.26 (m, 4H), 2.74 (s, 6H)

General Procedure for Preparation of Intermediate 4

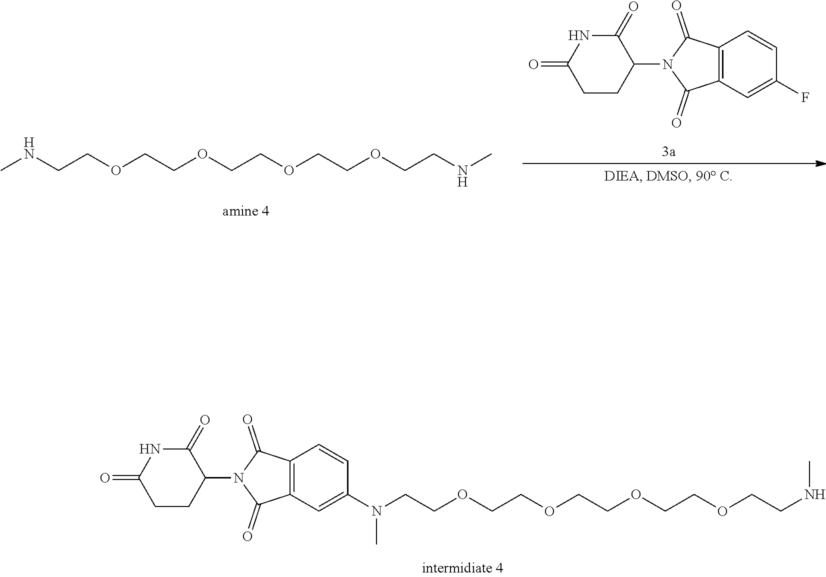

To a solution of compound 3a (250 mg, 905 umol) and amine 4 (250 mg, 946 umol) in DMSO (3 mL) was added DIEA (371 mg, 2.87 mmol, 0.5 mL), and the mixture was stirred at 90° C. for 1 hour. The reaction was diluted with AcOH (1 mL). The mixture was purified with pre-HPLC (column: Phenomenex Synergi C18 150*25*10 um; mobile phase: [water (0.225% FA)-ACN]; B %: 5%-35%, 10 min). Intermediate 4 (130 mg, 229.44 umol, 25% yield, FA) was obtained as yellow gum.

LCMS: RT=0.725 min, m/z (M+1)=521.2

¹H NMR: 400 MHz, DMSO; δ 8.35 (s, 1H), 7.63 (d, J=8.4 Hz, 1H), 7.11 (d, J=2.0 Hz, 1H), 7.02-6.99 (m, 1H), 5.08-5.03 (m, 1H), 3.71-3.68 (m, 2H), 3.63-3.60 (m, 2H), 3.55 (t, J=5.6 Hz, 2H), 3.50-3.48 (m, 12H), 3.09 (s, 3H), 2.93-2.84 (m, 3H), 2.60-2.54 (m, 2H), 2.42 (s, 3H), 2.01-1.99 (m, 1H).

General Procedure for Preparation of “A47”

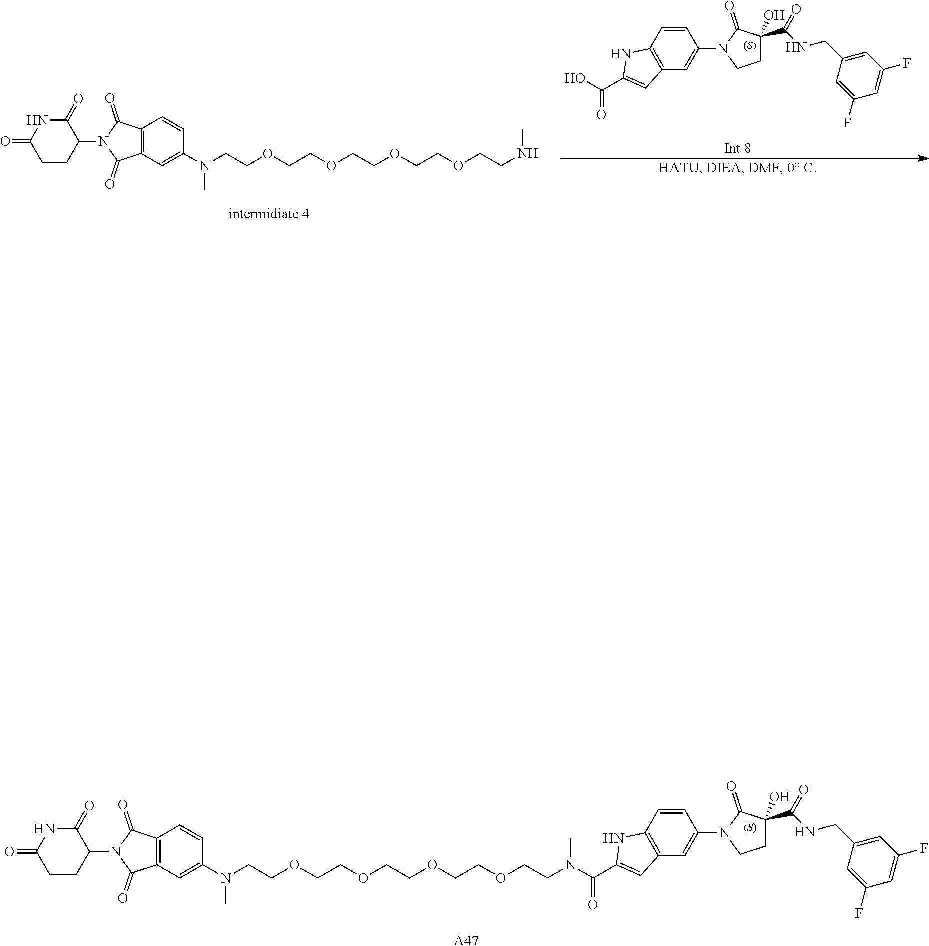

To a solution of Int 8 (40 mg, 93.2 μmol) in DMF (1 mL) was successively added DIEA (35 mg, 271 μmol) and HATU (38 mg, 99.9 μmol) at 0° C. under N₂, the mixture was stirred at 0° C. for 10 min, then a solution of intermediate 4 (50 mg, 88.3 μmol) in DMF (1 mL) was added into the mixture at 0° C., it was stirred for 1 hr under N₂ at 0° C. The reaction mixture was poured into ice water (30 mL) and extracted with EtOAc (20 mL*3). The combined organic layer was washed with brine (20 mL), dried over Na₂SO₄, filtered and concentrated. The mixture was purified by Prep-HPLC (column: 3_Phenomenex Luna C18 75*30 mm*3 um; mobile phase: [water (0.225% FA)-ACN]; B %: 32%-62%, 7 min). “A47” (30.46 mg, 30.9 umol, 35.1% yield, 99.3% purity, FA salt) was obtained as yellow solid.

¹H NMR: 400 MHz, DMSO-d₆; δ 11.56 (s, 1H), 11.18-10.85 (m, 1H), 8.69 (t, J=6.3 Hz, 1H), 7.78 (s, 1H), 7.66-7.51 (m, 2H), 7.42 (d, J=8.8 Hz, 1H), 7.12-6.95 (m, 5H), 6.89 (s, 1H), 6.72 (s, 1H), 5.07-5.02 (m, 1H), 4.44-4.39 (m, 1H), 4.29-4.23 (m, 1H), 3.89 (s, 2H), 3.80-3.46 (m, 23H), 3.05 (s, 3H), 2.94-2.85 (m, 1H), 2.67 (s, 3H), 2.22-2.12 (m, 1H), 2.03-1.97 (m, 1H).

LCMS: RT=0.908 min, m/z (M+1)=932.4;

HPLC: RT=1.948 min.

### (3S)—N-[(3,5-difluorophenyl)methyl]-1-{2-[4-(2-{2-[2-(2-{[2-(2,6-dioxopiperidin-3-yl)-1,3-dioxo-2,3-dihydro-1H-isoindol-5-yl]amino}ethoxy)ethoxy]ethoxy}ethyl)piperazine-1-carbonyl]-1H-indol-5-yl}-3-hydroxy-2-oxopyrrolidine-3-carboxamide (“A48”)

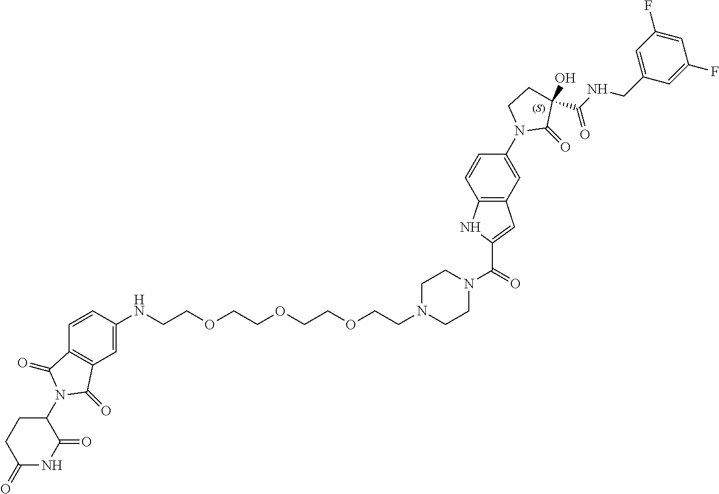

General Procedure for Preparation of Compound 5b—

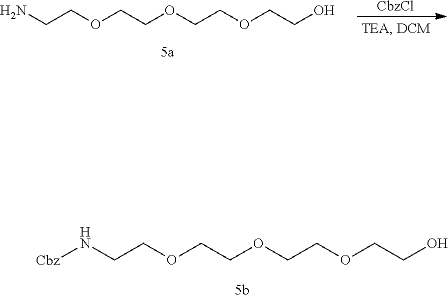

A solution of compound 5a (5 g, 25.9 mmol) and TEA (2.88 g, 28.5 mmol) in DCM (150 mL) was treated with CbzCl (4.80 g, 28.1 mmol) in one portion and the mixture was stirred at 20° C. for 10 hours. The mixture was concentrated and purified by column chromatography (SiO₂, Petroleum ether/Ethyl acetate=2/1 to 0/1), (Petroleum ether/Ethyl acetate=0/1, R_{f}=0.4). Compound 5a (2.50 g, 7.21 mmol, 94.4% purity) was obtained as colorless oil.

LCMS: RT=0.775 min, m/z (M+23)=550.3

General Procedure for Preparation of Compound 5c

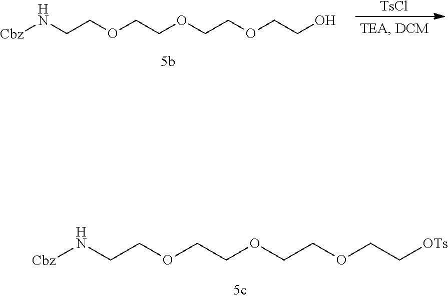 [SMILES: O=C(NCCOCCOCCOCCO)OCc1ccccc1 ; "TsCl" ; "TEA, DCM" ; Cc1ccc(S(=O)(=O)OCCOCCOCCOCCNC(=O)OCc2ccccc2)cc1]

To a solution of compound 5b (2.50 g, 7.64 mmol) and TEA (2.18 g, 21.5 mmol) in DCM (40 mL) was added 4-methylbenzenesulfonyl chloride (2.20 g, 11.5 mmol) at 20° C., the mixture was stirred at 40° C. for 10 hours under N₂. The mixture was concentrated. The residue was purified by column chromatography (SiO₂, Petroleum ether/Ethyl acetate=5/1 to 1/2), (Petroleum ether/Ethyl acetate=1/2, R_{f}=0.5). Compound 5c (2.00 g, 4.15 mmol, 54% yield) was obtained as yellow oil).

¹H NMR: 400 MHz, CDCl₃; δ 7.79 (d, J=8.2 Hz, 2H), 7.36-7.31 (m, 7H), 5.32 (s, 1H), 5.09 (s, 2H), 4.15-4.11 (m, 2H), 3.66-3.63 (m, 2H), 3.60-3.54 (m, 10H), 3.40-3.36 (m, 2H), 2.44 (s, 3H)

General Procedure for Preparation of Compound 5d

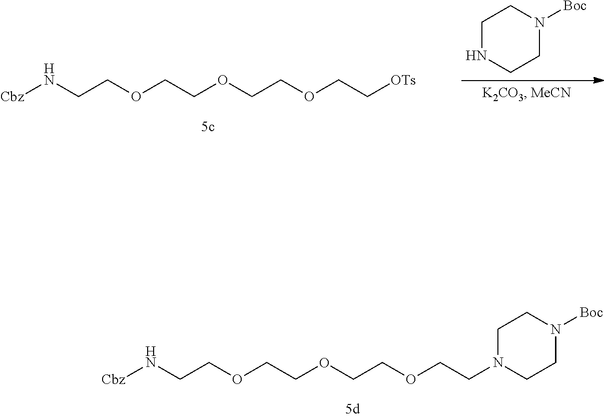

To a solution of compound 5c (1.50 g, 3.11 mmol) and tert-butyl piperazine-1-carboxylate (600 mg, 3.22 mmol) in MeCN (10 mL) was added KI (1.03 g, 6.23 mmol) and K₂CO₃ (1.29 g, 9.34 mmol), the mixture was stirred at 70° C. under N₂ for 12 hours. The mixture was filtered and the filtrate was concentrated. The residue was purified by Reversed-phase chromatographic column (0.1% NH₃.H₂O condition). Compound 5d (500 mg, 32% yield) was obtained as yellow oil.

LCMS: RT=0.989 min, m/z (M+1)=496.5

¹H NMR: 400 MHz, CDCl₃; 7.37-7.27 (m, 5H), 5.58 (s, 1H), 5.10 (s, 2H), 3.65-3.55 (m, 12H), 3.44-3.38 (m, 6H), 2.56 (t, J=5.6 Hz, 2H), 2.42 (d, J=4.4 Hz, 4H), 1.46 (s, 9H)

General Procedure for Preparation of Amine 5—

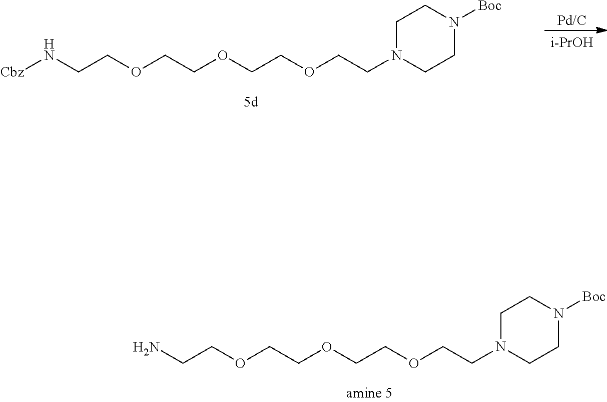 [SMILES: "i-PrOH" ; CC(C)(C)OC(=O)N1CCN(CCOCCOCCOCCNC(=O)OCc2ccccc2)CC1 ; "Pd/C" ; CC(C)(C)OC(=O)N1CCN(CCOCCOCCOCCN)CC1]

To a solution of compound 5d (500 mg, 1.01 mmol) in i-PrOH (20 mL) was added Pd/C (100 mg, 10% purity) under N₂, the suspension under vacuum and purge with H₂ three times, then stirred the mixture under H₂ (15 psi) at 25° C. for 10 hours. The mixture was filtered, and the filtrate was concentrated. Compound amine 5 (360 mg, crude) was obtained as yellow oil.

¹H NMR: 400 MHz, CDCl₃; δ 3.67-3.1 (m, 10H), 3.51 (t, J=5.2 Hz, 2H), 3.44-3.42 (m, 4H), 2.86 (t, J=5.2 Hz, 2H), 2.59 (t, J=5.8 Hz, 2H), 2.45-2.43 (m, 4H), 1.45 (s, 9H)

General procedure for preparation of 1;

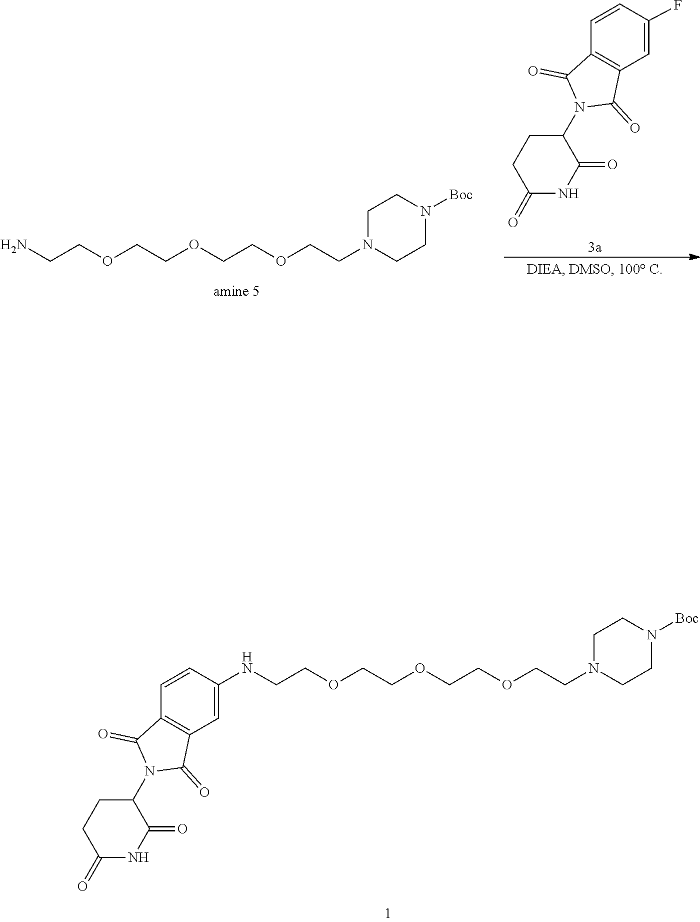 [SMILES: CC(C)(C)OC(=O)N1CCN(CCOCCOCCOCCN)CC1 ; O=C1CCC(N2C(=O)c3ccc(F)cc3C2=O)C(=O)N1 ; "3a" ; "DIEA, DMSO, 100° C." ; CC(C)(C)OC(=O)N1CCN(CCOCCOCCOCCNc2ccc3c(c2)C(=O)N(C2CCC(=O)NC2=O)C3=O)CC1]

To a solution of compound 3a (150 mg, 543 μmol) and DIEA (281 mg, 2.17 mmol, 378 μL) in DMSO (3 mL) was added amine 5 (350 mg, 968 μmol) at 100° C., and the mixture was stirred at 100° C. for 8 hours. LCMS showed desired mass (RT=0.758 min, m/z=618.3) was detected. The reaction mixture was purified with pre-HPLC (column: Phenomenex luna C 18 150*40 mm*15 um; mobile phase: [water (0.225% FA)-A CN]; B %: 9%-39%, 10 min). Compound 1 (85 mg, 121 μmol, 22% yield, 94.35% purity, FA) was obtained as yellow gum and confirmed with LCMS.

LCMS: RT=0.762 min, m/z (M+1)=618.3

General procedure for preparation of 2

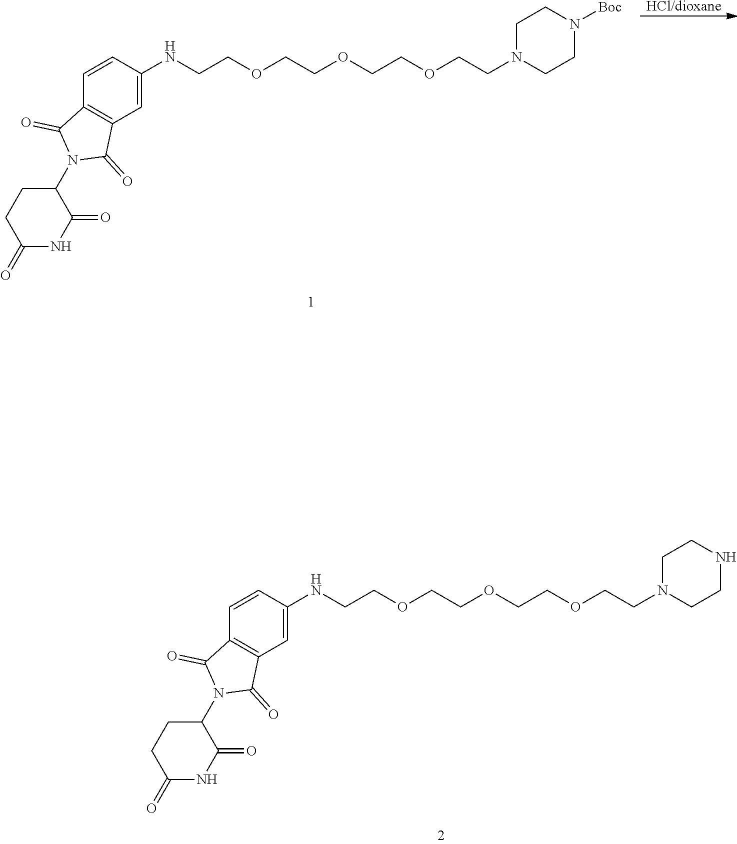 [SMILES: "HCl/dioxane" ; CC(C)(C)OC(=O)N1CCN(CCOCCOCCOCCNc2ccc3c(c2)C(=O)N(C2CCC(=O)NC2=O)C3=O)CC1 ; O=C1CCC(N2C(=O)c3ccc(NCCOCCOCCOCCN4CCNCC4)cc3C2=O)C(=O)N1]

To a solution of compound 1 (80 mg, 130 μmol) in dioxane (2 mL) was added HCl/dioxane (4 M, 2 mL) at 0° C., then the mixture was stirred at 20° C. for 10 mins. The reaction mixture was concentrated. Compound 2 (76 mg, crude, 2HCl) was obtained as yellow gum.

LCMS: RT=0.650 min, m/z (M+1)=518.3

General procedure for preparation of Compound “A48”

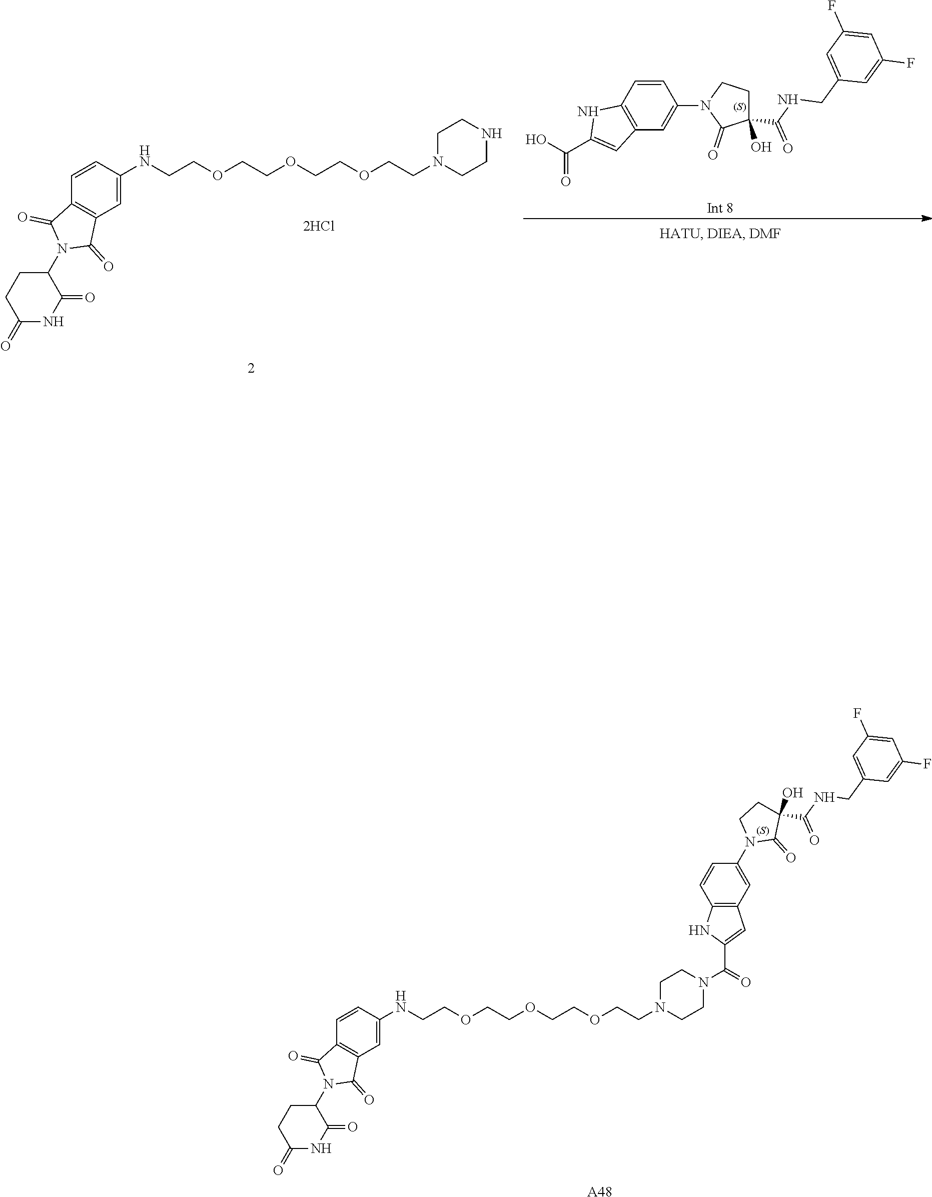

To a solution of Int 8 (52 mg, 120 μmol) in DMF (1 mL) was successively added DIEA (23 mg, 181 μmol, 31.6 μL) and HATU (59 mg, 155 μmol) at 0° C. under N₂, the mixture was stirred at 0° C. for 10 min, then a solution of compound 2 (76 mg, 129 μmol, 2HCl) in DMF (1 mL) was added into the mixture at 0° C., it was stirred for 1 hr under N₂ at 0° C. The reaction mixture was poured into ice water (30 mL) and extracted with EtOAc (20 mL*3). The combined organic layer was washed with brine (20 mL), dried over Na₂SO₄, filtered and concentrated. The mixture was purified by Prep-HPLC (column: Phenomenex Luna C 18 150*25 mm*10 um; mobile phase: [water (0.225% FA)-ACN]; B %: 15%-45%, 10 min). “A48” (35.04 mg, 35.3 μmol, 27% yield, 98% purity, FA) was obtained as yellow solid.

¹H NMR: 400 MHz, DMSO-d₆; δ 11.61 (s, 1H), 11.05 (s, 1H), 8.69 (t, J=6.4 Hz, 1H), 8.24 (s, 1H), 7.78 (s, 1H), 7.56-7.54 (m, 2H), 7.42 (d, J=8.8 Hz, 1H), 7.14 (t, J=5.6 Hz, 1H), 7.08-6.99 (m, 4H), 6.89 (d, J=8.6 Hz, 1H), 6.78 (s, 1H), 5.05-5.00 (m, 1H), 4.44-4.39 (m, 1H), 4.29-4.24 (m, 1H), 3.90-3.87 (m, 2H), 3.60-3.50 (m, 19H), 3.37-3.24 (m, 2H), 2.91-2.82 (m, 2H), 2.63-2.54 (m, 6H), 2.18-2.13 (m, 1H), 1.99-1.97 (m, 1H)

LCMS: RT=0.818 min, m/z (M+1)=932.4;

HPLC: RT=1.487 min.

### (3S)—N-[(3,5-difluorophenyl)methyl]-1-[2-(9-{2-[2-(2-{[2-(2,6-dioxopiperidin-3-yl)-1,3-dioxo-2,3-dihydro-1H-isoindol-5-yl]amino}ethoxy)ethoxy]ethyl}-3,9-diazaspiro[5.5]undecane-3-carbonyl)-1H-indol-5-yl]-3-hydroxy-2-oxopyrrolidine-3-carboxamide (“A49”)

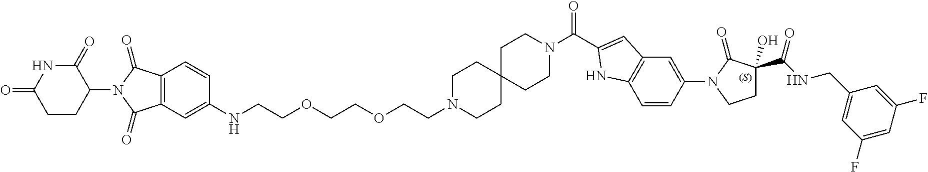

General Procedure for Preparation of Compound 6b

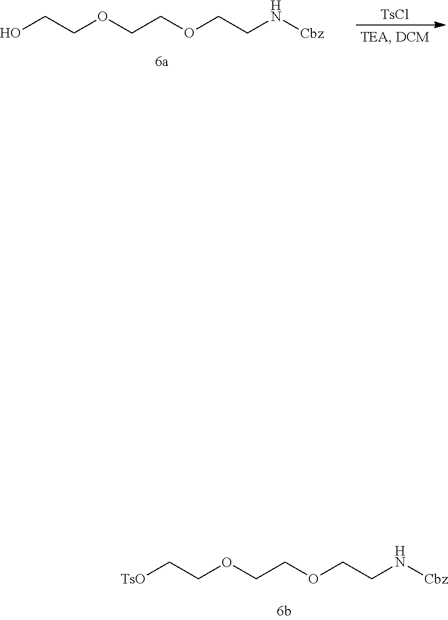 [SMILES: O=C(NCCOCCOCCO)OCc1ccccc1 ; "TsCl" ; "TEA, DCM" ; Cc1ccc(S(=O)(=O)OCCOCCOCCNC(=O)OCc2ccccc2)cc1]

To a solution of compound 6a (1.00 g, 3.53 mmol) and TEA (1 g, 9.88 mmol, 1.38 mL) in DCM (20 mL) was added 4-methylbenzenesulfonyl chloride (1.01 g, 5.29 mmol) at 20° C., the mixture was stirred at 40° C. for 10 hrs under N₂. The mixture was concentrated. The residue was purified by column chromatography (SiO₂, Petroleum ether/Ethyl acetate=5/1 to 1/1), (Petroleum ether/Ethyl acetate=1:1, R_{f}=0.5). Compound 6b (1.40 g, 3.20 mmol, 91% yield) was obtained as yellow oil and confirmed with next step.

General Procedure for Preparation of Amine 4

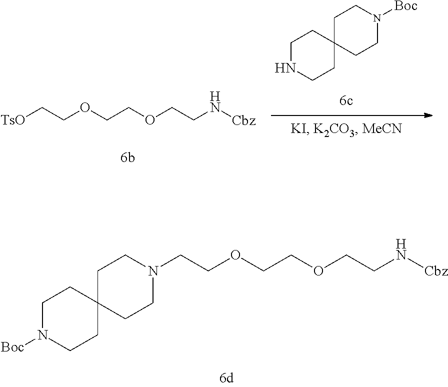 [SMILES: CC(C)(C)OC(=O)N1CCC2(CCNCC2)CC1 ; Cc1ccc(S(=O)(=O)OCCOCCOCCNC(=O)OCc2ccccc2)cc1 ; CC(C)(C)OC(=O)N1CCC2(CCN(CCOCCOCCNC(=O)OCc3ccccc3)CC2)CC1]

To a solution of compound 6b (800 mg, 1.83 mmol) and compound 6c (500 mg, 1.97 mmol) in MeCN (10 mL) was added KI (700 mg, 4.22 mmol) and K₂CO₃ (800 mg, 5.79 mmol), the mixture was stirred at 70° C. under N₂ for 12 hours. The mixture was cooled to 25° C., then poured into water (50 mL), extracted with ethyl acetate (30 mL×2), the combined organic phase was washed with brine (50 mL×3), separated and dried with Na₂SO₄, filtered, the filtrate was concentrated under reduced pressure to give brown oil. The oil was purified by reversed phase chromatography (CH₃CN:H₂O (0.1% NH₃.H₂O contained)=0:1-45:1), the fraction was collected and concentrated under reduced pressure to give colourless oil. Compound 6d (500 mg, 962 μmol, 49% yield) was obtained as yellow oil.

¹H NMR: 400 MHz, CDCl₃; δ 7.42-7.28 (m, 5H), 5.10 (s, 2H), 3.73-3.52 (m, 8H), 3.43-3.27 (m, 6H), 2.61 (t, J=5.4 Hz, 2H), 2.48 (s, 4H), 1.53 (t, J=5.3 Hz, 4H), 1.45 (s, 9H), 1.39 (s, 4H)

General Procedure for Preparation of Amine 6

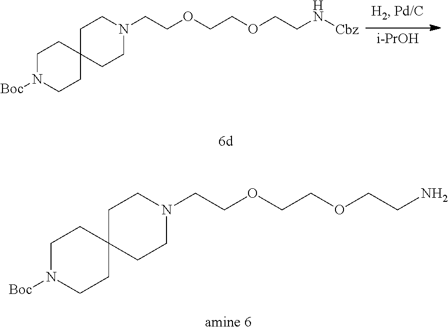 [SMILES: "i-PrOH" ; CC(C)(C)OC(=O)N1CCC2(CCN(CCOCCOCCNC(=O)OCc3ccccc3)CC2)CC1 ; CC(C)(C)OC(=O)N1CCC2(CCN(CCOCCOCCN)CC2)CC1]

To a solution of compound 6d (500 mg, 962 μmol) in i-PrOH (20 mL) was added Pd/C (100 mg, 10% purity) under N₂, the suspension under vacuum and purge with H₂ three times, then stirred the mixture under H₂ (15 psi) at 25° C. for 10 h. The mixture was filtered, and the filtrate was concentrated. Compound amine 6 (350 mg, 908 μmol, 94% yield) was obtained as yellow oil and confirmed with ¹H-NMR.

¹H NMR: 400 MHz, CDCl₃; δ 3.63-3.58 (m, 6H), 3.55-3.49 (m, 2H), 3.36-3.34 (m, 4H), 2.86 (t, J=5.2 Hz, 2H), 2.59 (t, J=6.0 Hz, 2H), 2.45 (s, 4H), 1.54-1.51 (m, 1H), 1.52 (t, J=5.6 Hz, 3H), 1.44 (s, 8H), 1.42-1.39 (m, 4H)

General Procedure for Preparation of Compound 1

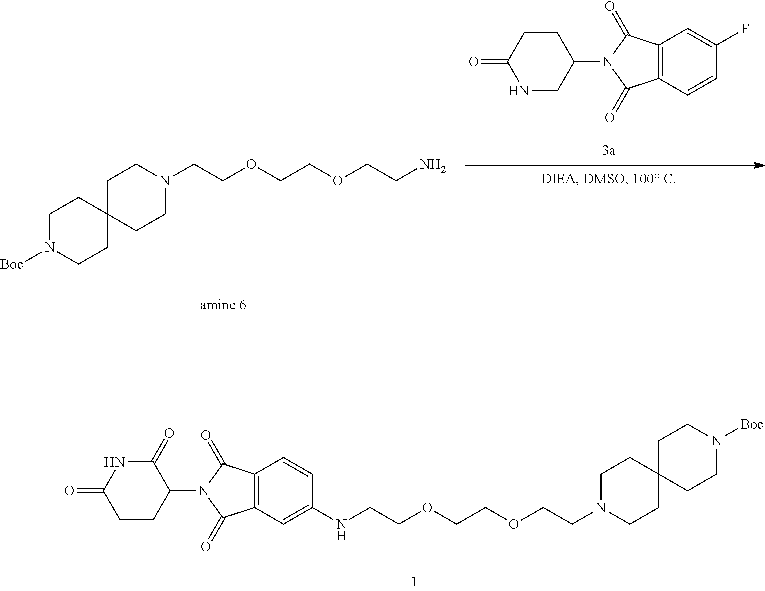 [SMILES: O=C1CCC(N2C(=O)c3ccc(F)cc3C2=O)CN1 ; CC(C)(C)OC(=O)N1CCC2(CCN(CCOCCOCCN)CC2)CC1 ; CC(C)(C)OC(=O)N1CCC2(CCN(CCOCCOCCNc3ccc4c(c3)C(=O)N(C3CCC(=O)NC3=O)C4=O)CC2)CC1]

To a solution of compound 3a (100 mg, 362 μmol) and DIEA (374 mg, 2.90 mmol, 504 μL) in DMSO (1 mL) was added amine 6 (250 mg, 648 μmol) at 100° C., and the mixture was stirred at 100° C. for 12 hours. The reaction mixture was purified by pre-HPLC (column: Phenomenex luna C18 150*25 mm*10 um; mobile phase: [water (0.225% FA)-ACN]; B %: 15%-45%, 10 min). Compound 1 (90 mg, 127 umol, 35% yield, 91% purity) was obtained as yellow solid.

LCMS: RT=0.636 min, m/z (M+1)=642.2

General Procedure for Preparation of Intermediate 6

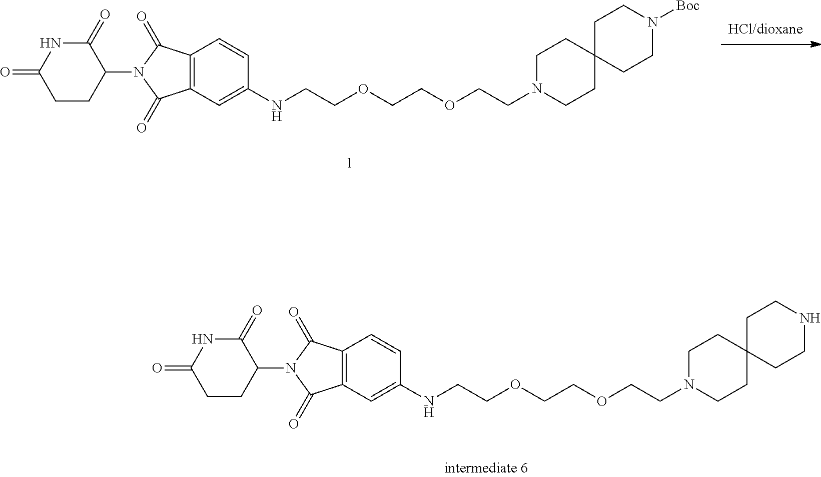 [SMILES: CC(C)(C)OC(=O)N1CCC2(CCN(CCOCCOCCNc3ccc4c(c3)C(=O)N(C3CCC(=O)NC3=O)C4=O)CC2)CC1 ; O=C1CCC(N2C(=O)c3ccc(NCCOCCOCCN4CCC5(CCNCC5)CC4)cc3C2=O)C(=O)N1]

To a solution of compound 1 (80 mg, 106 μmol) in dioxane (2 mL) was added HCl/dioxane (4 M, 1.70 mL) at 0° C. and the mixture was stirred at 20° C. for 1 hour. The reaction mixture was concentrated. Intermediate 6 (61 mg, crude, HCl) was obtained as yellow solid and confirmed with next step.

General Procedure for Preparation of Compound “A49”

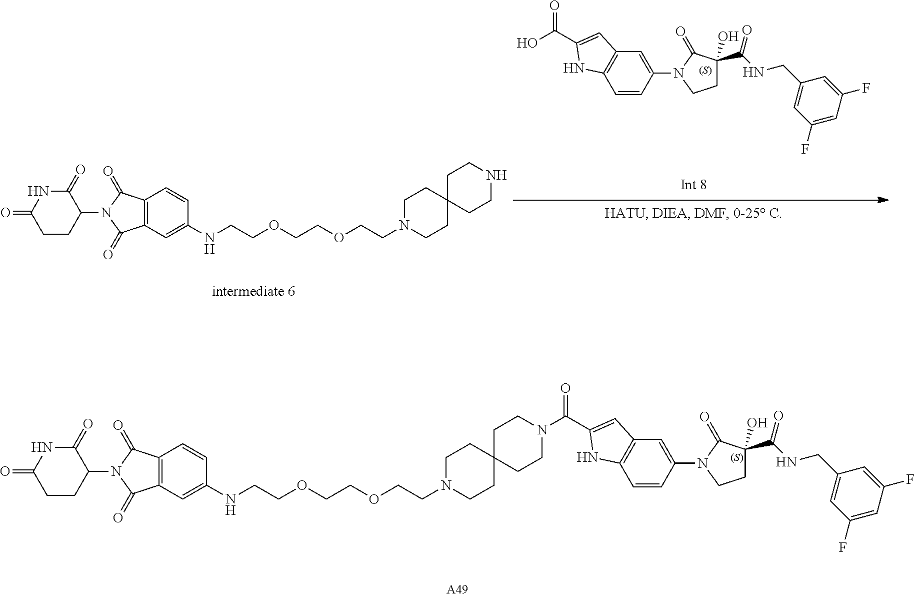

To a solution of Int 8 (45 mg, 105 μmol) in DMF (1 mL) was successively added DIEA (13.4 mg, 104 μmol, 18.1 μL) and HATU (39.5 mg, 104 μmol) at 0° C. under N₂, the mixture was stirred at 0° C. for 10 min, then a solution of intermediate 6 (60 mg, 104 μmol, HCl) in DMF (1 mL) was added into the mixture at 0° C., it was stirred for 1 hr under N₂ at 0° C. The reaction mixture was poured into ice water (30 mL) and extracted with EtOAc (20 mL*3). The combined organic layer was washed with brine (20 mL), dried over Na₂SO₄, filtered and concentrated. The mixture was purified by Prep-HPLC (column: Phenomenex luna C18 150*25 mm*10 um; mobile phase: [water (0.225% FA)-ACN]; B %: 18%-48%, 9 min). “A49” (40.4 mg, 38.4 μmol, 37% yield, 95% purity) was obtained as yellow solid.

¹H NMR: 400 MHz, DMSO-d₆; δ11.58 (s, 1H), 11.05 (s, 1H), 8.70 (t, J=6.4 Hz, 1H), 8.26 (s, 1H), 7.77 (s, 1H), 7.57-7.52 (m, 2H), 7.41 (d, J=8.8 Hz, 1H), 7.17 (t, J=5.4 Hz, 1H), 7.09-7.00 (m, 4H), 6.90 (d, J=8.8 Hz, 1H), 6.76 (s, 1H), 5.05-5.00 (m, 1H), 4.44-4.39 (m, 1H), 4.29-4.24 (m, 1H), 3.88 (d, J=7.2 Hz, 2H), 3.68-3.59 (m, 14H), 3.38-3.35 (m, 1H), 2.81-2.79 (m, 1H), 2.62-2.57 (m, 5H), 2.46-2.45 (m, 1H), 2.39 (s, 3H), 2.18-2.15 (m, 1H), 1.99-1.96 (m, 1H), 1.46 (s, 8H)

LCMS: RT=0.813 min, m/z (M+1)=953.8;

HPLC: RT=1.767 min.

### (3S)—N-[(3,5-difluorophenyl)methyl]-1-{2-[(13-{[2-(2,6-dioxopiperidin-3-yl)-1,3-dioxo-2,3-dihydro-1H-isoindol-5-yl]carbamoyl}tridecyl)carbamoyl]-1H-indol-5-yl}-3-hydroxy-2-oxopyrrolidine-3-carboxamide (“A50”)

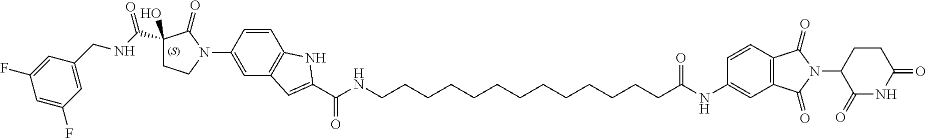

LC-MS or HPLC Method:

Method 1:

MS instrument type: SHIMADZU LCMS-2020, Column: Kinetex EVO C18 30*2.1 mm, Sum, mobile phase A: 0.0375% TFA in water (v/v), B: 0.01875% TFA in Acetonitrile (v/v), gradient: 0.0 min 0% B→0.8 min 95% B→1.2 min 95% B→1.21 min 5% B→1.55 min 5% B, flow rate: 1.5 mL/min, oven temperature: 50° C.; UV detection: 220 nm & 254 nm.

Method 2:

MS instrument type: SHIMADZU LCMS-2020, Column: Kinetex EVO C18 30*2.1 mm, 5 um, mobile phase A: 0.0375% TFA in water (v/v), B: 0.01875% TFA in acetonitrile (v/v), gradient: 0.0 min 0% B→3.0 min 95% B→3.5 min 95% B→3.51 min 5% B→4.0 min 5% B, flow rate: 0.8 mL/min, oven temperature: 50° C.; UV detection: 220 nm & 254 nm.

4. Experimental for Largest Scale Run:

General Procedure for Preparation of Compound 1b

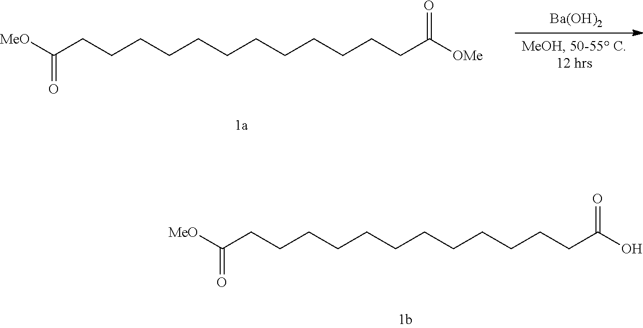 [SMILES: COC(=O)CCCCCCCCCCCCC(=O)OC ; "Ba(OH)2" ; COC(=O)CCCCCCCCCCCCC(=O)O]

To a solution of compound 1a (3 g, 10.5 mmol) in MeOH (100 mL) was added Ba(OH)₂ (1.08 g, 6.28 mmol) at 20-25° C., after addition, the mixture was stirred at 50-55° C. for 12 hrs. TLC (Petroleum ether: Ethyl acetate=5:1) showed trace of compound 1a (R_{f}=0.60) was not consumed up completely, a new spot was detected (R_{f}=0.10). The reaction solution was filtrated and the filter cake was washed with MeOH (20 mL) and stirred in 1N HCl (50 mL) for 30 mins. Then it was filtrated, the filter cake was concentrated under reduce pressure which was used to next step directly. Compound 1b (2.30 g, 8.44 mmol, 80.6% yield) was obtained as a white solid, which was confirmed by ¹H NMR.

¹H NMR: 400 MHz, CDCl₃ δ 3.69 (d, J=4.0 Hz, 3H), 2.25-2.54 (m, 5H), 1.29 (s, 19H).

General Procedure for Preparation of Compound 1c

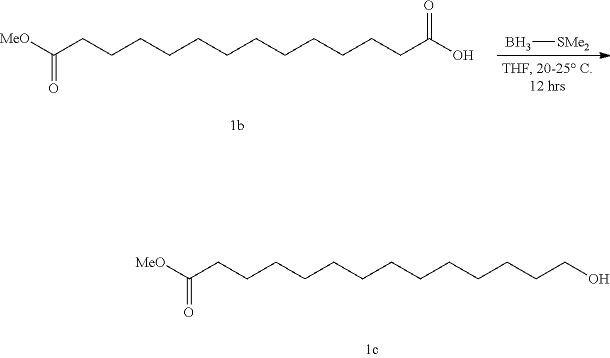

To a solution of compound 1b (2.30 g, 8.44 mmol) in THF (23 mL) was added BH₃-Me₂S (10 M, 1.01 mL) at 15-20° C. under N₂, after addition, the mixture was stirred at 20-25° C. for 12 hrs under N₂ TLC (Petroleum ether: Ethyl acetate=5:1) showed the compound 1b (R_{f}=0.10) was consumed up, a new spot was detected (R_{f}=0.20). The reaction solution was poured into saturated NH₄Cl (50 mL), then it was extracted with ethyl acetate (50 mL*5) and washed with brine (30 mL), dried over Na₂SO₄, filtrated and concentrated under reduce pressure to afford compound 1c (1.60 g, crude) as a white solid which was used to next step directly.

General Procedure for Preparation of Compound 1d

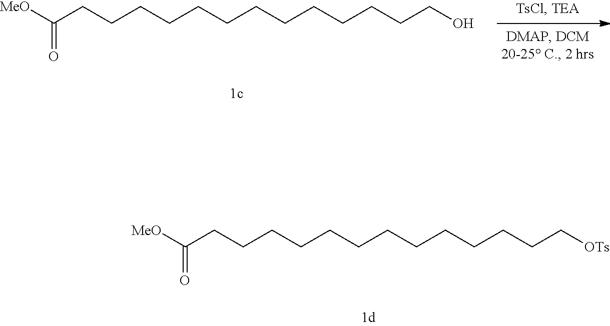 [SMILES: COC(=O)CCCCCCCCCCCCCO ; COC(=O)CCCCCCCCCCCCCOS(=O)(=O)c1ccc(C)cc1]

To a solution of compound 1c (1.60 g, 6.19 mmol) and TsCl (2.40 g, 12.6 mmol) in DCM (50 mL) was added Et₃N (1.92 g, 18.9 mmol) and DMAP (400 mg, 3.27 mmol) at 20-25° C., after addition the mixture solution was stirred at 20-25° C. for 2 hrs. TLC (Petroleum ether: Ethyl acetate=5:1) showed the compound 1c (R_{f}=0.25) was consumed up, a new spot was detected (R_{f}=0.65). The reaction solution was poured into water (200 mL), then it was extracted with ethyl acetate (50 mL*2) and the organic layer was washed with 1 N HCl to pH=6-7, and washed with brine (50 mL), dried over Na₂SO₄, filtrated and concentrated under reduce pressure to afford the crude product. It was purified by silica gel column chromatography (SiO₂, Petroleum ether:ethyl acetate=50:1-25:1-10:1), the spot of (R_{f}=0.65) was collected. Compound 1d (2 g, 4.85 mmol, 78.3% yield) was obtained as a white solid, which was confirmed by ¹H NMR.

¹H NMR: 400 MHz, CDCl₃ δ 7.81 (d, J=8.4 Hz, 2H), 7.37 (d, J=8.0 Hz, 2H), 4.04 (t, J=6.4 Hz, 2H), 3.69 (s, 3H), 2.47 (s, 3H), 2.32 (t, J=7.6 Hz, 2H), 1.61-1.73 (m, 4H), 1.17-1.41 (m, 18H).

General Procedure for Preparation of Compound 1e

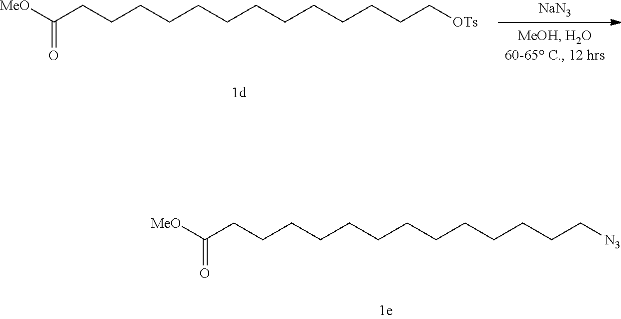 [SMILES: COC(=O)CCCCCCCCCCCCCOS(=O)(=O)c1ccc(C)cc1 ; COC(=O)CCCCCCCCCCCCCN=[N+]=[N-] ; "NaN3"]

To a solution of compound 1d (2 g, 4.85 mmol) in MeOH (20 mL) and H₂O (2 mL) was added NaN₃ (470 mg, 7.23 mmol) at 20-25° C., after addition, the mixture was stirred at 60-65° C. for 12 hrs under N₂. The crude ¹H NMR (0.5 mL solution poured into 1 mL water then extracted with 1 mL ethyl acetate, then concentrated) showed the compound 1e was detected.

The reaction solution was used to next step directly.

¹H NMR: 400 MHz, CDCl₃ δ 3.69 (s, 3H), 3.27 (t, J=7.2 Hz, 2H), 2.32 (t, J=7.6 Hz, 2H), 1.55-1.72 (m, 4H), 1.19-1.44 (m, 18H).

General Procedure for Preparation of Compound 1f

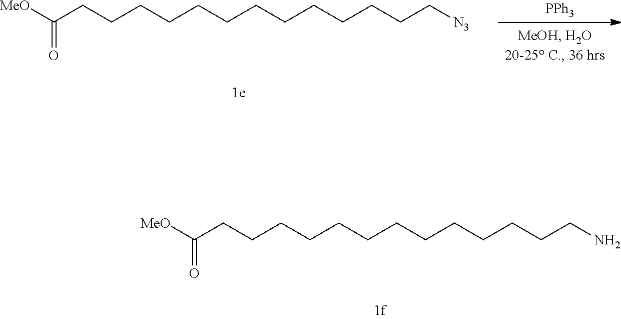 [SMILES: COC(=O)CCCCCCCCCCCCCN=[N+]=[N-] ; COC(=O)CCCCCCCCCCCCCN ; "PPh3"]

The solution of last step was added PPh₃ (2.41 g, 9.17 mmol) at 20-25° C., after addition, the mixture solution was stirred at 20-25° C. for 36 hrs. The crude ¹H NMR (0.5 mL reaction solution was poured into 1 mL water then extracted with 1 mL ethyl acetate, then concentrated) showed the compound 1f was detected. The reaction solution was poured into water (20 mL) and adjust with 1N HCl to pH=2, then it was extracted with ethyl acetate (20 mL*2), the aqueous phase was adjust with NaHCO₃ solution to pH=7 and extracted with ethyl acetate (20 mL*2), then it was concentrated under reduce pressure to afford the crude product.

Compound 1f (1 g, 3.88 mmol, 84.7% yield) was obtained as a white solid.

¹H NMR: 400 MHz, CDCl₃ δ 3.57 (s, 3H), 2.16-2.29 (m, 4H), 1.44-1.61 (m, 4H), 1.01-1.32 (m, 18H).

General Procedure for Preparation of Compound 1g

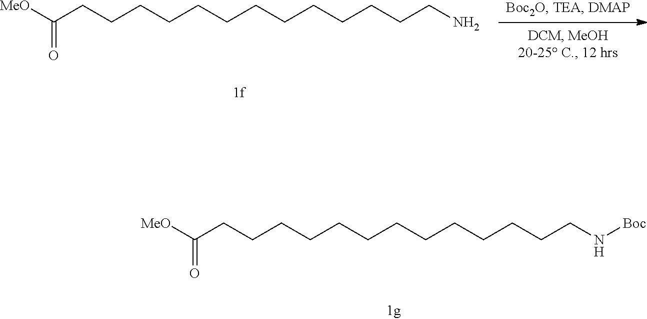 [SMILES: COC(=O)CCCCCCCCCCCCCN ; "Boc2O, TEA, DMAP" ; COC(=O)CCCCCCCCCCCCCNC(=O)OC(C)(C)C]

To a solution of compound 1f (1 g, 3.40 mmol) in DCM (10 mL) and MeOH (1 mL) was added Boc₂O (1.11 g, 5.10 mmol), DMAP (84 mg, 687 umol) and Et₃N (1.03 g, 10.2 mmol), then it was stirred at 20-25° C. for 12 hrs. TLC (Petroleum ether: Ethyl acetate=20:1) showed a new spot was detected (R_{f}=0.60), the compound 1f was consumed up (R_{f}=0.10). The reaction solution was poured into water (20 mL) and extracted with ethyl acetate (20 mL*2), then the organic layer was washed with 1 N HCl to pH=7 and washed with brine (20 mL), dried over Na₂SO₄, filtrated and concentrated under reduce pressure to afford Compound 1g (900 mg, crude) as a white solid, which was confirmed by ¹H NMR.

¹H NMR: 400 MHz, CDCl₃ δ 4.41 (s, 1H), 3.60 (s, 3H), 3.03 (q, J=7.2 Hz, 2H), 2.23 (t, J=7.6 Hz, 2H), 1.50-1.63 (m, 2H), 1.34-1.44 (m, 11H), 1.13-1.29 (m, 18H).

General Procedure for Preparation of Compound 1h

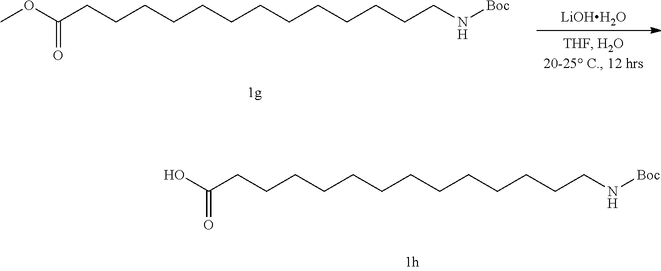 [SMILES: COC(=O)CCCCCCCCCCCCCNC(=O)OC(C)(C)C ; "LiOH·H2O" ; CC(C)(C)OC(=O)NCCCCCCCCCCCCCC(=O)O]

To a solution of compound 1g (900 mg, 2.52 mmol) in THE (10 mL) and H₂O (2 mL) was added LiOH.H₂O (126 mg, 3 mmol) at 20-25° C., after addition, the mixture was stirred at 20-25° C. for 12 hrs. TLC (Petroleum ether: Ethyl acetate=2:1) showed the compound 1g was consumed up (R_{f}=0.90), a new spot was detected (R_{f}=0.30). The reaction solution was poured into water (20 mL) and adjust with 1N HCl to pH=2, then it was extracted with ethyl acetate (10 mL*2), the organic layer was washed with brine (10 mL), dried over Na₂SO₄, filtrated and concentrated under reduce pressure. The crude was purified by silica gel column chromatography (SiO₂, Petroleum ether:ethyl acetate=10:1-5:1-2:1), the spot (R_{f}=0.30) was collected to afford Compound 1h (600 mg, 1.75 mmol, 69.4% yield) as a white solid.

General Procedure for Preparation of Compound 1m

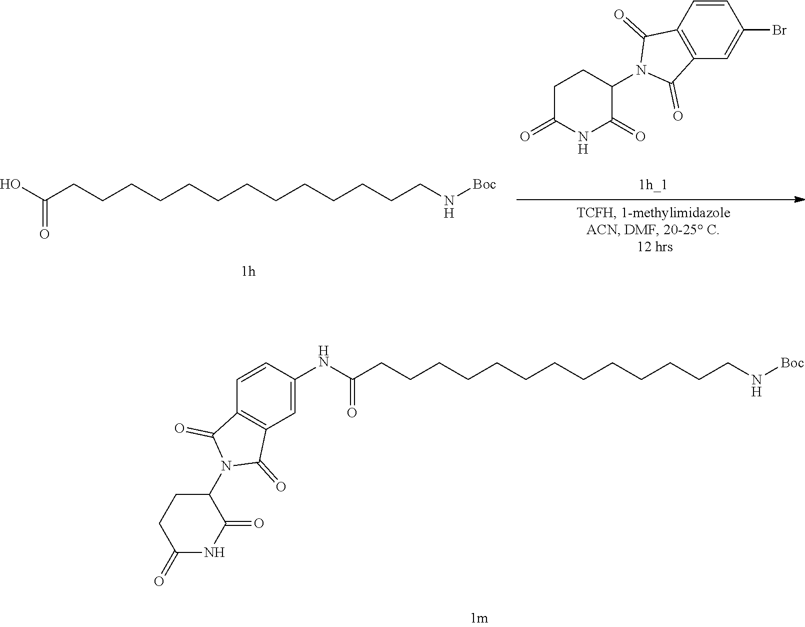 [SMILES: O=C1CCC(N2C(=O)c3ccc(Br)cc3C2=O)C(=O)N1 ; CC(C)(C)OC(=O)NCCCCCCCCCCCCCC(=O)O ; CC(C)(C)OC(=O)NCCCCCCCCCCCCCC(=O)Nc1ccc2c(c1)C(=O)N(C1CCC(=O)NC1=O)C2=O]

To a solution of compound 1h (80 mg, 232 umol) and Compound 1h_1 (60 mg, 219 umol) in ACN (4 mL) and DMF (2 mL) was added TCFH (151 mg, 538 umol) and NMI (125 mg, 1.52 mmol) under N₂ at 20-25° C., after addition, the mixture was stirred at 20-25° C. for 12 hrs. The reaction solution was quenched with AcOH (2 mL), then it was poured into water (10 mL) and extracted with ethyl acetate (10 mL*2), the organic layer was washed with brine (10 mL), dried over Na₂SO₄, filtrated and concentrated under reduce pressure. The crude was purified by Pre-HPLC (column: Phenomenex luna C18 150*25 mm*10 um; mobile phase: [water (0.1% TFA)-ACN]; B %: 59%-89%, 10 min), then it was concentrated under reduce pressure to afford Compound 1m (40 mg) as a colorless oil.

LCMS: (method 1), RT=1.125 min, m/z=543

General Procedure for Preparation of Compound 1n

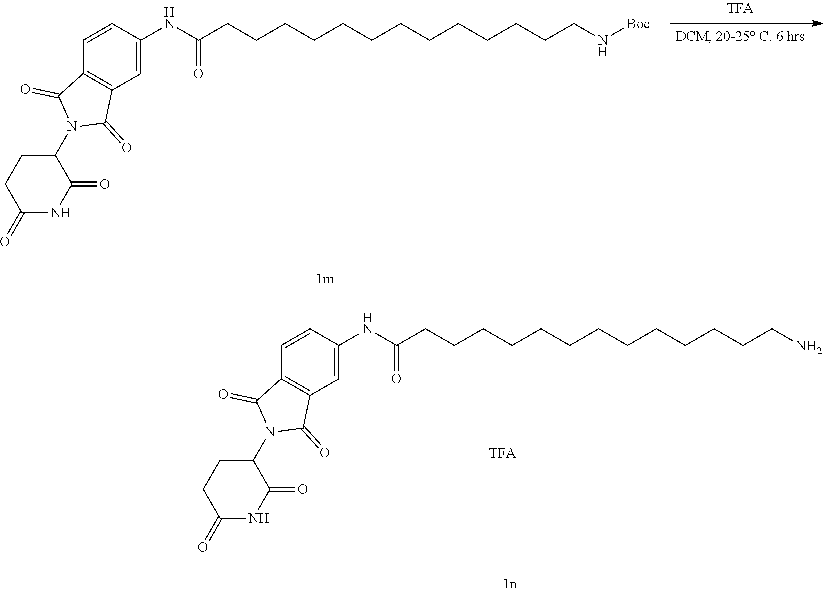 [SMILES: CC(C)(C)OC(=O)NCCCCCCCCCCCCCC(=O)Nc1ccc2c(c1)C(=O)N(C1CCC(=O)NC1=O)C2=O ; NCCCCCCCCCCCCCC(=O)Nc1ccc2c(c1)C(=O)N(C1CCC(=O)NC1=O)C2=O]

To a solution of compound 1m (40 mg, 66.8 umol) in DCM (5 mL) was added TFA (76 mg, 666 umol, 10.0 eq) at 20-25° C., after addition, the mixture was stirred at 20-25° C. for 6 hrs. The LCMS showed the compound 1n was detected (RT=0.853 min m/z=499). The reaction solution was concentrated under reduce pressure to afford compound 1n (40.0 mg, TFA) as brown oil.

LCMS: (method 1), RT=0.853 min, m/z=499

General Procedure for Preparation of “A50”

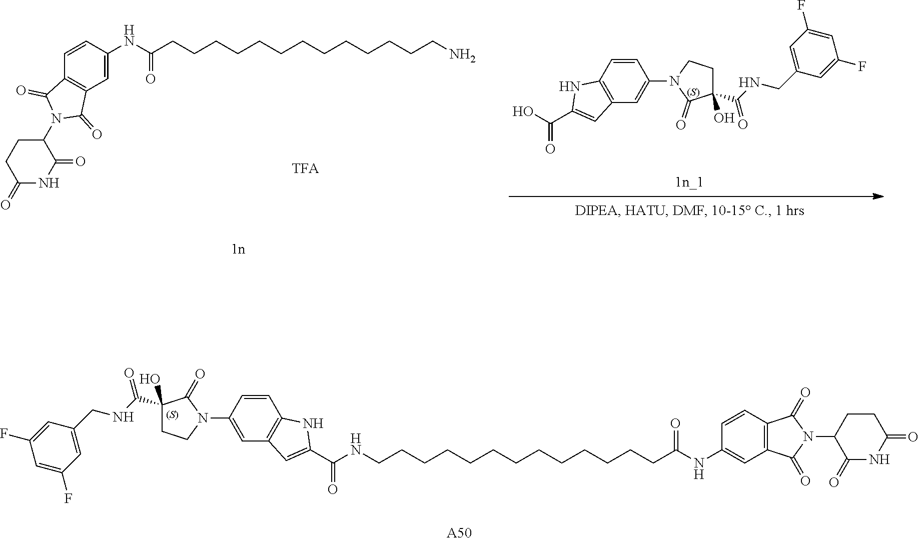

To a solution of compound 1n_1 (25 mg, 58.2 umol) in DMF (3 mL) was added DIPEA (40 mg, 309 umol) and HATU (35 mg, 92.1 umol) at 10-15° C., after addition, compound 1n (40 mg, 65.3 umol, TFA) dissolved in DMF (1 mL) was added to the solution at 10-15° C., then it was stirred at 10-15° C. for 1 hr. The reaction solution was quenched with AcOH to pH=3, then it was poured into water (10 mL), and extracted with ethyl acetate (10 mL*3), the organic layer was washed with brine (10 mL), dried over Na₂SO₄, filtrated and concentrated under reduce pressure. The crude was purified by Pre-HPLC (column: Phenomenex Gemini-NX C18 75*30 mm*3 um; mobile phase: [water (0.1% TFA)-ACN]; B %: 58%-68%, 7 min), then concentrated under reduce pressure to afford “A50” (27.86 mg, 29.6 umol, 50.9% yield, 96.8% purity) as white solid, which was confirmed by ¹H NMR, LCMS RT=1.045 min, m/z+1=910), HPLC (, RT=2.455 min)

¹H NMR: 400 MHz, DMSO-d₆ δ 11.59 (s, 1H), 11.11 (s, 1H), 10.53 (s, 1H), 8.70 (t, J=6.4 Hz, 1H), 8.46 (t, J=6.4 Hz, 1H), 8.26 (s, 1H), 7.82-7.99 (m, 2H), 7.78 (d, J=1.6 Hz, 1H), 7.52-7.57 (m, 1H), 7.43 (d, J=8.8 Hz, 1H), 6.96-7.15 (m, 4H), 6.71 (s, 1H), 5.05-5.14 (m, 1H) 4.39-4.45 (m, 1H), 4.22-4.32 (m, 1H), 3.90 (t, J=6.8 Hz, 2H), 3.22-3.28 (m, 2H), 2.77-2.99 (m, 1H), 2.59-2.70 (m, 2H), 2.38 (t, J=7.6 Hz, 2H), 2.02-2.21 (m, 2H), 1.89-1.93 (m, 1H), 1.49-1.74 (m, 4H), 1.22-1.44 (m, 18H).

LCMS: (method 1), RT=1.045 min, m/z=910

HPLC: (method 2), RT=2.455 min

### 5-[(3S)-3-{[(3,5-difluorophenyl)methyl]carbamoyl}-3-hydroxy-2-oxopyrrolidin-1-yl]-N-{4-[4-(4-{[2-(2,6-dioxopiperidin-3-yl)-1,3-dioxo-2,3-dihydro-1H-isoindol-5-yl]amino}butoxy)butoxy]butyl}-1H-indole-2-carboxamide (“A51”)

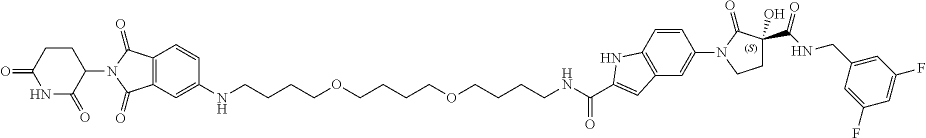

LC-MS or HPLC Method:

Method 1:

MS instrument type: SHIMADZU LCMS-2020, Column: Kinetex EVO C18 30*2.1 mm, 5 um, mobile phase A: 0.0375% TFA in water (v/v), B: 0.01875% TFA in Acetonitrile (v/v), gradient: 0.0 min 0% B→0.8 min 95% B→1.2 min 95% B→1.21 min 5% B→1.55 min 5% B, flow rate: 1.5 mL/min, oven temperature: 50° C.; UV detection: 220 nm & 254 nm.

Method 2:

MS instrument type: SHIMADZU LCMS-2020, Column: Kinetex EVO C18 30*2.1 mm, 5 um, mobile phase A: 0.0375% TFA in water (v/v), B: 0.01875% TFA in acetonitrile (v/v), gradient: 0.0 min 0% B→3.0 min 95% B→3.5 min 95% B→3.51 min 5% B→4.0 min 5% B, flow rate: 0.8 mL/min, oven temperature: 50° C.; UV detection: 220 nm & 254 nm.

4. Experimental for Largest Scale Run:

General Procedure for Preparation of Compound 7b

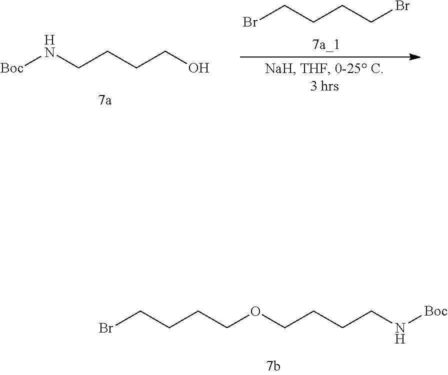 [SMILES: BrCCCCBr ; CC(C)(C)OC(=O)NCCCCO ; CC(C)(C)OC(=O)NCCCCOCCCCBr]

To a solution of compound 7a (1 g, 5.28 mmol) and compound 7a_1 (3.42 g, 15.8 mmol) in THE (10 mL) was added NaH (634 mg, 15.8 mmol, 60% purity) at 0-5° C. under N₂, then it was stirred at 20-25° C. for 3 hrs under N₂. TLC (Petroleum ether: Ethyl acetate=5:1) showed compound 7a was consumed up (R_{f}=0.05), a new spot was detected (R_{f}=0.40). The reaction solution was poured into water (10 mL), then it was extracted with ethyl acetate (10 mL*2), the combined organic layer was washed with brine (10 mL), dried over Na₂SO₄, filtrated and concentrated under reduce pressure to afford compound 7b (460 mg, 1.42 mmol, 26.8% yield) as a colorless oil, which was confirmed by ¹H NMR.

¹H NMR: 400 MHz, CDCl₃ δ 4.58 (s, 1H), 3.29-3.53 (m, 6H), 3.07 (d, J=6.0 Hz, 2H), 1.81-1.95 (m, 2H), 1.61-1.75 (m, 2H), 1.45-1.58 (m, 4H), 1.37 (s, 9H).

General Procedure for Preparation of Compound 7c

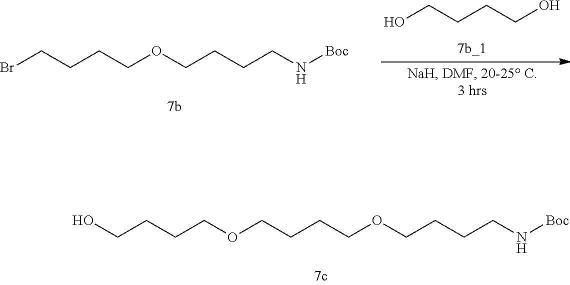 [SMILES: OCCCCO ; CC(C)(C)OC(=O)NCCCCOCCCCBr ; CC(C)(C)OC(=O)NCCCCOCCCCOCCCCO]

To a solution of compound 7b (350 mg, 1.08 mmol) and compound 7b_1 (486 mg, 5.39 mmol, 476 uL) in DMF (10 mL) was added NaH (560 mg, 14.0 mmol, 60% purity) at 20-25° C. under N₂ after addition, the mixture was stirred at 20-25° C. for 1 hr. TLC (Petroleum ether: Ethyl acetate=5:1) showed some new spots was detected (R_{f}=0.45, 0.10), compound 7b (R_{f}=0.70) was consumed up. The reaction solution was poured into water (100 mL), then it was extracted with ethyl acetate (30 mL*3), the organic layer was washed with brine (30 mL), dried over Na₂SO₄, filtrated and concentrated under reduce pressure. Then it was purified by silica gel column chromatography (SiO₂, Petroleum ether:ethyl acetate=20:1-10:1-3:1), the spot (R_{f}=0.10) was collected. Compound 7c (230 mg, 689 umol, 63.9% yield) was obtained as a colorless oil, which was confirmed by ¹H NMR.

¹H NMR: 400 MHz, CDCl₃ δ 4.65 (s, 1H), 3.54-3.64 (m, 2H), 3.29-3.46 (m, 8H), 3.06 (d, J=5.6 Hz, 2H), 1.47-1.73 (m, 12H), 1.37 (s, 9H).

General Procedure for Preparation of Compound 7d

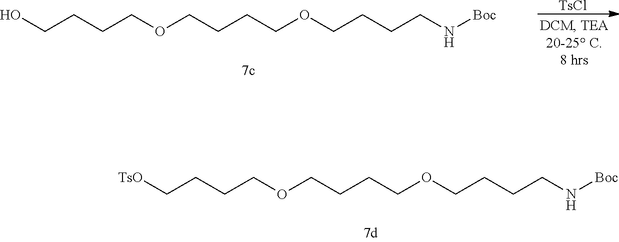 [SMILES: CC(C)(C)OC(=O)NCCCCOCCCCOCCCCO ; "TsCl" ; Cc1ccc(S(=O)(=O)OCCCCOCCCCOCCCCNC(=O)OC(C)(C)C)cc1]

To a solution of compound 7c (230 mg, 689 umol) and TsCl (200 mg, 1.05 mmol) in DCM (5 mL) was added Et₃N (210 mg, 2.08 mmol) at 20-25° C., after addition, the mixture solution was stirred at 20-25° C. for 8 hrs. TLC (Petroleum ether: Ethyl acetate=1:1) showed the compound 7c (R_{f}=0.10) was consumed up, a new spot (R_{f}=0.70) was detected. The reaction solution was poured into water (10 mL), then it was extracted with ethyl acetate (10 mL*2), the combined organic layer was washed with brine (10 mL), dried over Na₂SO₄, filtrated and concentrated under reduce pressure. The crude was purified by silica gel column chromatography (SiO₂, Petroleum ether:ethyl acetate=20:1-10:1-5:1), the spot (R_{f}=0.70) was collected. Compound 7d (120 mg, 246 umol, 35.6% yield) was obtained as colorless oil, which was confirmed by next step.

General Procedure for Preparation of Compound 7e

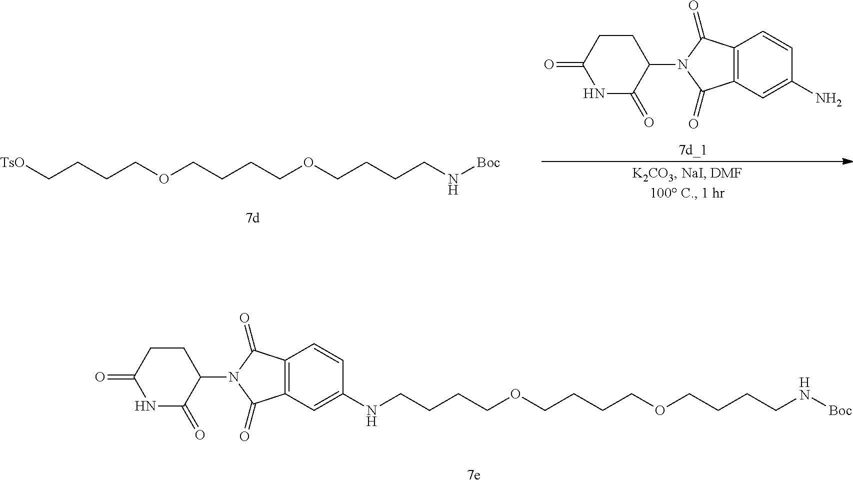

To a solution of compound 7d (90 mg, 184 umol) and Compound 7d_1 (56 mg, 204 umol) in DMF (5 mL) was added NaI (14 mg, 93.4 umol) and K₂CO₃ (51 mg, 369 umol, 2.00 eq) at 20-25° C., after addition, the mixture solution was stirred at 100° C. for 1 hr. The reaction solution was cooled to 15-20° C., then it was poured into water (20 mL) and extracted with ethyl acetate (10 mL*2), the combined organic layer washed with brine (10 mL), dried over Na₂SO₄, filtrated and concentrated under reduce pressure to afford the crude product the Crude was purified by Pre-HPLC (column: Phenomenex Gemini-NX C18 75*30 mm*3 um; mobile phase: [water (0.1% TFA)-ACN]; B %: 52%-62%, 7 min), then it was concentrated under reduce pressure to afford compound 7e (40 mg, 80.2 umol, 43.4% yield) as a pale green oil, which was confirmed by LCMS RT=0.967 min, m/z+1=489),

LCMS: (method 1), RT=0.967 min, m/z=489

General Procedure for Preparation of Compound 7f

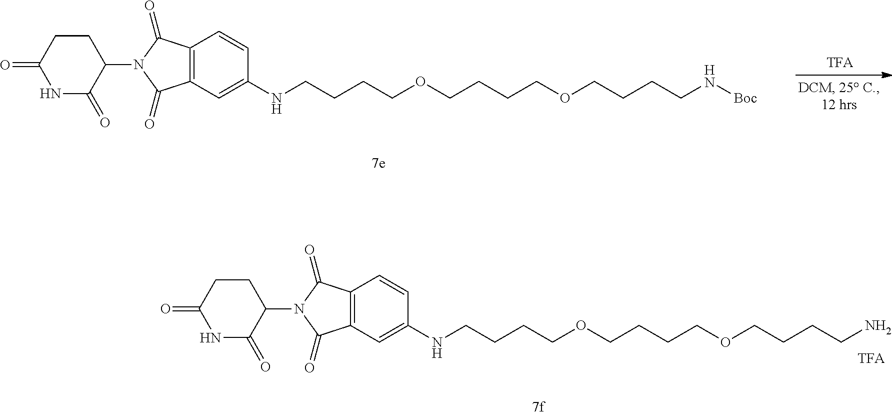 [SMILES: CC(C)(C)OC(=O)NCCCCOCCCCOCCCCNc1ccc2c(c1)C(=O)N(C1CCC(=O)NC1=O)C2=O ; NCCCCOCCCCOCCCCNc1ccc2c(c1)C(=O)N(C1CCC(=O)NC1=O)C2=O ; "TFA"]

To a solution of compound 7e (40 mg, 69.6 umol) in DCM (5 mL) was added TFA (154 mg, 1.35 mmol) at 25° C., then it was stirred at 25° C. for 12 hrs. LCMS showed the MS of compound 7f (RT=0.795 min, m/z=489) was detected. The reaction solution was concentrated under reduce pressure to afford compound 7f (40 mg, 66.3 umol, 95.3% yield, TFA) as a yellow oil, which confirmed by ¹H NMR

¹H NMR: 400 MHz, DMSO-d₆ δ 7.55 (s, 3H), 7.43 (d, J=8.4 Hz, 1H), 6.86 (d, J=2.0 Hz, 1H), 6.75 (dd, J=8.4, 2.0 Hz, 1H), 5.02 (dd, J=13.2, 5.4 Hz, 1H), 4.31 (t, J=6.4 Hz, 1H), 3.17-3.34 (m, 10H), 2.81-2.95 (m, 1H), 2.64-2.76 (m, 3H), 1.88-2.01 (m, 1H) 1.30-1.59 (m, 14H).

LCMS: (method 1), RT=0.795 min, m/z+1=489

General Procedure for Preparation of “A51”

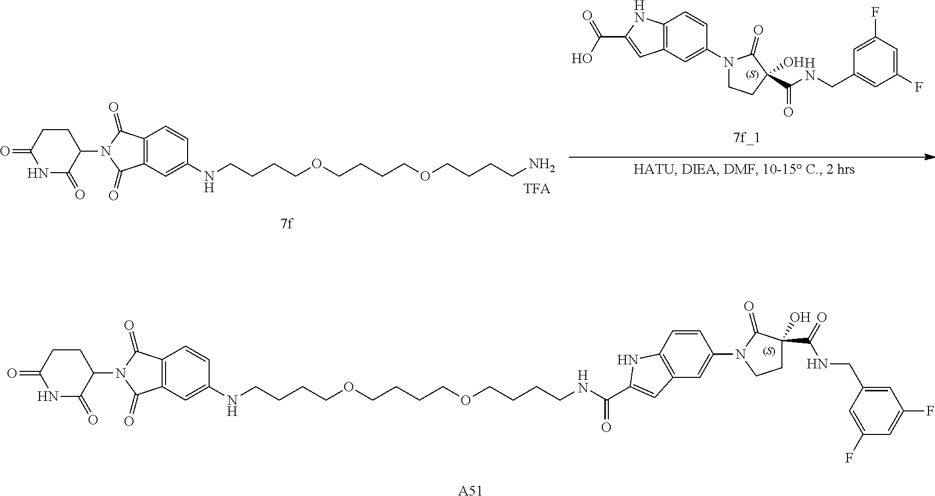

To a solution of compound 7f_1 (30 mg, 69.8 umol) in DMF (3 mL) was added HATU (85 mg, 105 umol, 1.51 eq) and DIEA (45 mg, 348 umol) at 10-15° C., after addition, the mixture was stirred at 10-15° C. for 30 min, then compound 7f (40 mg, 66.3 umol, TFA) in DMF (2 mL) was added to the solution at 10-15° C. under N₂, then it was stirred at 10-15° C. for 1 hrs. LCMS showed the MS of compound 7 was detected (RT=0.949 min, m/z=900). the reaction solution was quenched with AcOH (0.5 mL), then it was poured into water (20 mL) and extracted with ethyl acetate (10 mL*2), the combined organic layer was washed with brine (10 mL), dried over Na₂SO₄, filtrated and concentrated under reduce pressure. The crude was purified by Pre-HPLC (column: Phenomenex Gemini-NX C18 75*30 mm*3 um; mobile phase: [water (0.1% TFA)-ACN]; B %: 48%-58%, 7 min), then it was concentrated under reduce pressure to afford the product. “A51” (44.3 mg, 47.7 umol, 68.3% yield) was obtained as yellow gum, which was confirmed by ¹H NMR, LCMS RT=0.945 min, m/z=900), HPLC RT=2.292 min).

¹H NMR: 400 MHz, DMSO-d₆ δ 11.59 (s, 1H), 8.70 (t, J=6.4 Hz, 1H), 8.70 (t, J=6.4 Hz, 1H), 7.78 (s, 1H), 7.48-7.64, (m, 2H), 7.43 (d, J=8.8 Hz, 1H), 6.92-7.17 (m, 5H), 6.84 (dd, J=8.4, 2.0 Hz, 1H), 5.11 (dd, J=12.8, 5.2 Hz, 2H), 4.25-4.45 (m, 2H), 3.90 (t, J=6.4 Hz, 1H) (overlap H₂O signal), 3.64 (s, 1H), 3.18-3.48 (m, 10H), 2.86-3.07 (m, 1H), 2.56-2.81 (m, 4H) (overlap DMSO signal), 1.92-2.24 (m, 3H), 1.35-1.70 (m, 14H).

LCMS: (method 1), RT=0.945 min m/z=900

HPLC: (method 2), RT=2.292 min

### 5-[(3S)-3-{[(3,5-difluorophenyl)methyl]carbamoyl}-3-hydroxy-2-oxopyrrolidin-1-yl]-N-(14-{[2-(2,6-dioxopiperidin-3-yl)-1,3-dioxo-2,3-dihydro-1H-isoindol-5-yl]oxy}-3,6,9,12-tetraoxatetradecan-1-yl)-1H-indole-2-carboxamide (“A52”)

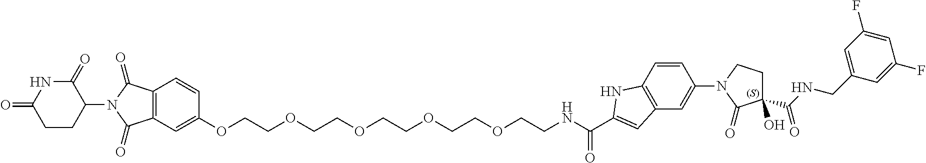

LC-MS or HPLC Method:

Method 1:

MS instrument type: SHIMADZU LCMS-2020, Column: Kinetex EVO C18 30*2.1 mm, Sum, mobile phase A: 0.0375% TFA in water (v/v), B: 0.01875% TFA in Acetonitrile (v/v), gradient: 0.0 min 0% B→0.8 min 95% B→1.2 min 95% B→1.21 min 5% B→1.55 min 5% B, flow rate: 1.5 mL/min, oven temperature: 50° C.: UV detection: 220 nm & 254 nm.

Method 2:

MS instrument type: SHIMADZU LCMS-2020, Column: Kinetex EVO C18 30*2.1 mm, 5 um, mobile phase A: 0.0375% TFA in water (v/v), B: 0.01875% TFA in acetonitrile (v/v), gradient: 0.0 min 0% B→3.0 min 95% B→3.5 min 95% B→3.51 min 5% B→4.0 min 5% B, flow rate: 0.8 mL/min, oven temperature: 50° C.; UV detection: 220 nm & 254 nm.

4. Experimental for Largest Scale Run:

General Procedure for Preparation of Compound 8-3

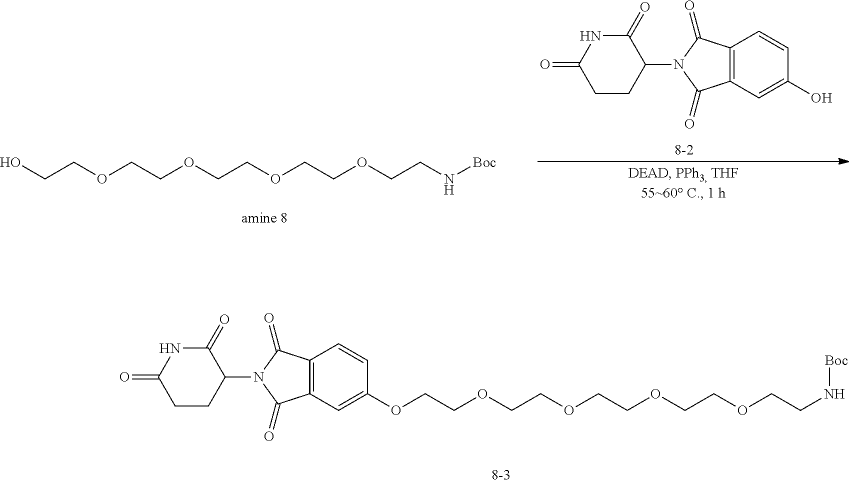

To a solution of compound 8-2 (90 mg, 328 umol), amine 8 (100 mg, 296 umol) and DEAD (70 mg, 402 umol) in THF (3.00 mL) was added PPh₃ (130 mg, 495 umol) at 0˜5° C. The mixture was stirred at 10-20° C. for 1 h. Then the mixture was stirred at 55-60° C. for 12 h. LCMS showed the MS of compound 8-2 was remained (RT=0.652 min, m/z (M+1)=275) and the MS of compound 8-3 was not found. To the mixture was added PPh₃ (172 mg, 656 umol) and DEAD (114 mg, 655 umol) at 55-60° C. and kept stirred for 1 h. LCMS showed the MS of compound 8-2 was remained (RT=0.643 min, m/z (M+1)=275) and the MS of compound 8-3 (RT=0.881 min, m/z (M+23)=616) was found. TLC (Petroleum ether/Ethyl acetate=0/1) showed compound 8-2 (R_{f}=0.68) was remained and a main new spot (R_{f}=0.25) was found. The reaction mixture adjust pH=6-7 with AcOH and concentrated under reduce pressure. The residue was purified by flash silica gel chromatography (Silica Flash Column, Eluent of 0˜100% Ethyl acetate/Petroleum ether gradient). The spot of R_{f}=0.25 was collected. Compound 8-3 (110 mg, 182 umol, 55.4% yield, 98.2% purity) was obtained as a yellow gum.

LCMS: (method 1), RT=0.881 min, m/z (M+23)=616

LCMS: (method 1), RT=0.877 min, m/z (M+1-100)=494

General Procedure for Preparation of Compound 8-4

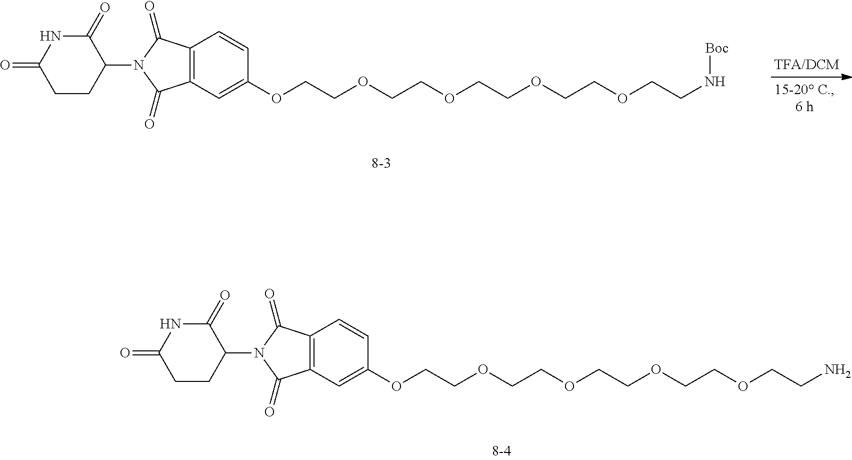 [SMILES: CC(C)(C)OC(=O)NCCOCCOCCOCCOCCOc1ccc2c(c1)C(=O)N(C1CCC(=O)NC1=O)C2=O ; NCCOCCOCCOCCOCCOc1ccc2c(c1)C(=O)N(C1CCC(=O)NC1=O)C2=O]

To a solution of compound 8-3 (110 mg, 185 umol) in DCM (5.00 mL) was added TFA (211 mg, 1.85 mmol). The mixture was stirred at 15-20° C. for 6 hr. TLC (Petroleum ether/Ethyl acetate=0/1) showed compound 8-3 (R_{f}=0.25) was consumed and a main new spot (R_{f}=0.00) was found. The reaction mixture was concentrated under reduce pressure. Compound 8-4 (98 mg, 155 umol, 83.8% yield, 96.3% purity, TFA salt) was obtained as a yellow gum.

LCMS: (method 1), RT=0.752 min, m/z (M+1)=494

General Procedure for Preparation of “A52”

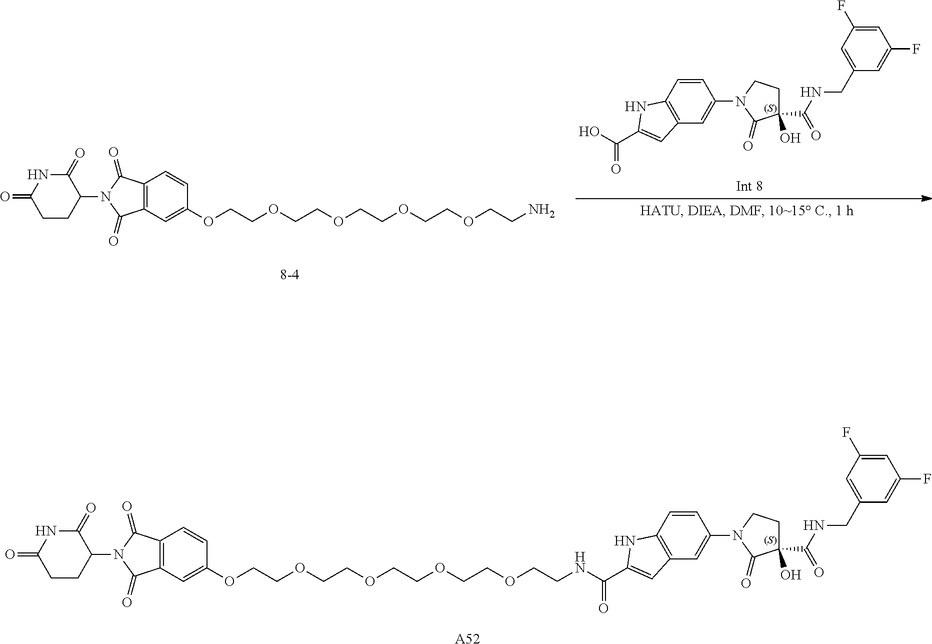

To a solution of Int 8 (30 mg, 69.8 umol) in DMF (3 mL) was added HATU (40 mg, 105 umol) and DIEA (45 mg, 348 umol) at 10-15° C., after addition, compound 8-4 (51 mg, 84.0 umol TFA salt) in DMF (1 mL) was added into the solution, then it was stirred at 10-15° C. for 1 hr. LCMS showed Int 8 was consumed and a main new peak with desired MS (RT=0.871 min, m/z (M+1)=905) was found. The reaction solution was poured into water (5 mL), then it was quenched with AcOH (1.00 mL) and extracted with ethyl acetate (10 mL*2), the organic layer was washed with brine (10 mL), dried over Na₂SO₄, filtrated and concentrated under reduce pressure. The residue was purified by Pre-HPLC (column: Phenomenex Luna C18 150*25 mm*10 um; mobile phase: [water (0.1% TFA)-ACN]; B %: 25%55%, 10 min), then it was concentrated under reduce pressure to afford the “A52” (51.4 mg, 56.8 umol, 81.3% yield, 100% purity), which was confirmed by ¹H-NMR and LCMS as a light yellow gum.

¹H NMR: 400 MHz, DMSO-d₆ δ 11.62 (s, 1H), 11.11 (s, 1H), 8.70 (t, J=6.4 Hz, 1H), 8.56 (t, J=5.6 Hz, 1H) 7.83 (d, J=8.4 Hz, 1H), 7.78 (d, J=1.6 Hz, 1H), 7.54 (dd, J=8.8, 2.0 Hz, 1H), 7.41-7.47 (m, 2H), 7.36 (dd, J=8.4, 2.4 Hz, 1H), 7.11-7.16 (m, 1H), 6.97-7.10 (m, 3H), 5.12 (dd, J=12.8, 5.6 Hz, 1H), 4.42 (dd, J=16.0, 6.8 Hz, 1H), 4.22-4.34 (m, 3H), 3.90 (t, J=6.8 Hz, 2H), 3.73-3.78 (m, 2H), 2.83-2.96 (m, 1H), 2.54-2.66 (m, 3H), 2.09-2.19 (m, 1H), 1.99-2.09 (m, 1H).

LCMS: (method 2), RT=2.144 min, m/z (M+1)=905

### 5-[(3S)-3-{[(3,5-difluorophenyl)methyl]carbamoyl}-3-hydroxy-2-oxopyrrolidin-1-yl]-N-(14-{[2-(2,6-dioxopiperidin-3-yl)-1,3-dioxo-2,3-dihydro-1H-isoindol-4-yl]oxy}-3,6,9,12-tetraoxatetradecan-1-yl)-1H-indole-2-carboxamide (“A53”)

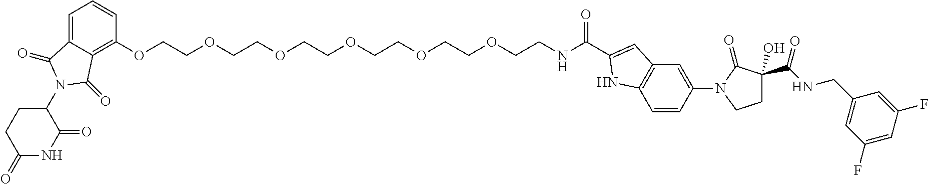 [SMILES: O=C1CCC(N2C(=O)c3cccc(OCCOCCOCCOCCOCCOCCNC(=O)c4cc5cc(N6CC[C@](O)(C(=O)NCc7cc(F)cc(F)c7)C6=O)ccc5[nH]4)c3C2=O)C(=O)N1]

LC-MS or HPLC Method:

Method 1:

MS instrument type: SHIMADZU LCMS-2020, Column: Kinetex EVO C18 30*2.1 mm, 5 um, mobile phase A: 0.0375% TFA in water (v/v), B: 0.01875% TFA in Acetonitrile (v/v), gradient: 0.0 min 0% B→0.8 min 95% B→1.2 min 95% B→1.21 min 5% B→1.55 min 5% B, flow rate: 1.5 mL/min, oven temperature: 50° C.; UV detection: 220 nm & 254 nm.

Method 2:

MS instrument type: SHIMADZU LCMS-2020, Column: Kinetex EVO C18 30*2.1 mm, 5 um, mobile phase A: 0.0375% TFA in water (v/v), B: 0.01875% TFA in acetonitrile (v/v), gradient: 0.0 min 0% B→3.0 min 95% B→3.5 min 95% B→3.51 min 5% B→4.0 min 5% B, flow rate: 0.8 mL/min, oven temperature: 50° C.; UV detection: 220 nm & 254 nm.

4. Experimental for Largest Scale Run:

General Procedure for Preparation of Amine 13—

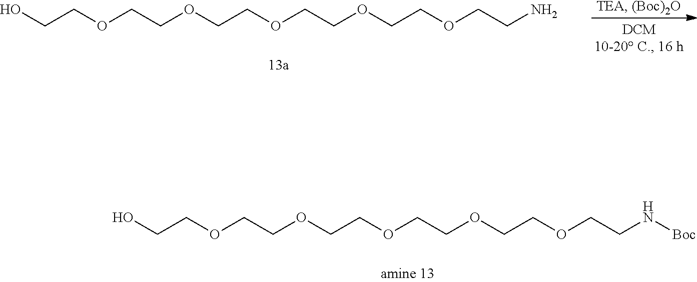 [SMILES: "TEA, (Boc)2O" ; NCCOCCOCCOCCOCCOCCO ; CC(C)(C)OC(=O)NCCOCCOCCOCCOCCOCCO]

To a solution of compound 13a (650 mg, 2.31 mmol) and TEA (468 mg, 4.63 mmol) in DCM (6.50 mL) was added dropwise (Boc)₂O (706 mg, 3.23 mmol) at 10-20° C. under N₂ atmosphere and kept stirred at 10-20° C. for 16 h under N₂ atmosphere. TLC (DCM/Methanol=10/1) showed compound 13a (R_{f}=0.00) was disappeared and new spot (R_{f}=0.52) was found. The reaction mixture was concentrated under reduce pressure. The residue was purified by flash silica gel chromatography (ISCO®; X g SepaFlash® Silica Flash Column, Eluent of 0˜2% Methanol/DCM). Amine 13 (610 mg, 1.60 mmol, 69.2% yield) was obtained as a colorless oil which was confirmed by ¹H NMR.

¹H NMR: 400 MHz, CDCl₃ δ 5.22 (s, 1H), 3.59-3.77 (m, 20H), 3.55 (t, J=5.2 Hz, 2H), 3.32 (d, J=5.2 Hz, 2H), 2.87-3.07 (m, 1H), 1.45 (s, 9H)

General Procedure for Preparation of Compound 13-2

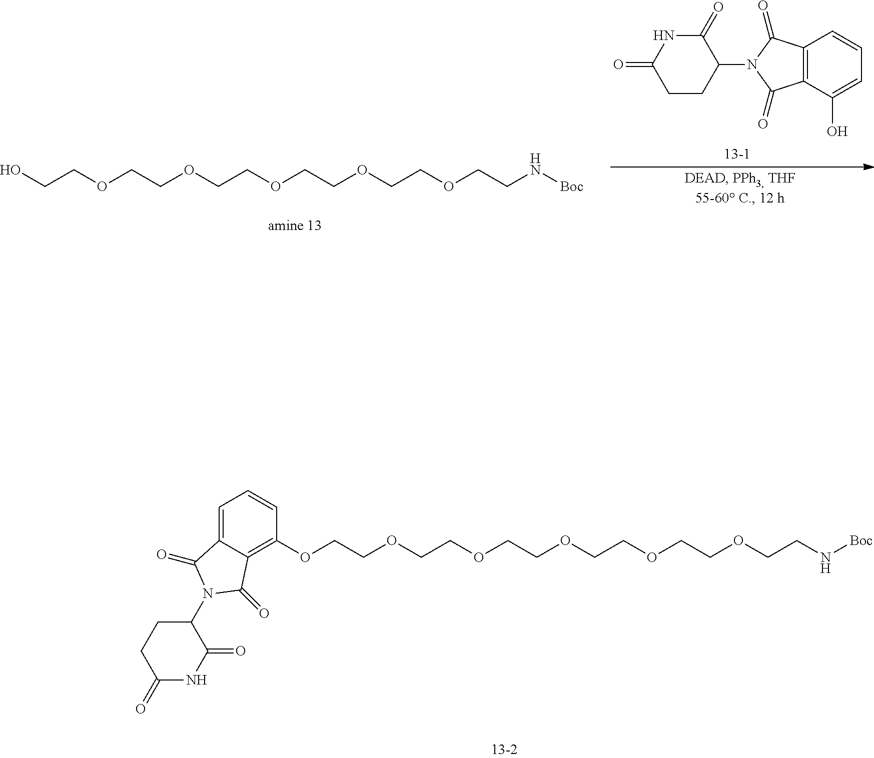 [SMILES: O=C1CCC(N2C(=O)c3cccc(O)c3C2=O)C(=O)N1 ; CC(C)(C)OC(=O)NCCOCCOCCOCCOCCOCCO ; CC(C)(C)OC(=O)NCCOCCOCCOCCOCCOCCOc1cccc2c1C(=O)N(C1CCC(=O)NC1=O)C2=O]

To a solution of amine 13 (265 mg, 694 umol) and compound 13-1 (200 mg, 729 umol) in THF (10.0 mL) was added PPh₃ (600 mg, 2.29 mmol) at 20-25° C. under N₂ atmosphere. Then to the mixture was added dropwise DEAD (320 mg, 1.84 mmol) at 55-60° C. under N₂ atmosphere and kept stirred at 55-60° C. for 12 hours under N₂ atmosphere. LC-MS (EW18817-71-P1A1) showed the MS of compound 13-2 (RT=0.867 min, m/z (M−100+1)=538) was detected. The mixture was concentrated under reduce pressure. The residue was purified by flash silica gel chromatography (SepaFlash® Silica Flash Column, Eluent of 0˜100% Ethyl acetate/Petroleum ether gradient). The residue was purified by prep-HPLC (TFA condition, column: Phenomenex Gemini-NX C18 75*30 mm*3 um; mobile phase: [water (0.1% TFA)-ACN]; B %: 35%-45%, 7 min). Compound 13-2 (190 mg, 294 umol, 40.4% yield, 99% purity) was obtained as a yellow gum, which was confirmed by LCMS (BOC cleaved MS: RT=0.711 mins).

LCMS: (method 1), RT=0.877 min, m/z (M−56+1)=538 (RT=0.711 min, m/z (M−100+1)=494)

General Procedure for Preparation of Compound 13-3

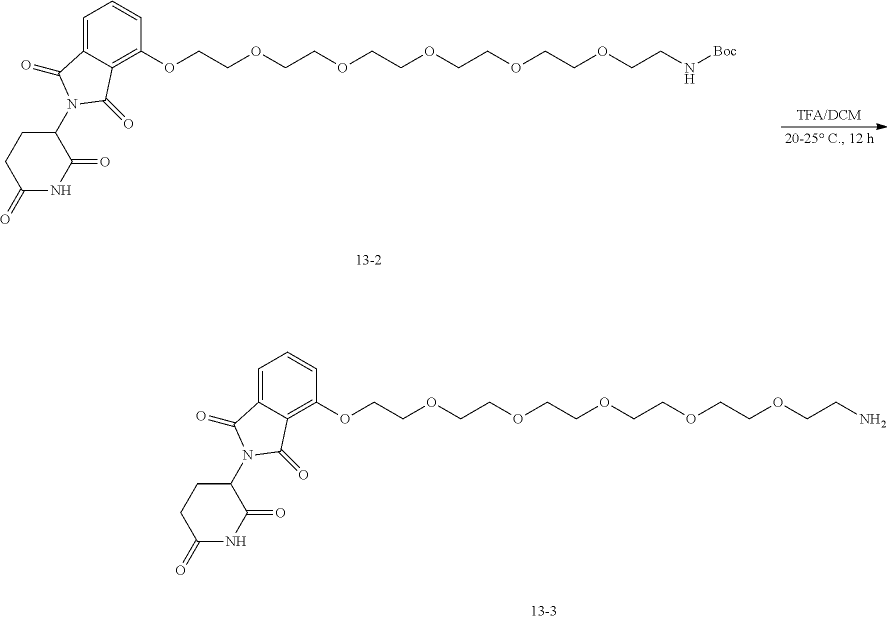 [SMILES: CC(C)(C)OC(=O)NCCOCCOCCOCCOCCOCCOc1cccc2c1C(=O)N(C1CCC(=O)NC1=O)C2=O ; NCCOCCOCCOCCOCCOCCOc1cccc2c1C(=O)N(C1CCC(=O)NC1=O)C2=O]

To a solution of compound 13-2 (190 mg, 298 umol) in DCM (5 mL) was added TFA (340 mg, 2.98 mmol) at 20-25° C. and kept stirred for 12 h. TLC (Petroleum ether/Ethyl acetate=0/1) showed compound 13-2 (R_{f}=0.28) was consumed and a main new spot (R_{f}=0.00) was formed. The mixture was concentrated under reduce pressure. Compound 13-3 (151 mg, 230 umol, 77.5% yield, 99.6% purity, TFA salt) was obtained as a yellow gum.

General Procedure for Preparation of Compound “A53”

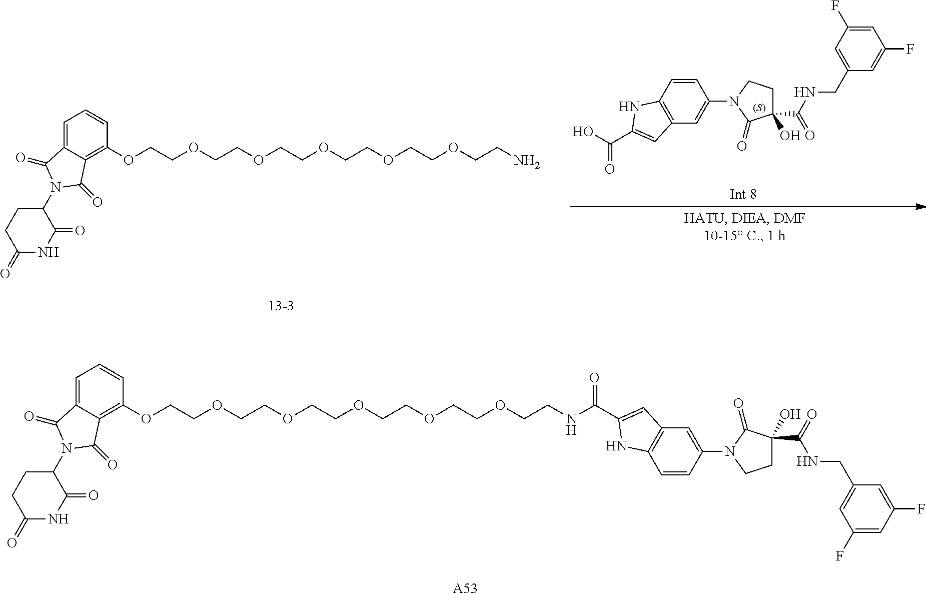

To a solution of Int 8 (30 mg, 69.8 umol) in DMF (3.00 mL) was added HATU (40 mg, 105 umol) and DIEA (45 mg, 348 umol) at 10-15° C., after addition, compound 13-3 (55 mg, 84.4 umol, TFA salt) in DMF (1.00 mL) was added into the solution, then it was stirred at 10-15° C. for 1 hr. LCMS showed Int 8 was remained (RT=0.750 min, M+1=538) and a main new peak with the MS of compound 13 (RT=0.904 min, M+1=949) was found. The reaction solution was poured into water (5.00 mL), then it was quenched with AcOH (1.00 mL) and extracted with ethyl acetate (10.0 mL*2), the organic layer was washed with brine (10.0 mL), dried over Na₂SO₄, filtrated and concentrated under reduce pressure. The crude was purified by Pre-HPLC (column: Phenomenex Gemini-NX C18 75*30 mm*3 um; mobile phase: [water (0.1% TFA)-ACN]; B %: 38%-48%, 7 min), then it was lyophilized to afford “A53” (28.32 mg, 29.8 umol, 42.7% yield, 100% purity), which was confirmed by ¹H NMR and LCMS as a yellow gum.

¹H NMR: 400 MHz, DMSO-d₆ δ 11.62 (s, 1H), 11.10 (s, 1H), 8.70 (t, J=6.4 Hz, 1H), 8.56 (t, J=5.6 Hz, 1H), 7.75-7.85 (m, 2H), 7.50-7.58 (m, 2H), 7.41-7.49 (m, 2H), 7.13 (d, J=1.2 Hz, 1H), 7.03-7.11 (m, 1H), 6.97-7.03 (m, 2H), 6.72 (s, 1H), 5.09 (dd, J=12.8, 5.4 Hz, 1H), 4.37-4.48 (m, 1H), 4.31-4.36 (m, 2H), 4.21-4.31 (m, 1H), 3.90 (t, J=6.8 Hz, 2H), 3.75-3.84 (m, 2H), 3.62 (dd, J=5.6, 3.6 Hz, 2H), 3.41-3.58 (m, 20H), 2.82-2.96 (m, 1H), 2.54-2.66 (m, 3H), 2.15 (dt, J=13.2, 7.6 Hz, 1H), 1.97-2.07 (m, 1H) (˜20H overlap H₂O signal, ˜3H overlap DMSO signal).

LCMS: (method 2), RT=2.101 min, m/z (M+1)=949

### 5-[(3S)-3-{[(3,5-difluorophenyl)methyl]carbamoyl}-3-hydroxy-2-oxopyrrolidin-1-yl]-N-(14-{[2-(2,6-dioxopiperidin-3-yl)-1,3-dioxo-2,3-dihydro-1H-isoindol-4-yl]oxy}-3,6,9,12-tetraoxatetradecan-1-yl)-1H-indole-2-carboxamide (“A54”)

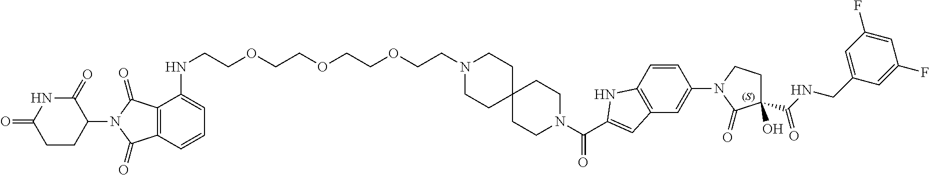

LC-MS or HPLC Method:

Method 1:

MS instrument type: SHIMADZU LCMS-2020, Column: Kinetex EVO C18 2.1×30 mm, 5 um, mobile phase A: 0.025% NH₃.H₂O in Water (v/v), B: Acetonitrile, gradient: 0.0 min 5% B→0.8 min 95% B→1.2 min 95% B→1.21 min 5% B→1.55 min 5% B, flow rate: 1.5 mL/min, oven temperature: 50° C.; UV detection: 220 nm & 254 nm.

Method 2:

MS instrument type: SHIMADZU LCMS-2020, Column: Kinetex EVO C18 30*2.1 mm, Sum, mobile phase A: 0.0375% TFA in water (v/v), B: 0.01875% TFA in Acetonitrile (v/v), gradient: 0.0 min 5% B→0.8 min 95% B→1.2 min 95% B→1.21 min 5% B→1.55 min 5% B, flow rate: 1.5 mL/min, oven temperature: 50° C.; UV detection: 220 nm & 254 nm.

4. Experimental for Largest Scale Run:

General Procedure for Preparation of Intermediate 16b

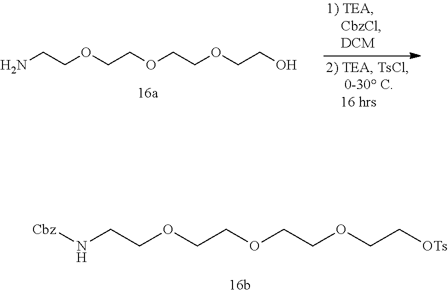 [SMILES: NCCOCCOCCOCCO ; Cc1ccc(S(=O)(=O)OCCOCCOCCOCCNC(=O)OCc2ccccc2)cc1]

To a solution of compound 16a (1.10 g, 5.69 mmol) in DCM (10 mL) was added TEA (1.32 g, 13.1 mmol, 1.82 mL) at 25-30° C. under N₂, then CbzCl (1.17 g, 6.83 mmol, 100 uL) was added into the mixture at 25-30° C. under N₂, the mixture was stirred for 10 hrs. The mixture was cooled to 0-10° C., TEA (5 g, 49.4 mmol) was added into the mixture at 0-10° C. under N₂, then TsCl (5.00 g, 26.2 mmol) was added into the mixture at 0 10° C. under N₂, it was stirred at 25-30° C. for 6 hrs under N₂. 0.5 mL reaction mixture was taken and poured into water (3 mL), extracted with ethyl acetate (1 mL), the organic phase was washed with water (3 mL×3), then was concentrated under reduced pressure to give colourless oil, LCMS Rt=0.915 min, m/z (M+1)=482.4) showed the MS of compound 16b was detected, HNMR (EW18785-17-P1A2) showed compound 16b was formed. The mixture was poured into water (100 mL), extracted with ethyl acetate (50 mL×2), the organic phase was washed with brine (100 mL×3), dried with Na₂SO₄, filtered, the filtrate was concentrated under reduced pressure to give brown oil. TLC 1 (Petroleum ether/Ethyl acetate=1/1, R_{f}=0.30). The oil was purified by column chromatography (SiO₂, Petroleum ether/Ethyl acetate=1/0-1/1, R_{f}=0.30) to give compound 16b (1.80 g, 3.74 mmol, 65.7% yield) as brown oil.

¹H NMR: 400 MHz, CDCl₃ δ 7.78 (d, J=8.4 Hz, 2H), 7.26-7.42 (m, 7H), 5.42 (s, 1H), 5.08 (s, 2H), 4.76 (s, 1H), 4.11 (d, J=4.4 Hz, 2H), 3.45-3.74 (m, 13H), 3.37 (d, J=5.2 Hz, 2H), 2.43 (s, 3H), 1.83 (s, 3H)

LCMS: (method 1), Rt=0.915 min, m/z (M+1)=482.4

General Procedure for Preparation of Compound 16d

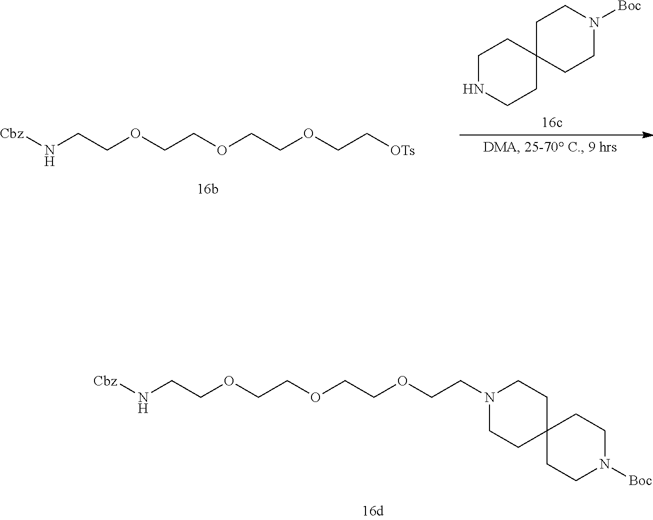 [SMILES: CC(C)(C)OC(=O)N1CCC2(CCNCC2)CC1 ; Cc1ccc(S(=O)(=O)OCCOCCOCCOCCNC(=O)OCc2ccccc2)cc1 ; CC(C)(C)OC(=O)N1CCC2(CCN(CCOCCOCCOCCNC(=O)OCc3ccccc3)CC2)CC1]

To a solution of compound 16b (1 g, 2.08 mmol) and compound 16c (600 mg, 2.36 mmol) in DMA (20 mL) was added TEA (2.18 g, 21.5 mmol, 3 mL, 10.4 eq) at 25° C., the mixture was stirred at 70° C. for 9 hrs. The mixture was cooled to 25-30° C., poured into water (50 mL), extracted with ethyl acetate (20 mL×3), the organic phase was washed with brine (30 mL×3), then separated and dried with Na₂SO₄, filtered, the filtrate was concentrated under reduced pressure to give brown oil. The oil was purified by Prep-HPLC (column: Waters Xbridge C18 150*50 mm*10 um; mobile phase: [water (10 mM NH₄HCO₃)-ACN]; B %: 38%-68%, 11.5 min), the fraction was concentrated under reduced pressure to give compound 16d (500 mg, 886 umol, 42.7% yield) as brown oil.

¹H NMR: 400 MHz CDCl₃ δ 7.30 (m, 5H), 5.92 (s, 1H), 5.09 (s, 2H), 3.58 (m, 12H), 3.38 (m, 6H), 2.54 (m, 2H), 2.40 (m, 4H), 1.49 (m, 4H), 1.44 (s, 9H).

LCMS: (method 1), Rt=0.844 min, m/z (M+1)=564.5

General Procedure for Preparation of Amine 16

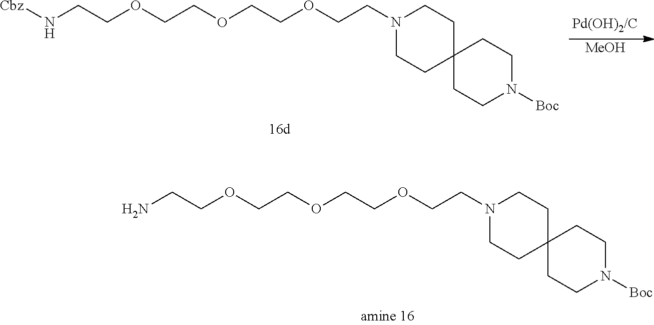 [SMILES: "MeOH" ; CC(C)(C)OC(=O)N1CCC2(CCN(CCOCCOCCOCCNC(=O)OCc3ccccc3)CC2)CC1 ; CC(C)(C)OC(=O)N1CCC2(CCN(CCOCCOCCOCCN)CC2)CC1]

To a solution of Compound 16d (180 mg, 319 umol) in MeOH (3 mL) was added Pd(OH)₂ (225 mg, 320 umol, 20% purity). The mixture was stirred at 20° C. for 1 hr under H₂ (15 psi). The reaction mixture was concentrated under reduced pressure to give amine 16 (120 mg, 279 umol, 87.4% yield) was obtained as yellow oil.

¹H NMR: 400 MHz, CDCl₃ δ 3.71-3.59 (m, 11H), 3.39 (s, 4H), 3.35 (s, 1H), 3.32 (s, 1H), 3.04-2.95 (m, 1H), 2.69-2.62 (m, 2H), 2.56 (s, 4H), 1.57 (s, 4H), 1.45 (s, 13H)

General Procedure for Preparation of Amine 16

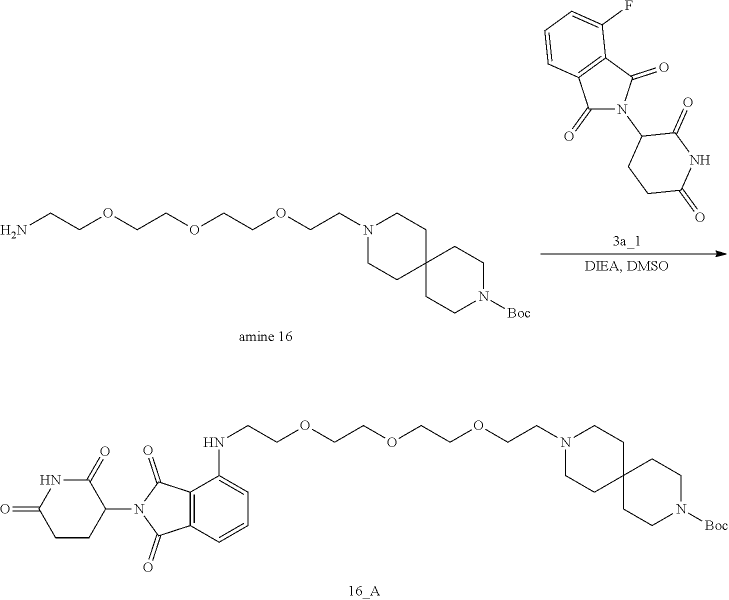 [SMILES: CC(C)(C)OC(=O)N1CCC2(CCN(CCOCCOCCOCCN)CC2)CC1 ; O=C1CCC(N2C(=O)c3cccc(F)c3C2=O)C(=O)N1 ; CC(C)(C)OC(=O)N1CCC2(CCN(CCOCCOCCOCCNc3cccc4c3C(=O)N(C3CCC(=O)NC3=O)C4=O)CC2)CC1]

To a solution of Compound 3a_1 (80 mg, 289 mmol) in DMSO (3 mL) was added DIEA (140 mg, 1.08 mmol, 188 uL) and amine 16 (120 mg, 279 umol). The mixture was stirred at 90° C. for 1 hr. The reaction mixture was acidified to pH=5 by addition AcOH. The crude product was purified by reversed-phase HPLC (0.1% FA condition) to give Compound 16_A (50 mg, 72.9 umol, 26.1% yield) was obtained as a red solid.

General Procedure for Preparation of Compound 16_B

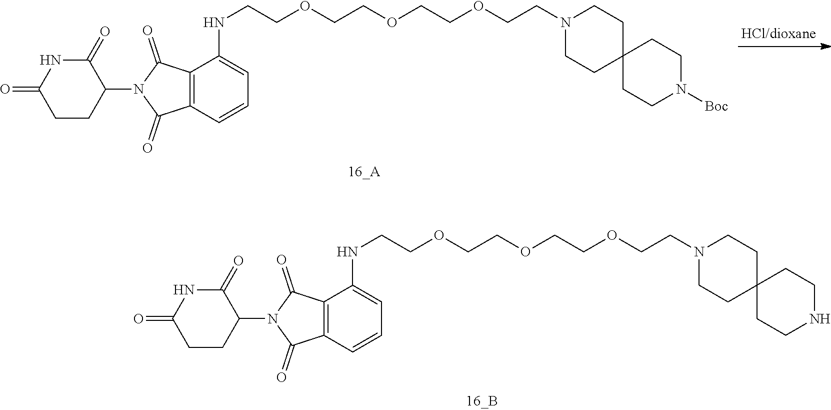 [SMILES: CC(C)(C)OC(=O)N1CCC2(CCN(CCOCCOCCOCCNc3cccc4c3C(=O)N(C3CCC(=O)NC3=O)C4=O)CC2)CC1 ; O=C1CCC(N2C(=O)c3cccc(NCCOCCOCCOCCN4CCC5(CCNCC5)CC4)c3C2=O)C(=O)N1]

A mixture of Compound 16_A (40 mg, 58.3 umol) in HCl/dioxane (4 M, 5 mL) was stirred at 20° C. for 1 hr. The reaction mixture was concentrated under reduced pressure to give Compound 16_B (30 mg, 51.2 umol, 87.8% yield) was obtained as yellow oil.

LCMS: Rt=0.539 min, m/z (M+1)=586.2

General Procedure for Preparation of “A54”

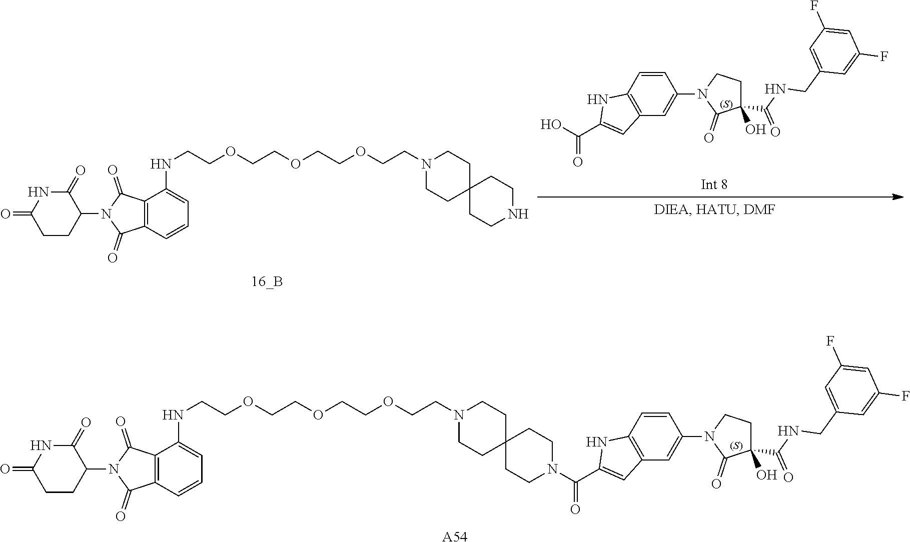

To a solution of Int 8 (30 mg, 60.8 umol) in DMF (3 mL) was successively added DIEA (24 mg, 185 umol) and HATU (30 mg, 78.9 umol) at 0° C. under N₂, the mixture was stirred at 0° C. for 10 min, then a solution of Compound 16_B (30 mg, 51.2 umol, FA) in DMF (3.00 mL) was added into the mixture at 0° C., it was stirred for 20 min under N₂ at 0° C. The mixture was poured into icy water (20.0 mL), extracted with ethyl acetate (10.0 mL×2), the organic phase was separated and washed with brine (20.0 mL×2), dried over Na₂SO₄, filtered. The filtrate was concentrated under reduced pressure to give brown oil. The crude product was purified by Prep-HPLC (column: Phenomenex Synergi C18 75*30*3 um; mobile phase: [water (0.225% FA)-ACN]; B %: 20%-40%, 8 min), solvent of the fraction was removed by lyophilization to give “A54” (8.38 mg, 7.93 umol, 15.4% yield, 94.2% purity) as yellow solid.

¹H NMR: 400 MHz, DMSO-d₆; δ 11.58 (s, 1H), 11.07 (s, 1H), 8.80-8.61 (m, 1H), 8.32 (s, 1H), 7.77 (s, 1H), 7.65-7.51 (m, 2H), 7.41 (d, J=8.4 Hz, 1H), 7.32-7.20 (m, 1H), 7.18-7.06 (m, 1H), 7.06-6.96 (m, 3H), 6.76 (s, 1H), 6.72 (s, 1H), 6.60 (s, 1H), 5.14-4.97 (m, 1H), 4.41 (d, J=6.4 Hz, 1H), 4.26 (d, J=8.0 Hz, 1H), 3.88 (d, J=8.4 Hz, 3H), 3.76-3.61 (m, 8H), 3.55 (d, J=4.4 Hz, 4H), 3.47 (s, 4H), 3.05 (s, 2H), 2.86 (d, J=12.4 Hz, 3H), 2.61 (d, J=4.8 Hz, 3H), 2.37 (s, 3H), 2.19-2.11 (m, 1H), 2.02 (s, 1H), 1.46 (s, 8H).

LCMS: Rt=0.846 min, m/z (M+1)=997.5;

HPLC: Rt=1.675 min.

### (3S)—N-[(3,5-difluorophenyl)methyl]-1-{2-[(1-{[2-(2,6-dioxopiperidin-3-yl)-1,3-dioxo-2,3-dihydro-1H-isoindol-5-yl]carbamoyl}-2,5,8,11,14-pentaoxahexadecan-16-yl)carbamoyl]-1H-indol-5-yl}-3-hydroxy-2-oxopyrrolidine-3-carboxamide (“A55”)

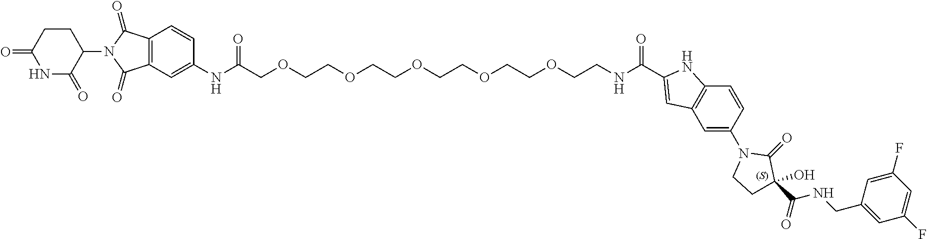

Method 1:

MS instrument type: SHIMADZU LCMS-2020, Column: Kinetex EVO C18 30*2.1 mm, 5 um, mobile phase A: 0.0375% TFA in water (v/v), B: 0.01875% TFA in Acetonitrile (v/v), gradient: 0.0 min 0% B→0.8 min 95% B→1.2 min 95% B→1.21 min 5% B→1.55 min 5% B, flow rate: 1.5 mL/min, oven temperature: 50° C.; UV detection: 220 nm & 254 nm.

Method 2:

MS instrument type: SHIMADZU LCMS-2020, Column: Kinetex EVO C18 30*2.1 mm, 5 um, mobile phase A: 0.0375% TFA in water (v/v), B: 0.01875% TFA in acetonitrile (v/v), gradient: 0.0 min 0% B→3.0 min 95% B→3.5 min 95% B→3.51 min 5% B→4.0 min 5% B, flow rate: 0.8 mL/min, oven temperature: 50° C.; UV detection: 220 nm & 254 nm.

4. Experimental for Largest Scale Run:

General Procedure for Preparation of Compound 2b

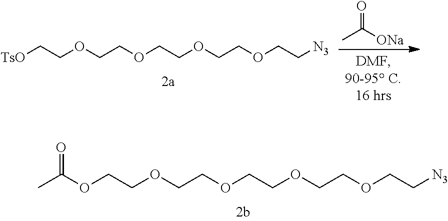

A mixture of compound 2a (5.00 g, 12.0 mmol), sodium acetate (10.0 g, 122 mmol) in DMF (75 mL) was degassed and purged with N₂ for 3 times, and then the mixture was stirred at 95-100° C. for 16 hrs under N₂ atmosphere. TLC (Petroleum ether/Ethyl acetate=1/2) showed compound 2a (R_{f}=0.65) was consumed and a main spot (R_{f}=0.62) was formed. The reaction mixture was poured into saturated sodium bicarbonate aqueous solution (300 mL), extracted with MTBE (100 mL×3).

The combined organic layers were washed with brine (60 mL×2), the organic phase was separated and dried with Na₂SO₄, filtered and filtrate was concentrated under reduced pressure to give compound 2b (4.27 g, crude) as a yellow oil.

¹H NMR: 400 MHz, CDCl₃ δ 4.16-4.25 (m, 2H), 3.64-3.70 (m, 16H), 3.38 (t, J=5.2 Hz, 2H), 2.07 (s, 3H).

General Procedure for Preparation of Compound 2c

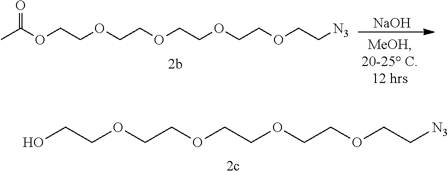

A mixture of compound 2b (3.66 g, 12.0 mmol), NaOH (2 M, 35 mL) in MeOH (35 mL) was stirred at 20-25° C. for 12 hrs under N₂ atmosphere. TLC (Petroleum ether/Ethyl acetate=1/1) indicated compound 2b (R_{f}=0.50) was consumed completely and one new spot formed (R_{f}=0.20). The reaction was clean according to TLC. The reaction mixture was diluted with H₂O (200 mL), washed with MTBE (50×2 mL). Then to the aqueous layer was saturated with NaCl (s) and extracted with EtOAc (100 mL×2). The combined organic layer was dried with Na₂SO₄, filtered and filtration was concentrated under reduced pressure to give compound 2c (1.96 g, 7.44 mmol, 62.1% yield) as a yellow oil.

¹H NMR: 400 MHz, CDCl₃ δ 3.69-3.73 (m, 2H), 3.62-3.68 (m, 14H), 3.57-3.61 (m, 2H), 3.33-3.42 (m, 2H).

General Procedure for Preparation of Compound 2d

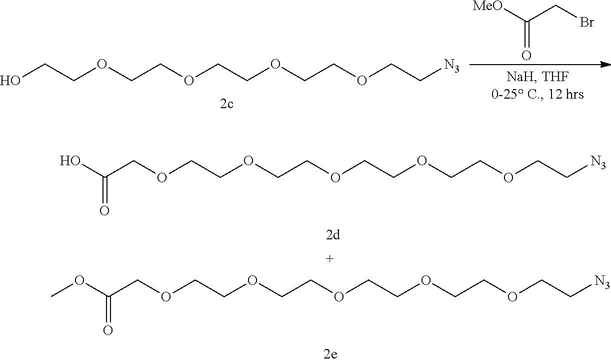 [SMILES: COC(=O)CBr ; [N-]=[N+]=NCCOCCOCCOCCOCCO ; [N-]=[N+]=NCCOCCOCCOCCOCCOCC(=O)O ; COC(=O)COCCOCCOCCOCCOCCN=[N+]=[N-]]

To a solution of NaH (1.83 g, 45.8 mmol, 60% purity) in THF (30 mL) was added compound 2c (1.96 g, 7.44 mmol) in THF (10 mL) at 0-5° C. under N₂ and kept stirred for 1 h at 20-25° C. under N₂. To the mixture was added methyl 2-bromoacetate (2.28 g, 14.9 mmol, 1.40 mL) in THE (10 mL) at 0-5° C. under N₂ and kept stirred for 12 hrs at 20-25° C. under N₂. TLC (Petroleum ether/Ethyl acetate=1/1) indicated compound 2c (R_{f}=0.50) was consumed completely. The reaction mixture was poured into saturated NH₄Cl (120 mL), extracted with EtOAc (30 mL×3), the combined organic layers were washed with brine (15 mL), the organic phase was separated and dried with Na₂SO₄, filtered and filtration was concentrated under reduced pressure to give compound 2e (200 mg, crude) as a brown oil, which checked by 1H NMR. The aqueous layer was adjusted pH=5-6 with citric acid (s). Then the mixture was extracted with MeCN (thrice, 100 mL, 50 mL, 50 mL). The combined organic layer (compound 2d in MeCN solution) checked by 1H NMR and used to next step directly.

¹H NMR: 400 MHz, CDCl₃ δ 3.48-3.63 (m, 20H), 3.25-3.30 (m, 2H).

General Procedure for Preparation of Compound 2e

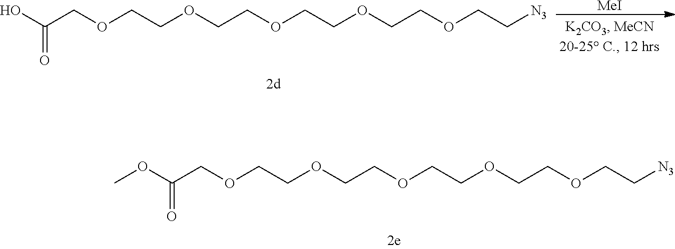 [SMILES: "MeI" ; [N-]=[N+]=NCCOCCOCCOCCOCCOCC(=O)O ; COC(=O)COCCOCCOCCOCCOCCN=[N+]=[N-]]

To a solution of compound 2d (2.39 g, 7.44 mmol) in MeCN (200 mL) was added K₂CO₃ (4.11 g, 29.8 mmol) at 20-25° C. under stirred for 1 h at 20-25° C. under N₂ atmosphere. Then to the mixture was added MeI (10.6 g, 74.4 mmol, 4.63 mL) at 20-25° C. under N₂ atmosphere. The mixture was stirred at 20-25° C. for 12 hrs under N₂. 1H NMR showed compound 2d was disappeared and compound 2e was found (The reaction mixture was concentrated and directed for 1H NMR). The reaction mixture was concentrated under reduce pressure. The residue was dissolved with EtOAc (200 mL), then the solution was dried over Na₂SO₄, filtered and filtrate was concentrated under reduce pressure to give compound 2e (890 mg, 2.65 mmol, 35.7% yield) as a brown oil.

¹H NMR: 400 MHz, CDCl₃ δ 4.18 (s, 2H), 3.75 (s, 3H), 3.62-3.74 (m, 18H), 3.40 (t, J=5.2 Hz, 2H).

General Procedure for Preparation of Compound 2f

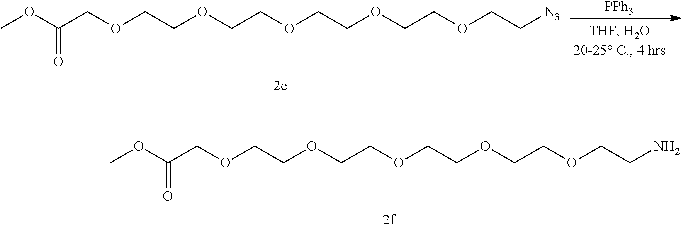 [SMILES: COC(=O)COCCOCCOCCOCCOCCN=[N+]=[N-] ; COC(=O)COCCOCCOCCOCCOCCN ; "PPh3"]

A mixture of compound 2e (800 mg, 2.39 mmol), PPh₃ (1.12 g, 4.27 mmol) in H₂O (3 mL) and THF (15 mL) was degassed and purged with N₂ for 3 times, and then the mixture was stirred at 20-25° C. for 4 hrs under N₂ atmosphere. TLC (Petroleum ether/Ethyl acetate=0/1) showed compound 2e (R_{f}=0.60) was consumed (the reactant solution was directed used for next step.

General Procedure for Preparation of Compound 2g

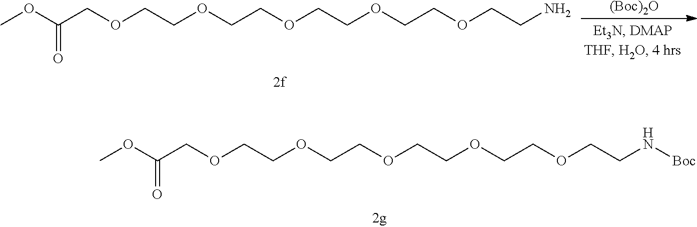 [SMILES: "(Boc)2O" ; COC(=O)COCCOCCOCCOCCOCCN ; COC(=O)COCCOCCOCCOCCOCCNC(=O)OC(C)(C)C]

A mixture of compound 2f (800 mg, 2.59 mmol), TEA (730 mg, 7.21 mmol, 1.00 mL), (Boc)₂O (1.30 g, 5.96 mmol, 1.37 mL), DMAP (145 mg, 1.19 mmol) in H₂O (3 mL) and THF (15 mL) was degassed and purged with N₂ for 3 times, and then the mixture was stirred at 20-25° C. for 12 hrs under N₂ atmosphere. TLC (Petroleum ether/Ethyl acetate=0/1) showed compound 2g (R_{f}=0.45) was formed. The reaction mixture was concentrated under reduce pressure. The residue was purified with flash silica gel chromatography (Silica Flash Column, Eluent of 0˜100% Ethyl acetate/Petroleum ether gradient), the spot (R_{f}=0.45) was collected to give compound 2g (145 mg, 354 umol, 13.7% yield) as a yellow oil.

¹H NMR: 400 MHz, CDCl₃ δ 5.06 (s, 1H), 4.17 (s, 2H), 3.75 (s, 3H), 3.59-3.74 (m, 16H), 3.54 (t, J=5.2 Hz, 2H), 3.31 (d, J=4.8 Hz, 2H), 1.44 (s, 9H).

General Procedure for Preparation of Compound 2h

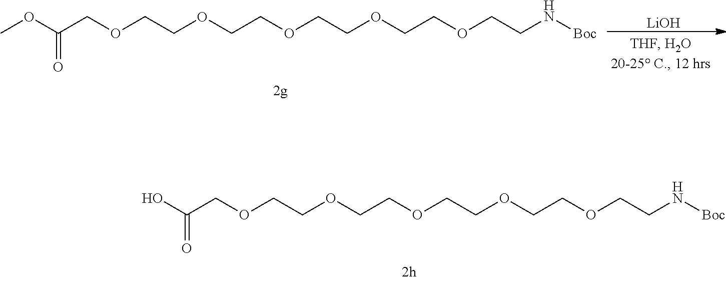 [SMILES: COC(=O)COCCOCCOCCOCCOCCNC(=O)OC(C)(C)C ; "LiOH" ; CC(C)(C)OC(=O)NCCOCCOCCOCCOCCOCC(=O)O]

A mixture of compound 2g (145 mg, 354 umol), LiOH.H₂O (30 mg, 715 umol) in THF (4 mL) and H₂O (1 mL) was stirred at 20-25° C. for 12 hrs under N₂ atmosphere. TLC (Dichloromethane/Methanol=10/1) showed compound 2g (R_{f}=0.60) was consumed and a main new spot (R_{f}=0.00) was formed. The reaction mixture was diluted with H₂O (30 mL). The mixture was adjusted pH=3-4 with citric acid (4 M in H₂O) and added NaCl(s) until saturated. Then the mixture was extracted with EtOAc (10 mL×3), the organic layers were combined and dried over Na₂SO₄, filtered and filtrate was concentrated under reduce pressure to give compound 2h (137 mg, 346 umol, 97.8% yield) as a yellow oil, which confirmed by 1H NMR.

¹H NMR: 400 MHz, DMSO-d₆ δ 12.53 (s, 1H), 6.74 (s, 1H), 4.01 (s, 2H), 3.44-3.62 (m, 16H), 3.35-3.38 (m, 2H), 3.05 (q, J=6.0 Hz, 2H), 1.37 (s, 9H).

General Procedure for Preparation of Compound 2i

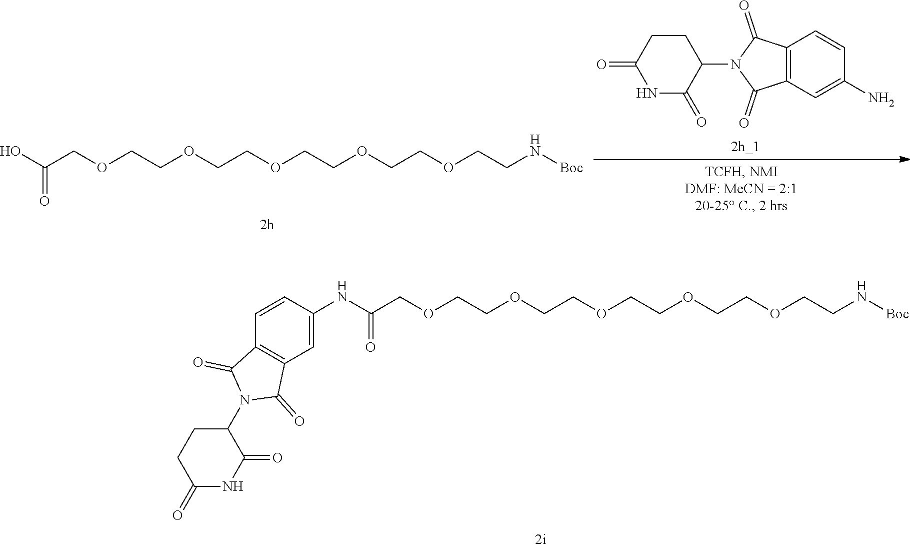 [SMILES: Nc1ccc2c(c1)C(=O)N(C1CCC(=O)NC1=O)C2=O ; CC(C)(C)OC(=O)NCCOCCOCCOCCOCCOCC(=O)O ; CC(C)(C)OC(=O)NCCOCCOCCOCCOCCOCC(=O)Nc1ccc2c(c1)C(=O)N(C1CCC(=O)NC1=O)C2=O]

To a solution of compound 2h (70 mg, 177 umol) and compound 2h_1 (51 mg, 186 umol) in ACN (4 mL) and DMF (2 mL) was added TCFH (121 mg, 431 umol) and NMI (100 mg, 1.22 mmol) under N₂ at 20-25° C., after addition, the mixture was stirred at 20-25° C. for 2 hrs. LCMS showed the compound 2i was detected (RT=0.876 min, m/z (M+1−100=551), TLC (Petroleum ether/Ethyl acetate=0/1) showed compound 2i was consumed up (R_{f}=0.00), a new spot was detected (R_{f}=0.10). The reaction solution was quenched with AcOH (1 mL), then it was poured into water (10 mL) and extracted with ethyl acetate (10 mL*2), the organic layer was washed with brine (10 mL), dried over Na₂SO₄, filtrated and concentrated under reduce pressure. The crude was purified by silica gel column chromatography (SiO₂, Petroleum ether/ethyl acetate=5/1-1/1-0/1, the spot R_{f}=0.10 was collected) then it was concentrated under reduce pressure to afford compound 2i (90 mg, 137 umol, 77.4% yield) as a pale yellow oil.

LCMS: (method 1), RT=0.875 min, m/z (M+1-100)=551

General Procedure for Preparation of Compound 2j

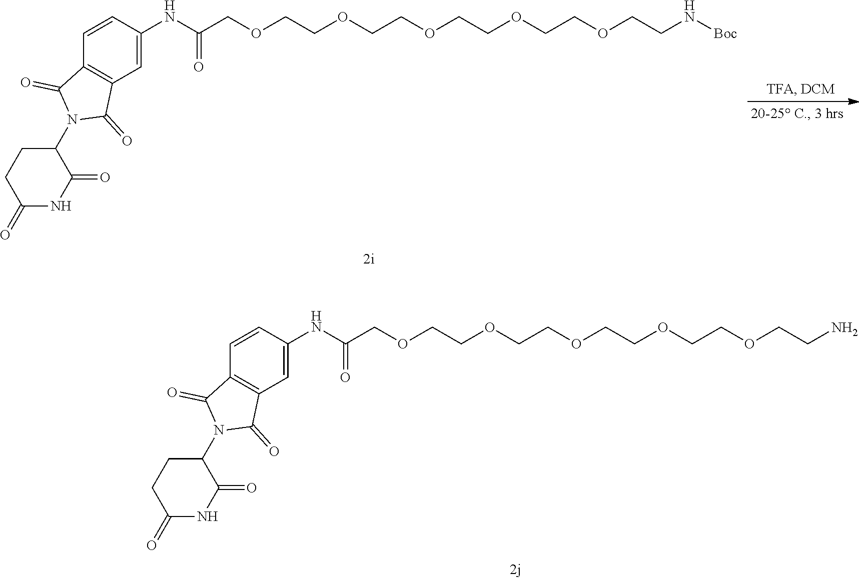 [SMILES: CC(C)(C)OC(=O)NCCOCCOCCOCCOCCOCC(=O)Nc1ccc2c(c1)C(=O)N(C1CCC(=O)NC1=O)C2=O ; NCCOCCOCCOCCOCCOCC(=O)Nc1ccc2c(c1)C(=O)N(C1CCC(=O)NC1=O)C2=O]

To a solution of compound 2i (90 mg, 138 umol) in DCM (5 mL) was added TFA (158 mg, 1.39 mmol) at 20-25° C., after addition, the mixture was stirred at 20-25° C. for 3 hrs. LCMS showed the MS of compound 2j was detected (Rt=0.733 min m/z (M+1)=551). The reaction was concentrated under reduce pressure to afford compound 2j (90 mg, crude, TFA) as a yellow oil.

LCMS: (method 1), RT=0.733 min, m/z (M+1)=551

General Procedure for Preparation of “A55”

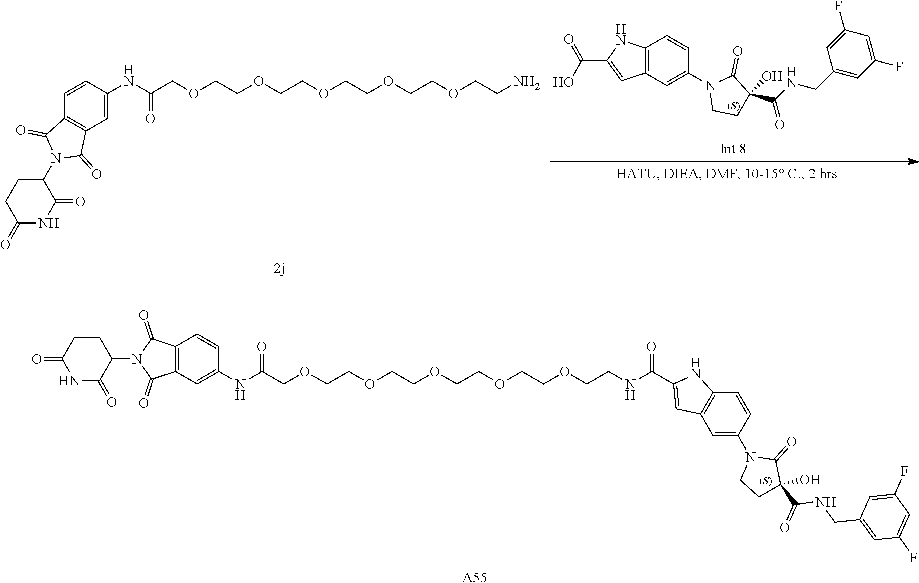

To a solution of compound int 8 (30 mg, 69.8 umol) in DMF (3 mL) was added HATU (40 mg, 105 umol) and DIEA (45 mg, 348 umol) at 10-15° C. under N₂, after addition, the compound 2j (55 mg, 82.7 umol TFA) dissolved in DMF (1 mL) was added to the solution at 10-15° C., then it was stirred at 10-15° C. for 1 hr under N₂. LCMS showed the mass of compound 2 (RT=0.890 min, m/z (M+1)=962) was detected. The reaction solution was quenched with AcOH (1 mL) and poured into water (5 mL), then it was extracted with ethyl acetate (10 mL*2), the organic layer was washed with brine (10 mL), dried over Na₂SO₄, filtrated and concentrated under reduce pressure. The crude was purified by Pre-HPLC (column: Phenomenex Luna C18 150*25 mm*10 um; mobile phase: [water (0.1% TFA)-ACN]; B %: 25%-55%, 10 min), then it was concentrated under reduce pressure to afford “A55” (33.96 mg, 33.6 umol, 95.2% purity) as a yellow gum.

LCMS: (method 1), RT=0.885 min, m/z (M+1)=962

HPLC: (method 2), RT=1.885 min

¹H NMR: 400 MHz, DMSO-d₆ δ 11.68 (s, 1H), 11.18 (s, 1H), 10.39 (s, 1H), 8.76 (t, J=6.4 Hz, 1H), 8.61 t, J=5.6 Hz, 1H), 8.36 (d, J=1.6 Hz, 1H), 8.09 (dd, J=8.4, 1.6 Hz, 1H), 7.95 (d, J=8.4 Hz, 1H), 7.84 (d, J=1.6 Hz, 1H), 7.60 (dd, J=8.8, 2.0 Hz, 1H), 7.49 (d, J=8.8 Hz, 1H), 7.19 (s, 1H), 7.01-7.15 (m, 3H), 5.19 (dd, J=12.8, 5.2 Hz, 1H), 4.48 (dd, J=16.0, 6.8 Hz, 1H), 4.33 (dd, J=16.0, 6.0 Hz, 1H), 4.23 (s, 2H), 3.96 (t, J=6.8 Hz, 1H), 3.83-3.43 (m, 24H overlap H₂O signal), 2.89-3.02 (m, 1H), 2.64-2.76 (m, 2H), 2.05-2.27 (m, 2H).

### (3S)—N-[(3,5-difluorophenyl)methyl]-1-{2-[4-(14-{[2-(2,6-dioxopiperidin-3-yl)-1,3-dioxo-2,3-dihydro-1H-isoindol-4-yl]amino}-3,6,9,12-tetraoxatetradecan-1-yl)piperazine-1-carbonyl]-1H-indol-5-yl}-3-hydroxy-2-oxopyrrolidine-3-carboxamide (“A56”)

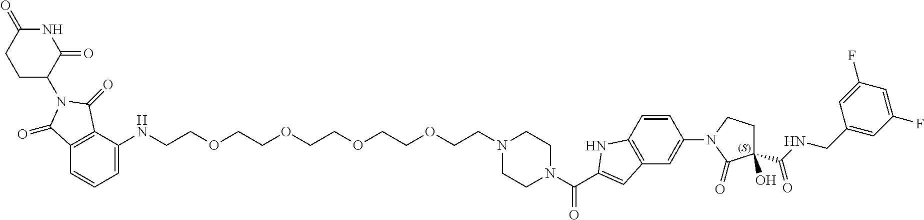

LC-MS or HPLC Method:

Method 1:

MS instrument type: SHIMADZU LCMS-2020, Column: Kinetex EVO C18 2.1×30 mm, 5 um, mobile phase A: 0.025% NH₃H₂O in Water (v/v), B: Acetonitrile, gradient: 0.0 min 0% B→0.8 min 60% B→1.2 min 60% B→1.21 min 0% B→1.55 min 0% B, flow rate: 1.5 mL/min, oven temperature: 40° C.; UV detection: 220 nm & 254 nm.

Method 2:

MS instrument type: SHIMADZU LCMS-2020, Column: Kinetex EVO C18 30*2.1 mm, 5 um, mobile phase A: 0.0375% TFA in water (v/v), B: 0.01875% TFA in Acetonitrile (v/v), gradient: 0.0 min 0% B→0.8 min 60% B→1.2 min 60% B→1.21 min 0% B→1.55 min 0% B, flow rate: 1.5 mL/min, oven temperature: 50° C.; UV detection: 220 nm & 254 nm. 4.0 min 0% B, flow rate: 0.8 mL/min, oven temperature: 50° C.; UV detection: 220 nm & 254 nm.

Experimental for Largest Scale Run:

General Procedure for Preparation of Compound 15a

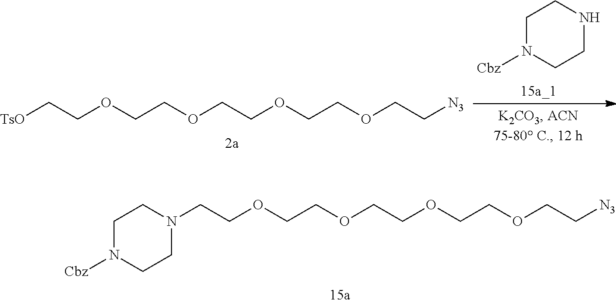 [SMILES: O=C(OCc1ccccc1)N1CCNCC1 ; Cc1ccc(S(=O)(=O)OCCOCCOCCOCCOCCN=[N+]=[N-])cc1 ; [N-]=[N+]=NCCOCCOCCOCCOCCN1CCN(C(=O)OCc2ccccc2)CC1]

A mixture of compound 2a (5 g, 12 mmol), compound 15a_1 (2.90 g, 13.2 mmol), K₂CO₃ (3.31 g, 24.0 mmol) in ACN (30.0 mL) was degassed and purged with N₂ for 3 times, and then the mixture was stirred at 75-80° C. for 12 hrs under N₂ atmosphere. TLC (Petroleum ether: Ethyl acetate=1/2) showed compound 2a (R_{f}=0.75) was consumed and a main new spot (R_{f}=0.05) was found. The reaction mixture was poured into H₂O (140 mL), the mixture was extracted with MTBE (30.0 mL×3). The organic layer were combined and used to next step.

General Procedure for Preparation of Compound 15_A

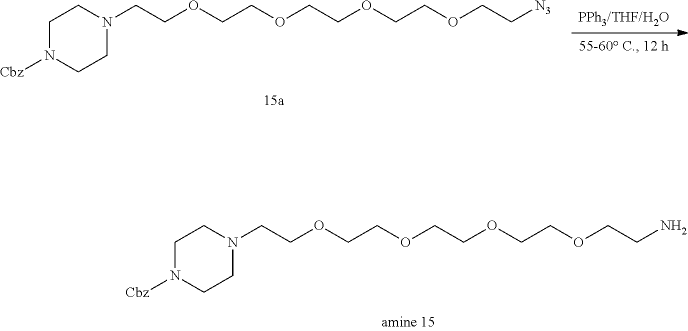 [SMILES: [N-]=[N+]=NCCOCCOCCOCCOCCN1CCN(C(=O)OCc2ccccc2)CC1 ; NCCOCCOCCOCCOCCN1CCN(C(=O)OCc2ccccc2)CC1]

A mixture of compound 15a (5.58 g, 12 mmol), PPh₃ (6.30 g, 24.0 mmol) in THF (80 mL) and H₂O (10 mL) was degassed and purged with N₂ for 3 times, and then the mixture was stirred at 55-60° C. for 12 h under N₂ atmosphere. TLC (Petroleum ether: Ethyl acetate=1/2) showed compound 15a (R_{f}=0.60) was remained. The mixture was kept stirred at 55-60° C. for 16 hrs. LCMS showed compound 15a was consumed, a main new peaks with MS value of amine 15 (RT=0.941 min, M+1=440) was detected. The reaction mixture was concentrated under reduce pressure. Half the residue was purified by column chromatography (SiO₂, DCM/MeOH=1/0 to 10/1) to give amine 15 (430 mg, 978 umol, 8.16% yield) as a yellow oil which confirmed by 1H NMR and LCMS.

¹H NMR: 400 MHz, CDCl₃ δ 7.28-7.41 (m, 5H), 5.13 (s, 2H), 3.59-3.67 (m, 13H) 3.51-3.58 (m, 5H), 2.90 (t, J=5.2 Hz, 2H), 2.61 (t, J=5.6 Hz, 2H), 2.48 (s, 4H), 2.18 (s, 2H) (overlap H₂O signal)

LCMS: (method 1), RT=0.966 min, m/z (M+1)=440

General Procedure for Preparation of Compound 15_A

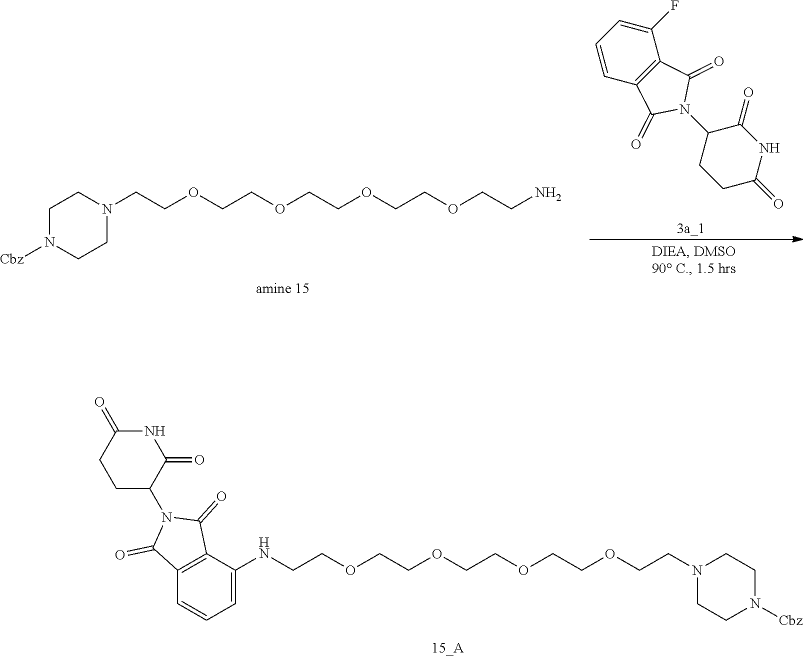 [SMILES: NCCOCCOCCOCCOCCN1CCN(C(=O)OCc2ccccc2)CC1 ; O=C1CCC(N2C(=O)c3cccc(F)c3C2=O)C(=O)N1 ; O=C1CCC(N2C(=O)c3cccc(NCCOCCOCCOCCOCCN4CCN(C(=O)OCc5ccccc5)CC4)c3C2=O)C(=O)N1]

A solution of compound 3a_1 (50 mg, 181 umol), amine 15 (100 mg, 227 umol) and DIEA (148 mg, 1.15 mmol, 200 uL) in DMSO (3 mL) was stirred at 90° C. under N₂ for 1.5 hr. Three batches were carried in parallel. The mixture was cooled to 25-30° C., pH value of the mixture was adjusted to 6-7 with AcOH. The mixture was purified by Prep-HPLC (column: Phenomenex Synergi C18 150*25*10 um; mobile phase: [water (0.225% FA)-ACN]; B %: 18%-48%, 10 min), the fraction was concentrated under reduced pressure to give compound 15_A (160 mg, 204 umol, 37.7% yield, 94.9% purity, FA) as yellow oil.

LCMS: EW18785-68-P1A, Rt=0.799 min. m/z (M+1)=696.4

General Procedure for Preparation of Compound 15-1

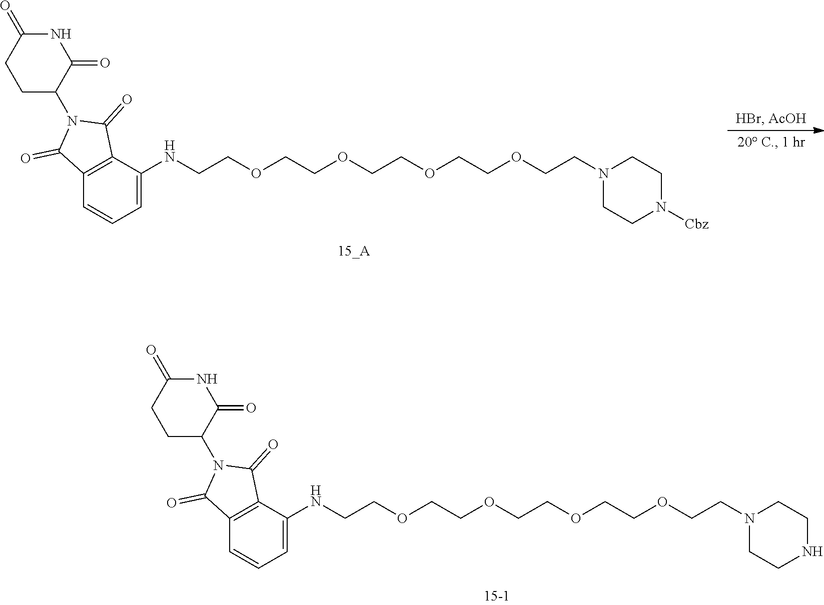 [SMILES: O=C1CCC(N2C(=O)c3cccc(NCCOCCOCCOCCOCCN4CCN(C(=O)OCc5ccccc5)CC4)c3C2=O)C(=O)N1 ; O=C1CCC(N2C(=O)c3cccc(NCCOCCOCCOCCOCCN4CCNCC4)c3C2=O)C(=O)N1]

A mixture of Compound 15_A (40 mg, 57.4 umol) in HBr (10 mL) and HOAc (5 mL) was stirred at 20° C. for 1 hr under N₂ atmosphere. The reaction mixture was acidified to pH=7 by addition NaHCO₃(aq), and then was concentrated under reduced pressure to give a residue. The crude product was purified by reversed-phase HPLC (0.1% FA condition) to give compound 15-1 (30 mg, 53.4 umol, 92.9% yield) was obtained as a yellow solid and confirmed by next step.

LCMS: Rt=0.543 min, m/z (M+1)=562.0

General Procedure for Preparation of “A56”

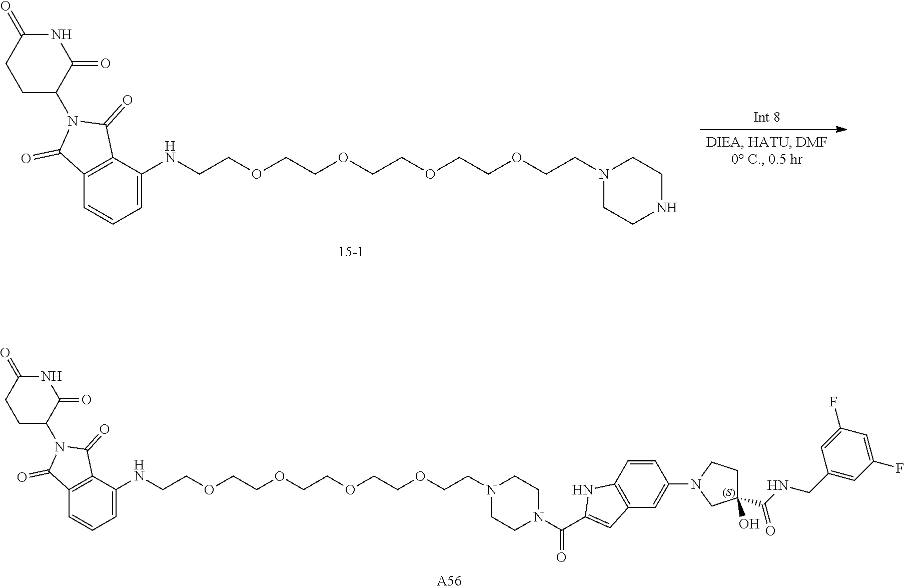

To a solution of Int 8 (20 mg, 46.5 umol) in DMF (3 mL) was successively added DIEA (20 mg, 154 umol) and HATU (30 mg, 78.9 umol) at 0° C. under N₂, the mixture was stirred at 0° C. for 10 min, then a solution of Compound 15-1 (30 mg, 53.4 umol, FA) in DMF (3.00 mL) was added into the mixture at 0° C., it was stirred for 20 min under N₂ at 0° C. The mixture was poured into icy water (20.0 mL), extracted with ethyl acetate (10.0 mL×2), the organic phase was separated and washed with brine (20.0 mL×2), dried with Na₂SO₄, filtered, the filtrate was concentrated under reduced pressure to give brown oil. It was purified by Prep-HPLC (column: Phenomenex Synergi C18 150*50*3 um; mobile phase: [water (0.225% FA)-ACN]; B %: 20%-50%, 10 min), solvent of the fraction was removed by lyophilization to give “A56” (10 mg, 10.1 umol, 18.9% yield, 98.3% purity) as yellow solid.

¹H NMR: 400 MHz, DMSO-d₆; δ 11.61 (s, 1H), 11.09 (s, 1H), 8.69 (t, J=6.4 Hz, 1H), 8.25 (s, 1H), 7.89-7.72 (m, 1H), 7.60-7.50 (m, 2H), 7.42 (d, J=9.2 Hz, 1H), 7.13 (d, J=8.4 Hz, 1H), 7.09-6.98 (m, 4H), 6.79 (s, 1H), 6.72 (s, 1H), 6.59 (s, 1H), 5.10-5.00 (m, 1H), 4.41 (d, J=6.4, 15.5 Hz, 1H), 4.31-4.23 (m, 1H), 3.89 (s, 3H), 3.72 (s, 5H), 3.60 (t, J=5.6 Hz, 3H), 3.53 (d, J=4.0 Hz, 5H), 3.50 (s, 7H), 3.49-3.46 (m, 6H), 2.64-2.60 (m, 3H), 2.14 (d, J=12.4 Hz, 1H), 2.04 (s, 1H).

LCMS: Rt=0.671 min, m/z (M+1)=973.3;

HPLC: Rt=1.637 min.

### (3S)—N-[(3,5-difluorophenyl)methyl]-1-{2-[(16-{[2-(2,6-dioxopiperidin-3-yl)-1,3-dioxo-2,3-dihydro-1H-isoindol-4-yl]carbamoyl}hexadecyl)carbamoyl]-1H-indol-5-yl}-3-hydroxy-2-oxopyrrolidine-3-carboxamide (“A57”)

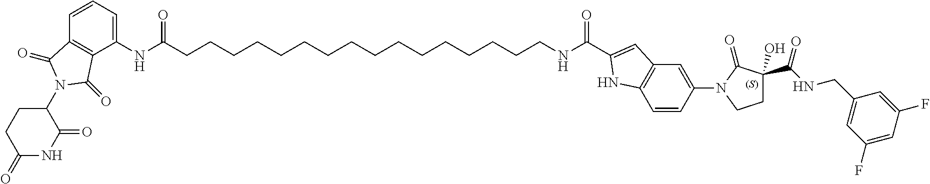

LC-MS or HPLC Method:

Method 1:

MS instrument type: SHIMADZU LCMS-2020, Column: Kinetex EVO C18 30*2.1 mm, 5 um, mobile phase A: 0.0375% TFA in water (v/v), B: 0.01875% TFA in Acetonitrile (v/v), gradient: 0.0 min 5% B→0.8 min 95% B→1.2 min 95% B→1.21 min 5% B→1.55 min 5% B, flow rate: 1.5 mL/min, oven temperature: 50° C.; PDA detection: 220 nm & 254 nm.

Method 2:

MS instrument type: SHIMADZU LCMS-2020, Column: Kinetex EVO C18 30*2.1 mm, 5 um, mobile phase A: 0.0375% TFA in water (v/v), B: 0.01875% TFA in Acetonitrile (v/v), gradient: 0.0 min 5% B→3.0 min 95% B→3.5 min 95% B→3.51 min 5% B→4.0 min 5% B, flow rate: 0.8 mL/min, oven temperature: 50° C.; PDA detection: 220 nm & 254 nm.

Method 3:

Instrument type: SHIMADZU LC-20AD, Column: Kinetex C18 LC Column 4.6×50 mm, Sum, mobile phase A: 0.0375% TFA in water (v/v), B: 0.01875% TFA in Acetonitrile (v/v), gradient: 0.0 min 10% B→2.40 min 80% B→3.7 min 80% B→3.71 min 10% B→4.00 min 10% B, flow rate: 1.5 mL/min, oven temperature: 50° C.; PDA detection: (220 nm&215 nm&254 nm)

4. Experimental for Largest Scale Run:

General Procedure for Preparation of Compound 10b

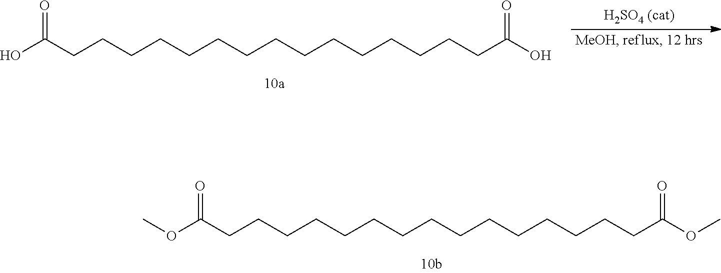

A mixture of compound 10a (3 g, 9.99 mmol) and H₂SO₄ (8 drops, 98% purity) in MeOH (30 mL) was stirred at 64-65° C. for 12 hours under N₂ atmosphere. 1H NMR showed the compound 10a was disappeared and compound 10b was found (The solution was determined by 1H NMR directly). The reaction mixture was cooled to 10-20° C. and kept stirred at 10-20° C. for 30 mins, then filtered and the filter cake was dried under reduced pressure to give compound 10b (3.02 g, 9.19 mmol, 92.1% yield) as a white solid, which was confirmed by 1H NMR.

¹H NMR: 400 MHz, CDCl₃ δ 3.69 (s, 6H), 2.32 (t, J=7.2 Hz, 4H), 1.59-1.69 (m, 4H), 1.22-1.37 (m, 22H).

General Procedure for Preparation of Compound 10c

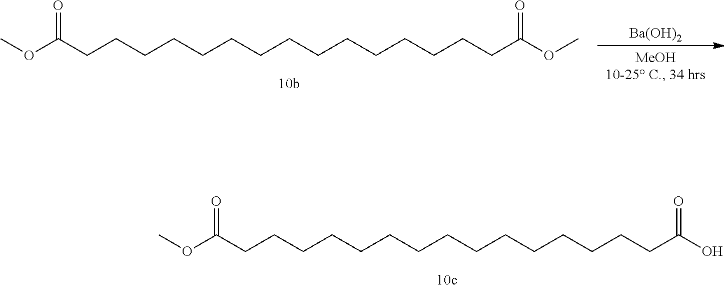

To a suspension of compound 10b (3.02 g, 9.19 mmol) in MeOH (12.0 mL) was added a solution of Ba(OH)₂ (866 mg, 5.05 mmol) in MeOH (12 mL) at 10-20° C. under N₂ atmosphere and kept stirred at 15-25° C. for 22 hours under N₂ atmosphere. 1H NMR showed the compound 10b was retained (one drop reaction solution was added to a drop 12 M HCl, then determined by 1H NMR). The reaction mixture was stirred at 15-25° C. for 12 hours under N₂ atmosphere. 1H NMR showed the most compound 10c was found (one drop reaction solution was added to a drop 12 M HCl, then determined by 1H NMR). The reaction mixture was filtered and the filter cake was suspension in 4 M HCl solution (15 mL) at 15-25° C. and kept stirred at 15-25° C. for 30 mins, then the mixture was filtered and filter cake was dried under reduced pressure to give compound 10c (2.42 g, 7.70 mmol, 83.7% yield) as a white solid, which was confirmed by 1H NMR.

¹H NMR: 400 MHz, CDCl₃ δ 3.60 (s, 3H), 2.19-2.33 (m, 4H), 1.48-1.63 (m, 4H), 1.12-1.33 (m, 22H).

General Procedure for Preparation of Compound 10d

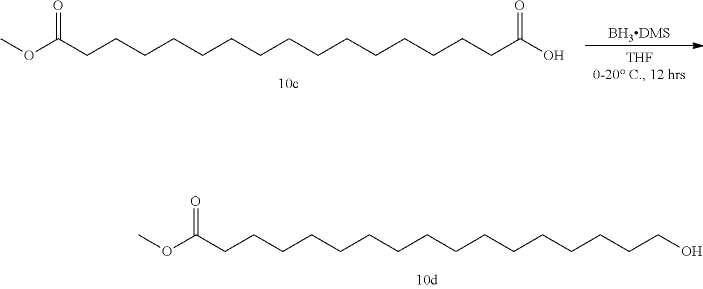 [SMILES: COC(=O)CCCCCCCCCCCCCCCC(=O)O ; "BH3·DMS" ; COC(=O)CCCCCCCCCCCCCCCCO]

To a suspension of compound 10c (2.42 g, 7.70 mmol) in THF (25.0 mL) was added dropwise BH₃-Me₂S (10.0 M, 940 uL) at 0-10° C. under N₂ atmosphere and kept stirred at 10-20° C. for 12 hours under N₂ atmosphere. 1H NMR showed the most compound 10d was found (one drop reaction solution was added to a drop 12 M HCl, then determined by 1H NMR). The reaction mixture was poured into saturated NH₄Cl solution (25 mL) at 0-10° C. and kept stirred at 10-20° C. for 15 mins, the aqueous phase was extracted with EtOAc (50 mL×2), the combined organic phase was dried over anhydrous Na₂SO₄, filtered and filtrate was concentrated under reduced pressure. The residue was checked by 1H NMR. TLC 1 (Petroleum ether/Ethyl acetate=3/1). The residue was purified by silica gel column (Petroleum ether/Ethyl acetate, 20/1 to 3/1), the spot (R_{f}=0.32) was collected to give compound 10d (1.98 g, 6.59 mmol, 85.6% yield) as a white solid, which was confirmed by 1H NMR.

¹H NMR: 400 MHz, CDCl₃ δ 3.60 (s, 3H), 3.57 (t, J=7.2 Hz, 2H), 2.23 (t, J=7.2 Hz, 2H), 1.44-1.61 (m, 4H), 1.13-1.33 (m, 22H).

General Procedure for Preparation of Compound 10e

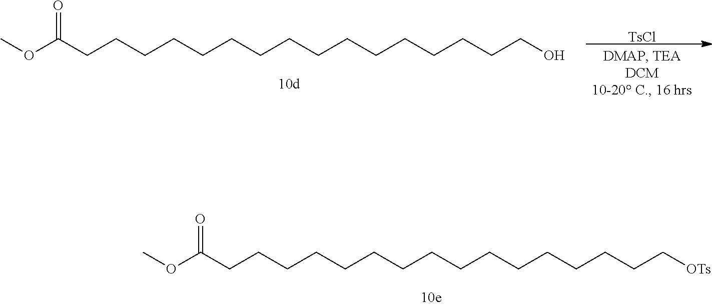 [SMILES: COC(=O)CCCCCCCCCCCCCCCCO ; "TsCl" ; COC(=O)CCCCCCCCCCCCCCCCOS(=O)(=O)c1ccc(C)cc1]

To a solution of compound 10d (1.98 g, 6.59 mmol) in DCM (20.0 mL) was added TEA (1.33 g, 13.2 mmol, 1.83 mL), DMAP (80 mg, 655 umol) and 4-methylbenzenesulfonyl chloride (1.63 g, 8.57 mmol) at 10-20° C. under N₂ atmosphere and kept stirred at 10-20° C. for 16 hours under N₂ atmosphere. To the mixture was added water (50.0 mL) at 10-20° C. and the organic phase was dried over anhydrous Na₂SO₄, filtered and filtrated was concentrated under reduced pressure. The residue was purified by silica gel column (Petroleum ether/Ethyl acetate, 1/0 to 3/1), the spot (R_{f}=0.83) was collected to give compound 10e (1.43 g, 3.15 mmol, 47.7% yield) as a white solid.

¹H NMR: 400 MHz, CDCl₃; δ 7.72 (d, J=8.4 Hz, 2H), 7.27 (d, J=8.4 Hz, 2H), 3.95 (t, J=6.4 Hz, 2H), 3.60 (s, 3H), 2.38 (s, 3H), 2.23 (t, J=7.6 Hz, 2H), 1.52-1.60 (m, 4H), 1.10-1.28 (m, 24H).

General Procedure for Preparation of Compound 10f

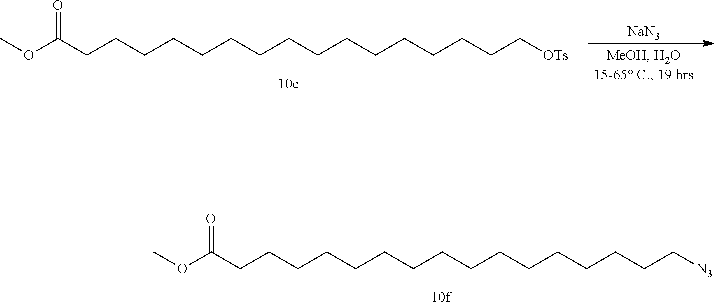 [SMILES: COC(=O)CCCCCCCCCCCCCCCCOS(=O)(=O)c1ccc(C)cc1 ; COC(=O)CCCCCCCCCCCCCCCCN=[N+]=[N-] ; "NaN3"]

To a solution of methyl compound 10e (1.43 g, 3.15 mmol) in MeOH (15.0 mL) and H₂O (1.50 mL) was added NaN₃ (310 mg, 4.77 mmol) at 15-25° C., then the mixture was heated to 64-65° C. and kept stirred at 64-65° C. for 19 hours. 1H NMR showed the compound 10e was disappeared and compound 10f was found (To the 0.3 mL of the reaction solution was added saturated NaHCO₃ (5 mL) and DCM (4.00 mL), the organic phase was concentrated under reduced pressure at 25° C., then determined by 1H NMR). The reaction mixture was cooled to 15-25° C. The reaction mixture was not purified and used for next step directly.

¹H NMR: 400 MHz, CDCl₃; δ 3.69 (s, 3H), 3.28 (t, J=6.8 Hz, 2H), 2.34 (t, J=6.8 Hz, 2H), 1.56-1.72 (m, 4H), 1.24-1.43 (m, 22H).

General Procedure for Preparation of Compound 10g

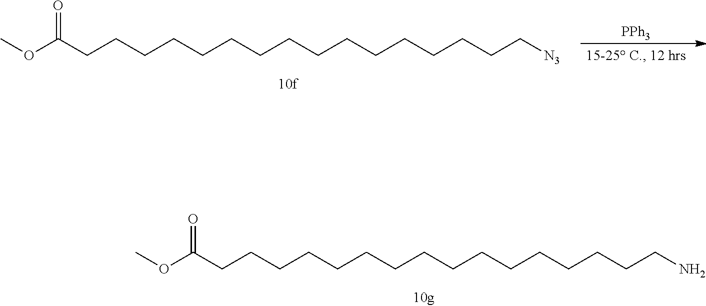 [SMILES: COC(=O)CCCCCCCCCCCCCCCCN=[N+]=[N-] ; COC(=O)CCCCCCCCCCCCCCCCN ; "PPh3"]

To the mixture of compound 10f (1.02 g, 3.13 mmol) was added PPh₃ (1.48 g, 5.64 mmol) at 15-25° C. and kept stirred at 15-25° C. for 12 hours. 1H NMR showed the compound 10f was disappeared and the compound 10g was found (The reactant solution was determined by 1H NMR directly). To the mixture was added 1 M HCl solution until pH=1-2 at 15-25° C., then the mixture was concentrated under reduced pressure. To the residue was added EtOAc (15.0 mL) at 15-25° C. and kept stirred at 15-25° C. for 15 mins, filtered and filter cake was dried under reduced pressure to give 10 g (1.15 g, TsOH and HCl salt) as a white solid.

¹H NMR: 400 MHz, DMSO-d₆; δ 7.70 (s, 3H), 7.48 (d, J=8.0 Hz, 1H), 7.12 (d, J=8.0 Hz, 1H), 3.58 (s, 3H), 2.70-2.83 (m, 2H), 2.29 (t, J=7.6 Hz, 2H), 2.29 (s, 1.5H), 1.45-1.58 (m, 4H), 1.19-1.34 (m, 24H).

General Procedure for Preparation of Compound 10h

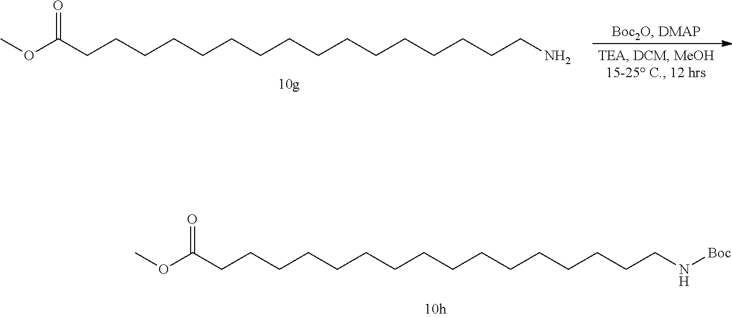

To the mixture of compound 10g (1.15 g, TsOH and HCl salt) in DCM (10 mL) and MeOH (1 mL) was added Boc₂O (1.03 g, 4.71 mmol, 1.08 mL), DMAP (39 mg, 319 umol) and TEA (794 mg, 7.84 mmol, 1.09 mL) at 15-25° C. and kept stirred at 15-25° C. for 12 hours under N₂ atmosphere. MS showed the MS value of compound 10g was disappeared. The reaction mixture was concentrated under reduced pressure. The residue was purified by silica gel column (Petroleum ether/Ethyl acetate, 1/0 to 20/1) to give compound 10h (341 mg, 853 umol, 27.2% yield) as a white solid.

¹H NMR: 400 MHz, CDCl₃; δ 4.42 (s, 1H), 3.60 (s, 3H), 3.03 (q, J=6.4 Hz, 2H), 2.23 (t, J=7.6 Hz, 2H), 1.49-1.61 (m, 4H), 1.37 (s, 9H), 1.11-1.29 (m, 24H).

General Procedure for Preparation of Compound 10i

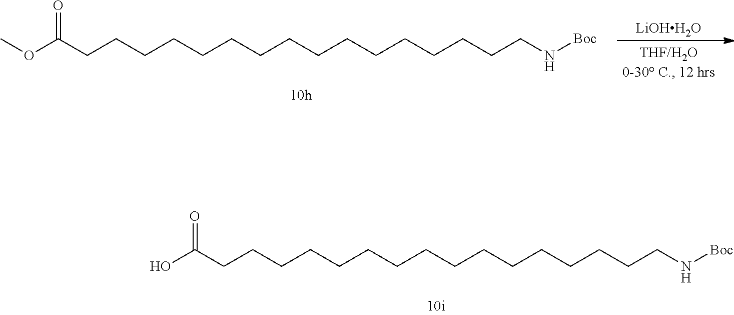 [SMILES: COC(=O)CCCCCCCCCCCCCCCCNC(=O)OC(C)(C)C ; "LiOH·H2O" ; CC(C)(C)OC(=O)NCCCCCCCCCCCCCCCCC(=O)O]

To the mixture of compound 10h (341 mg, 853 umol) in THE (4 mL) and H₂O (1 mL) was added LiOH.H₂O (46 mg, 1.10 mmol) at 0-10° C. and kept stirred at 20-30° C. for 12 hours. To the mixture was added water (20.0 mL) and EtOAc (50 mL), then 10% citric acid solution until pH=3 at 0-10° C. The organic phase was dried over anhydrous Na₂SO₄, filtered and filtrate was concentrated under reduced pressure, which was confirmed by 1H NMR. The residue was purified by silica gel column (Petroleum ether/Ethyl acetate, 1/0 to 5/1). The spot (R_{f}=0.22) was collected to give compound 10i (228 mg, 591 umol, 69.3% yield) as a white solid.

¹H NMR: 400 MHz, CDCl₃ δ 4.54 (s, 1H), 3.00-3.20 (m, 2H), 2.37 (t, J=7.2 Hz, 2H), 1.60-1.70 (m, 4H), 1.46 (s, 9H), 1.21-1.40 (m, 24H).

General Procedure for Preparation of Compound 10j

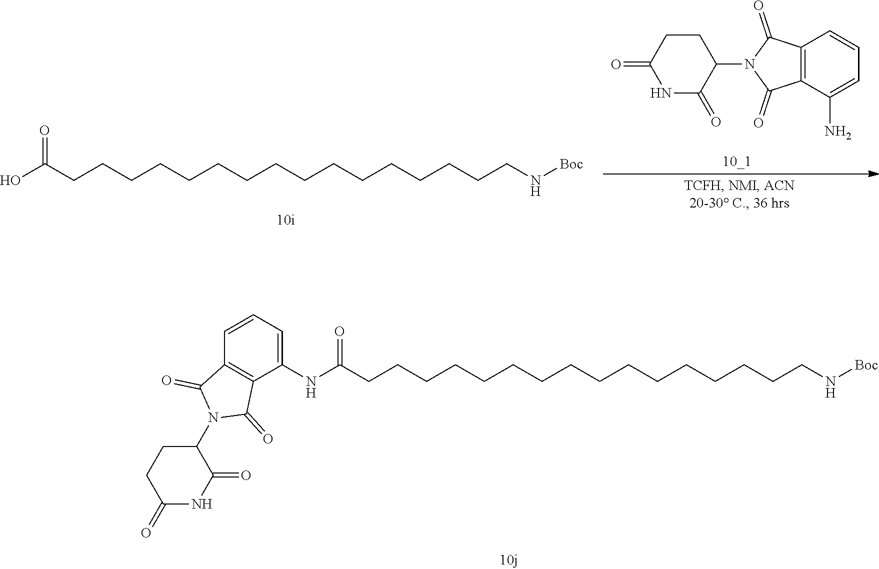 [SMILES: CC(C)(C)OC(=O)NCCCCCCCCCCCCCCCCC(=O)O ; Nc1cccc2c1C(=O)N(C1CCC(=O)NC1=O)C2=O ; CC(C)(C)OC(=O)NCCCCCCCCCCCCCCCCC(=O)Nc1cccc2c1C(=O)N(C1CCC(=O)NC1=O)C2=O]

To the solution of compound 10i (128 mg, 332 umol) and compound 10_1 (118 mg, 432 umol) in ACN (30.0 mL) was added NMI (190 mg, 2.31 mmol, 184 uL) and TCFH (240 mg, 855 umol) at 20-30° C. under N₂ atmosphere and kept stirred at 20-30° C. for 20 hours under N₂ atmosphere. LCMS showed the MS value of compound 10j was not found, to the mixture was added TCFH (240 mg, 855 umol) and NMI (190 mg, 2.31 mmol, 184 uL) at 20-30° C. under N₂ atmosphere and kept stirred at 20-30° C. for 16 hours under N₂ atmosphere. LCMS showed the MS value of compound 10j (RT=1.208 mins, MS=641.5) was found. The reaction mixture was poured into water (30 mL) at 0-10° C., the aqueous phase was saturated with NaCl solid and extracted with EtOAc (80 mL×2), the combined organic phase was dried over anhydrous Na₂SO₄, filtered and filtrate was concentrated under reduced pressure at 30° C., which was confirmed by LCMS RT=1.215 mins, MS=641.5). The residue was purified by reverse phase (TFA), the collected fraction (RT=1.215 mins) was concentrated under reduced pressure directly to give compound 10j (42 mg, 65.5 umol, 19.7% yield, 100% purity) as a white solid.

LCMS: (method 1), RT=1.213 mins, m/z (M+1)=641.5

General Procedure for Preparation of Compound 10k

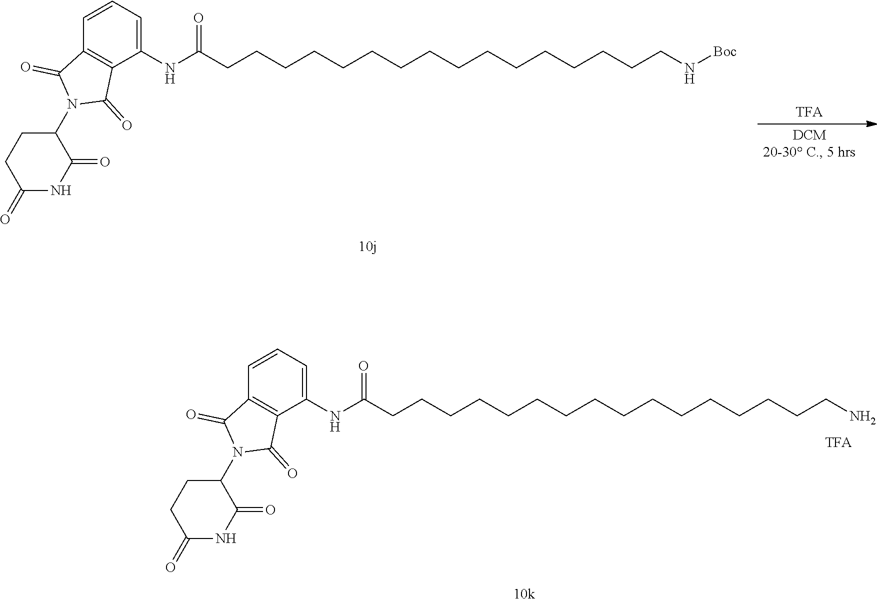 [SMILES: CC(C)(C)OC(=O)NCCCCCCCCCCCCCCCCC(=O)Nc1cccc2c1C(=O)N(C1CCC(=O)NC1=O)C2=O ; NCCCCCCCCCCCCCCCCC(=O)Nc1cccc2c1C(=O)N(C1CCC(=O)NC1=O)C2=O]

To the solution of compound 10j (42 mg, 65.5 umol) in DCM (3.00 mL) was added TFA (154 mg, 1.35 mmol, 100 uL) at 20-30° C. under N₂ atmosphere and kept stirred at 20-30° C. for 5 hours under N₂ atmosphere. LCMS showed the MS value of compound 10k (RT=0.938 mins, MS=541.3) was found. The reaction mixture was concentrated under reduced pressure at 30° C. to give compound 10k (40 mg, 58.8 umol, 89.8% yield, 96.3% purity) as a yellow solid, which was confirmed by LCMS, RT=0.933 mins, m/z (M+1)=541.4).

LCMS: (method 1), RT=0.933 mins, m/z (M+1)=541.4

General Procedure for Preparation of “A57”

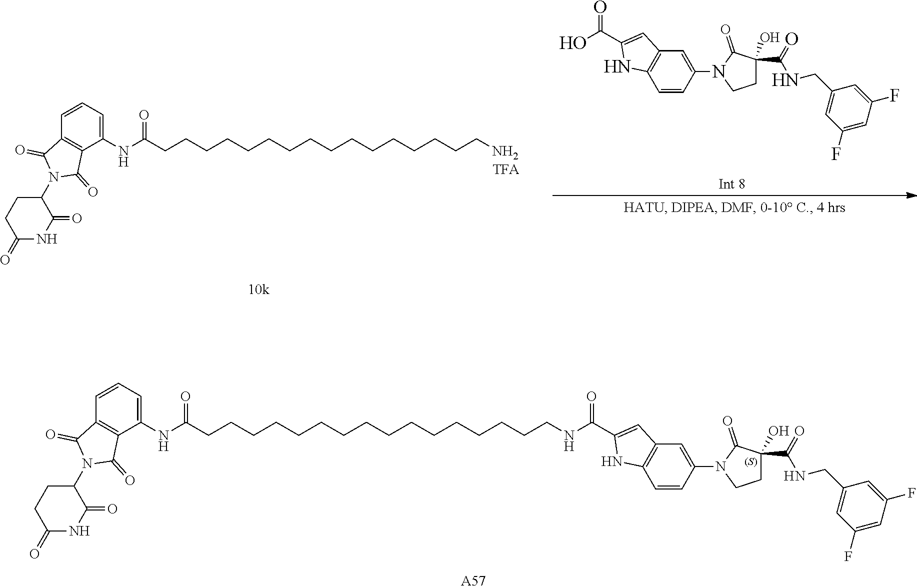

To the solution of compound int 8 (30 mg, 69.9 umol) and DIPEA (37 mg, 286 umol, 49.9 uL) in DMF (2.00 mL) was added HATU (40 mg, 105 umol) and a solution of compound 10k (48 mg, 73.3 umol, TFA) in DMF (2.00 mL) at 0-10° C. under N₂ atmosphere and kept stirred at 0-10° C. for 4 hours under N₂ atmosphere. To the reaction mixture was added glacial acetic acid until pH=5-6 at 0-5° C., then the mixture was poured into water (20.0 mL) at 0-5° C., the aqueous phase was extracted with EtOAc (30.0 mL×3), the combined organic phase was dried over anhydrous Na₂SO₄, filtered and filtrate was concentrated under reduced pressure, the residue was checked by LCMS. The residue was purified by prep-HPLC (column: Phenomenex luna C18 150*40 mm*15 um; mobile phase: [water (0.1% TFA)-ACN]; B %: 62%-92%, 11 min), the collected fraction was concentrated under reduced pressure directly. The residue was checked by LCMS (RT=2.946 mins, MS=952.5). The residue was further purified by prep-HPLC (column: Phenomenex Gemini-NX C18 75*30 mm*3 um; mobile phase: [water (0.1% TFA)-ACN]; B %: 70%-80%, 7 min), the collected fraction was concentrated under reduced pressure directly to give “A57” (7.02 mg, 7.25 umol, 10.4% yield, 98.3% purity) as a brown solid.

¹H NMR: 400 MHz, DMSO-d₆ δ 11.58 (s, 1H), 11.15 (s, 1H), 9.68 (s, 1H), 8.69 (t, J=6.0 Hz, 1H), 8.42-8.52 (m, 2H), 7.83 (t, J=7.6 Hz, 1H), 7.78 (d, J=1.6 Hz, 1H), 7.61 (d, J=6.8 Hz, 1H), 7.53 (dd, J=8.8, 2.0 Hz, 1H), 7.43 (d, J=8.8 Hz, 1H), 7.11 (d, J=1.6 Hz, 1H), 6.97-7.09 (m, 3H), 6.72 (s, 1H), 5.14 (dd, J=12.8, 6.4 Hz, 1H), 4.43 (dd, J=15.6, 6.8 Hz, 1H), 4.28 (dd, J=16.4, 6.0 Hz, 1H), 3.87-3.93 (m, 1H) 3.28 (q, J=6.4 Hz, 3H), 3.05 (q, J=6.0 Hz, 1H), 2.83-2.97 (m, 2H), 2.55-2.70 (m, 7H), 2.46 (t, J=7.6 Hz, 2H), 2.13-2.20 (m, 1H), 1.99-2.10 (m, 3H), 1.46-1.67 (m, 5H), 1.15-1.41 (m, 46H).

¹⁹F NMR: 400 MHz, DMSO-d₆ δ −110.35.

LCMS: (method 2), RT=2.946 mins, m/z (M+1)=952.5

LCMS: (method 1), RT=1.139 mins, m/z (M+1)=952.5

HPLC: (method 3), RT=2.715 min

2. Exemplary Testing of the Compound

1. Cell Seeding

HCT116 cells were sown in 6-well plates. Pipette 1 mL fresh medium to every well and incubate for at least 1 h (37° C., 5% CO₂, 95% rH).

Harvest and count cells in Vi-Cell. Equinr. 70198475 Adjust cell concentration to 0.35e⁶ cells/mL and add 1 mL of cell suspension (total 0,35e⁶ cells/well).

Incubate cells for 24 hours (37° C., 5% CO₂, 95% rH)

2. Compound Treatment

HCT116: 24 h: ˜50% confluent before treatment

Treatment with Selected Compounds

Compounds will be thawed the first time, stock: 10 mM; final concentration: 10 μM & 1 μM & 0.1 μM

Treatment with DMSO based solution with Digital Dispenser: HCT116 platte 2A1.jpg_meta.xml
create new dispensing protocol for compound
apply name of test compound and its stock concentration (in mM) for testing
3×1:10 dilution series
final 0.3% DMSO, DMSO normalization for every well
shaking after compound/DMSO addition

Incubate cells for further 24 hours (37° C., 5% C02, 95% rH)

3. Cell Lysis

HCT116: 70-80% confluent before lysis
after incubation cells were lysed with RIPA lysis buffer including Halt™

Protease and Phosphatase Inhibitor

Cocktail (1×)

all steps performed on ice or at 4° C.
aspirate supernatant from cells
wash cells twice with 2.0 mL ice cold PBS buffer
aspirate supernatant from cells after each washing step
lyse cells with 100 μL Ripa lysis buffer per well
scrape cells with cell scraper and transfer lysates to Eppendorf Cups
incubate for 15 min on ice
centrifuge 150 min at 13000×g and 4° C.
transfer supernatants to fresh Eppendorf cups
removal of 20 μL lysate for BCA assay
aliquot and freeze at −20° C.

4. BCA Assay of Protein Extracts

Determination of protein concentration of lysates with BCA Protein Assay Kit. BSA supplied with the kit is 2 mg/mL.

Dilute the standard (Stock BSA concentration=2 mg/mL) in a 96 well plate: Samples were run 1:4. Dilute in lysing buffer, lysing buffer alone was run to determine the background from detergents in the assay.

BCA reagent: Dilute Reagent A and Reagent B in a 1:50 ratio. To 9.8 mL A add 200 μL B.

Assay Procedure: Pipette 5 μL/well of standard or samples to 96 wells (see plate layout). Samples were run in duplicate. Blanks=5 μL/well PBS or 5 μL/well lysing buffer. Add 100 μL/well of the prepared BCA reagent. Tap plate to mix or shake for 1-2 min on the Eppendorf mixer. Incubate at 37° C. for 30 min.

Read the OD at 562 nm. Plate was read on EnVision 2104 Multilabel Reader (Perkin Elmer, Equipment Number 70160330) last verification of instrument 17.08.2017 (once a year) (A25/531). AEH181012_BCA.csv

5. Data Calculation:

In EXCEL the duplicates were averaged and the average background PBS was subtracted for the standard curve.

A trend line was created, the intercept (b) was set at 0. The R² should be >0.950 for acceptable results. The lysate buffer blank was subtracted from the test values and the averaged, background-corrected O.D. values were used with the equation to calculate mg/ml and then were corrected for the dilution factor for the final mg/mL value. Only results from OD values within the range of the standard curve are acceptable. Values outside of the standard curve range are ignored. BCA AEH00350 Berechnung.xlsx

6. Sample Calculation and Preparation
thaw lysates of AEH00350 on ice
calculate sample volume for Western Blot loading 10 μg total protein/13 μL per slot:
dilute samples with lysis buffer and 10× reducing agent & 4×LDS buffer to 10 μg.
heat them at 70° C. for 10 min and 1000 rmp

7. WB Analysis
insert one NuPAGE 4-12% Bis-Tris Midi Gel (26 well) and fix in Criterion Cell Device electrophoresis chamber (BIO-RAD, #165-6001)
fill chamber with 1×NuPAGE MES SDS Running-Buffer
pipette samples, see Blue Plus 2 Pre-Stained Protein Standard and MagicMark XP per comb:
3 μL Magic Marker and 7 μL Seeblue Marker, 13 μL sample per slotlayout.xlsx
two gels with following layout
2D-SDS-Page run: 45 min with const. 200 V (right power supply)
soak nitrocellulose membrane in Methanol and blot paper in 1× transfer buffer
preparation of the blot sandwich: 2× blot paper, gel, membrane, 2× blot paper at a Fast Blot B44 (Biometra, #015-200)
Western Blot run: 1 h 35 min with const. 150 mA Blocking
transfer the membrane to Blocking-Buffer and shake for 1 h at RT
Blocking solution: Odyssey Blocking Buffer

Incubation with primary antibody MetAP2 clone EPR6887 RabMAb Abcam ab134124 (1:5000)
Primary antibody solution: 0.5× Odyssey Blocking Buffer+0.5×PBS+0.1% Tween20
incubation with primary antibody over night at 4° C. with gentle shaking:

Incubation with secondary antibody (Goat anti-rabbit (700 nm), Molecular Probes A21076 39542A (1:20000)
wash membranes 4 times for 10 minutes at room temperature
Wash solution: 1×PBS+0.1% Tween20
Incubate membrane with secondary antibody for 1 h on a shaker at room temperature in the dark
Secondary antibody solution: 0.5× Odyssey Blocking Buffer+0.5×PBS+0.1% Tween20+0.01% SDS
last washing step only in 1×PBS
scan membranes on Odyssey Imager

TABLE 1
IC₅₀ of compounds of the formula I
according to the invention

| Compound | IC₅₀ enzyme | IC₅₀ HUVEC |
|---|---|---|
| No. | [μM] | [μM] |
| “A1” | 0.19 | 25 |
| “A2” | 0.25 | 30 |
| “A3” | 0.55 | 30 |
| “A4” | 0.48 | 1.6 |
| “A5” | 0.33 | 5.8 |
| “A6” | 0.37 | 30 |
| “A7” | 0.32 | 7.3 |
| “A8” | 0.33 | 30 |
| “A42” | | 4.6 |
| “A43” | | 7.1 |
| “A44” | | 7 |
| “A45” | | 0.61 |
| “A46” | | >30 |
| “A47” | | 0.24 |
| “A48” | | 0.085 |
| “A49” | | 0.051 |
| “A50” | | 0.18 |
| “A51” | | 0.4 |
| “A52” | | 4.9 |
| “A53” | | 2.8 |
| “A54” | | 0.11 |
| “A55” | | >30 |
| “A56” | | 0.12 |
| “A57” | | >30 |

The following examples relate to medicaments:

### Example A: Injection Vials

A solution of 100 g of an active ingredient of the formula I and 5 g of disodium hydrogenphosphate in 3 l of bidistilled water is adjusted to pH 6.5 using 2 N hydrochloric acid, sterile filtered, transferred into injection vials, lyophilised under sterile conditions and sealed under sterile conditions. Each injection vial contains 5 mg of active ingredient.

### Example B: Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### Example C: Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example D: Ointment

500 mg of an active ingredient of the formula I are mixed with 99.5 g of Vaseline under aseptic conditions.

### Example E: Tablets

A mixture of 1 kg of active ingredient of the formula I, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### Example F: Dragees

Tablets are pressed analogously to Example E and subsequently coated in a conventional manner with a coating of sucrose, potato starch, talc, tragacanth and dye.

### Example G: Capsules

2 kg of active ingredient of the formula I are introduced into hard gelatine capsules in a conventional manner in such a way that each capsule contains 20 mg of the active ingredient.

### Example H: Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 ma of active ingredient.

1. A compound of the formula I
Q¹-Q²-Q³ I
wherein
Q¹ denotes 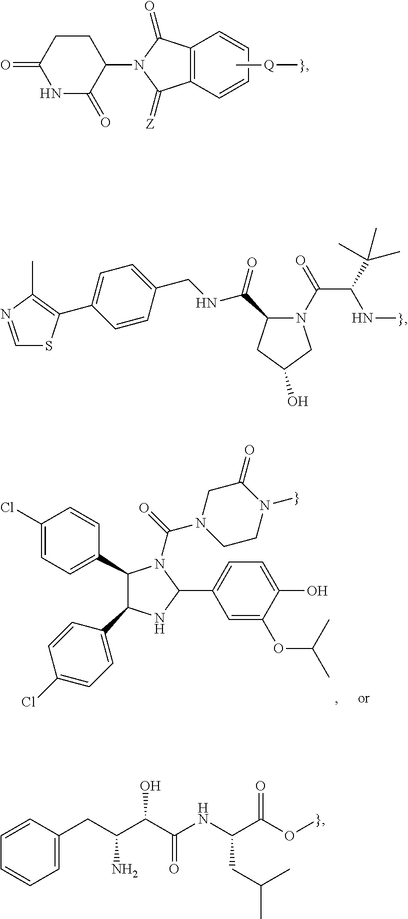
Z denotes O or CH₂,
Q denotes O, NH, NCH₃, or CH₂,
Q² denotes unbranched alkylene having 4-25 C, atoms, in which 1-8 nonadjacent CH₂ groups may be replaced by O, CONH, and/or NHCO ad in which one CH₂ group may be replaced by 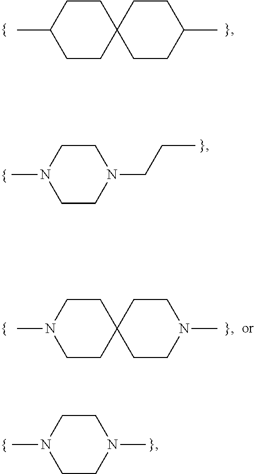
Q³ denotes 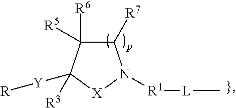
L denotes NR⁴CO, CONR⁴, NH, O, CO, S, SO₂, SO(═NH), NHCONH, SO₂NH or NHSO₂,
R denotes NR²R⁴, Alk, C(═CH₂)[C(R⁴)₂]ₙAr², Het², O[C(R⁴)₂]ₙAr², or OA,
X denotes CO or CH₂,
Y denotes CO or CH₂,
R¹ denotes (CH₂)ₙ, [C(R⁴)₂]ₙAr¹—, (CH₂)ₙHet-, (CH₂)ₙCyc-, [C(R⁴)₂]ₙCONHAr¹—, [C(R⁴)₂]ₙNA-, O[C(R⁴)₂]ₙAr¹—, or [C(R⁴)₂]ₙCOO(CH₂)ₙAr¹—,
wherein substituent L directly is connected to Ar¹, Het, or Cyc,
R² denotes H, [C(R⁴)₂]ₙAr², (CH₂)ₙCOHet¹, (CH₂)ₙCOAr², (CH₂)ₘNA₂, (CH₂)ₙCyc, or (CH₂)ₙHet¹,
R³ denotes OH or OCOA,
R⁴ denotes H or alkyl having 1, 2, 3, or 4 C-atoms,
R² and R⁴ together also denote alkylene having 2, 3, 4 or 5 C-atoms, where a CH₂ group may also be replaced by N(CH₂)ₘOH or SO₂,
R⁵, R⁶ each, independently of one another H, F, or A,
R⁵ and R⁶ together also denote alkylene having 2, 3, 4, or 5 C-atoms, where a CH₂ group may also be replaced by NCOA or O,
R⁷ denotes H, Hal, or A,
Ar¹ denotes phenyl which is unsubstituted or mono-, di- tri-, tetra-, or pentasubstituted by Hal, OH, OA, CONH₂, CONHA, CONA₂, NHSO₂A, CONHCyc, NHSO₂Cyc, CONHAr², Het¹, COHet¹, and/or NASO₂A,
Ar² denotes phenyl which is unsubstituted or mono-, di-, tri-, tetra-, or pentasubstituted by Hal, A, CONH₂, and/or OAr³,
Ar³ denotes phenyl which is unsubstituted or monosubstituted by NH₂,
Het denotes a mono- or bicyclic saturated, unsaturated or aromatic heterocycle having 1 to 4 N, and/or O, and/or S atoms which is unsubstituted or mono-, di-, or trisubstituted by Hal, A, OA, CN, NH₂, NHA, NA₂, NO₂, CN, COOH, COOA, (CH₂)ₙCONH₂, (CH₂)ₙCONHA, (CH₂)ₙCONA₂, NHCOA, COA, CHO, Het¹, SO₂A, SO₂NH₂, SO₂NHA, SO₂NA₂, CONHNH₂, CONHAr³, ═O, and/or Ar³,
Het¹ denotes pyridazinyl, pyrazolyl, pyridyl, piperazinyl, morpholinyl, pyrimidinyl, furyl, thienyl, imidazolyl, pyrrolyl, oxazolyl, oxadiazolyl, isoxazolyl, thiazolyl, triazolyl, tetrazolyl, thiadiazole, piperidin-1-yl, pyrrolidin-1-yl, tetrahydropyranyl, 1,2-oxazinan-2-yl, 1,2,5-oxadiazinan-2-yl, 1,3-oxazinan-3-yl, or hexahydropyrimidinyl, each of which is unsubstituted or mono-, di-, or trisubstituted by A and/or OA,
Het² denotes isoindolyl,
A denotes unbranched or branched alkyl having 1-10 C atoms, in which 1-7H atoms may be replaced by F, Cl, Br, OH, CHO, COA, COOA, CN, CONA₂, CONHA, and/or CONH₂,
and/or in which one or two non-adjacent CH and/or CH₂ groups may be replaced by C,
or Cyc,
Alk denotes alkenyl having 2, 3, 4, 5, or 6 C atoms
Cyc denotes cyclic alkyl having 3-7 C atoms which is unsubstituted or mono-, di- or trisubstituted by NHCOA, NHSO₂, OH, OA, A, NH₂, NHA, NA₂, COOA, COOH, and/or CONHA,
Hal denotes F, Cl, Br, or I,
m denotes 1, 2, 3, or 4,
n denotes 0, 1, 2, 3, or 4,
p denotes 1, 2, or 3,
and pharmaceutically acceptable salts, tautomers and stereoisomers thereof, including mixtures thereof in all ratios.

2. The compound according to claim 1, wherein
Het denotes pyrazinyl, pyrazolyl, benzimidazolyl, pyridyl, thienyl, furanyl, indolyl, dihydroindolyl, benzofuranyl, tetrahydropyranyl, dihydroquinolinyl, dihydroisoquinolinyl, tetrahydroquinolinyl, tetrahydroisoquinolinyl, indazolyl, imidazolyl, pyrrolyl, oxazolyl, oxadiazolyl, isoxazolyl, benzothiazolyl, piperidin-1-yl, pyrrolidin-1-yl, 3,4-dihydro-2H-pyrido[3,2-b]-1,4-oxazinyl, 3,4-dihydro-2H-benzo-1,4-oxazinyl, benzofuranyl, azetidinyl, 1H-pyrrolo[2,3-b]pyridinyl, 2H-chromenyl, 3-azabicylo[3.2.0]hexyl, pyrrolo[2,3-b]pyridinyl, tetrahydrofuranyl, tetrahydro-1,8-naphthyridinyl 2,3-dihydro-benzoisothiazolyl, 1,2,3,4-tetrahydrobenzothiazinyl, or hexahydrobenzo-1,3-dioxolyl, each of which is unsubstituted or mono-, di-, or trisubstituted by Hal, A, OA, CN, NH₂, NHA, NA₂, NO₂, CN, COOH, COOA, (CH₂)ₙCONH₂, (CH₂)ₙCONHA, (CH₂)ₙCONA₂, NHCOA, COA, CHO, Het¹, SO₂A, SO₂NH₂, SO₂NHA, SO₂NA₂, CONHNH₂, CONHAr³, ═O, and/or Ar³,
and pharmaceutically acceptable salts, tautomers, and stereoisomers thereof, including mixtures thereof in all ratios.

3. The compound according to claim 1, wherein
Q¹ denotes 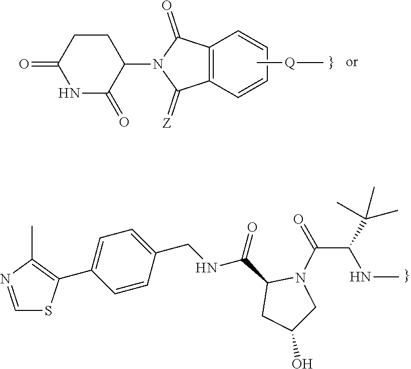
and pharmaceutically acceptable solvates, salts, tautomers, and stereoisomers thereof, including mixtures thereof in all ratios.

4. The compound according to claim 1, wherein
R denotes NR²R⁴,
and pharmaceutically acceptable solvates, salts, tautomers, and stereoisomers thereof, including mixtures thereof in all ratios.

5. The compound according to claim 1, wherein
R¹ denotes (CH₂)ₙ, [C(R⁴)₂]ₙAr¹—, or (CH₂)ₙHet-,
and pharmaceutically acceptable solvates, salts, tautomers, and stereoisomers thereof, including mixtures thereof in all ratios.

6. The compound according to claim 1, wherein
R² denotes [C(R⁴)₂]ₙAr², (CH₂)ₙCyc, or (CH₂)ₙHet¹,
and pharmaceutically acceptable solvates, salts, tautomers, and stereoisomers thereof, including mixtures thereof in all ratios.

7. The compound according to claim 1, wherein
Het denotes benzimidazolyl or indolyl, each of which is unsubstituted or monosubstituted by Hal,
and pharmaceutically acceptable solvates, salts, tautomers, and stereoisomers thereof, including mixtures thereof in all ratios.

8. The compound according to claim 1, wherein
A denotes unbranched or branched alkyl having 1-6 C atoms, in which 1-5H atoms may be replaced by F, Cl, and/or OH,
and pharmaceutically acceptable solvates, salts, tautomers, and stereoisomers thereof, including mixtures thereof in all ratios.

9. The compound according to claim 1, wherein
Q¹ denotes 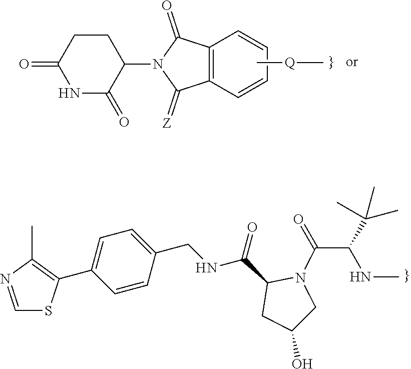
Z denotes O or CH₂,
Q denotes O, NH, NCH₃, or CH₂,
Q² denotes unbranched alkylene having 4-25 C atoms, in which 1-8 nonadjacent CH₂ groups may be replaced by O, CONH, and/or NHCO and in which one CH₂ group may be replaced by 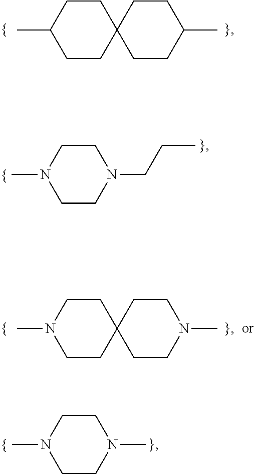
Q³ denotes 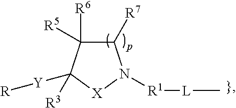
L denotes NR⁴CO, CONR⁴, NH, O, CO, S, SO₂, SO(═NH), NHCONH, SO₂NH, or NHSO₂,
R denotes NR²R⁴,
X denotes CO or CH₂,
Y denotes CO or CH₂,
R¹ denotes (CH₂)ₙ, [C(R⁴)₂]ₙAr¹—, or (CH₂)ₙHet-,
wherein substituent L directly is connected to Ar¹, Het, or Cyc,
R² denotes [C(R⁴)₂]ₙAr², (CH₂)ₙCyc, or (CH₂)ₙHet¹,
R³ denotes OH,
R⁴ denotes H or alkyl having 1, 2, 3, or 4 C-atoms,
R⁵, R⁶ denote H,
R⁷ denotes H, Hal, or A,
Ar¹ denotes phenyl,
Ar² denotes phenyl which is unsubstituted or mono-, di- tri-, or tetra-substituted by Hal,
Het denotes pyrazinyl, pyrazolyl, benzimidazolyl, pyridyl, thienyl, furanyl, indolyl, dihydroindolyl, benzofuranyl, tetrahydropyranyl, dihydroquinolinyl, dihydroisoquinolinyl, tetrahydroquinolinyl, tetrahydroisoquinolinyl, indazolyl, imidazolyl, pyrrolyl, oxazolyl, oxadiazolyl, isoxazolyl, benzothiazolyl, piperidin-1-yl, pyrrolidin-1-yl, 3,4-dihydro-2H-pyrido[3,2-b]-1,4-oxazinyl, 3,4-dihydro-2H-benzo-1,4-oxazinyl, benzofuranyl, azetidinyl, 1H-pyrrolo[2,3-b]pyridinyl, 2H-chromenyl, 3-azabicylo[3.2.0]hexyl, pyrrolo[2,3-b]pyridinyl, tetrahydrofuranyl, tetrahydro-1,8-naphthyridinyl 2,3-dihydro-benzoisothiazolyl, 1,2,3,4-tetrahydrobenzothiazinyl, or hexahydrobenzo-1,3-dioxolyl, each of which is unsubstituted or mono-, di-, or trisubstituted by Hal, A, OA, CN, NH₂, NHA, NA₂, NO₂, CN, COOH, COOA, (CH₂)ₙCONH₂, (CH₂)ₙCONHA, (CH₂)ₙCONA₂, NHCOA, COA, CHO, Het¹, SO₂A, SO₂NH₂, SO₂NHA, SO₂NA₂, CONHNH₂, CONHAr³, ═O, and/or Ar³,
Het¹ denotes pyridyl, furyl, thienyl, imidazolyl, or pyrrolyl,
A denotes unbranched or branched alkyl having 1-6 C atoms, in which 1-5H atoms may be replaced by F, Cl, and/or 01-1,
Cyc denotes cyclic alkyl having 3-7 C atoms,
Hal denotes F, Cl, Br, or I,
n denotes 0, 1, 2, 3, or 4,
p denotes 1, 2, or 3,
and pharmaceutically acceptable solvates, salts, tautomers, and stereoisomers thereof, including mixtures thereof in all ratios.

10. The compound according to claim 1, wherein the compound is selected from the group consisting of

| No. | chemical structure |
|---|---|
| “A 1” | 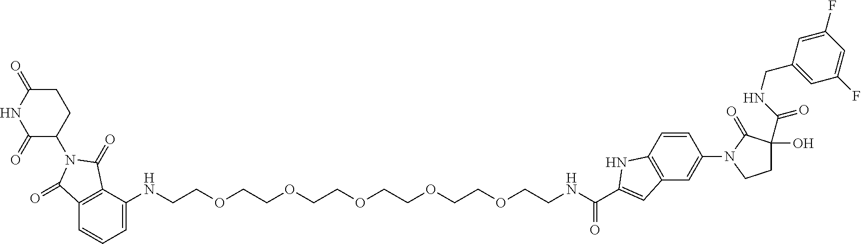 [SMILES: O=C1CCC(N2C(=O)c3cccc(NCCOCCOCCOCCOCCOCCNC(=O)c4cc5cc(N6CCC(O)(C(=O)NCc7cc(F)cc(F)c7)C6=O)ccc5[nH]4)c3C2=O)C(=O)N1] |
| “A 2” | 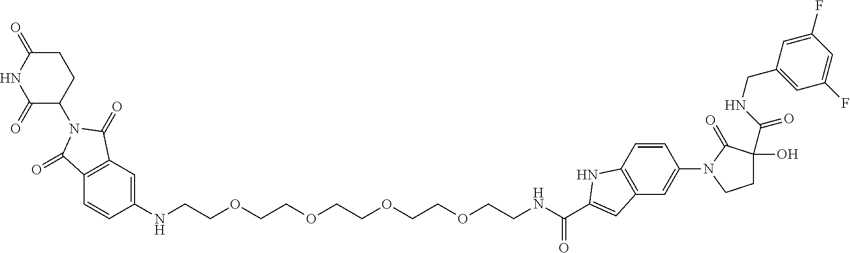 [SMILES: O=C1CCC(N2C(=O)c3ccc(NCCOCCOCCOCCOCCNC(=O)c4cc5cc(N6CCC(O)(C(=O)NCc7cc(F)cc(F)c7)C6=O)ccc5[nH]4)cc3C2=O)C(=O)N1] |
| “A 3” | 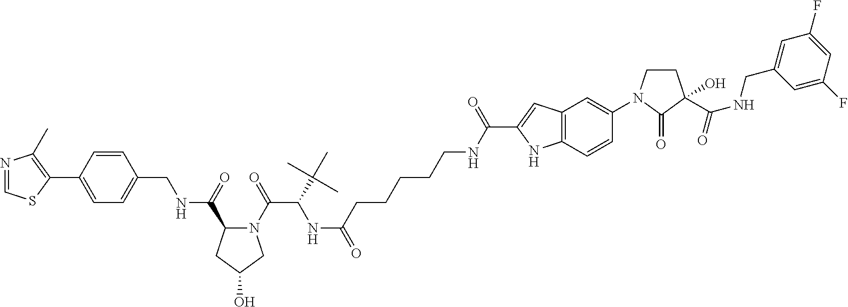 [SMILES: Cc1ncsc1-c1ccc(CNC(=O)[C@@H]2C[C@@H](O)CN2C(=O)[C@@H](NC(=O)CCCCCNC(=O)c2cc3cc(N4CC[C@@](O)(C(=O)NCc5cc(F)cc(F)c5)C4=O)ccc3[nH]2)C(C)(C)C)cc1] |
| “A 4” | 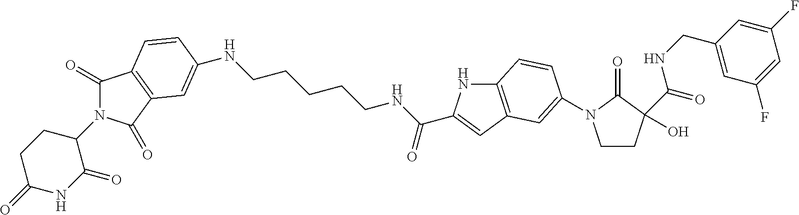 [SMILES: O=C1CCC(N2C(=O)c3ccc(NCCCCCNC(=O)c4cc5cc(N6CCC(O)(C(=O)NCc7cc(F)cc(F)c7)C6=O)ccc5[nH]4)cc3C2=O)C(=O)N1] |
| “A 5” | 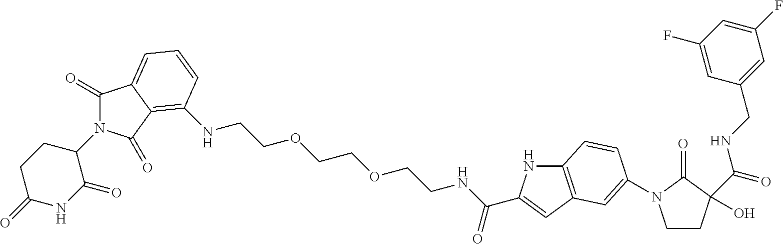 [SMILES: O=C1CCC(N2C(=O)c3cccc(NCCOCCOCCNC(=O)c4cc5cc(N6CCC(O)(C(=O)NCc7cc(F)cc(F)c7)C6=O)ccc5[nH]4)c3C2=O)C(=O)N1] |
| “A 6” | 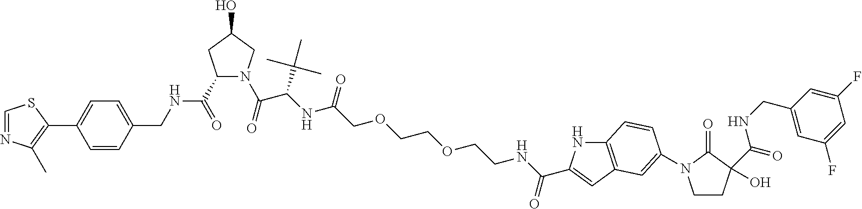 [SMILES: Cc1ncsc1-c1ccc(CNC(=O)[C@@H]2C[C@@H](O)CN2C(=O)[C@@H](NC(=O)COCCOCCNC(=O)c2cc3cc(N4CCC(O)(C(=O)NCc5cc(F)cc(F)c5)C4=O)ccc3[nH]2)C(C)(C)C)cc1] |
| “A 7” | 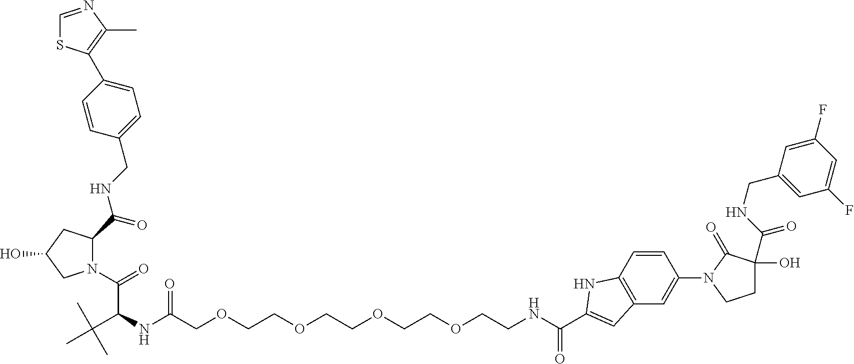 [SMILES: Cc1ncsc1-c1ccc(CNC(=O)[C@@H]2C[C@@H](O)CN2C(=O)[C@@H](NC(=O)COCCOCCOCCOCCNC(=O)c2cc3cc(N4CCC(O)(C(=O)NCc5cc(F)cc(F)c5)C4=O)ccc3[nH]2)C(C)(C)C)cc1] |
| “A 8” | 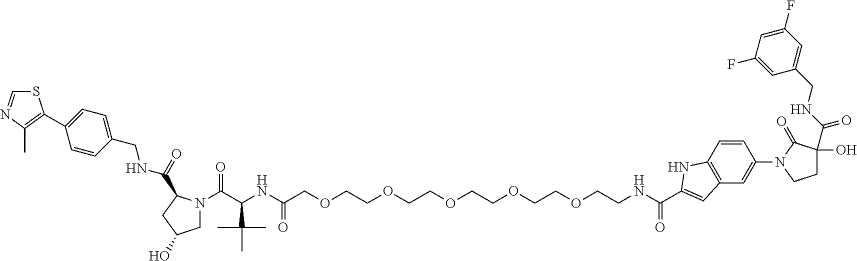 [SMILES: Cc1ncsc1-c1ccc(CNC(=O)[C@@H]2C[C@@H](O)CN2C(=O)[C@@H](NC(=O)COCCOCCOCCOCCOCCNC(=O)c2cc3cc(N4CCC(O)(C(=O)NCc5cc(F)cc(F)c5)C4=O)ccc3[nH]2)C(C)(C)C)cc1] |
| “A 9” | 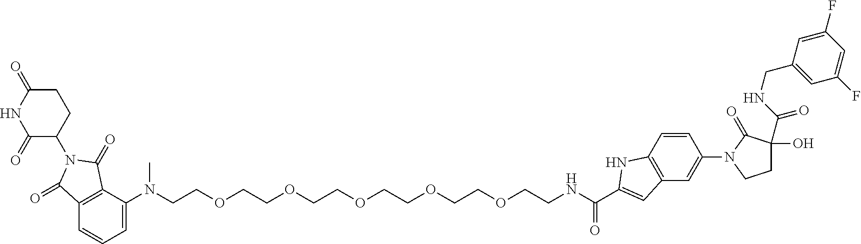 [SMILES: CN(CCOCCOCCOCCOCCOCCNC(=O)c1cc2cc(N3CCC(O)(C(=O)NCc4cc(F)cc(F)c4)C3=O)ccc2[nH]1)c1cccc2c1C(=O)N(C1CCC(=O)NC1=O)C2=O] |
| “A 10” | 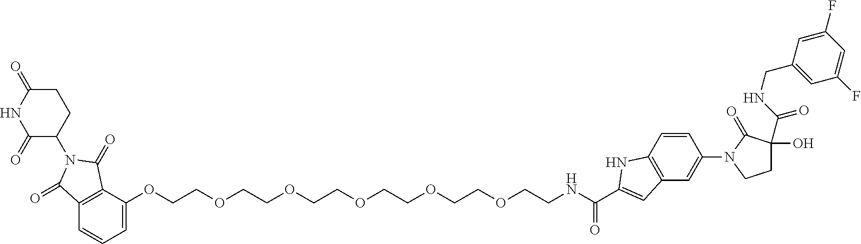 [SMILES: O=C1CCC(N2C(=O)c3cccc(OCCOCCOCCOCCOCCOCCNC(=O)c4cc5cc(N6CCC(O)(C(=O)NCc7cc(F)cc(F)c7)C6=O)ccc5[nH]4)c3C2=O)C(=O)N1] |
| “A 11” | 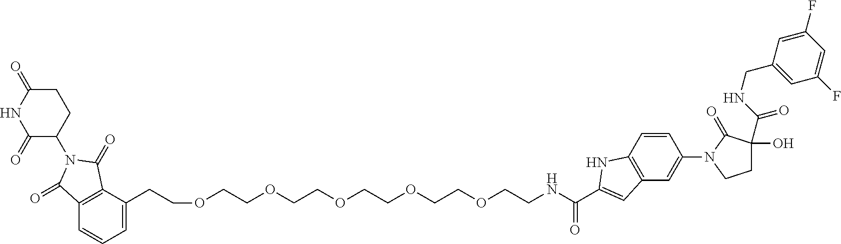 [SMILES: O=C1CCC(N2C(=O)c3cccc(CCOCCOCCOCCOCCOCCNC(=O)c4cc5cc(N6CCC(O)(C(=O)NCc7cc(F)cc(F)c7)C6=O)ccc5[nH]4)c3C2=O)C(=O)N1] |
| “A 12” | 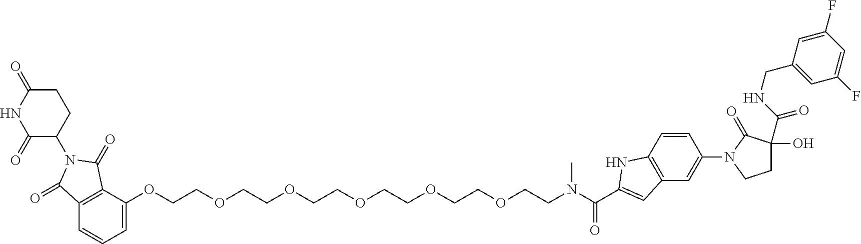 [SMILES: CN(CCOCCOCCOCCOCCOCCOc1cccc2c1C(=O)N(C1CCC(=O)NC1=O)C2=O)C(=O)c1cc2cc(N3CCC(O)(C(=O)NCc4cc(F)cc(F)c4)C3=O)ccc2[nH]1] |
| “A 13” | 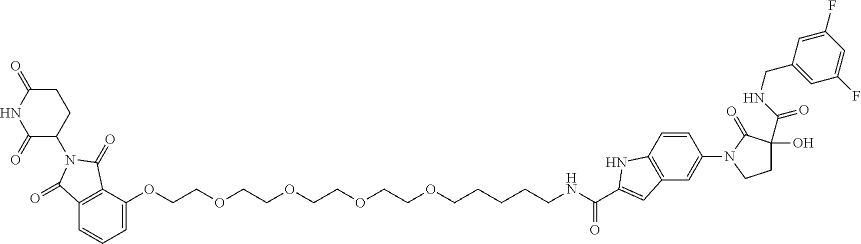 [SMILES: O=C1CCC(N2C(=O)c3cccc(OCCOCCOCCOCCOCCCCCNC(=O)c4cc5cc(N6CCC(O)(C(=O)NCc7cc(F)cc(F)c7)C6=O)ccc5[nH]4)c3C2=O)C(=O)N1] |
| “A 14” | 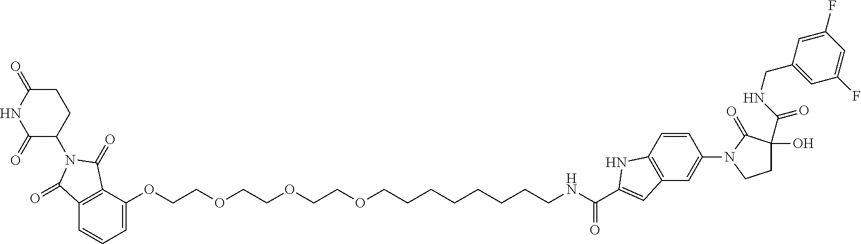 [SMILES: O=C1CCC(N2C(=O)c3cccc(OCCOCCOCCOCCCCCCCCNC(=O)c4cc5cc(N6CCC(O)(C(=O)NCc7cc(F)cc(F)c7)C6=O)ccc5[nH]4)c3C2=O)C(=O)N1] |
| “A 15” | 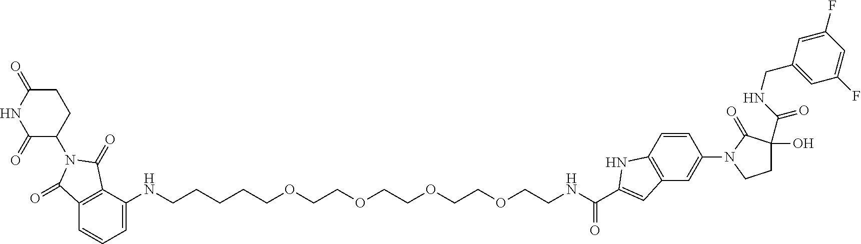 [SMILES: O=C1CCC(N2C(=O)c3cccc(NCCCCCOCCOCCOCCOCCNC(=O)c4cc5cc(N6CCC(O)(C(=O)NCc7cc(F)cc(F)c7)C6=O)ccc5[nH]4)c3C2=O)C(=O)N1] |
| “A 16” | 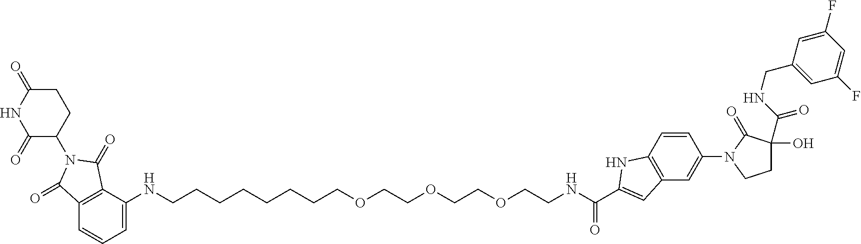 [SMILES: O=C1CCC(N2C(=O)c3cccc(NCCCCCCCCOCCOCCOCCNC(=O)c4cc5cc(N6CCC(O)(C(=O)NCc7cc(F)cc(F)c7)C6=O)ccc5[nH]4)c3C2=O)C(=O)N1] |
| “A 17” | 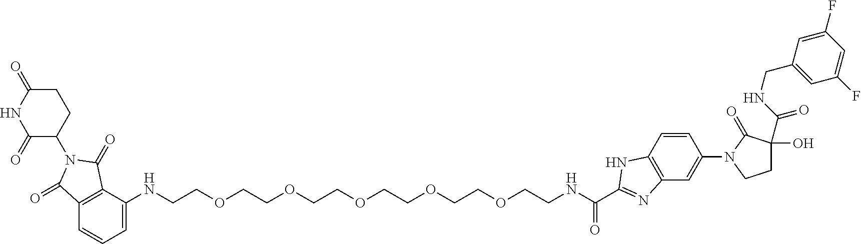 [SMILES: O=C1CCC(N2C(=O)c3cccc(NCCOCCOCCOCCOCCOCCNC(=O)c4nc5cc(N6CCC(O)(C(=O)NCc7cc(F)cc(F)c7)C6=O)ccc5[nH]4)c3C2=O)C(=O)N1] |
| “A 18” | 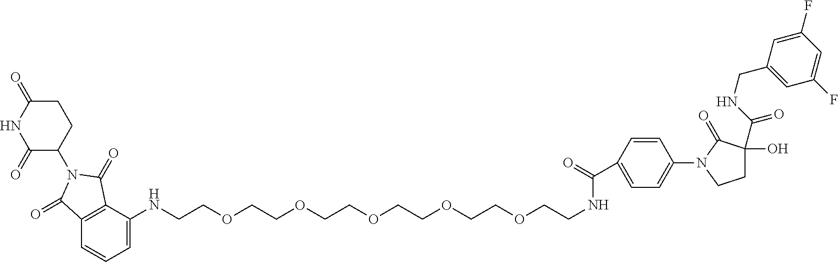 [SMILES: O=C1CCC(N2C(=O)c3cccc(NCCOCCOCCOCCOCCOCCNC(=O)c4ccc(N5CCC(O)(C(=O)NCc6cc(F)cc(F)c6)C5=O)cc4)c3C2=O)C(=O)N1] |
| “A 19” | 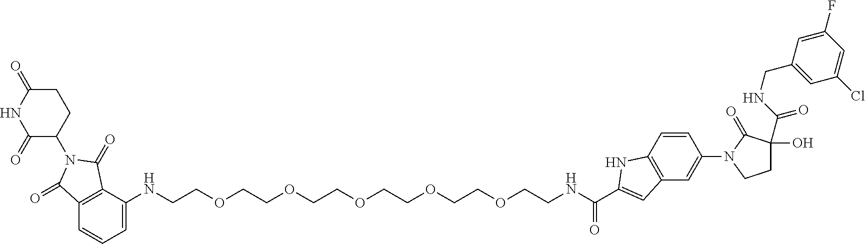 [SMILES: O=C1CCC(N2C(=O)c3cccc(NCCOCCOCCOCCOCCOCCNC(=O)c4cc5cc(N6CCC(O)(C(=O)NCc7cc(F)cc(Cl)c7)C6=O)ccc5[nH]4)c3C2=O)C(=O)N1] |
| “A 20” | 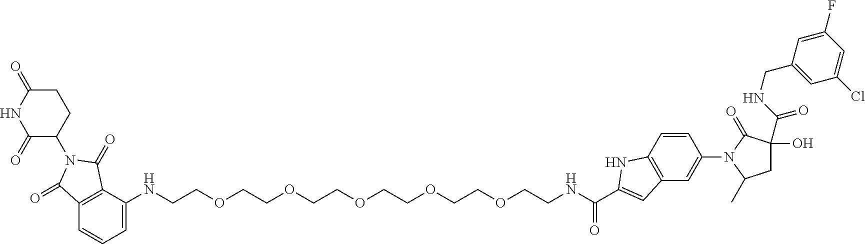 [SMILES: CC1CC(O)(C(=O)NCc2cc(F)cc(Cl)c2)C(=O)N1c1ccc2[nH]c(C(=O)NCCOCCOCCOCCOCCOCCNc3cccc4c3C(=O)N(C3CCC(=O)NC3=O)C4=O)cc2c1] |
| “A 21” | 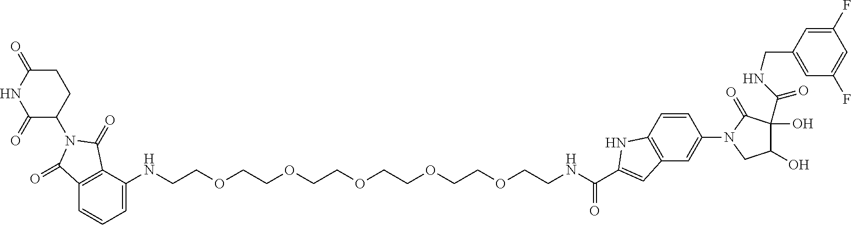 [SMILES: O=C1CCC(N2C(=O)c3cccc(NCCOCCOCCOCCOCCOCCNC(=O)c4cc5cc(N6CC(O)C(O)(C(=O)NCc7cc(F)cc(F)c7)C6=O)ccc5[nH]4)c3C2=O)C(=O)N1] |
| “A 22” | 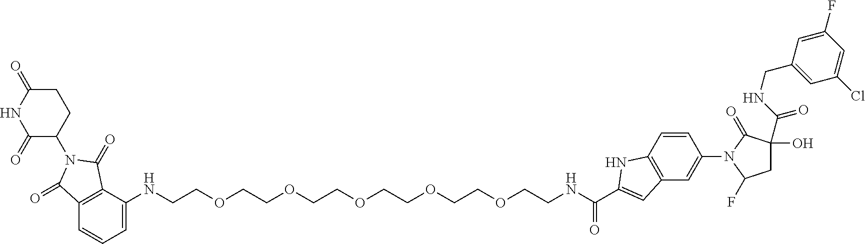 [SMILES: O=C1CCC(N2C(=O)c3cccc(NCCOCCOCCOCCOCCOCCNC(=O)c4cc5cc(N6C(=O)C(O)(C(=O)NCc7cc(F)cc(Cl)c7)CC6F)ccc5[nH]4)c3C2=O)C(=O)N1] |
| “A 23” | 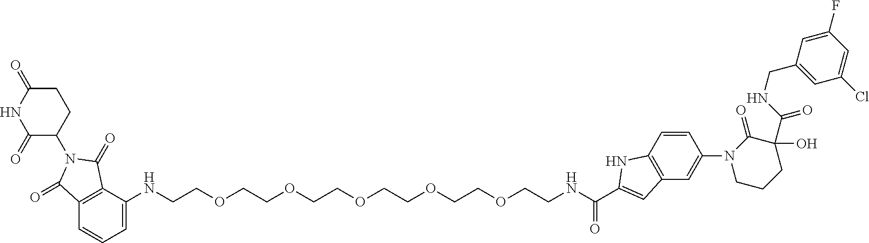 [SMILES: O=C1CCC(N2C(=O)c3cccc(NCCOCCOCCOCCOCCOCCNC(=O)c4cc5cc(N6CCCC(O)(C(=O)NCc7cc(F)cc(Cl)c7)C6=O)ccc5[nH]4)c3C2=O)C(=O)N1] |
| “A 24” | 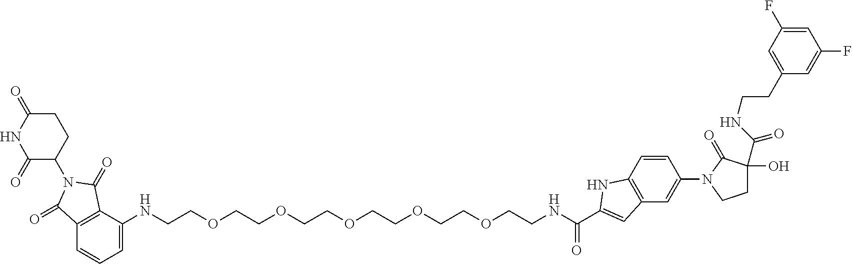 [SMILES: O=C1CCC(N2C(=O)c3cccc(NCCOCCOCCOCCOCCOCCNC(=O)c4cc5cc(N6CCC(O)(C(=O)NCCc7cc(F)cc(F)c7)C6=O)ccc5[nH]4)c3C2=O)C(=O)N1] |
| “A 25” | 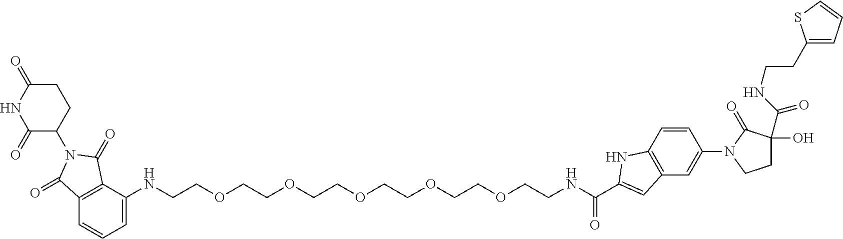 [SMILES: O=C1CCC(N2C(=O)c3cccc(NCCOCCOCCOCCOCCOCCNC(=O)c4cc5cc(N6CCC(O)(C(=O)NCCc7cccs7)C6=O)ccc5[nH]4)c3C2=O)C(=O)N1] |
| “A 26” | 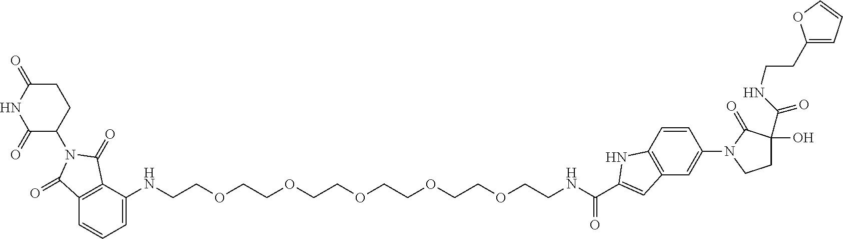 [SMILES: O=C1CCC(N2C(=O)c3cccc(NCCOCCOCCOCCOCCOCCNC(=O)c4cc5cc(N6CCC(O)(C(=O)NCCc7ccco7)C6=O)ccc5[nH]4)c3C2=O)C(=O)N1] |
| “A 27” | 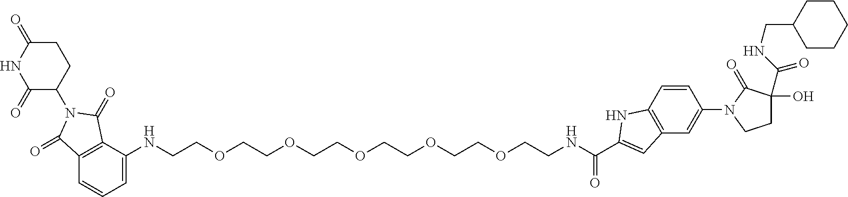 [SMILES: O=C1CCC(N2C(=O)c3cccc(NCCOCCOCCOCCOCCOCCNC(=O)c4cc5cc(N6CCC(O)(C(=O)NCC7CCCCC7)C6=O)ccc5[nH]4)c3C2=O)C(=O)N1] |
| “A 28” | 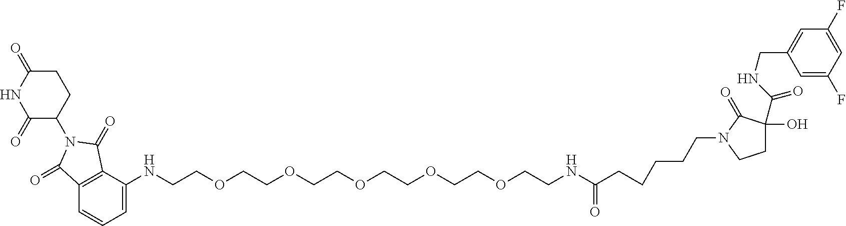 [SMILES: O=C(CCCCCN1CCC(O)(C(=O)NCc2cc(F)cc(F)c2)C1=O)NCCOCCOCCOCCOCCOCCNc1cccc2c1C(=O)N(C1CCC(=O)NC1=O)C2=O] |
| “A 29” | 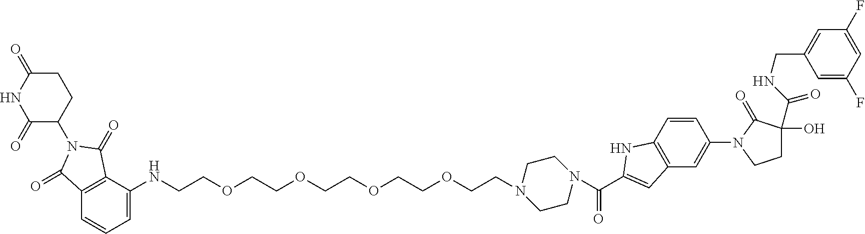 [SMILES: O=C1CCC(N2C(=O)c3cccc(NCCOCCOCCOCCOCCN4CCN(C(=O)c5cc6cc(N7CCC(O)(C(=O)NCc8cc(F)cc(F)c8)C7=O)ccc6[nH]5)CC4)c3C2=O)C(=O)N1] |
| “A 30” | 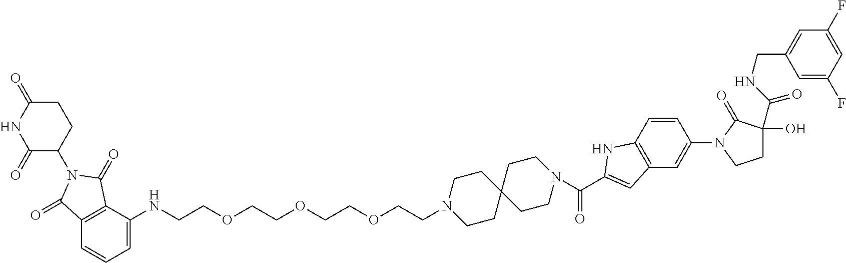 [SMILES: O=C1CCC(N2C(=O)c3cccc(NCCOCCOCCOCCN4CCC5(CC4)CCN(C(=O)c4cc6cc(N7CCC(O)(C(=O)NCc8cc(F)cc(F)c8)C7=O)ccc6[nH]4)CC5)c3C2=O)C(=O)N1] |
| “A 31” | 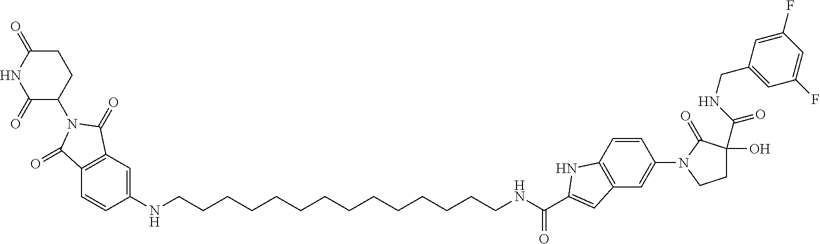 [SMILES: O=C1CCC(N2C(=O)c3ccc(NCCCCCCCCCCCCCCNC(=O)c4cc5cc(N6CCC(O)(C(=O)NCc7cc(F)cc(F)c7)C6=O)ccc5[nH]4)cc3C2=O)C(=O)N1] |
| “A 32” | 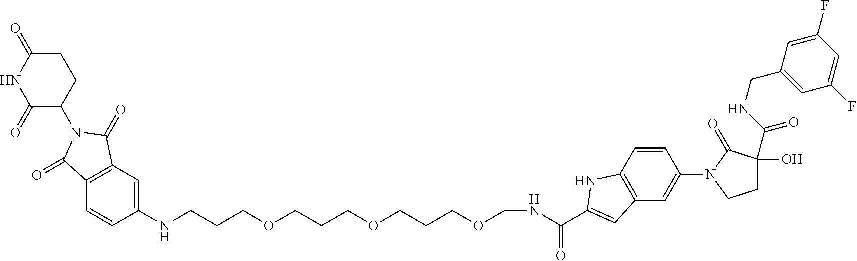 [SMILES: O=C1CCC(N2C(=O)c3ccc(NCCCOCCCOCCCOCNC(=O)c4cc5cc(N6CCC(O)(C(=O)NCc7cc(F)cc(F)c7)C6=O)ccc5[nH]4)cc3C2=O)C(=O)N1] |
| “A 33” | 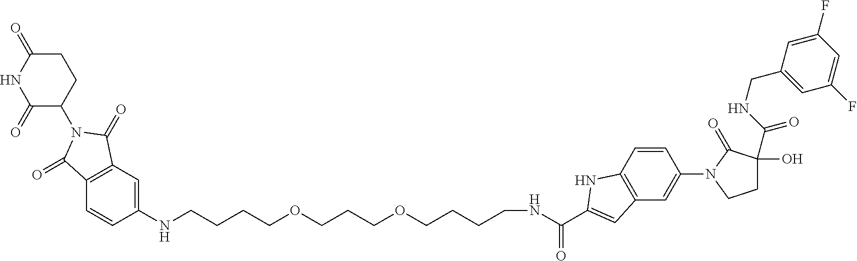 [SMILES: O=C1CCC(N2C(=O)c3ccc(NCCCCOCCCOCCCCNC(=O)c4cc5cc(N6CCC(O)(C(=O)NCc7cc(F)cc(F)c7)C6=O)ccc5[nH]4)cc3C2=O)C(=O)N1] |
| “A 34” | 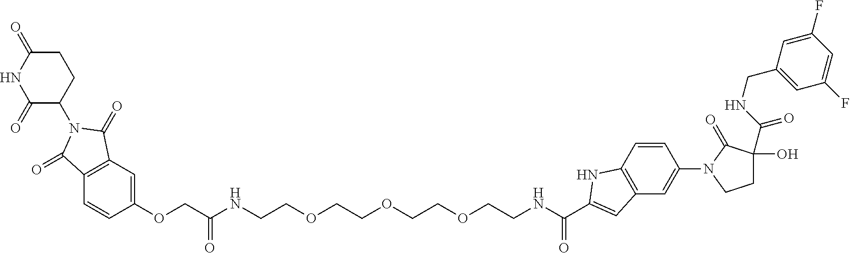 [SMILES: O=C(COc1ccc2c(c1)C(=O)N(C1CCC(=O)NC1=O)C2=O)NCCOCCOCCOCCNC(=O)c1cc2cc(N3CCC(O)(C(=O)NCc4cc(F)cc(F)c4)C3=O)ccc2[nH]1] |
| “A 35” | 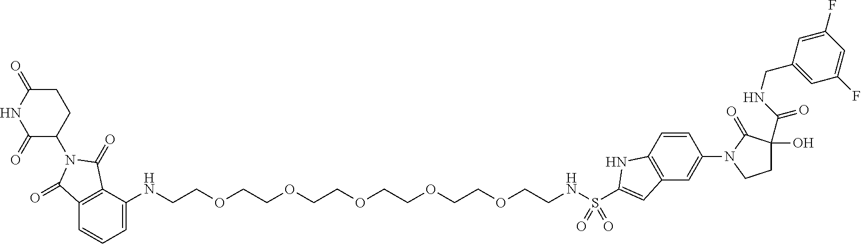 [SMILES: O=C1CCC(N2C(=O)c3cccc(NCCOCCOCCOCCOCCOCCNS(=O)(=O)c4cc5cc(N6CCC(O)(C(=O)NCc7cc(F)cc(F)c7)C6=O)ccc5[nH]4)c3C2=O)C(=O)N1] |
| “A 36” | 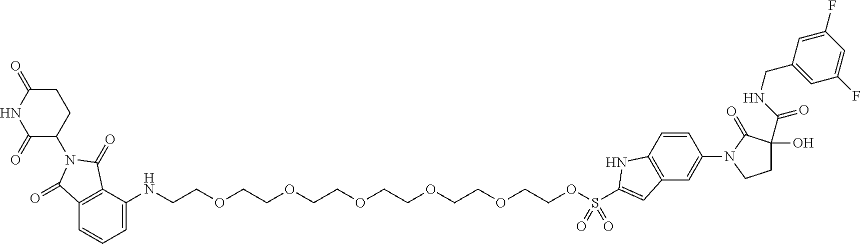 [SMILES: O=C1CCC(N2C(=O)c3cccc(NCCOCCOCCOCCOCCOCCOS(=O)(=O)c4cc5cc(N6CCC(O)(C(=O)NCc7cc(F)cc(F)c7)C6=O)ccc5[nH]4)c3C2=O)C(=O)N1] |
| “A 37” | 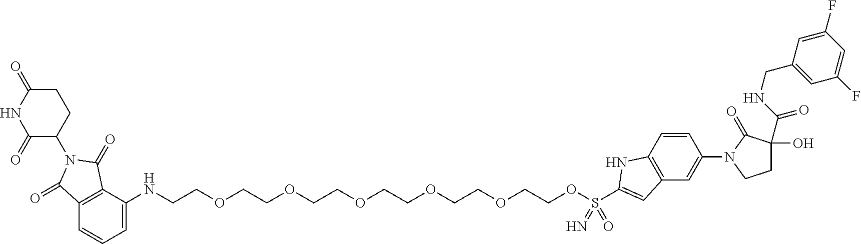 [SMILES: N=S(=O)(OCCOCCOCCOCCOCCOCCNc1cccc2c1C(=O)N(C1CCC(=O)NC1=O)C2=O)c1cc2cc(N3CCC(O)(C(=O)NCc4cc(F)cc(F)c4)C3=O)ccc2[nH]1] |
| “A 38” | 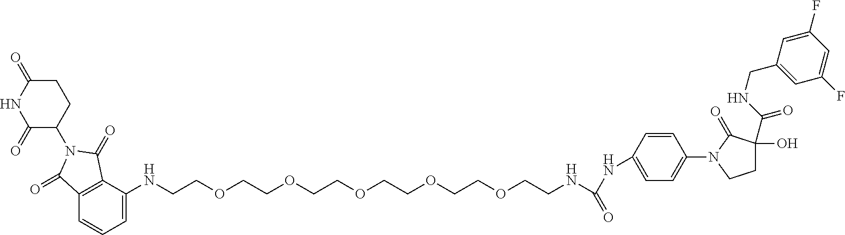 [SMILES: O=C1CCC(N2C(=O)c3cccc(NCCOCCOCCOCCOCCOCCNC(=O)Nc4ccc(N5CCC(O)(C(=O)NCc6cc(F)cc(F)c6)C5=O)cc4)c3C2=O)C(=O)N1] |
| “A 39” | 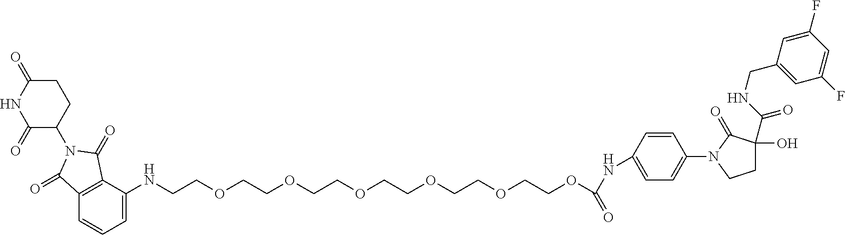 [SMILES: O=C1CCC(N2C(=O)c3cccc(NCCOCCOCCOCCOCCOCCOC(=O)Nc4ccc(N5CCC(O)(C(=O)NCc6cc(F)cc(F)c6)C5=O)cc4)c3C2=O)C(=O)N1] |
| “A 40” | 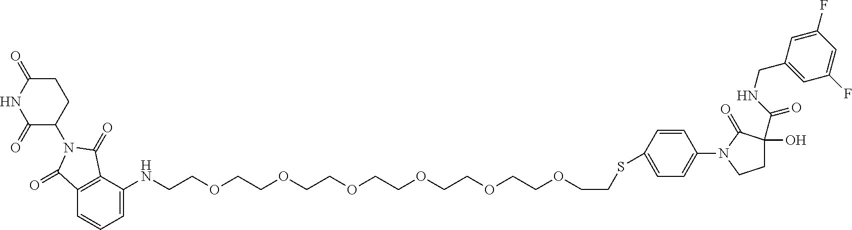 [SMILES: O=C1CCC(N2C(=O)c3cccc(NCCOCCOCCOCCOCCOCCOCCSc4ccc(N5CCC(O)(C(=O)NCc6cc(F)cc(F)c6)C5=O)cc4)c3C2=O)C(=O)N1] |
| “A 41” | 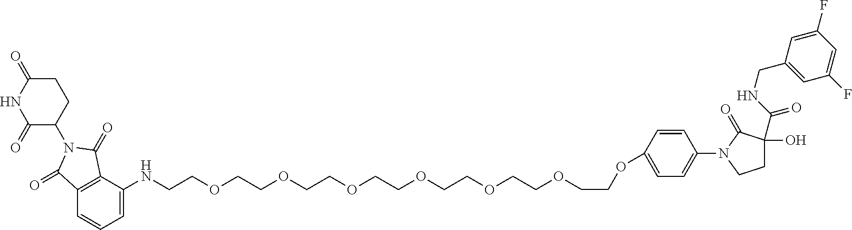 [SMILES: O=C1CCC(N2C(=O)c3cccc(NCCOCCOCCOCCOCCOCCOCCOc4ccc(N5CCC(O)(C(=O)NCc6cc(F)cc(F)c6)C5=O)cc4)c3C2=O)C(=O)N1] |
| “A 42” | 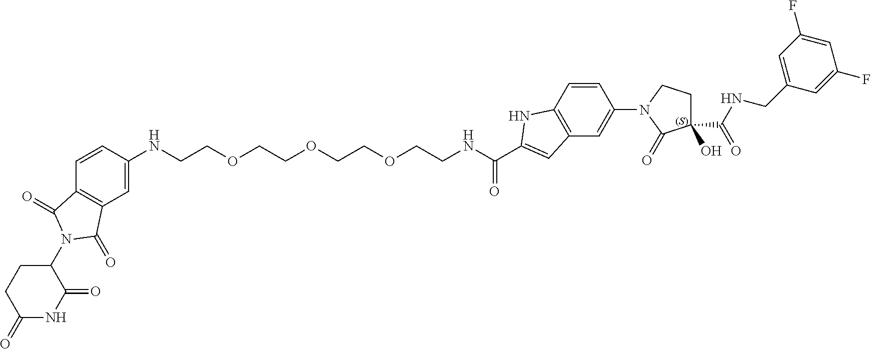 |
| “A 43” | 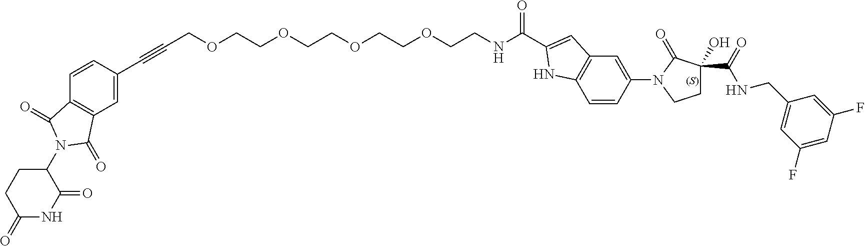 |
| “A 44” | 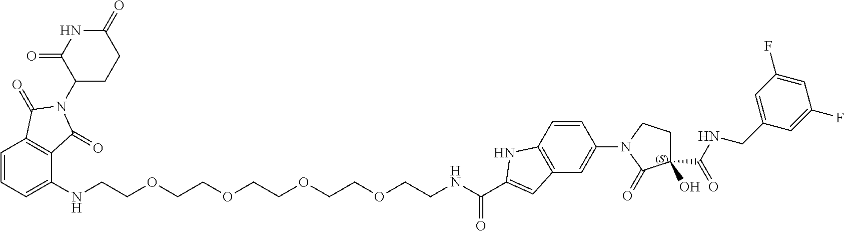 |
| “A 45” | 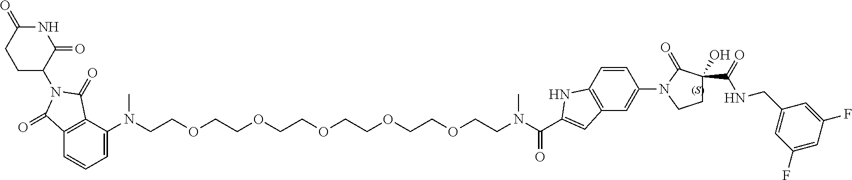 |
| “A 46” | 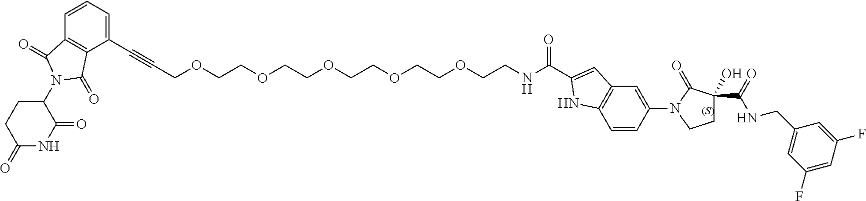 |
| “A 47” | 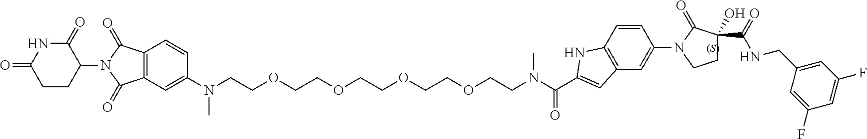 |
| “A 48” | 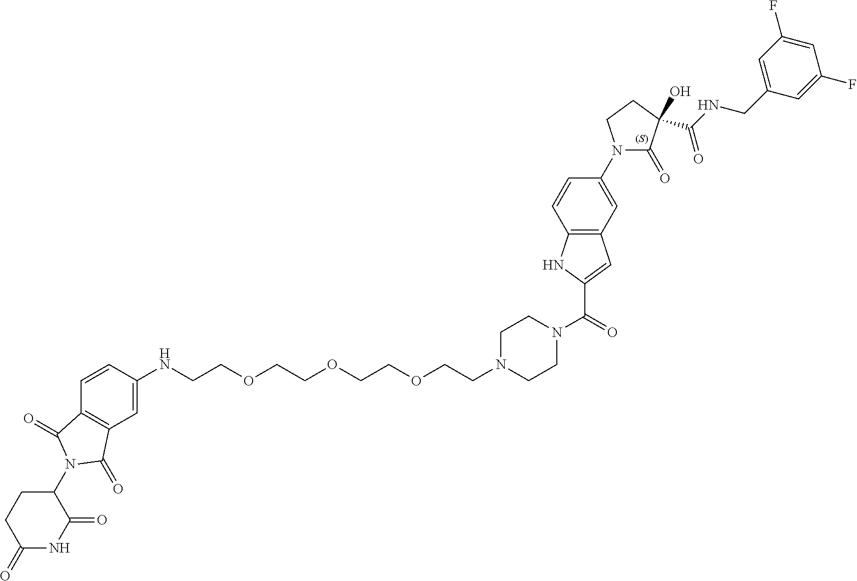 |
| “A 49” | 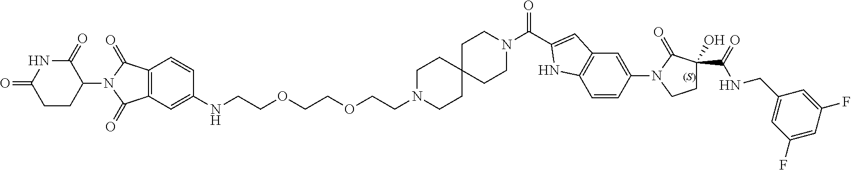 |
| “A 50” | 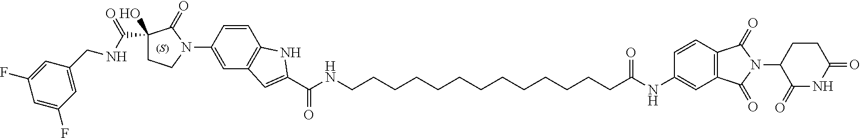 |
| “A 51” | 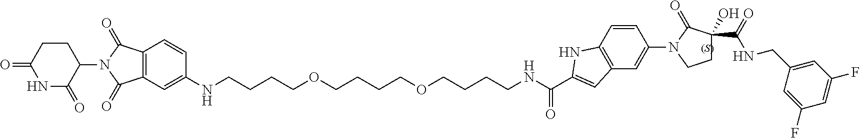 |
| “A 52” | 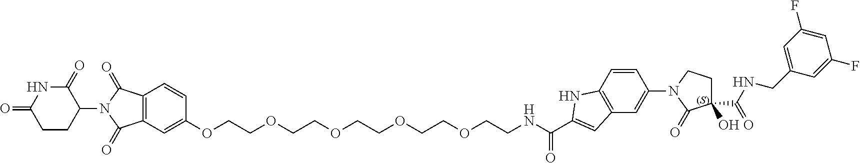 |
| “A 53” | 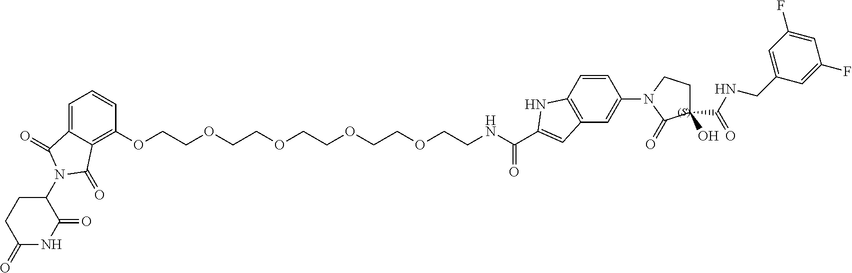 |
| “A 54” | 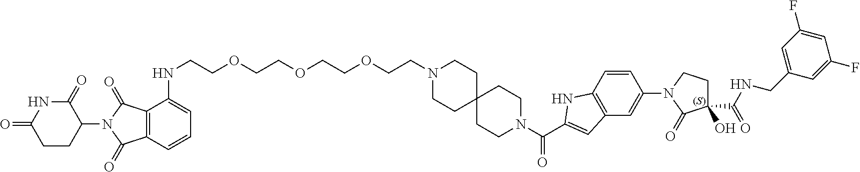 |
| “A 55” | 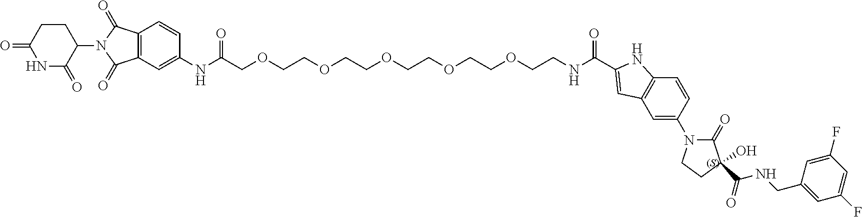 |
| “A 56” | 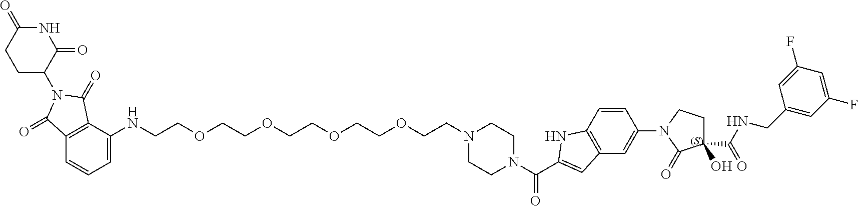 |
| “A 57” | 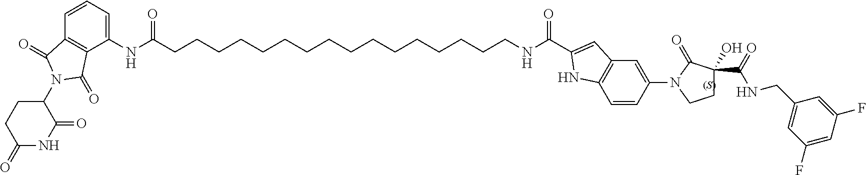 |

and pharmaceutically acceptable solvates, salts, tautomers, and stereoisomers thereof, including mixtures thereof in all ratios.

11. A method for preparing compounds of the formula I according to claim 1 and pharmaceutically acceptable salts, solvates, tautomers, and stereoisomers thereof, wherein L denotes CONR⁴, the method comprising:
reacting a compound of formula II 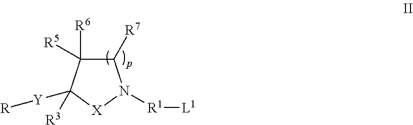
wherein X, R, R¹, R³, R⁵, R⁶, R⁷, and p have the meanings indicated in claim 1,
and L¹ denotes Cl, Br, I, or a free or reactively functionally modified OH group,
with a compound of the formula III
Q¹-Q²-NH₂ III
wherein Q¹ and Q² have the meanings indicated in claim 1,
and/or
a base or acid of the formula I is converted into one of its salts.

12. A medicament comprising at least one compound of the formula I according to claim 1 and/or pharmaceutically acceptable salts, solvates, tautomers, and stereoisomers thereof, including mixtures thereof in all ratios, and optionally an pharmaceutically acceptable carrier, excipient, or vehicle.

13. A method, comprising:
administering to a patient in need thereof the compound of formula I according to claim 1 and pharmaceutically acceptable salts, solvates, tautomers, and stereoisomers thereof, including mixtures thereof in all ratios to treat and/or prevent a tumor, a tumor metastasis, a proliferative disease of mesangial cells, haemangioma, proliferative retinopathy, rheumatoid arthritis, atherosclerotic neovascularisation, psoriasis, ocular neovascularisation, osteoporosis, diabetes, obesity, lymphoid leukaemia, lymphoma, malaria, and prostate hypertrophy.

14. The method according to claim 13, wherein the tumor is selected from the group consisting of a tumor of the squamous epithelium, of the bladder, of the stomach, of the kidneys, of head and neck, of the oesophagus, of the cervix, of the thyroid, of the intestine, of the liver, of the brain, of the prostate, of the urogenital tract, of the lymphatic system, of the stomach, of the larynx, of the lung, and/or of the skin; monocytic leukaemia; lung adenocarcinoma; small-cell lung carcinoma; pancreatic cancer; glioblastoma; breast carcinoma; acute myeloid leukaemia; chronic myeloid leukaemia; acute lymphatic leukaemia; chronic lymphatic leukaemia; Hodgkin's lymphoma; and non-Hodgkin's lymphoma.

15. The medicament according to claim 12, further comprising at least one further medicament active ingredient.

16. A kit consisting of separate packs of
(a) an effective amount of a compound of the formula I according to claim 1 and/or pharmaceutically acceptable salts, solvates, salts, and stereoisomers thereof, including mixtures thereof in all ratios,
and
(b) an effective amount of a further medicament active ingredient.
